(19) United States
(12) Patent Application Publication
(10) Pub. No.: US 2023/0102309 A1
(43) Pub. Date: Mar. 30, 2023

(54) TYROSINE KINASE INHIBITOR CONJUGATES

(71) Applicant: Ascendis Pharma Oncology Division A/S, Hellerup (DK)

(72) Inventors: Nicola BISEK, Heidelberg (DE); Samuel WEISBROD, Heidelberg (DE)

(21) Appl. No.: 17/596,830

(22) PCT Filed: Jun. 19, 2020
(86) PCT No.: PCT/EP2020/067155

§ 371 Date: Dec. 20, 2021

(30) Foreign Application Priority Data
Jun. 21, 2019 (EP) 19181798.0
Jan. 3, 2020 (EP) 20150239.0

(51) Int. Cl. A61K 47/69; A61K 47/60; A61K 47/61; A61K 47/54; A61P 35/00

(52) U.S. Cl. CPC A61K 47/6903; A61K 47/60; A61K 47/61; A61K 47/545; A61P 35/00

(57) ABSTRACT

The present invention relates to a tyrosine kinase inhibitor (“TKI”) conjugate or a pharmaceutically acceptable salt thereof, wherein said conjugate comprises a plurality of TKI moieties -D covalently conjugated via at least one moiety -L¹-L²- to a polymeric moiety Z, wherein -L¹- is covalently and reversibly conjugated to -D and -L²- is covalently conjugated to Z and wherein -L¹- is a linker moiety and -L²- is a chemical bond or a spacer moiety; and to related aspects.

The present invention relates to a tyrosine kinase inhibitor (“TKI”) conjugate or a pharmaceutically acceptable salt thereof, wherein said conjugate comprises a plurality of TKI moieties -D covalently conjugated via at least one moiety -L¹-L²- to a polymeric moiety Z, wherein -L¹- is covalently and reversibly conjugated to -D and -L²- is covalently conjugated to Z and wherein -L¹- is a linker moiety and -L²- is a chemical bond or a spacer moiety; and to related aspects.

Tyrosine kinases (TKs) are a sub-class of protein kinases that mediate transfer of a phosphate group from adenosine triphosphate (ATP) to specific tyrosine residues of target proteins. This protein phosphorylation serves to modulate protein activity and protein signaling involved in processes such as cell proliferation, differentiation, migration, function, or metabolism. Two types of TKs exist—non-receptor tyrosine kinases (NRTKs) and receptor tyrosine kinases (RTKs). NRTKs are intracellular TKs that propagate signaling cascades induced by RTKs or by other cell surface receptors (e.g. immune cell-associated receptors or G protein-coupled receptors). RTKs are transmembrane glycoproteins that bind extracellular ligands (e.g. VEGF, FGF, EGF, PDGF). Following ligand binding, they become activated, and either auto-phosphorylate tyrosine residues on their intracellular domains or phosphorylate intracellular protein substrates (Hubbard and Hill. Annu Rev Biochem. 2000; 69:373-98).

Tyrosine kinases regulate many different signaling pathways depending on the cell type and have been implicated in several disease indications, which make them widely pursued for therapeutic purposes. For example, vascular endothelial growth factor (VEGF) receptors (VEGFRs) are a type of RTK involved in angiogenesis and vascularization of tissues. Aberrant expression of VEGF in the tumor environment may promote tumor vascularization via VEGFR signaling. Inhibition of the VEGFR pathway, via small molecule tyrosine kinase inhibitors (TKIs) or biologic antagonists, has been successfully evaluated in preclinical and clinical studies for anti-cancer effects (Takahashi. Biol Pharm Bul. 2011; 34(12):1785-8).

Small molecule TKIs have typically been orally administered while biologic tyrosine kinase antagonists are administered intravenously, leading to systemic exposure of the inhibitor/antagonist. Although the efficacy, toxicity, bioavailability and other pharmacokinetic parameters vary greatly depending on the route of administration, systemic inhibition of tyrosine kinases can lead to adverse events, thus limiting their tolerability. For instance, VEGFR inhibition is associated with dose limiting hypertension which can lead to sub-optimal drug exposure for cancer treatment (Agarwal et al. Curr Oncol Rep. 2018 Jun. 21; 20(8):65. doi: 10.1007/s11912-018-0708-8).

The lack of clinical anti-tumor efficacy or sustained anti-tumor effect following systemic administration of TKIs or TK antagonists may be related to a failure of delivering the drug to the proposed site of action at efficacious concentrations. As these drugs are meant to inhibit TK activity at the site of the tumor, drug distribution following systemic administration may only serve to exacerbate global side effects due to undesirable systemic exposure to active drug while limiting bioavailability of the active compound in the tumor environment, thus precluding robust anti-tumor benefit. This may be particularly true for small molecule TKIs, which often have broad kinase inhibiting activity, thus affecting multiple kinase pathways (Agarwal et al. Curr Oncol Rep. 2018 Jun. 21; 20(8):65. doi: 10.1007/s11912-018-0708-8).

From the limited number of intra-tumoral application of TKIs in early stage development, most of them are disclosed with sustained release formulations comprising PLGA particles. Despite several advantages of PLGA-based drug delivery systems, inconsistent drug release and potential toxicity from dose dumping still remain as the drawback of this technology.

To our knowledge, no published reports are available describing clinical intra-tumoral injection of TKIs or TK antagonists, possibly due to the lack of appropriate slow release formulations. Indeed, rapid diffusion of these soluble TKIs or biologic tyrosine kinase antagonists out from the tumor may lead to substantial systemic exposure and undesirable side effects (e.g. hypertension). Furthermore, frequent intra-tumoral dosing of these compounds would be required for prolonged continuous exposure of the tumor tissue to TKIs or biologic tyrosine kinase antagonists, making effective intra-tumoral TKI or biologic tyrosine kinase antagonist therapy impractical or unfeasible for patients.

Although there have been substantial efforts in developing new and improved TKIs and biologic tyrosine kinase antagonists that have better specificity for their intended target, there remains a need to identify more effective TKIs and biologic tyrosine kinase antagonists. Furthermore, a need remains to modify TKI or biologic tyrosine kinase antagonist treatment regimens such that they overcome the shortcomings of prior art compounds and their related treatment methodologies whilst also providing a favorable anti-tumoral response and reducing adverse events related to systemic exposure.

In summary, there is a need for a more efficacious treatment.

It is an object of the present invention to at least partially overcome the above-described shortcomings.

This object is achieved with a tyrosine kinase inhibitor (“TKI”) conjugate or a pharmaceutically acceptable salt thereof, wherein said conjugate comprises a plurality of TKI moieties -D covalently conjugated via at least one moiety -L¹-L²- to a polymeric moiety Z, wherein -L¹- is covalently and reversibly conjugated to -D and -L²- is covalently conjugated to Z and wherein -L¹- is a linker moiety and -L²- is a chemical bond or a spacer moiety.

It was surprisingly found that the conjugates of the present invention result in high local and low systemic TKI drug concentrations that provide an improved treatment option for cell-proliferation disorders with a reduced risk of side-effects, such as hypertension. Such lower systemic exposure allows for more aggressive multi-agent therapies, facilitates treatment with otherwise poorly tolerated drug combinations and enables treatment of also hard-to-inject tumors that cannot be injected frequently enough with the corresponding free drug molecules.

It is understood that each moiety -D is covalently conjugated via at least one moiety -L¹-L²- to a polymeric moiety Z.

Within the present invention the terms are used having the meaning as follows.

As used herein the term “tyrosine kinase inhibitor” or “TKI” refers to a molecule that binds to and inhibits one or more cell-associated receptor or non-receptor tyrosine kinases that are activated via polypeptide growth factors, cytokines, hormones, or phosphorylation, and are involved in cellular signaling, cellular development, cellular proliferation, cellular maturation, cellular metabolism, angiogenesis, and in certain instances, tumorigenesis. Tyrosine kinases are ubiquitously expressed by virtually all cells. TKIs inhibit activation of tyrosine kinases by multiple mechanisms such as competing with, or allosterically antagonizing, binding of adenosine triphosphate (ATP) to the tyrosine kinase ATP-binding site, or by inhibiting enzymatic phosphorylation of said binding site, or inhibiting enzymatic kinase activity. In the case of receptor tyrosine kinases (RTKs), receptor TKIs may bind one or more RTKs and inhibit RTK activation as described above or by antagonizing activating ligand interactions, thus preventing receptor tyrosine kinase activation.

As used herein the term “pattern recognition receptor agonist” (“PRRA”) refers to a molecule that binds to and activates one or more immune cell-associated receptor that recognizes pathogen-associated molecular patterns (PAMPs) or damage-associated molecular patterns (DAMPs), leading to immune cell activation and/or pathogen- or damage-induced inflammatory responses. Pattern recognition receptors (PRRs) are typically expressed by cells of the innate immune system such as monocytes, macrophages, dendritic cells (DCs), neutrophils, and epithelial cells, as well as cells of the adaptive immune system.

As used herein the terms “cytotoxic agent” and “chemotherapeutic agent” are used synonymously and refer to compounds that are toxic to cells, which prevent cellular replication or growth, leading to cellular destruction/death. Examples of cytotoxic agents include chemotherapeutic agents and toxins, such as small molecule toxins or enzymatically active toxins of bacterial, fungal, plant or animal origin, including synthetic analogues and derivatives thereof.

As used herein the terms “immune checkpoint inhibitor” and “immune checkpoint antagonist” are used synonymously and refer to compounds that interfere with the function of, or inhibit binding of ligands that induce signaling through, cell-membrane expressed receptors that inhibit inflammatory immune cell function upon receptor activation. Such compounds may for example be biologics, such as antibodies, nanobodies, probodies, anticalins or cyclic peptides, or small molecule inhibitors.

As used herein the term “immune agonist” refers to compounds that directly or indirectly activate cell-membrane expressed receptors that stimulate immune cell function upon receptor activation.

As used herein the terms “multi-specific” and “multi-specific drugs” refer to compounds that simultaneously bind to two or more different antigens and can mediate antagonistic, agonistic, or specific antigen binding activity in a target-dependent manner.

As used herein the term “antibody-drug conjugate” (ADC) refers to compounds typically consisting of an antibody linked to a biologically active cytotoxic payload, radiotherapy, or other drug designed to deliver cytotoxic agents to the tumor environment. ADCs are particularly effective for reducing tumor burden without significant systemic toxicity and may act to improve the effectiveness of the immune response induced by checkpoint inhibitor antibodies.

As used herein the term “radionuclides” refers to radioactive isotopes that emit ionizing radiation leading to cellular destruction/death. Radionuclides conjugated to tumor targeting carriers are referred to as “targeted radionuclide therapeutics”.

As used here in the term “DNA damage repair inhibitor” refers to a drug that targets DNA damage repair elements, such as for example CHK1, CHK2, ATM, ATR and PARP. Certain cancers are more susceptive to targeting these pathways due to existing mutations, such as BRCA1 mutated patients to PARP inhibitors due to the concept of synthetic lethality.

As used herein, the term “tumor metabolism inhibitor” refers to a compound that interferes with the function of one or more enzymes expressed in the tumor environment that produce metabolic intermediates that may inhibit immune cell function.

As used herein the term “protein kinase inhibitor” refers to compounds that inhibit the activity of one or more protein kinases. Protein kinases are enzymes that phosphorylate proteins, which in turn can modulate protein function. It is understood that a protein kinase inhibitor may target more than one kinase and any classification for protein kinase inhibitors used herein refers to the main or most characterized target.

As used herein the term “chemokine receptor and chemoattractant receptor agonist” refers to compounds that activate chemokine or chemoattractant receptors, a subset of G-protein coupled receptors or G-protein coupled-like receptors that are expressed on a wide variety of cells and are primarily involved in controlling cell motility (chemotaxis or chemokinesis). These receptors may also participate in non-cell migratory processes, such as angiogenesis, cell maturation or inflammation.

As used herein the term “cytokine receptor agonist” refers to soluble proteins which control immune cell activation and proliferation. Cytokines include for example interferons, interleukins, lymphokines, and tumor necrosis factor.

As used herein the term “death receptor agonist” refers to a molecule which is capable of inducing pro-apoptotic signaling through one or more of the death receptors, such as DR4 (TRAIL-R1) or DR5 (TRAIL-R2). The death receptor agonist may be selected from the group consisting of antibodies, death ligands, cytokines, death receptor agonist expressing vectors, peptides, small molecule agonists, cells (such as for example stem cells) expressing the death receptor agonist, and drugs inducing the expression of death ligands.

As used herein the term “intra-tissue administration” refers to a type of administration, for example local injection, of a drug into a tissue of interest such as intra-tumoral, intra-muscular, subdermal or subcutaneous injections or injection into or adjacent to a normal or diseased tissue or organ. In certain embodiments intra-tissue administration is intravenous administration.

As used herein, the term “intra-tumoral administration” refers to a mode of administration, in which the drug is administered directly into tumor tissue. The term “intra-tumoral administration” also refers to administration pre- or post-resection into or onto the tumor bed. When tumor boundary is not well defined, it is also understood that intra-tumoral administration includes administration to tissue adjacent to the tumor cells (“peritumoral administration”). Exemplary tumors for intra-tumoral administration are solid tumors and lymphomas. Administration may occur via injection.

As used herein the term “baseline tissue” refers to a tissue sample taken from, or adjacent to, the area to be treated prior to treatment. For example, a biopsy of tissue to be treated can be taken immediately prior to treatment. It is understood that it may not always be possible to take a reference sample from the respective area prior to treatment, so the term “baseline tissue” may also refer to a non-treated control tissue that may be taken from a comparable location from the same animal or may be taken from a comparable location of a different animal of the same species. It is understood that in general the term “animal” also covers human and in certain embodiments means mouse, rat, non-human primate or human.

As used herein the term “local” or “locally” refers to a volume of tissue within a distance of 2 times the radius (r) from an injection site in any direction, wherein r is the distance in centimeters (cm) calculated from the volume (V) of conjugate of the present invention injected in cubic centimeters (cm³) following the spheroid equation

$$V = ( \frac{4}{3} ) \times π {r}^{3} .$$

For example, if 0.5 cm³ conjugate of the present invention is injected into a given tissue, a sample of tissue weighing at least 0.025 g taken within 0.98 cm in any direction of the injection site is referred to as a local sample.

As used herein the term “anti-tumor activity” means the ability to reduce the speed of tumor growth by at least 20%, such as by at least 25%, by at least 30%, by at least 35%, by at least 40%, by at least 45%, or by at least 50%; the ability to inhibit tumors from growing larger, i.e. tumor growth inhibition or tumor stasis; or the ability to cause a reduction in the size of a tumor, i.e. tumor regression. Anti-tumor activity may be determined by comparing the mean relative tumor volumes between control and treatment conditions. Relative volumes of individual tumors (individual RTVs) for day “x” may be calculated by dividing the absolute individual tumor volume on day “x” (Tₓ) following treatment initiation by the absolute individual tumor volume of the same tumor on the day treatment started (To) multiplied by 100:

$$R T {V}_{x} [ \% ] = \frac{{T}_{x}}{{T}_{0}} \times 100$$

Anti-tumor activity may be observed between 7 to 21 days following treatment initiation.

Tumor size, reported in mm3, may be measured physically by measuring the length (L) measured in mm and width (W) measured in mm of the tumors, which may include injected and non-injected tumors. Tumor volume can be determined by methods such as ultrasound imaging, magnetic resonance imaging, computed tomography scanning, or approximated by using the equation

$$V = \frac{1}{2} \times ( L \times {W}^{2} ) ,$$

with V being the tumor volume. Tumor burden, i.e. the total number of cancer cells in an individuum, can also be measured in the case of an experimental tumor model that expresses a reporter, such as luciferase enzyme or a fluorescent protein or another measurable protein or enzyme, by measuring the reporter element, i.e. luminescence or fluorescence, or the expressed reporter protein or enzyme product as a measure of the total number of tumor cells present and total tumor size. The latter reporter models can be useful for tumors that are not readily measurable on the surface of the animals (i.e. orthotopic tumors). It is understood that in general the term “animal” also covers human and in certain embodiments means mouse, rat, non-human primate or human. In certain embodiments “animal” means human.

As used herein the term “systemic molar concentration of TKI drug” refers to the molar concentration of TKI drug present in plasma. As TKI drug molecules may be bound to plasma proteins, such as for example albumin, the amount of TKI drug in plasma refers to the total amount of both unbound TKI molecules, i.e. TKI molecules not bound to plasma proteins, and bound TKI molecules, i.e. TKI molecules bound to plasma proteins. The concentration of total TKI drug in plasma may for example be determined by digesting a plasma sample with one or more proteases or other relevant methods that degrade plasma and/or tissue proteins and subsequently determining the concentration of TKI molecules present in the sample using suitable assays.

As used herein the term “local inhibition of angiogenesis” refers to an inhibition of angiogenesis that is restricted to an area near the site of administration of the conjugate of the present invention. The specific size of the area of angiogenesis inhibition will depend on the amount of TKI administered, the diffusion rate within the tissue, the time at which the signal is measured following injection, the rate of drug uptake by neighboring cells and the cellular expression of tyrosine kinases at and around the treated site, but would typically be detectable within a distance of 2 times the radius (r) from the injection site in any direction, wherein r is the distance in centimeters (cm) calculated from the volume (V) of conjugate of the present invention injected in cubic centimeters (cm³) following the spheroid equation

$$V = ( \frac{4}{3} ) \times π {r}^{3} .$$

For example, if 0.5 cm³ conjugate of the present invention is injected into a given tissue, a sample of tissue weighing at least 0.025 g taken within 0.98 cm in any direction of the injection site displays a measurable inhibition of angiogenesis when compared to baseline tissue. Within a volume of 2 times r tissue samples are to be taken for determining the presence of a specific set of markers for angiogenesis inhibition. However, this does not mean that expression of said angiogenesis inhibition markers outside a volume of 2 times r may not be disregulated, meaning up- or downregulated, by at least a factor of 1.5. In general, angiogenesis inhibition intensity decreases with increasing distance from the administration site. However, the person skilled in the art understands that providing an outer boundary of such localized inhibition of angiogenesis is not feasible, because the extent of angiogenesis inhibition depends on various factors, such as for example tumor type.

As used herein, the term “water-insoluble” refers to the property of a compound of which less than 1 g can be dissolved in one liter of water at 20° C. to form a homogeneous solution. Accordingly, the term “water-soluble” refers to the property of a compound of which 1 g or more can be dissolved in one liter of water at 20° C. to form a homogeneous solution.

As used herein, the term “a n-electron-pair-donating heteroaromatic N-comprising moiety” refers to the moiety which after cleavage of the linkage between -D and -L¹- results in a drug D-H and wherein the drug moiety -D and analogously the corresponding D-H comprises at least one, such as one, two, three, four, five, six, seven, eight, nine or ten heteroaromatic nitrogen atoms that donate a π-electron pair to the aromatic π-system. Examples of chemical structures comprising such heteroaromatic nitrogens that donate a π-electron pair to the aromatic Tc-system include, but are not limited to, pyrrole, pyrazole, imidazole, isoindazole, indole, indazole, purine, tetrazole, triazole and carbazole. For example, in the imidazole ring below the heteroaromatic nitrogen which donates a π-electron pair to the aromatic π-system is marked with “#”.

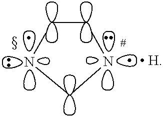

The π-electron-pair-donating heteroaromatic nitrogen atoms do not comprise heteroaromatic nitrogen atoms which only donate one electron (i.e. not a pair of π-electrons) to the aromatic π-system, such as for example the nitrogen that is marked with “§ ” in the abovementioned imidazole ring structure. The drug D-H may exist in one or more tautomeric forms, such as with one hydrogen atom moving between at least two heteroaromatic nitrogen atoms. In all such cases, the linker moiety is covalently and reversibly attached at a heteroaromatic nitrogen that donates a π-electron pair to the aromatic n-system.

As used herein, the term “drug” refers to a substance used in the treatment, cure, prevention or diagnosis of a disease or used to otherwise enhance physical or mental well-being of a patient. If a drug is conjugated to another moiety, the moiety of the resulting product that originated from the drug is referred to as “drug moiety”.

Any reference to a biologic drug herein, i.e. to a drug manufactured in, extracted from, or semisynthesized from biological sources such as a protein drug, also covers biosimilar versions of said drug.

As used herein the term “prodrug” refers to a drug moiety reversibly and covalently connected to a specialized protective group through a reversible prodrug linker moiety which is a linker moiety comprising a reversible linkage with the drug moiety and wherein the specialized protective group alters or eliminates undesirable properties in the parent molecule. This also includes the enhancement of desirable properties in the drug and the suppression of undesirable properties. The specialized non-toxic protective group may also be referred to as “carrier”. A prodrug releases the reversibly and covalently bound drug moiety in the form of its corresponding drug. In other words, a prodrug is a conjugate comprising a drug moiety, which is covalently and reversibly conjugated to a carrier moiety via a reversible linker moiety, which covalent and reversible conjugation of the carrier to the reversible linker moiety is either directly or through a spacer. The reversible linker may also be referred to as “reversible prodrug linker”. Such conjugate may release the formerly conjugated drug moiety in the form of a free drug, in which case the reversible linker or reversible prodrug linker is a traceless linker.

As used herein, the term “free form” of a drug means the drug in its unmodified, pharmacologically active form.

As used herein the term “spacer” or “linker” refers to a moiety that connects at least two other moieties with each other.

As used herein, the term “reversible”, “reversibly”, “degradable” or “degradably” with regard to the attachment of a first moiety to a second moiety means that the linkage that connects said first and second moiety is cleavable under physiological conditions, which physiological conditions are aqueous buffer at pH 7.4 and 37° C., with a half-life ranging from two days to three months, such as from two days to two months, such as from three days to one month. Such cleavage is in certain embodiments non-enzymatically, i.e. independent of enzymatic activity. Accordingly, the term “stable” with regard to the attachment of a first moiety to a second moiety means that the linkage that connects said first and second moiety exhibits a half-life of more than three months under physiological conditions.

As used herein, the term “reagent” means a chemical compound, which comprises at least one functional group for reaction with the functional group of another chemical compound or drug. It is understood that a drug comprising a functional group is also a reagent.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “—” indicates attachment to another moiety. Accordingly, a drug moiety, such as a TKI moiety, is released from a reversible linkage as a drug, such as TKI drug.

It is understood that if the chemical structure of a group of atoms is provided and if this group of atoms is attached to two moieties or is interrupting a moiety, said sequence or chemical structure can be attached to the two moieties in either orientation, unless explicitly stated otherwise. For example, a moiety “—C(O)N(R¹)—” can be attached to two moieties or interrupting a moiety either as “—C(O)N(R¹)—” or as “—N(R¹)C(O)—”. Similarly, a moiety

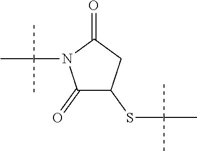

can be attached to two moieties or can interrupt a moiety either as

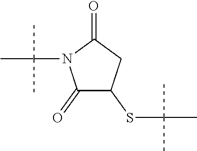

or as

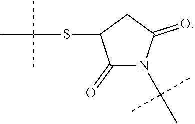

The term “substituted” as used herein means that one or more —H atom(s) of a molecule or moiety are replaced by a different atom or a group of atoms, which are referred to as “substituent”.

As used herein, the term “substituent” in certain embodiments refers to a moiety selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

—R^{x1}, —R^{x1a}, —R^{x1b} are independently of each other selected from the group consisting of —H, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—; —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different;

each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;

each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

As used herein, the term “hydrogel” means a hydrophilic or amphiphilic polymeric network composed of homopolymers or copolymers, which is insoluble due to the presence of hydrophobic interactions, hydrogen bonds, ionic interactions and/or covalent chemical crosslinks. The crosslinks provide the network structure and physical integrity. In certain embodiments the hydrogel is insoluble due to the presence of covalent chemical crosslinks.

As used herein the term “crosslinker” refers to a moiety that is a connection between different elements of a hydrogel, such as between two or more backbone moieties or between two or more hyaluronic acid strands.

As used herein the term “continuous gel” refers to a hydrogel in a flexible shape, i.e. a shape that is not pre-formed, but adjusts its shape to fit its surrounding. Upon administration, such as via injection, such continuous gel may in certain embodiments fragment into smaller sized particles. In certain embodiments such continuous gel does not fragment upon administration, such as via injection, and remains essentially the same volume, but may temporarily or permanently change its shape as required to pass through a needle, for example.

As used herein the term “about” in combination with a numerical value is used to indicate a range ranging from and including the numerical value plus and minus no more than 25% of said numerical value, such as no more than plus and minus 20% of said numerical value or such as no more than plus and minus 10% of said numerical value. For example, the phrase “about 200” is used to mean a range ranging from and including 200+/−25%, i.e. ranging from and including 150 to 250; such as 200+/−20%, i.e. ranging from and including 160 to 240; such as ranging from and including 200+/−10%, i.e. ranging from and including 180 to 220. It is understood that a percentage given as “about 50%” does not mean “50%+/−25%”, i.e. ranging from and including 25 to 75%, but “about 50%” means ranging from and including 37.5 to 62.5%, i.e. plus and minus 25% of the numerical value which is 50.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. The monomers may be identical, in which case the polymer is a homopolymer, or may be different, in which case the polymer is a heteropolymer. A heteropolymer may also be referred to as a “copolymer” and includes, for example, alternating copolymers in which monomers of different types alternate, periodic copolymers, in which monomers of different types are arranged in a repeating sequence; statistical copolymers, in which monomers of different types are arranged randomly; block copolymers, in which blocks of different homopolymers consisting of only one type of monomers are linked by a covalent bond; and gradient copolymers, in which the composition of different monomers changes gradually along a polymer chain. In certain embodiments a soluble polymer has a molecular weight of at least 0.5 kDa, e.g. a molecular weight of at least 1 kDa, a molecular weight of at least 2 kDa, a molecular weight of at least 3 kDa or a molecular weight of at least 5 kDa. If the polymer is soluble, it preferably has a molecular weight of at most 1000 kDa, such as at most 750 kDa, such as at most 500 kDa, such as at most 300 kDa, such as at most 200 kDa, such as at most 100 kDa. It is understood that a polymer may also comprise one or more other moieties, such as, for example, one or more functional groups. The term “polymer” also relates to a peptide or protein, even though the side chains of individual amino acid residues may be different. It is understood that for covalently crosslinked polymers, such as hydrogels, no meaningful molecular weight ranges can be provided.

As used herein, the term “polymeric” refers to a reagent or a moiety comprising one or more polymers or polymer moieties. A polymeric reagent or moiety may optionally also comprise one or more other moieties, which in certain embodiments are selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group comprising

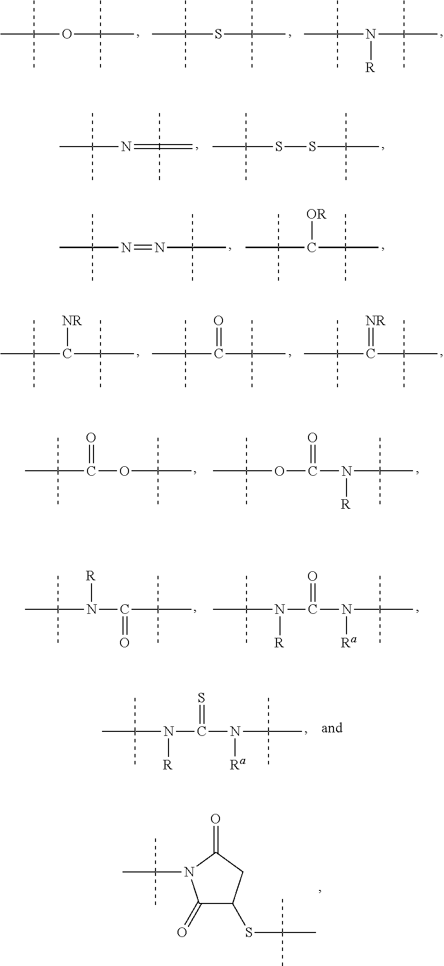

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl,
2,3-dimethylbutyl and 3,3-dimethylpropyl; and
which moieties and linkages are optionally further substituted.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lies. An integer for “x” given as “about x” means that the arithmetic mean numbers of monomers lies in a range of integers of x+/−25%, such as x+/−20% or such as x+/−10%.

As used herein, the term “number average molecular weight” means the ordinary arithmetic mean of the molecular weights of the individual polymers.

As used herein, the term “PEG-based” in relation to a moiety or reagent means that said moiety or reagent comprises PEG. Such PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60% (w/w) PEG, such as at least 70% (w/w) PEG, such as at least 80% (w/w) PEG, such as at least 90% (w/w) PEG, or such as at least 95% (w/w) PEG. The remaining weight percentage of the PEG-based moiety or reagent may be other moieties, such as those selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group consisting of

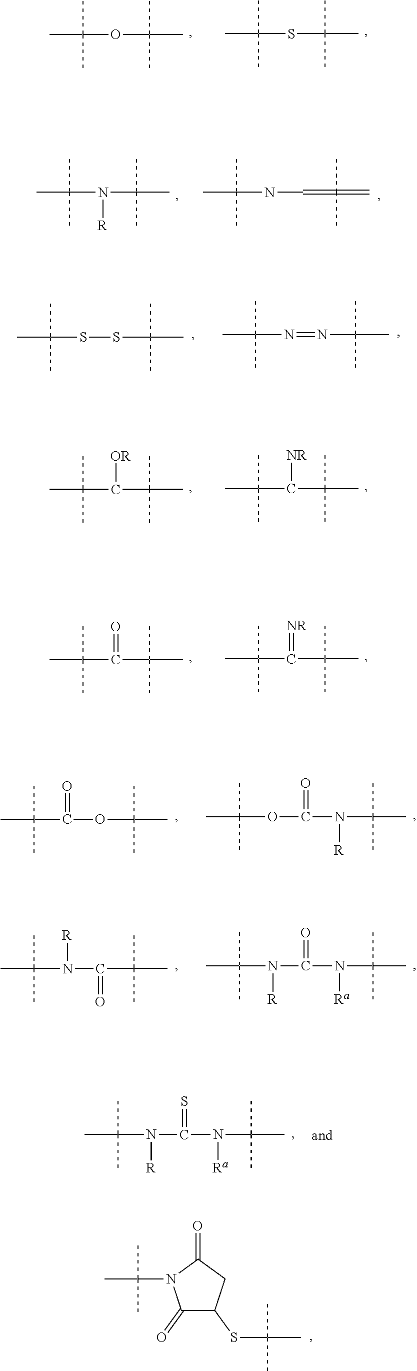

wherein
dashed lines indicate attachment to the remainder of the moiety or reagent, and —R and —R^{a} are independently of each other selected from the group consisting of —H, and C₁₋₆ alkyl; and
which moieties and linkages are optionally further substituted.

The terms “poly(alkylene glycol)-based”, “poly(propylene glycol)-based” and “hyaluronic acid-based” are used accordingly.

The term “interrupted” means that a moiety is inserted between two carbon atoms or—if the insertion is at one of the moiety's ends—between a carbon or heteroatom and a hydrogen atom.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₅₀ carbon may optionally be replaced by a substituent as defined above. Optionally, a C₁₋₁₀ or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example for such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the terms “C₂₋₁₀ alkenyl”, “C₂₋₂₀ alkenyl” or “C₂₋₅₀ alkenyl” alone or in combination mean a straight-chain or branched hydrocarbon moiety comprising at least one carbon-carbon double bond having 2 to 10, 2 to 20 or 2 to 50 carbon atoms, respectively. Each hydrogen atom of a C₂₋₁₀ alkenyl, C₂₋₂₀ alkenyl or C₂₋₅₀ alkenyl group may optionally be replaced by a substituent as defined above. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined above. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which may be selected from the group consisting of

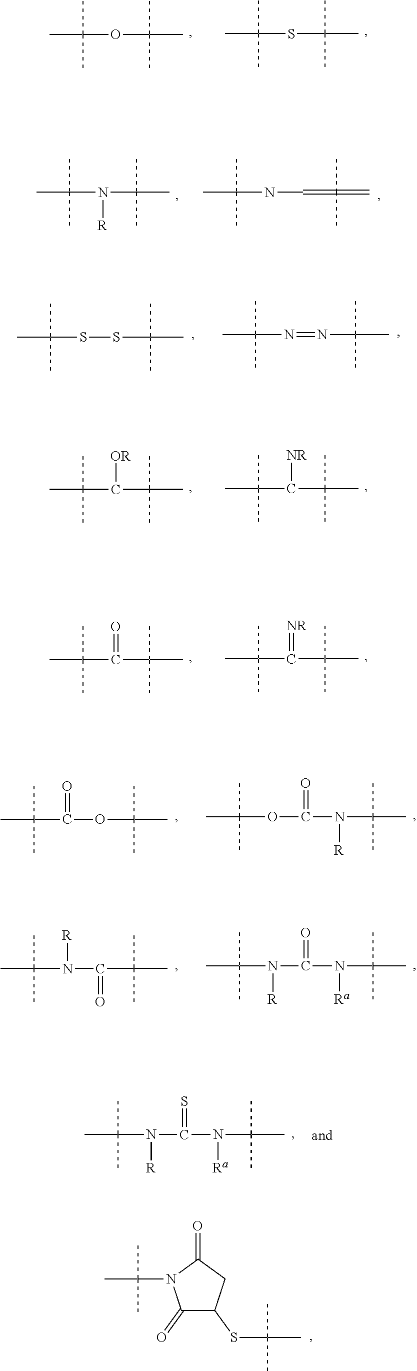

wherein
dashed lines indicate attachment to the remainder of the moiety or reagent; and
—R and —R^{a} are independently of each other selected from the group consisting of —H and C₁₋₆ alkyl.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined above. The term “C₃₋₁₀ cycloalkyl” also includes bridged bicycles like norbornane or norbornene.

The term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In one embodiment a 8- to 30-membered carbopolycyclyl means a cyclic moiety of two, three, four or five rings. In another embodiment a 8- to 30-membered carbopolycyclyl means a cyclic moiety of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent.

Similarly, the term “8- to 30-membered heteropolycyclyl” or “8- to 30-membered heteropolycycle” means a heterocyclic moiety of more than two rings with 8 to 30 ring atoms, such as of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

It is understood that the phrase “the pair R^{x}/R^{y} is joined together with the atom to which they are attached to form a C₃₋₁₀ cycloalkyl or a 3- to 10-membered heterocyclyl” in relation with a moiety of the structure

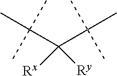

means that R^{x} and R^{y} form the following structure:

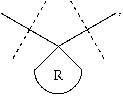

wherein R is a C₃₋₁₀ cycloalkyl or a 3- to 10-membered heterocyclyl.

It is also understood that the phrase “the pair R^{x}/R^{y} is joined together with the atoms to which they are attached to form a ring A” in relation with a moiety of the structure

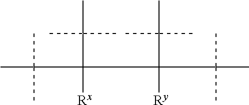

means that R^{x} and R^{y} form the following structure:

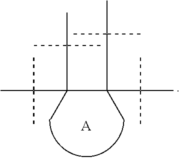

It is also understood that the phrase “—R¹ and an adjacent —R² form a carbon-carbon double bond provided that n is selected from the group consisting of 1, 2, 3 and 4” in relation with a moiety of the structure:

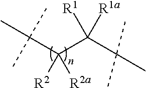

means that for example when n is 1, —R¹ and the adjacent —R² form the following structure:

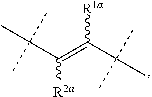

and if for example, n is 2, R¹ and the adjacent —R² form the following structure:

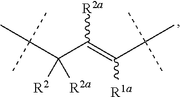

wherein the wavy bond means that —R^{1a} and —R^{2a} may be either on the same side of the double bond, i.e. in cis configuration, or on opposite sides of the double bond, i.e. in trans configuration and wherein the term “adjacent” means that —R¹ and —R² are attached to carbon atoms that are next to each other.

It is also understood that the phrase “two adjacent —R² form a carbon-carbon double bond provided that n is selected from the group consisting of 2, 3 and 4” in relation with a moiety of the structure:

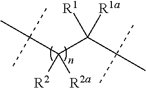

means that for example when n is 2, two adjacent —R² form the following structure:

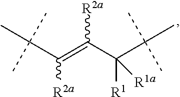

wherein the wavy bond means that each —R^{2a} may be either on the same side of the double bond, i.e. in cis configuration, or on opposite sides of the double bond, i.e. in trans configuration and wherein the term “adjacent” means that two —R² are attached to carbon atoms that are next to each other.

It is understood that the “N” in the phrase “π-electron-pair-donating heteroaromatic N” refers to nitrogen.

It is understood that “N⁺” in the phrases “an electron-donating heteroaromatic N⁺-comprising moiety” and “attachment to the N⁺ of -D⁺” refers to a positively charged nitrogen atom.

As used herein, “halogen” means fluoro, chloro, bromo or iodo. In certain embodiments halogen is fluoro or chloro.

As used herein the term “alkali metal ion” refers to Na⁺, K⁺, Li⁺, Rb⁺ and Cs⁺. In certain embodiments “alkali metal ion” refers to Na⁺, K⁺ and Li⁺.

As used herein the term “alkaline earth metal ion” refers to Mg²⁺, Ca²⁺, Sr²⁺ and Ba²⁺. In certain embodiments an alkaline earth metal ion is Mg²⁺ or Ca²⁺.

As used herein, the term “functional group” means a group of atoms which can react with other groups of atoms. Exemplary functional groups are carboxylic acid, primary amine, secondary amine, tertiary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane, and aziridine.

In case the compounds of the present invention comprise one or more acidic or basic groups, the invention also comprises their corresponding pharmaceutically or toxicologically acceptable salts, in particular their pharmaceutically utilizable salts. Thus, the compounds of the present invention comprising acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine, amino acids, and quaternary ammonium salts, like tetrabutylammonium or cetyl trimethylammonium. Compounds of the present invention comprising one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, trifluoroacetic acid, and other acids known to the person skilled in the art. For the person skilled in the art further methods are known for converting the basic group into a cation like the alkylation of an amine group resulting in a positively-charge ammonium group and an appropriate counterion of the salt. If the compounds of the present invention simultaneously comprise acidic and basic groups, the invention also includes, in addition to the salt forms mentioned, inner salts or betaines (zwitterions). The respective salts can be obtained by customary methods, which are known to the person skilled in the art like, for example by contacting these prodrugs with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

The term “pharmaceutically acceptable” means a substance that does not cause harm when administered to a patient and in certain embodiments means approved by a regulatory agency, such as the EMA (Europe), the FDA (US) or any other national regulatory agency for use in animals, such as for use in humans.

As used herein, the term “excipient” refers to a diluent, adjuvant, or vehicle with which the therapeutic, such as a drug or the conjugate of the present invention, is administered. Such pharmaceutical excipient may be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, hyaluronic acid, propylene glycol, water, ethanol and the like. The pharmaceutical composition, if desired, may also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid), or may contain detergents, like Tween®, poloxamers, poloxamines, CHAPS, Igepal®, or amino acids like, for example, glycine, lysine, or histidine. These pharmaceutical compositions may take the form of solutions, suspensions, emulsions, tablets, pills, capsules, powders, sustained-release formulations and the like. The pharmaceutical composition may be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions may contain a therapeutically effective amount of the drug, such as the conjugate of the present invention, together with a suitable amount of excipient so as to provide the form for proper administration to the patient. The formulation should suit the mode of administration.

The term “peptide” as used herein refers to a chain of at least 2 and up to and including 50 amino acid monomer moieties, which may also be referred to as “amino acid residues”, linked by peptide (amide) linkages, which may be linear, branched or cyclic. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties.

As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein the term “small molecule drug” refers to drugs that are organic compounds with a molecular weight of no more than 1 kDa, such as up to 900 Da.

As used herein the term “biologics” or “biopharmaceutical” refers to any pharmaceutical drug manufactured in, extracted from, or semi-synthesized from biological sources. Different from totally synthesized pharmaceuticals, they may include vaccines, blood, blood components, allergenics, somatic cells, gene therapies, tissues, recombinant therapeutic protein, and living cells used in cell therapy. Biologics may be composed of sugars, proteins, or nucleic acids or complex combinations of these substances, or may be living cells or tissues. They or their precursors or components are isolated from living sources, such as from human, animal, plant, fungal or microbial sources.

In general, the terms “comprise” or “comprising” also encompasses “consist of” or “consisting of”.

In certain embodiments -D is selected from the group consisting of receptor tyrosine kinase inhibitors, intracellular kinase inhibitors, cyclin dependent kinase inhibitors, phosphoinositide-3-kinase (PI3K) inhibitors, mitogen-activated protein kinase inhibitors, inhibitors of nuclear factor kappa-β kinase (IKK), and Wee-1 inhibitors. In certain embodiments -D is selected from the group consisting of receptor tyrosine kinase inhibitors, intracellular kinase inhibitors, cyclin dependent kinase inhibitors, mitogen-activated protein kinase inhibitors, inhibitors of nuclear factor kappa-β kinase (IKK), and Wee-1 inhibitors.

In certain embodiments -D is a receptor tyrosine kinase inhibitor. Examples for such receptor tyrosine kinase inhibitors are EGF receptor inhibitors, VEGF receptor inhibitors, C-KIT Receptor inhibitors, ERBB2 (HER2) inhibitors, ERBB3 receptor inhibitors, FGF receptor inhibitors, AXL receptor inhibitors and MET receptor inhibitors.

In certain embodiments -D is an EGF receptor inhibitor, such as afatinib, cetuximab, erlotinib, gefitinib, pertuzumab and margetuximab.

In certain embodiments -D is a VEGF receptor inhibitor, such as axitinib, lenvatinib, pegaptanib and linifanib (ABT-869). In certain embodiments -D is axitinib. In certain embodiments -D is lenvatinib.

In certain embodiments -D is a C-KIT Receptor inhibitor such as CDX0158 (KTN0158).

In certain embodiments -D is an ERBB2 (HER2) inhibitor, such as herceptin (trastuzumab).

In certain embodiments -D is an ERBB3 receptor inhibitor, such as CDX3379 (MED13379, KTN3379) and AZD8931 (sapitinib).

In certain embodiments -D is an FGF receptor inhibitor such as erdafitinib.

In certain embodiments -D is an AXL receptor inhibitor such as BGB324 (BGB 324, R 428, R428, bemcentinib) and SLC391.

In certain embodiments -D is a MET receptor inhibitor, such as CGEN241 or tivantinib. In certain embodiments -D is tivantinib.

In certain embodiments -D is an intracellular kinase inhibitor. Examples for such intracellular kinase inhibitors are Bruton's tyrosine kinase (BTK) inhibitors, spleen tyrosine kinase inhibitors, Bcr-Abl tyrosine kinase inhibitors, Janus kinase inhibitors and multi-specific tyrosine kinase inhibitors.

In certain embodiments -D is a BTK inhibitor, such as ibrutinib, acalabrutinib, GS-4059, spebrutinib, BGB-3111, HM71224, zanubrutinib, ARQ531, BI-BTK1 and vecabrutinib.

In certain embodiments -D is a spleen tyrosine kinase inhibitor, such as fostamatinib.

In certain embodiments -D is a Bcr-Abl tyrosine kinase inhibitor, such as imatinib and nilotinib.

In certain embodiments -D is a Janus kinase inhibitor, such as ruxolitinib, tofacitinib and fedratinib.

In certain embodiments -D is a multi-specific tyrosine kinase inhibitor, such as bosutinib, crizotinib, cabozantinib, dasatinib, entrectinib, lapatinib, mubritinib, pazopanib, sorafenib, sunitinib, SU6656 and vandetanib. In certain embodiments -D is crizotinib. In certain embodiments -D is cabozantinib which is an inhibitor of c-Met, VEGFR2, AXL and RET.

In certain embodiments -D is a cyclin dependent kinase inhibitor. Examples for cyclin dependent kinase inhibitors are copanlisib, ribociclib, palbociclib, abemaciclib, trilaciclib, purvalanol A, olomucine II and MK-7965. In certain embodiments -D is copanlisib.

In certain embodiments -D is a phophoinositide-3-kinase inhibitor. Examples for phophoinositide-3-kinase inhibitors are IP1549, GDc-0326, pictilisib, serabelisib, IC-87114, AMG319, seletalisib, idealisib and CUDC907.

In certain embodiments -D is a mitogen-activated protein kinase inhibitor. Examples for mitogen-activated protein kinase inhibitors are Ras/famesyl transferase inhibitors, Raf inhibitors, MEK inhibitors and ERK inhibitors.

In certain embodiments -D is a Ras/famesyl transferase inhibitor, such as tipirafinib and LB42708.

In certain embodiments -D is a Raf inhibitor, such as regorafenib, encorafenib, vemurafenib, dabrafenib, sorafenib, PLX-4720, GDC-0879, AZ628, lifirafenib, PLX7904 and RO5126766.

In certain embodiments -D is a MEK inhibitor, such as cobimetinib, trametinib, binimetinib, selumetinib, pimasertib, refametinib and PD0325901. In certain embodiments -D or drug is cobimetinib.

In certain embodiments -D is an ERK inhibitor, such as MK-8353, GDC-0994, ulixertinib and SCH772984.

In certain embodiments -D is an inhibitors of nuclear factor IKK. Examples for inhibitors of nuclear factor kappa-β kinase (IKK) are BPI-003 and AS602868.

In certain embodiments -D is a Wee-1 inhibitor. An example of a Wee-1 inhibitor is adavosertib.

In certain embodiments -D is selected from the group consisting of lenvatinib, axitinib, cobimetinib, crizotinib, tivantinib, copanlisib and cabozantinib.

In certain embodiments -D is a non-indolinone-based tyrosine kinase inhibitor.

In certain embodiments all moieties -D of a conjugate of the present invention are identical. It is understood that this does not exclude the occurrence of changes in the chemical structure of individual TKI drug molecules or TKI moieties due to for example molecular rearrangements or degradation, as may for example occur during storage. In certain embodiments the conjugate of the present invention comprises more than one type of -D, i.e. two or more different types of -D, such as two different types of -D, three different types of -D, four different types of -D or five different types of -D.

If the conjugate of the present invention comprises more than one type of -D, all -D may be connected to the same type of -L¹- or may be connected to different types of -L¹-, i.e. a first type of -D may be connected to a first type of -L¹-, a second type of -D may be connected to a second type of -L¹- and so on. Using different types of -L¹- may in certain embodiments allow different release kinetics for different types of -D, such as for example a faster release for a first type of -D, a medium release for a second type of -D and a slow release for a third type of -D. Likewise, two different types of -D may be connected to the same type of -L¹-, allowing for release of both types of -D with the same release kinetics. Accordingly, in certain embodiments the conjugates of the present invention comprise one type of -L¹-. In certain embodiments the conjugates of the present invention comprise two types of -L¹-. In certain embodiments the conjugates of the present invention comprise three types of -L¹-. In certain embodiments the conjugates of the present invention comprise four types of -L¹-. In certain embodiments the conjugates of the present invention comprise five types of -L¹-.

In certain embodiments the conjugates of the present invention comprise one type of -D and one type of -L¹-. In certain embodiments the conjugates of the present invention comprise two types of -D and two types of -L¹-. In certain embodiments the conjugates of the present invention comprise three types of -D and three types of -L¹-. In certain embodiments the conjugates of the present invention comprise four types of -D and four types of -L¹-. In certain embodiments the conjugates of the present invention comprise two types of -D and one type of -L¹-. In certain embodiments the conjugates of the present invention comprise three types of -D and one or two types of -L¹-.

In certain embodiments at least 10% of all moieties -D of the conjugate are axitinib, such as at least 20% of all moieties -D, such as at least 30% of all moieties -D, such as at least 40% of all moieties -D, such as at least 50% of all moieties -D, such as at least 60% of all moieties -D, such as at least 70% of all moieties -D, such as at least 80% of all moieties -D, such as at least 90% of all moieties -D. In certain embodiments all moieties -D of the conjugate are axitinib.

In certain embodiments at least 10% of all moieties -D of the conjugate are lenvatinib, such as at least 20% of all moieties -D, such as at least 30% of all moieties -D, such as at least 40% of all moieties -D, such as at least 50% of all moieties -D, such as at least 60% of all moieties -D, such as at least 70% of all moieties -D, such as at least 80% of all moieties -D, such as at least 90% of all moieties -D. In certain embodiments all moieties -D of the conjugate are lenvatinib.

In certain embodiments at least 10% of all moieties -D of the conjugate are cobimetinib, such as at least 20% of all moieties -D, such as at least 30% of all moieties -D, such as at least 40% of all moieties -D, such as at least 50% of all moieties -D, such as at least 60% of all moieties -D, such as at least 70% of all moieties -D, such as at least 80% of all moieties -D, such as at least 90% of all moieties -D. In certain embodiments all moieties -D of the conjugate are cobimetinib.

In certain embodiments at least 10% of all moieties -D of the conjugate are crizotinib, such as at least 20% of all moieties -D, such as at least 30% of all moieties -D, such as at least 40% of all moieties -D, such as at least 50% of all moieties -D, such as at least 60% of all moieties -D, such as at least 70% of all moieties -D, such as at least 80% of all moieties -D, such as at least 90% of all moieties -D. In certain embodiments all moieties -D of the conjugate are crizotinib.

In certain embodiments at least 10% of all moieties -D of the conjugate are tivantinib, such as at least 20% of all moieties -D, such as at least 30% of all moieties -D, such as at least 40% of all moieties -D, such as at least 50% of all moieties -D, such as at least 60% of all moieties -D, such as at least 70% of all moieties -D, such as at least 80% of all moieties -D, such as at least 90% of all moieties -D. In certain embodiments all moieties -D of the conjugate are tivantinib.

In certain embodiments at least 10% of all moieties -D of the conjugate are copanlisib, such as at least 20% of all moieties -D, such as at least 30% of all moieties -D, such as at least 40% of all moieties -D, such as at least 50% of all moieties -D, such as at least 60% of all moieties -D, such as at least 70% of all moieties -D, such as at least 80% of all moieties -D, such as at least 90% of all moieties -D. In certain embodiments all moieties -D of the conjugate are copanlisib.

In certain embodiments at least 10% of all moieties -D of the conjugate are cabozantinib, such as at least 20% of all moieties -D, such as at least 30% of all moieties -D, such as at least 40% of all moieties -D, such as at least 50% of all moieties -D, such as at least 60% of all moieties -D, such as at least 70% of all moieties -D, such as at least 80% of all moieties -D, such as at least 90% of all moieties -D. In certain embodiments all moieties -D of the conjugate are cabozantinib.

In certain embodiments the conjugate further comprises non-TKI moieties -D, i.e. the conjugate comprises at least one moiety -D in the form of a TKI moiety and one or more drug moieties -D of at least one different class of drugs, such that some of the moieties -D of the conjugate are TKI moieties as described above and in addition the conjugate comprises moieties -D that are from one or more different classes of drugs or non-TKI moieties.

In certain embodiments these non-TKI moieties -D in the form of a different class of drugs are selected from the group consisting of cytotoxic/chemotherapeutic agents, immune checkpoint inhibitors or antagonists, immune agonists, multi-specific drugs, antibody-drug conjugates (ADC), radionuclides or targeted radionuclide therapeutics, DNA damage repair inhibitors, tumor metabolism inhibitors, pattern recognition receptor agonists, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, and hormones including hormone peptides and synthetic hormones.

In certain embodiments the one or more non-TKI moieties -D are cytotoxic/chemotherapeutic agents. In certain embodiments the one or more non-TKI moieties -D are immune checkpoint inhibitors or antagonists. In certain embodiments the one or more non-TKI moieties -D are multi-specific drugs. In certain embodiments the one or more non-TKI moieties -D are antibody-drug conjugates (ADC). In certain embodiments the one or more non-TKI moieties -D are targeted radionuclide therapeutics. In certain embodiments the one or more non-TKI moieties -D are DNA damage repair inhibitors. In certain embodiments the one or more non-TKI moieties -D are tumor metabolism inhibitors. In certain embodiments the one or more non-TKI moieties -D are pattern recognition receptor agonists. In certain embodiments the one or more non-TKI moieties -D are chemokines and chemoattractant receptor agonists. In certain embodiments the one or more non-TKI moieties -D are chemokines or chemokine receptor antagonists. In certain embodiments the one or more non-TKI moieties -D are cytokine receptor agonists. In certain embodiments the one or more non-TKI moieties -D are death receptor agonists. In certain embodiments the one or more non-TKI moieties -D are CD47 antagonists. In certain embodiments the one or more non-TKI moieties -D are SIRPα antagonists. In certain embodiments the one or more non-TKI moieties -D are oncolytic drugs. In certain embodiments the one or more non-TKI moieties -D are signal converter proteins. In certain embodiments the one or more non-TKI moieties -D are epigenetic modifiers. In certain embodiments the one or more non-TKI moieties -D are tumor peptides or tumor vaccines. In certain embodiments the one or more non-TKI moieties -D are heat shock protein (HSP) inhibitors. In certain embodiments the one or more non-TKI moieties -D are proteolytic enzymes. In certain embodiments the one or more non-TKI moieties -D are ubiquitin and proteasome inhibitors. In certain embodiments the one or more non-TKI moieties -D are adhesion molecule antagonists. In certain embodiments the one or more non-TKI moieties -D are hormones including hormone peptides and synthetic hormones.

Examples for cytotoxic or chemotherapeutic agent are alkylating agents, anti-metabolites, anti-microtubule agents, topoisomerase inhibitors, cytotoxic antibiotics, auristatins, enediynes, lexitropsins, duocarmycins, cyclopropylpyrroloindoles, puromycin, dolastatins, maytansine derivatives, alkylsufonates, triazenes and piperazine.

Example for an alkylating agent are nitrogen mustards, such as mechlorethamine, cyclophosphamide, melphalan, chlorambucil, ifosfamide and busulfan; nitrosoureas, such as N-nitroso-N-methylurea, carmustine, lomustine, semustine, fotemustine and streptozotocin; tetrazines, such as dacarbazine, mitozolomide and temozolomide; ethylenimines, such as altretamine; aziridines, such as thiotepa, mitomycin and diaziquone; cisplatin and derivatives, such as cisplatin, carboplatin, oxaliplatin; and non-classical alkylating agents, such as procarbazine and hexamethylmelamine.

Examples for an anti-metabolite are anti-folates, such as methotrexate and pemetrexed; fluoropyrimidines, such as fluorouracil and capecitabine; deoxynucleoside analogues, such as cytarabine, gemcitabine, decitabine, azacytidine, fludarabine, nelarabine, cladribine, clofarabine and pentostatin; and thiopurines, such as thioguanine and mercaptopurine.

Examples for an anti-microtubule agent are Vinca alkaloids, such as vincristine, vinblastine, vinorelbine, vindesine and vinflunine; taxanes, such as paclitaxel and docetaxel; podophyllotoxins and derivatives, such as podophyllotoxin, etoposide and teniposide; stilbenoid phenol and derivatives, such as zybrestat (CA4P); and BNC105.

Examples for a topoisomerase inhibitor are topoisomerase I inhibitors, such as irinotecan, topotecan and camptothecin; and topoisomerase II inhibitors, such as etoposide, doxorubicin, mitoxantrone, teniposide, novobiocin, merbarone and aclarubicin.

Examples for a cytotoxic antibiotic are anthracyclines, such as doxorubicin, daunorubicin, epirubicin and idarubicin; pirarubicin, aclarubicin, bleomycin, mitomycin C, mitoxantrone, actinomycin, dactinomycin, adriamycin, mithramycin and tirapazamine.

Examples for an auristatin are monomethyl auristatin E (MMAE) and monomethyl auristatin F (MMAF).

Examples for an enediyne are neocarzinostatin, lidamycin (C-1027), calicheamicins, esperamicins, dynemicins and golfomycin A.

Examples for a maytansine derivative are ansamitocin, mertansine (emtansine, DM1) and ravtansine (soravtansine, DM4).

Examples for an immune checkpoint inhibitor or antagonist are inhibitors of CTLA-4 (cytotoxic T-lymphocyte-associated protein 4), such as ipilimumab, tremelimumab, MK-1308, FPT155, PRSO10, BMS-986249, BPI-002, CBT509, JS007, ONC392, TE1254, IBI310, BR02001, CG0161, KN044, PBI5D3H5, BCD145, ADU1604, AGEN1884, AGEN1181, CS1002 and CP675206; inhibitors of PD-1 (programmed death 1), such as pembrolizumab, nivolumab, pidilizumab, AMP-224, BMS-936559, cemiplimab and PDR001; inhibitors of PD-L1 (programmed cell death protein 1), such as MDX-1105, MED14736, atezolizumab, avelumab, BMS-936559 and durvalumab; inhibitors of PD-L2 (programmed death-ligand 2); inhibitors of KIR (killer-cell immunoglobulin-like receptor), such as lirlumab (IPH2102) and IPH2101; inhibitors of B7-H3, such as MGA271; inhibitors of B7-H4, such as FPA150; inhibitors of BTLA (B- and T-lymphocyte attenuator); inhibitors of LAG3 (lymphocyte-activation gene 3), such as IMP321 (eftilagimod alpha), relatlimab, MK-4280, AVA017, BI754111, ENUM006, GSK2831781, INCAGN2385, LAG3Ig, LAG525, REGN3767, Sym016, Sym022, TSR033, TSR075 and XmAb22841; inhibitors of TIM-3 (T-cell immunoglobulin and mucin-domain containing-3), such as LY3321367, MBG453, and TSR-022; inhibitors of VISTA (V-domain Ig suppressor of T cell activation), such as JNJ-61610588; inhibitors of ILT2/LILRB1 (Ig-like transcript 2/leukocyte Ig-like receptor 1); inhibitor of ILT3/LILRB4 (Ig-like transcript 3/leukocyte Ig-like receptor 4); inhibitors of ILT4/LILRB2 (Ig-like transcript 4/leukocyte Ig-like receptor 2), such as MK-4830; inhibitors of TIGIT (T cell immunoreceptor with Ig and ITIM domains), such as MK-7684, PTZ-201, RG6058 and COM902; inhibitors of NKG2A, such as IPH-2201; and inhibitors of PVRIG, such as COM701.

In certain embodiments said one or more non-TKI moiety -D is an inhibitor of PD-1. In certain embodiments said one or more non-TKI drug moiety -D is an inhibitor of PD-L1.

Examples for an immune agonist are CD27, such as recombinant CD70, such as HERA-CD27L, and varlilumab (CDX-1127); agonists of CD28, such as recombinant CD80, recombinant CD86, TGN1412 and FPT155; agonists of CD40, such as recombinant CD40L, CP-870,893, dacetuzumab (SGN-40), Chi Lob 7/4, ADC-1013 and CDX1140; agonists of 4-1BB (CD137), such as recombinant 4-1BBL, urelumab, utomilumab and ATOR-1017; agonists of OX40, such as recombinant OX40L, MEDI0562, GSK3174998, MOXR0916 and PF-04548600; agonists of GITR, such as recombinant GITRL, TRX518, MEDI1873, INCAGN01876, MK-1248, MK-4166, GWN323 and BMS-986156; and agonists of ICOS, such as recombinant ICOSL, JTX-2011 and GSK3359609.

Examples for a multi-specific drug are biologics and small molecule immune checkpoint inhibitors. Examples for biologics are multi-specific immune checkpoint inhibitors, such as CD137/HER2 lipocalin, PD1/LAG3, FS118, XmAb22841 and XmAb20717; and multi-specific immune agonists. Such multi-specific immune agonists may be selected from the group consisting of Ig superfamily agonists, such as ALPN-202; TNF superfamily agonists, such as ATOR-1015, ATOR-1144, ALG.APV-527, lipocalin/PRS-343, PRS344/ONC0055, FAP-CD40 DARPin, MP0310 DARPin, FAP-0X40 DARPin, EGFR-CD40 DARPin, EGFR41BB/CD137 DARPin, EGFR-0X40/DARFPin, HER2-CD40 DARPin, HER2-41BB/CD137 DARPin, HER2-0X40 DARPin, FIBRONECTIN ED-B-CD40 DARPin, FIBRONECTIN ED-B-41BB/CD137 and FIBRONECTIN ED-B-0X40 DARPin; CD3 multispecific agonists, such as blinatumomab, solitomab, MEDI-565, ertumaxomab, anti-HER2/CD3 1Fab-immunoglobulin G TDB, GBR 1302, MGD009, MGD007, EGFRBi, EGFR-CD Probody, RG7802, PF-06863135, PF-06671008, MOR209/ES414, AMG212/BAY2010112 and CD3-5T4; and CD16 multispecific agonists, such as 1633 BiKE, 161533 TriKE, OXS-3550, OXS-C3550, AFM13 and AFM24.

An example for a small molecule immune checkpoint inhibitor is CA-327 (TIM3/PD-L1 antagonist).

Examples for an antibody-drug conjugate are ADCs targeting hematopoietic cancers, such as gemtuzumab ozogamicin, brentuximab vedotin, inotuzumab ozogamicin, SAR3419, BT062, SGN-CD19A, IMGN529, MDX-1203, polatuzumab vedotin (RG7596), pinatuzumab vedotin (RG7593), RG7598, milatuzumab-doxorubicin and OXS-1550; and ADCs targeting solid tumor antigens, such as trastuzumab emtansine, glembatumomab vedotin, SAR56658, AMG-172, AMG-595, BAY-94-9343, BIIB015, vorsetuzumab mafodotin (SGN-75), ABT-414, ASG-5ME, enfortumab vedotin (ASG-22ME), ASG-16M8F, IMGN853, indusatumab vedotin (MLN-0264), vadortuzumab vedotin (RG7450), sofituzumab vedotin (RG7458), lifastuzumab vedotin (RG7599), RG7600, DEDN6526A (RG7636), PSMA TTC, 1095 from Progenics Pharmaceuticals, lorvotuzumab mertansine, lorvotuzumab emtansine, IMMU-130, sacituzumab govitecan (IMMU-132), PF-06263507 and MEDI0641.

Examples for radionuclides are β-emitters, such as ¹⁷⁷Lutetium, ¹⁶⁶Holmium, ¹⁸⁶Rhenium ¹⁸⁸Rhenium, ⁶⁷Copper, ¹⁴⁹Promethium, ¹⁹⁹Gold, ⁷⁷Bromine, ¹⁵³Samarium, ¹⁰⁵Rhodium, ⁸⁹Strontium, ⁹⁰Yttrium, ¹³¹Iodine; α-emitters, such as ²¹³Bismuth, ²²³Radium, ²²⁵Actinium, ²¹¹Astatine; and Auger electron-emitters, such as ⁷⁷Bromine, ¹¹¹Indium, ¹²³Iodine and ¹²⁵Iodine.

Examples for targeted radionuclide therapeutics are zevalin (⁹⁰Y-ibritumomab tiuxetan), bexxar (¹³¹I-tositumomab), oncolym (¹³¹I-Lym 1), lymphocide (⁹⁰Y-epratuzumab), cotara (¹³¹I-chTNT-1/B), labetuzumab (⁹⁰Y or ¹³¹I-CEA), theragyn (⁹⁰Y-pemtumomab), licartin (¹³¹I-metuximab), radretumab (¹³¹I-L19) PAM4 (⁹⁰Y-clivatuzumab tetraxetan), xofigo (²²³Ra dichloride), lutathera (¹⁷⁷Lu-DOTA-Tyr³-Octreotate) and ¹³¹I-MIBG.

Examples for a DNA damage repair inhibitor are poly (ADP-ribose) polymerase (PARP) inhibitors, such as olaparib, rucaparib, niraparib, veliparib, CEP 9722 and E7016; CHK1/CHK2 dual inhibitors, such as AZD7762, V158411, CBP501 and XL844; CHK1 selective inhibitors, such as PF477736, MK8776/SCH900776, CCT244747, CCT245737, LY2603618, LY2606368/prexasertib, AB-IsoG, ARRY575, AZD7762, CBP93872, ESP01, GDC0425, SAR020106, SRA737, V158411 and VER250840; CHK2 inhibitors, such as CCT241533 and PV1019; ATM inhibitors, such as AZD0156, AZD1390, KU55933, M3541 and SX-RDS1; ATR inhibitors, such as AZD6738, BAY1895344, M4344 and M6620 (VX-970); and DNA-PK inhibitors, such as M3814.

Examples for a tumor metabolism inhibitor are inhibitors of the adenosine pathway, inhibitors of the tryptophan metabolism and inhibitors of the arginine pathway.

Examples for an inhibitor of the adenosine pathway are inhibitors of A2AR (adenosine A2A receptor), such as ATL-444, istradefylline (KW-6002), MSX-3, preladenant (SCH-420,814), SCH-58261, SCH412,348, SCH-442,416, ST-1535, caffeine, VER-6623, VER-6947, VER-7835, vipadenant (BIIB-014), ZM-241,385, PBF-509 and V81444; inhibitors of CD73, such as IPH53 and SRF373; and inhibitors of CD39, such as IPH52.

Examples for an inhibitor of the tryptophane metabolism are inhibitors of IDO, such as indoximod (NLG8189), epacadostat, navoximod, BMS-986205 and MK-7162; inhibitors of TDO, such as 680C91; and IDO/TDO dual inhibitors.

Examples for inhibitors of the arginine pathway are inhibitors of arginase, such as INCB001158.

Examples for a pattern recognition agonist are Toll-like receptor agonists, NOD-like receptors, RIG-I-like receptors, cytosolic DNA sensors, STING, and aryl hydrocarbon receptors (AhR).

Examples for Toll-like receptor agonists are agonists of TLR1/2, such as peptidoglycans, lipoproteins, Pam3CSK4, Amplivant, SLP-AMPLIVANT, HESPECTA, ISA101 and ISA201; agonists of TLR2, such as LAM-MS, LPS-PG, LTA-BS, LTA-SA, PGN-BS, PGN-EB, PGN-EK, PGN-SA, CL429, FSL-1, Pam2CSK4, Pam3CSK4, zymosan, CBLB612, SV-283, ISA204, SMP105, heat killed Listeria monocytogenes; agonists of TLR3, such as poly(A:U), poly(I:C) (poly-ICLC), rintatolimod, apoxxim, IPH3102, poly-ICR, PRV300, RGCL2, RGIC.1, Riboxxim (RGC100, RGIC100), Riboxxol (RGIC50) and Riboxxon; agonists of TLR4, such as lipopolysaccharides (LPS), neoceptin-3, glucopyranosyl lipid adjuvant (GLA), GLA-SE, G100, GLA-AF, clinical center reference endotoxin (CCRE), monophosphoryl lipid A, grass MATA MPL, PEPA10, ONT-10 (PET-Lipid A, oncothyreon), G-305, ALD046, CRX527, CRX675 (RC527, RC590), GSK1795091, OM197MPAC, OM294DP and SAR439794; agonists of TLR2/4, such as lipid A, OM174 and PGN007; agonists of TLR5, such as flagellin, entolimod, mobilan, protectan CBLB501; agonists of TLR6/2, such as diacylated lipoproteins, diacylated lipopeptides, FSL-1, MALP-2 and CBLB613; agonists of TLR7, such as CL264, CL307, imiquimod (R837), TMX-101, TMX-201, TMX-202, TMX-302, gardiquimod, S-27609, 851, UC-IV150, 852A (3M-001, PF-04878691), loxoribine, polyuridylic acid, GSK2245035, GS-9620, R06864018 (ANA773, RG7795), R07020531, isatoribine, AN0331, ANA245, ANA971, ANA975, DSP0509, DSP3025 (AZD8848), GS986, MBS2, MBS5, RG7863 (R06870868), sotirimod, SZU101 and TQA3334; agonists of TLR8, such as ssPolyUridine, ssRNA40, TL8-506, XG-1-236, VTX-2337 (motolimod), VTX-1463, TMX-302, VTX-763, DN1508052 and GS9688; agonists of TLR7/8, such as CL075, CL097, poly(dT), resiquimod (R-848, VML600, S28463), MED19197 (3M-052), NKTR262, DV1001, IM04200, IPH3201 and VTX1463; agonists of TLR9, such as CpG DNA, CpG ODN, lefitolimod (MGN1703), SD-101, QbG10, CYT003, CYT003-QbG10, DUK-CpG-001, CpG-7909 (PF-3512676), GNKG168, EMD 1201081, IMO-2125, IMO-2055, CpG10104, AZD1419, AST008, IM02134, MGN1706, IRS 954, 1018 ISS, actilon (CPG10101), ATP00001, AVE0675, AVE7279, CMP001, DIMS0001, DIMS9022, DIMS9054, DIMS9059, DV230, DV281, EnanDIM, heplisav (V270), kappaproct (DIMS0150), NJP834, NPI503, SAR21609 and tolamba; and agonists of TLR7/9, such as DV 1179.

In certain embodiments the non-TKI moiety -D is an agonist of TLR7/8 as described in EP 19150384. In particular a non-TKI moiety -D is in certain embodiments of formula (1)

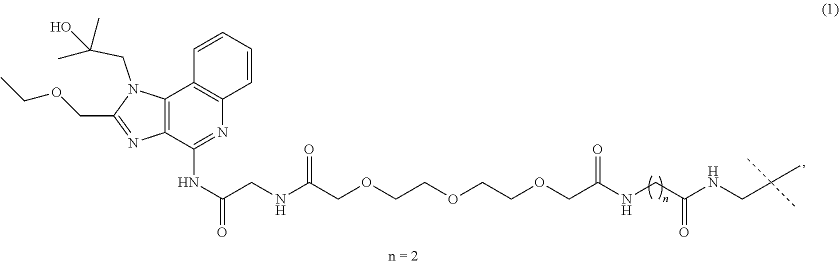

wherein the dashed line indicates attachment to a PEG hydrogel. It is understood that a multitude of the moieties of formula (1) are conjugated to said hydrogel.

Examples for CpG ODN are ODN 1585, ODN 2216, ODN 2336, ODN 1668, ODN 1826, ODN 2006, ODN 2007, ODN BW006, ODN D-SL01, ODN 2395, ODN M362 and ODN D-SL03.

Examples for NOD-like receptors are agonists of NOD1, such as C12-iE-DAP, C14-Tri-LAN-Gly, iE-DAP, iE-Lys, and Tri-DAP; and agonists of NOD2, such as L18-MDP, MDP, M-TriLYS, murabutide and N-glycolyl-MDP.

Examples for RIG-I-like receptors are 3p-hpRNA, 5′ppp-dsRNA, 5′ppp RNA (M8), 5′OH RNA with kink (CBS-13-BPS), 5′PPP SLR, KIN100, KIN 101, KIN1000, KIN1400, KIN1408, KIN1409, KIN1148, KIN131A, poly(dA:dT), SB9200, RGT100 and hiltonol.

Examples for cytosolic DNA sensors are cGAS agonists, dsDNA-EC, G3-YSD, HSV-60, ISD, ODN TTAGGG (A151), poly(dG:dC) and VACV-70.

Examples for STING are MK-1454, ADU-S100 (MIW815), 2′3′-cGAMP, 3′3′-cGAMP, c-di-AMP, c-di-GMP, cAIMP (CL592), cAIMP difluor (CL614), cAIM(PS)₂ difluor (Rp/Sp) (CL656), 2′2′-cGAMP, 2′3′-cGAM(PS)₂ (Rp/Sp), 3′3′-cGAM fluorinated, c-di-AMP fluorinated, 2′3′-c-di-AMP, 2′3′-c-di-AM(PS)₂ (Rp,Rp), c-di-GMP fluorinated, 2′3′-c-di-GMP, c-di-IMP, c-di-UMP and DMXAA (vadimezan, ASA404).

Examples for an aryl hydrocarbon receptor (AhR) are of FICZ, ITE and L-kynurenine.

Examples for a chemokine receptor and chemoattractant receptor agonist are CXC chemokine receptors, CC chemokine receptors, C chemokine receptors, CX3C chemokine receptors and chemoattractant receptors.

Examples for a CXC chemokine receptor are CXCR1 agonists, such as recombinant CXCL8 and recombinant CXCL6; CXCR2 agonists, such as recombinant CXCL8, recombinant CXCL1, recombinant CXCL2, recombinant CXCL3, recombinant CXCL5, recombinant CXCL6, MGTA 145 and SB251353; CXCR3 agonists, such as recombinant CXCL9, recombinant CXCL10, recombinant CXCL11 and recombinant CXCL4; CXCR4 agonists, such as recombinant CXCL12, ATI2341, CTCE0214, CTCE0324 and NNZ4921; CXCR5 agonists, such as recombinant CXCL13; CXCR6 agonists, such as recombinant CXCL16; and CXCL7 agonists, such as recombinant CXCL11.

Examples for a CC chemokine receptor are CCR1 agonists, such as recombinant CCL3, ECI301, recombinant CCL4, recombinant CCL5, recombinant CCL6, recombinant CCL8, recombinant CCL9/10, recombinant CCL14, recombinant CCL15, recombinant CCL16, recombinant CCL23, PB103, PB105 and MPIF1; CCR2 agonists, such as recombinant CCL2, recombinant CCL8, recombinant CCL16, PB103 and PB105; CCR3 agonists, such as recombinant CCL11, recombinant CCL26, recombinant CCL7, recombinant CCL13, recombinant CCL15, recombinant CCL24, recombinant CCL5, recombinant CCL28 and recombinant CCL18; CCR4 agonists, such as recombinant CCL3, ECI301, recombinant CCL5, recombinant CCL17 and recombinant CCL22; CCR5 agonists, such as recombinant CCL3, ECI301, recombinant CCL5, recombinant CCL8, recombinant CCL11, recombinant CCL13, recombinant CCL14, recombinant CCL16, PB103 and PB105; CCR6 agonists, such as recombinant CCL20; CCR7 agonists, such as recombinant CCL19 and recombinant CCL21; CCR8 agonists, such as recombinant CCL1, recombinant CCL16, PB103 and PB105; CCR9 agonists, such as recombinant CCL25; CCR10 agonists, such as recombinant CCL27 and recombinant CCL28; and CCR11 agonists, such as recombinant CCL19, recombinant CCL21 and recombinant CCL25.

Examples for C chemokine receptors are XCR1 agonist, such as recombinant XCL1 or recombinant XCL2.

Examples for CX3C chemokine receptors are CX3CR1 agonist, such as recombinant CX3CL1.

Examples for chemoattractant receptors are formyl peptide receptor agonists, such as N-formyl peptides, N-formylmethionine-leucyl-phenylalanine, enfuvirtide, T21/DP107, annexin A1, Ac2-26 and Ac9-25; C5a receptor agonists; and chemokine-like receptor 1 agonists, such as chemerin.

Examples for chemokine antagonists are inhibitors of CXCL chemokines, such as UNBS5162; inhibitors of CXCL8, such as BMS986253 and PA620; inhibitors of CXCL10, such as TM110, eldelumab and NI0801; inhibitors of CXCL12, such as NOX-A12 and JVS100; inhibitors of CXCL13, such as VX5; inhibitors of CCL2, such as PA508, ABN912, AF2838, BN83250, BN83470, C243, CGEN54, CNT0888, NOXE36, VT224 and SSR150106; inhibitors of CCL5, such as HGS1025 and NI0701; inhibitors of CCL2/CCL5, such as BKTP46; inhibitors of CCL5/FMLP receptor, such as RAP160; inhibitors of CCL11, such as bertilimumab and RAP701; inhibitors of CCL5/CXCL4, such as CT2008 and CT2009; inhibitors of CCL20, such as GSK3050002; and inhibitors of CX3CL1, such as quetmolimab.

Examples for chemokine receptor antagonists are inhibitors of CXCR1, such as repertaxin, CCX832, FX68 and KB03; inhibitors of CXCR2, such as AZD5069, AZD5122, AZD8309, GSK1325756, GSK1325756H, PS291822, SB332235 and SB656933; inhibitors of CXCR1/CXCR2, such as DF1970, DF2156A, DF2162, DF2755A, reparixin, SX576, SX682, PACG31P, AZD4721 and PA401; inhibitors of CXCR3; inhibitors of CXCR4, such as BL8040; inhibitors of CXCR4/E-selectin, such as GMI1359; inhibitors of CXCR6, such as CCX5224; inhibitors of CCR1, such as AZD4818, BAY865047, BMS817399, CCX354, CCX634, CCX9588, CP481715, MLN3701, MLN3897, PS031291, PS375179 and PS386113; inhibitors of CCR2, such as AZD2423, BL2030, BMS741672, CCX140, CCX598, CCX872, CCX915, CNTX6970, INCB3284, INCB3344, INCB8696, JNJ17166864, JNJ27141491, MK0812, OPLCCL2LPM, PF4136309, serocion, STIB0201, STIB0211, STIB0221, STIB0232, STIB0234, TAK202, TPI526; inhibitors of CCR2/CCR5, such as PF04634817, RAP103 and TBR652; inhibitors of CCR2/CCR5/CCR8, such as RAP310; inhibitors of CCR3, such as ASM8, AXP1275, BMS639623, CM101, DPC168, GW766994, GW824575, MT0814, OPLCCL11LPM and QAP642; inhibitors of CCR4, such as AT008, AZD2098, CCX6239, FLX193, FLX475, GBV3019, GSK2239633, IC487892 and poteligeo; inhibitors of CCR5, such as 5P12-RANTES, AZD5672, AZD8566, CMPD167, ESN196, GSK706769, GW873140, HGS004, INCB15050, INCB9471, L872, microbicide, PF232798, PRO140, RAP101, SAR113244, SCH350634, SCH351125, SCH417690, selzentry, TAK779, TBR220, TD0232 and VX286; inhibitors of CCR5/CXCR4, such as AMD887, ND401 and SPO1A; inhibitors of CCR6, such as CCX507, CCX9664 and STIB100X; inhibitors of CCR6, such as CCX025, CCX507, CCX807, eut22, MLN3126, POL7085, traficet-EN; inhibitors of CXCR3, such as AMG487, AT010, STIA120X; inhibitors of CXCR4, such as AD114, AD214, ALX0651, ALX40-4C, AMD070, AT007, AT009, BKT170, BMS936564, celixafor, CTCE9908, GBV4086, GSK812397, KRH2731, KRH3140, LY2510924, LY2624587, mozobil, OPLCXCL12LPM, PF06747143, POL6326, Q122, revixil, TG0054, USL311, X4P001 and X4P002; and inhibitors of CXCR7, such as CCX650 and agonists of IFNα/β receptor, agonists of IFN γ receptor, agonists of FLT3 receptor.

Examples for a cytokine receptor agonist are mRNAs, DNAs or plasmids encoding the genes for IL-2, IL-15, IL-7, IL-10, IL-12, IL-21, IFNα 1-17, IFNβ, IFNγ, IL-18, IL-27, TNFα, GM-CSF, FLT3L and TRAIL and recombinant proteins, such as agonists of IL-2/IL-15 β/γ receptors, agonists of IL-10 receptor, agonists of IL-12 receptor, agonists of IL-18 receptor, agonists of IL-21 receptor, agonists of IL-7 receptor, and agonists of TNFα receptor.

Examples for agonists of IL-2/IL-15 β/γ receptor are recombinant IL-2, recombinant IL-15, ALKS4230, ALT803, APN301, MDNA109, NKTR214, RG7461, RG7813, AM0015, NIZ985, NKTR255, RTX-212, SO-C101, XmAb24306, L19-IL2, THOR-707 and PB101.

In certain embodiments a non-TKI moiety -D is as described in PCT/EP2019/057709, which is herewith incorporated by reference in its entirety. In particular such non-TKI moiety -D is in certain embodiments a conjugate comprising an IL-2 protein of SEQ ID NO:1

PTSSSTKKTQ LQLEHLLLDL QMILNGINNY KNPKLTCMLT FKFYMPKKAT ELKHLQCLEE ELKPLEEVLN LAQSKNFHLR PRDLISNINV IVLELKGSET TFMCEYADET ATIVEFLNRW ITFSQSIIST LT,

wherein the sulfur of the cysteine at position 37 of SEQ ID NO:1 is conjugated to a moiety of formula (2)

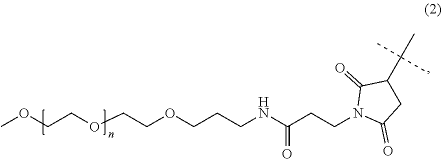

wherein the dashed line indicates attachment to said sulfur, and
n is about 113 or about 226;

and wherein the nitrogen of the amine of the side chain of any one of the lysine residues, i.e. one of the lysine residues selected from the group consisting of the lysine residues at position 7, 8, 31, 34, 42, 47, 48, 53, 63, 75 and 96 of SEQ ID NO:1, is conjugated to a moiety of formula (3)

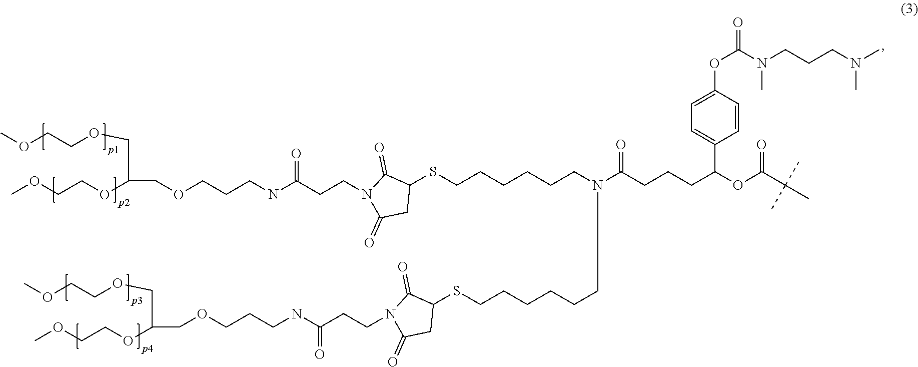

wherein the dashed line indicates attachment to said nitrogen of the side chain of said lysine residue; and
p1, p2, p3 and p4 are independently an integer ranging from 200 to 250.

In certain embodiments the sequence of the IL-2 protein varies by at least one amino acid from the sequence of SEQ ID NO:1, such as by one amino acid, by two amino acids, by three amino acids, by four amino acids or by five amino acids.

In certain embodiments the sequence of the IL-2 protein is of SEQ ID NO:2:

APTSSSTKKTQLQLEHLLLDLQMILNGINNYKNPKLTCMLTFKFYMPKKA
TELKHLQCLEEELKPLEEVLNLAQSKNFHLRPRDLISNINVIVLELKGSE
TTFMCEYADETATIVEFLNRWITFSQSIISTLT

Accordingly, a non-TKI moiety -D is in certain embodiments a conjugate comprising an IL-2 protein of SEQ ID NO:2

APTSSSTKKTQLQLEHLLLDLQMILNGINNYKNPKLTCMLTFKFYMPKKA
TELKHLQCLEEELKPLEEVLNLAQSKNFHLRPRDLISNINVIVLELKGSE
TTFMCEYADETATIVEFLNRWITFSQSIISTLT,

wherein the sulfur of the cysteine at position 38 of SEQ ID NO:2 is conjugated to a moiety of formula (2)

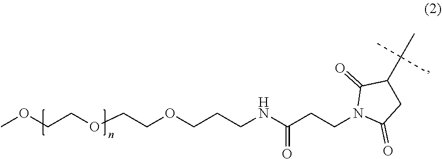

wherein the dashed line indicates attachment to said sulfur, and
n is about 113 or about 226;

and wherein the nitrogen of the amine of the side chain of any one of the lysine residues, i.e. one of the lysine residues selected from the group consisting of the lysine residues at position 8, 9, 32, 35, 43, 48, 49, 54, 64, 76 and 97 of SEQ ID NO:2, is conjugated to a moiety of formula (3)

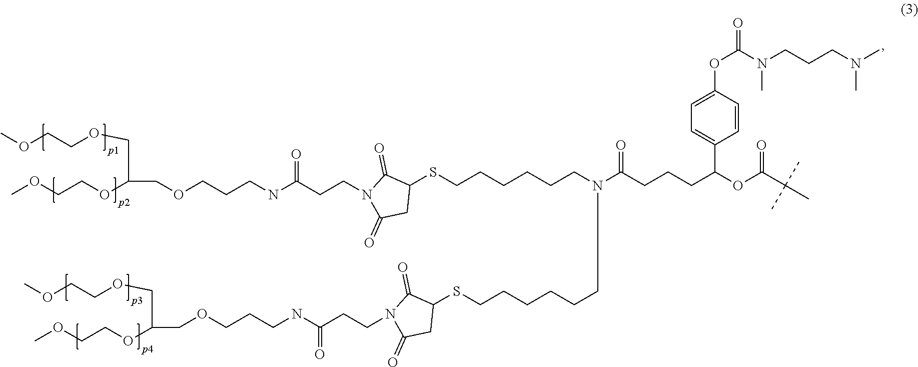

wherein the dashed line indicates attachment to said nitrogen of the side chain of said lysine residue; and
p1, p2, p3 and p4 are independently an integer ranging from 200 to 250.

In certain embodiments n of formula (2) is about 113. In certain embodiments n of formula (2) is about 226.

In certain embodiments p1, p2, p3 and p4 are independently an integer ranging from 220 to 240. In certain embodiments p1, p2, p3 and p4 are the same integer.

Examples for agonists of IL-10 receptor are AG011, dekavil, EG10, IL10Nanocap, Ilodecakin, AM0010, tenovil and VT310 VIRON.

Examples for agonists of IL-12 receptor are AM0012, AS1409, dodekin, HemaMax, LipoVIL12, MSB0010360N and NHS-IL12.

An example for an agonist of IL-18 receptor is SB485232.

An example for an agonist of IL-21 receptor is BMS982470 (denenicokin).

Examples for agonists of IL-7 receptor are CYT107, CYT99007 and GX-I7.

Examples for agonist of TNFα receptor are L19-TNFα, aurimune, beromun, BreMel/TNFα, fibromun, refnot and TNFPEG20.

Examples for death receptor agonists are TRAILR1/DR4 agonists, such as AMG951 (dulanermin), APG350, APG880, HGSETR1 (mapatumumab) and SL231; and TRAILR2/DR5 agonists, such as AMG655, DS8273, HGSETR2 (lexatumumab), HGSTR2J, IDD004/GEN1029, INBRX109, LBY135, MEDI3039, PR095780, RG7386 and TAS266.

Examples for CD47 antagonists are ALX148, CC-90002, Hu5F9G4, SRF231, TI061, TTI-621, TTI-622, A0176, IB1188, IMC002 and LYN00301.

An example for a SIRPα antagonist is FST89.

Examples for oncolytic drugs are CAVATAK, BCG, mobilan, TG4010, Pexa-Vec (JX-594), JX-900, JX-929 and JX-970.

Examples for signal converter proteins are Fn14-TRAIL (KAHR101), CTLA4-FasL (KAHR102), PD1-41BBL (DSP 105), PD1-CD70 (DSP 106) and SIRPα-41BBL (DSP 107).

Examples for epigenetic modifiers are DNA methyltransferase inhibitors, lysine-specific demethylase 1 inhibitors, Zeste homolog 2 inhibitors, bromodomain and extra-terminal motif (BET) protein inhibitors such as GSK525762, and histone deacetylase (HDAC) inhibitors such as beleodaq, SNDX275 and CKD-M808.

Examples for tumor peptides/vaccines are NY-ESO, WT1, MART-1, 10102 and PF-06753512.

Examples for heat shock protein (HSP) inhibitors are inhibitors of HSP90, such as PF-04929113 (SNX-5422).

Examples of proteolytic enzymes are recombinant hyaluronidase, such as rHuPH20 and PEGPH20.

Examples for ubiquitin and proteasome inhibitors are ubiquitin-specific protease (USP) inhibitors, such as P005091; 20S proteasome inhibitors, such as bortezimib, carfilzomib, ixazomib, oprozomib, delanzomib and celastrol; and immunoproteasome inhibitors, such as ONX-0914.

Examples for adhesion molecule antagonists are 02-integrin antagonists, such as imprime PGG; and selectin antagonists.

Examples for hormones are hormone receptor agonists and hormone receptor antagonists.

Examples for a hormone receptor agonist are somatostatin receptor agonists, such as somatostatin, lanreotide, octreotide, FX125L, FX141L and FX87L.

Examples for hormone receptor antagonists are anti-androgens, anti-estrogens and anti-progestogens. Examples for anti-androgens are steroidal antiandrogens, such as cyproterone acetate, megestrol acetate, chlormadinone acetate, spironolactone, oxendolone and osaterone acetate; nonsteroidal anti-androgens, such as flutamide, bicalutamide, nilutamide, topilutamide, enzalutamide and apalutamide; androgen synthesis inhibitors, such as ketoconazole, abiraterone acetate, seviteronel, aminoglutethimide, finasteride, dutasteride, epristeride and alfatradiol. Examples for anti-estrogens are selective estrogen receptor modulators (SERMs), such as tamoxifen, clomifene, Fareston and raloxifene; ER silent antagonists and selective estrogen receptor degrader (SERD), such as fulvestrant; aromatase inhibitors, such as anastrozole, letrozole, exemestane, vorozole, formestane and fadrozole; and anti-gonadotropins, such as testosterone, progestogens and GnRH analogues. Examples for anti-progestogens are mifepristone, lilopristone and onapristone.

In certain embodiments such cytotoxic or chemotherapeutic agents are selected from the group consisting of alkylating agents, anti-metabolites, anti-microtubule agents, topoisomerase inhibitors, cytotoxic antibiotics, auristatins, enediynes, lexitropsins, duocarmycins, cyclopropylpyrroloindoles, puromycin, dolastatins, maytansine derivatives, alkylsufonates, triazenes and piperazine.

The alkylating agent is in certain embodiments selected from the group consisting of nitrogen mustards, such as mechlorethamine, cyclophosphamide, melphalan, chlorambucil, ifosfamide and busulfan; nitrosoureas, such as N-nitroso-N-methylurea, carmustine, lomustine, semustine, fotemustine and streptozotocin; tetrazines, such as dacarbazine, mitozolomide and temozolomide; ethylenimines, such as altretamine; aziridines, such as thiotepa, mitomycin and diaziquone; cisplatin and derivatives, such as cisplatin, carboplatin, oxaliplatin; and non-classical alkylating agents, such as procarbazine and hexamethylmelamine.

The anti-metabolite is in certain embodiments selected from the group consisting of anti-folates, such as methotrexate and pemetrexed; fluoropyrimidines, such as fluorouracil and capecitabine; deoxynucleoside analogues, such as cytarabine, gemcitabine, decitabine, azacytidine, fludarabine, nelarabine, cladribine, clofarabine and pentostatin; and thiopurines, such as thioguanine and mercaptopurine.

The anti-microtubule agent is in certain embodiments selected from the group consisting of Vinca alkaloids, such as vincristine, vinblastine, vinorelbine, vindesine and vinflunine; taxanes, such as paclitaxel and docetaxel; podophyllotoxins and derivatives, such as podophyllotoxin, etoposide and teniposide; stilbenoid phenol and derivatives, such as zybrestat (CA4P); and BNC105.

The topoisomerase inhibitor is in certain embodiments selected from the group consisting of topoisomerase I inhibitors, such as irinotecan, topotecan and camptothecin; and topoisomerase II inhibitors, such as etoposide, doxorubicin, mitoxantrone, teniposide, novobiocin, merbarone and aclarubicin.

The cytotoxic antibiotic is in certain embodiments selected from the group consisting of anthracyclines, such as doxorubicin, daunorubicin, epirubicin and idarubicin; pirarubicin, aclarubicin, bleomycin, mitomycin C, mitoxantrone, actinomycin, dactinomycin, adriamycin, mithramycin and tirapazamine.

The auristatin is in certain embodiments selected from the group consisting of monomethyl auristatin E (MMAE) and monomethyl auristatin F (MMAF).

The enediyne is in certain embodiments selected from the group consisting of neocarzinostatin, lidamycin (C-1027), calicheamicins, esperamicins, dynemicins and golfomycin A.

The maytansine derivative is in certain embodiments selected from the group consisting of ansamitocin, mertansine (emtansine, DM1) and ravtansine (soravtansine, DM4).

The immune checkpoint inhibitor or antagonist is in certain embodiments selected from the group consisting of inhibitors of CTLA-4 (cytotoxic T-lymphocyte-associated protein 4), such as ipilimumab, tremelimumab, MK-1308, FPT155, PRSO10, BMS-986249, BPI-002, CBT509, JS007, ONC392, TE1254, IB1310, BR02001, CG0161, KN044, PBI5D3H5, BCD145, ADU1604, AGEN1884, AGEN1181, CS1002 and CP675206; inhibitors of PD-1 (programmed death 1), such as pembrolizumab, nivolumab, pidilizumab, AMP-224, BMS-936559, cemiplimab and PDR001; inhibitors of PD-L1 (programmed cell death protein 1), such as MDX-1105, MEDI4736, atezolizumab, avelumab, BMS-936559 and durvalumab; inhibitors of PD-L2 (programmed death-ligand 2); inhibitors of KIR (killer-cell immunoglobulin-like receptor), such as lirlumab (IPH2102) and IPH2101; inhibitors of B7-H3, such as MGA271; inhibitors of B7-H4, such as FPA150; inhibitors of BTLA (B- and T-lymphocyte attenuator); inhibitors of LAG3 (lymphocyte-activation gene 3), such as IMP321 (eftilagimod alpha), relatlimab, MK-4280, AVA017, BI754111, ENUM006, GSK2831781, INCAGN2385, LAG3Ig, LAG525, REGN3767, Sym016, Sym022, TSR033, TSR075 and XmAb22841; inhibitors of TIM-3 (T-cell immunoglobulin and mucin-domain containing-3), such as LY3321367, MBG453, and TSR-022; inhibitors of VISTA (V-domain Ig suppressor of T cell activation), such as JNJ-61610588; inhibitors of ILT2/LILRB1 (Ig-like transcript 2/leukocyte Ig-like receptor 1); inhibitor of ILT3/LILRB4 (Ig-like transcript 3/leukocyte Ig-like receptor 4); inhibitors of ILT4/LILRB2 (Ig-like transcript 4/leukocyte Ig-like receptor 2), such as MK-4830; inhibitors of TIGIT (T cell immunoreceptor with Ig and ITIM domains), such as MK-7684, PTZ-201, RG6058 and COM902; inhibitors of NKG2A, such as IPH-2201; and inhibitors of PVRIG, such as COM701.

The immune agonist is in certain embodiments selected from the group consisting of agonists of CD27, such as recombinant CD70, such as HERA-CD27L, and varlilumab (CDX-1127); agonists of CD28, such as recombinant CD80, recombinant CD86, TGN1412 and FPT155; agonists of CD40, such as recombinant CD40L, CP-870,893, dacetuzumab (SGN-40), Chi Lob 7/4, ADC-1013 and CDX1140; agonists of 4-1BB (CD137), such as recombinant 4-1BBL, urelumab, utomilumab and ATOR-1017; agonists of OX40, such as recombinant OX40L, MED10562, GSK3174998, MOXR0916 and PF-04548600; agonists of GITR, such as recombinant GITRL, TRX518, MEDI1873, INCAGN01876, MK-1248, MK-4166, GWN323 and BMS-986156; and agonists of ICOS, such as recombinant ICOSL, JTX-2011 and GSK3359609.

The multi-specific drug is in certain embodiments selected from the group consisting of biologics and small molecule immune checkpoint inhibitors. Examples for biologics are multi-specific immune checkpoint inhibitors, such as CD137/HER2 lipocalin, PD1/LAG3, FS118, XmAb22841 and XmAb20717; and multi-specific immune agonists. Such multi-specific immune agonists may be selected from the group consisting of Ig superfamily agonists, such as ALPN-202; TNF superfamily agonists, such as ATOR-1015, ATOR-1144, ALG.APV-527, lipocalin/PRS-343, PRS344/ONC0055, FAP-CD40 DARPin, MP0310 DARPin, FAP-0X40 DARPin, EGFR-CD40 DARPin, EGFR41BB/CD137 DARPin, EGFR-0X40/DARFPin, HER2-CD40 DARPin, HER2-41BB/CD137 DARPin, HER2-0X40 DARPin, FIBRONECTIN ED-B-CD40 DARPin, FIBRONECTIN ED-B-41BB/CD137 and FIBRONECTIN ED-B-0X40 DARPin; CD3 multispecific agonists, such as blinatumomab, solitomab, MEDI-565, ertumaxomab, anti-HER2/CD3 1Fab-immunoglobulin G TDB, GBR 1302, MGD009, MGD007, EGFRBi, EGFR-CD Probody, RG7802, PF-06863135, PF-06671008, MOR209/ES414, AMG212/BAY2010112 and CD3-5T4; and CD16 multispecific agonists, such as 1633 BiKE, 161533 TriKE, OXS-3550, OXS-C3550, AFM13 and AFM24.

Such immune checkpoint inhibitor or antagonist is in certain embodiments selected from the group consisting of inhibitors of CTLA-4 (cytotoxic T-lymphocyte-associated protein 4), such as ipilimumab, tremelimumab, MK-1308, FPT155, PRS010, BMS-986249, BPI-002, CBT509, JS007, ONC392, TE1254, 1B1310, BR02001, CG0161, KN044, PBI5D3H5, BCD145, ADU1604, AGEN1884, AGEN1181, CS1002 and CP675206; inhibitors of PD-1 (programmed death 1), such as pembrolizumab, nivolumab, pidilizumab, AMP-224, BMS-936559, cemiplimab and PDR001; inhibitors of PD-L1 (programmed cell death protein 1), such as MDX-1105, MEDI4736, atezolizumab, avelumab, BMS-936559 and durvalumab; inhibitors of PD-L2 (programmed death-ligand 2); inhibitors of KIR (killer-cell immunoglobulin-like receptor), such as lirlumab (IPH2102) and IPH2101; inhibitors of B7-H3, such as MGA271; inhibitors of B7-H4, such as FPA150; inhibitors of BTLA (B- and T-lymphocyte attenuator); inhibitors of LAG3 (lymphocyte-activation gene 3), such as IMP321 (eftilagimod alpha), relatlimab, MK-4280, AVA017, BI754111, ENUM006, GSK2831781, INCAGN2385, LAG3Ig, LAG525, REGN3767, Sym016, Sym022, TSR033, TSR075 and XmAb22841; inhibitors of TIM-3 (T-cell immunoglobulin and mucin-domain containing-3), such as LY3321367, MBG453, and TSR-022; inhibitors of VISTA (V-domain Ig suppressor of T cell activation), such as JNJ-61610588; inhibitors of ILT2/LILRB1 (Ig-like transcript 2/leukocyte Ig-like receptor 1); inhibitor of ILT3/LILRB4 (Ig-like transcript 3/leukocyte Ig-like receptor 4); inhibitors of ILT4/LILRB2 (Ig-like transcript 4/leukocyte Ig-like receptor 2), such as MK-4830; inhibitors of TIGIT (T cell immunoreceptor with Ig and ITIM domains), such as MK-7684, PTZ-201, RG6058 and COM902; inhibitors of NKG2A, such as IPH-2201; and inhibitors of PVRIG, such as COM701.

A moiety -L¹- is conjugated to -D via a functional group of -D, which functional group is in certain embodiments selected from the group consisting of carboxylic acid, primary amine, secondary amine, thiol, sulfonic acid, carbonate, carbamate, hydroxyl, aldehyde, ketone, hydrazine, isothiocyanate, phosphoric acid, phosphonic acid, acryloyl, hydroxylamine, sulfate, vinyl sulfone, vinyl ketone, diazoalkane, guanidine, aziridine, amide, imide, imine, urea, amidine, guanidine, sulfonamide, phosphonamide, phosphoramide, hydrazide and selenol. In certain embodiments -L¹- is conjugated to -D via a functional group of -D selected from the group consisting of carboxylic acid, primary amine, secondary amine, thiol, sulfonic acid, carbonate, carbamate, hydroxyl, aldehyde, ketone, hydrazine, isothiocyanate, phosphoric acid, phosphonic acid, acryloyl, hydroxylamine, sulfate, vinyl sulfone, vinyl ketone, diazoalkane, guanidine, amidine and aziridine. In certain embodiments -L¹- is conjugated to -D via a functional group of -D selected from the group consisting of hydroxyl, primary amine, secondary amine, amidine and carboxylic acid.

In certain embodiments -L¹- is conjugated to -D via a hydroxyl group of -D. In certain embodiments -L¹- is conjugated to -D via a primary amine group of -D. In certain embodiments -L¹- is conjugated to -D via a secondary amine group of -D. In certain embodiments -L¹- is conjugated to -D via a carboxylic acid group of -D. In certain embodiments -L¹- is conjugated to -D via an amidine group of -D.

The moiety -L¹- may be connected to -D through any type of linkage, provided that it is reversible. In certain embodiments -L¹- is connected to -D through a linkage selected from the group consisting of amide, ester, carbamate, acetal, aminal, imine, oxime, hydrazone, disulfide, acylguanidine, acylamidine, carbonate, phosphate, sulfate, urea, hydrazide, thioester, thiophosphate, thiosulfate, sulfonamide, sulfoamidine, sulfaguanidine, phosphoramide, phosphoamidine, phosphoguanidine, phosphonamide, phosphonamidine, phosphonguanidine, phosphonate, borate and imide. In certain embodiments -L¹- is connected to -D through a linkage selected from the group consisting of amide, ester, carbonate, carbamate, acetal, aminal, imine, oxime, hydrazone, disulfide, acylamidine and acylguanidine. In certain embodiments -L¹- is connected to -D through a linkage selected from the group consisting of amide, ester, carbonate, acylamide and carbamate. It is understood that some of these linkages may not be reversible per se, but that in the present invention neighboring groups present in -L¹- render these linkages reversible.

In certain embodiments -L¹- is connected to -D through an ester linkage. In certain embodiments -L¹- is connected to -D through a carbonate linkage. In certain embodiments -L¹ is connected to -D through an acylamidine linkage. In certain embodiments -L¹- is connected to -D through a carbamate linkage. In certain embodiments -L¹- is connected to -D through an amide linkage.

The moiety -L¹- is a linker moiety from which -D is released in its free form, i.e. frequently in the form of D-H or D-OH. Such moieties are also referred to as “prodrug linkers” or “reversible prodrug linkers” and are known in the art, such as for example the reversible linker moieties disclosed in WO 2005/099768 A2, WO 2006/136586 A2, WO 2011/089216 A1, WO 2013/024053 A1, WO 2011/012722 A1, WO 2011/089214 A1, WO 2011/089215 A1, WO 2013/024052 A1 and WO 2013/160340 A1, which are incorporated by reference herewith.

In certain embodiments the moiety -L¹- is as disclosed in WO 2009/095479 A2. Accordingly, in certain embodiments the moiety -L¹- is of formula (I):

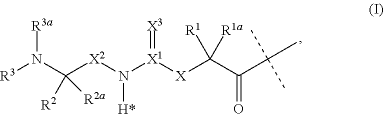

wherein the dashed line indicates the attachment to a nitrogen, hydroxyl or thiol of -D;
—X— is selected from the group consisting of —C(R⁴R4^{a})—, —N(R⁴)—, —O—, —C(R⁴R^{4a})—C(R⁵R^{5a})—, —C(R⁵R^{5a})—C(R⁴R^{4a})—, —C(R⁴R^{4a})—N(R⁶)—, —N(R⁶)—C(R⁴R^{4a})—, —C(R⁴R^{4a})—O—, —O—C(R⁴R^{4a})—, and —C(R⁷R^{7a})—,
X¹ is selected from the group consisting of C and S(O);
X²— is selected from the group consisting of —C(R⁸R^{8a})— and —C(R⁸R^{8a})—C(R⁹R^{9a})—.
═X³ is selected from the group consisting of ═O, ═S, and ═N—CN;
—R¹, —R^{1a}, —R², —R^{2a}, —R⁴, —R^{4a}, —R⁵, —R^{5a}, —R⁶, —R⁸, —R^{8a}, —R⁹ and —R^{9a} are independently selected from the group consisting of —H and C₁₋₆ alkyl;
—R³ and —R^{3a} are independently selected from the group consisting of —H and C₁₋₆ alkyl, provided that in case one or both of —R³ and —R^{3a} are other than —H they are connected to N to which they are attached through an sp³-hybridized carbon atom;
—R⁷ is selected from the group consisting of —N(R¹⁰R^{10a}) and —NR¹⁰—(C═O)—R¹¹;
—R^{7a}, —R¹⁰, —R^{10a} and —R¹¹ are independently selected from the group consisting of —H and C₁₋₆ alkyl;
alternatively, one or more of the pairs —R^{1a}/—R^{4a}, —R^{1a}/—R^{5a}, —R^{1a}/—R^{7a}, —R^{4a}/—R^{5a} and —R^{8a}/—R^{9a} form a chemical bond;
alternatively, one or more of the pairs —R¹/—R^{1a}, —R²/—R^{2a}, —R⁴/—R^{4a}, —R⁵/—R^{5a}, —R⁵/—R^{5a} and —R⁹/—R^{9a} are joined together with the atom to which they are attached to form a C₃₋₁₀ cycloalkyl or 3- to 10-membered heterocyclyl;
alternatively, one or more of the pairs —R¹/—R⁴, —R¹/—R⁵, —R¹/—R⁶, —R¹/—R^{7a}, —R⁴/—R⁵, —R⁴/—R⁶, —R⁸/—R⁹ and —R²/—R³ are joined together with the atoms to which they are attached to form a ring A;
alternatively, R³/R^{3a} are joined together with the nitrogen atom to which they are attached to form a 3- to 10-membered heterocycle;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein -L¹- is substituted with -L²- and wherein -L¹- is optionally further substituted, provided that the hydrogen marked with the asterisk in formula (I) is not replaced by -L²- or a substituent.

The optional further substituents of -L¹- of formula (I) are as described elsewhere herein.

In certain embodiments -L¹- of formula (I) is not further substituted.

It is understood that if —R³/—R^{3a} of formula (I) are joined together with the nitrogen atom to which they are attached to form a 3- to 10-membered heterocycle, only such 3- to 10-membered heterocycles may be formed in which the atoms directly attached to the nitrogen are sp³-hybridized carbon atoms. In other words, such 3- to 10-membered heterocycle formed by —R³/—R^{3a} together with the nitrogen atom to which they are attached has the following structure:

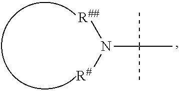

wherein
the dashed line indicates attachment to the rest of -L¹-;
the ring comprises 3 to 10 atoms comprising at least one nitrogen; and
R^{#} and R^{##} represent an sp³-hybridized carbon atom.

It is also understood that the 3- to 10-membered heterocycle may be further substituted.

Exemplary embodiments of suitable 3- to 10-membered heterocycles formed by —R³/—R^{3a} of formula (I) together with the nitrogen atom to which they are attached are the following:

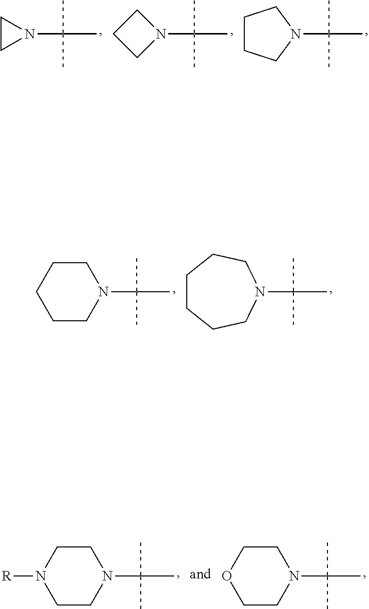

wherein
dashed lines indicate attachment to the rest of the molecule; and
—R is selected from the group consisting of —H and C₁₋₆ alkyl.

-L¹- of formula (I) may optionally be further substituted. In general, any substituent may be used as far as the cleavage principle is not affected, i.e. the hydrogen marked with the asterisk in formula (I) is not replaced and the nitrogen of the moiety

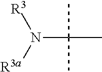

of formula (I) remains part of a primary, secondary or tertiary amine, i.e. —R³ and —R^{3a} are independently of each other —H or are connected to —N< through an sp³-hybridized carbon atom.

In certain embodiments —X— of formula (I) is —C(R⁴R^{4a})—. In certain embodiments —X— of formula (I) is —N(R⁴). In certain embodiments —X— of formula (I) is —O—. In certain embodiments —X— of formula (I) is C(R⁴R^{4a})—C(R⁵R^{5a})—. In certain embodiments —X— of formula (I) is —C(R⁵R^{5a})—C(R⁴R^{4a})—. In certain embodiments —X— of formula (I) is —C(R⁴R^{4a})—N(R⁶)—. In certain embodiments —X— of formula (I) is —N(R⁶)—C(R⁴R^{4a})—. In certain embodiments —X— of formula (I) is —C(R⁴R^{4a})—O—. In certain embodiments —X— of formula (I) is —O—C(R⁴R^{4a})— In certain embodiments —X— of formula (I) is —O—C(R⁴R^{4a})—. In certain embodiments —X— of formula (I) is —C(R⁷R^{7a})—.

In certain embodiments X¹ of formula (I) is C. In certain embodiments X¹ of formula (I) is S(O).

In certain embodiments —X²— of formula (I) is —C(R⁸R^{8a})—. In certain embodiments —X²— of formula (I) is —C(R⁸R^{8a})—.

In certain embodiments ═X³ of formula (I) is ═O. In certain embodiments ═X³ of formula (I) is ═S. In certain embodiments ═X³ of formula (I) is ═N—CN.

In certain embodiments —R¹ of formula (I) is —H. In certain embodiments —R¹ of formula (I) is methyl. In certain embodiments —R¹ of formula (I) is ethyl. In certain embodiments —R^{1a} of formula (I) is —H. In certain embodiments —R^{1a} of formula (I) is methyl. In certain embodiments —R^{1a} of formula (I) is ethyl. In certain embodiments —R² of formula (I) is —H. In certain embodiments —R² of formula (I) is methyl. In certain embodiments —R² of formula (I) is ethyl. In certain embodiments —R^{2a} of formula (I) is —H. In certain embodiments —R^{2a} of formula (I) is methyl. In certain embodiments —R^{2a} of formula (I) is ethyl. In certain embodiments —R³ of formula (I) is —H. In certain embodiments —R³ of formula (I) is methyl. In certain embodiments —R³ of formula (I) is ethyl. In certain embodiments —R^{3a} of formula (I) is —H. In certain embodiments —R^{3a} of formula (I) is methyl. In certain embodiments —R^{3a} of formula (I) is ethyl. In certain embodiments —R⁴ of formula (I) is —H. In certain embodiments —R⁴ of formula (I) is methyl. In certain embodiments —R⁴ of formula (I) is ethyl. In certain embodiments —R^{4a} of formula (I) is —H. In certain embodiments —R^{4a} of formula (I) is methyl. In certain embodiments —R^{4a} of formula (I) is ethyl. In certain embodiments —R⁵ of formula (I) is —H. In certain embodiments —R⁵ of formula (I) is methyl. In certain embodiments —R⁵ of formula (I) is ethyl. In certain embodiments —R^{5a} of formula (I) is —H. In certain embodiments —R^{5a} of formula (I) is methyl. In certain embodiments —R^{5a} of formula (I) is ethyl. In certain embodiments —R⁶ of formula (I) is —H. In certain embodiments —R⁶ of formula (I) is methyl. In certain embodiments —R⁶ of formula (I) is ethyl. In certain embodiments —R⁷ of formula (I) is —N(R¹⁰R^{10a}). In certain embodiments —R⁷ of formula (I) is —NR¹⁰—(C═O)—R¹¹. In certain embodiments —R^{7a} of formula (I) is —H. In certain embodiments —R^{7a} of formula (I) is methyl. In certain embodiments —R^{7a} of formula (I) is ethyl. In certain embodiments —R⁸ of formula (I) is —H. In certain embodiments —R⁸ of formula (I) is methyl. In certain embodiments —R⁸ of formula (I) is ethyl. In certain embodiments —R^{8a} of formula (I) is —H. In certain embodiments —R^{8a} of formula (I) is methyl. In certain embodiments —R^{8a} of formula (I) is ethyl. In certain embodiments —R⁹ of formula (I) is —H. In certain embodiments —R⁹ of formula (I) is methyl. In certain embodiments —R⁹ of formula (I) is ethyl. In certain embodiments —R^{9a} of formula (I) is —H. In certain embodiments —R^{9a} of formula (I) is methyl. In certain embodiments —R^{9a} of formula (I) is ethyl. In certain embodiments —R¹⁰ of formula (I) is —H. In certain embodiments —R¹⁰ of formula (I) is methyl. In certain embodiments —R¹⁰ of formula (I) is ethyl. In certain embodiments —R^{10a} of formula (I) is —H. In certain embodiments —R^{10a} of formula (I) is methyl. In certain embodiments —R^{10a} of formula (I) is ethyl. In certain embodiments —R¹¹ of formula (I) is —H. In certain embodiments —R¹¹ of formula (I) is methyl. In certain embodiments —R¹¹ of formula (I) is ethyl.

In certain embodiments —R¹ of formula (I) is —H, which —H is substituted with -L²-. In certain embodiments —R^{1a} of formula (I) is —H, which —H is substituted with -L²-. In certain embodiments —R² of formula (I) is —H, which —H is substituted with -L²-. In certain embodiments —R^{2a} of formula (I) is —H, which —H is substituted with -L²-. In certain embodiments —R³ of formula (I) is —H, which —H is substituted with -L²-. In certain embodiments —R^{3a} of formula (I) is —H, which —H is substituted with -L²-. In certain embodiments —R⁴ of formula (I) is —H, which —H is substituted with -L²-. In certain embodiments —R⁵ of formula (I) is —H, which —H is substituted with -L²-. In certain embodiments —R^{5a} of formula (I) is —H, which —H is substituted with -L²-. In certain embodiments —R⁶ of formula (I) is —H, which —H is substituted with -L²-. In certain embodiments —R⁷ of formula (I) is —H, which —H is substituted with -L²-. In certain embodiments —R^{7a} of formula (I) is —H, which —H is substituted with -L²-. In certain embodiments —R⁸ of formula (I) is —H, which —H is substituted with -L²-. In certain embodiments —R^{8a} of formula (I) is —H, which —H is substituted with -L²-. In certain embodiments —R⁹ of formula (I) is —H, which —H is substituted with -L²-. In certain embodiments —R^{9a} of formula (I) is —H, which —H is substituted with -L²-. In certain embodiments —R¹⁰ of formula (I) is —H, which —H is substituted with -L²-. In certain embodiments —R¹¹ of formula (I) is —H, which —H is substituted with -L²-.

In certain embodiments —R¹ of formula (I) is —H, which —H is substituted with -L²-. In certain embodiments —R^{1a} of formula (I) is —H, which —H is substituted with -L²-. In certain embodiments —R² of formula (I) is —H, which —H is substituted with -L²-. In certain embodiments —R^{2a} of formula (I) is —H, which —H is substituted with -L²-. In certain embodiments —R³ of formula (I) is —H, which —H is substituted with -L²-. In certain embodiments —R^{3a} of formula (I) is —H, which —H is substituted with -L²-. In certain embodiments —R⁴ of formula (I) is —H, which —H is substituted with -L²-. In certain embodiments —R⁵ of formula (I) is —H, which —H is substituted with -L²-. In certain embodiments —R^{5a} of formula (I) is —H, which —H is substituted with -L²-. In certain embodiments —R⁶ of formula (I) is —H, which —H is substituted with -L²-. In certain embodiments —R⁷ of formula (I) is —H, which —H is substituted with -L²-. In certain embodiments —R^{7a} of formula (I) is —H, which —H is substituted with -L²-. In certain embodiments —R⁸ of formula (I) is —H, which —H is substituted with -L²-. In certain embodiments —R^{8a} of formula (I) is —H, which —H is substituted with -L²-. In certain embodiments —R⁹ of formula (I) is —H, which —H is substituted with -L²-. In certain embodiments —R^{9a} of formula (I) is —H, which —H is substituted with -L²-. In certain embodiments —R¹⁰ of formula (I) is —H, which —H is substituted with -L²-. In certain embodiments —R¹¹ of formula (I) is —H, which —H is substituted with -L²-.

Another moiety -L¹- is disclosed in WO 2016/020373 A1. Accordingly, in certain embodiments the moiety -L¹- is of formula (II):

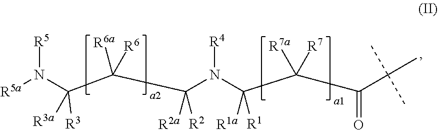

wherein
the dashed line indicates attachment to a primary or secondary amine or hydroxyl of -D by forming an amide or ester linkage, respectively;
—R¹, —R^{1a}, —R², R^{2a},—R³ and —R^{3a} are independently of each other selected from the group consisting of —H, —C(R⁸R^{8a}R^{8b}), —C(═O)R⁸, —C≡N, —C(═NR)R^{8a}, —CR⁸(═CR^{8a}R^{8b}), —C≡CR⁸ and -T;
—R⁴, —R⁵ and —R^{5a} are independently of each other selected from the group consisting of —H, —C(R⁹R^{9a}R^{9b}) and -T;
a1 and a2 are independently of each other 0 or 1;
each —R⁶, —R^{6a}, —R⁷, —R^{7a}, —R⁸, —R^{8a}, R^{8b}, —R⁹, R^{9a}, —R^{9b} are independently of each other selected from the group consisting of —H, halogen, —CN, —COOR¹⁰, —OR¹⁰, —C(O)R¹⁰, —C(O)N(R¹⁰R^{10a}), —S(O)₂N(R¹⁰R^{10a}), —S(O)N(R¹⁰R^{10a}), —S(O)₂R¹⁰, —S(O)R¹⁰, —N(R¹⁰)S(O)₂N(R^{10a}R^{10b}), —SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O)OR^{10a}, —N(R¹⁰)C(O)N(R^{10a}R^{10b}), —OC(O)N(R¹⁰R^{10a}), -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each —R¹⁰, —R^{10a}, —R^{10b} is independently selected from the group consisting of —H, -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T is independently optionally substituted with one or more —R¹¹, which are the same or different;
each —R¹¹ is independently of each other selected from halogen, —CN, oxo (═O), —COOR¹³, —OR¹³, —C(O)R¹³, —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{13a}, —N(R¹³)C(O)OR^{13a}, —N(R¹³)C(O)N(R^{13a}R^{13b}), —OC(O)N(R¹³R^{13a}), and C₁₋₆ alkyl; wherein C₁₋₆ alkyl is optionally substituted with one or more halogen, which are the same or different;
each —R¹², —R^{12a}, —R¹³, —R^{13a}, —R^{13b} is independently selected from the group consisting of —H, and C₁₋₆ alkyl; wherein C₁₋₆ alkyl is optionally substituted with one or more halogen, which are the same or different;
optionally, one or more of the pairs —R¹/—R^{1a}, —R²/—R^{2a}, —R³/—R^{3a}, —R⁶/—R^{6a}, —R⁷/—R^{7a} are joined together with the atom to which they are attached to form a C₃₋₁₀ cycloalkyl or a 3- to 10-membered heterocyclyl;
optionally, one or more of the pairs —R¹/—R², —R¹/—R³, —R¹/—R⁴, —R¹/—R⁵, —R¹/—R⁶, —R¹/—R⁷, —R²/—R³, —R²/—R⁴, —R²/—R⁵, —R²/—R⁶, —R²/—R⁷, —R³/—R⁴, —R³/—R⁵, —R³/—R⁶, —R³/—R⁷, —R⁴/—R⁵, —R⁴/—R⁶, —R⁴/—R⁷, —R⁵/—R⁶, —R⁵/—R⁷, —R⁶/—R⁷ are joined together with the atoms to which they are attached to form a ring A;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein -L¹- is substituted with -L²- and wherein -L¹- is optionally further substituted.

The optional further substituents of -L¹- of formula (II) are as described elsewhere herein.

In certain embodiments -L¹- of formula (II) is not further substituted.

Additional embodiments for -L¹- are disclosed in EP1536334B1, WO2009/009712A1, WO2008/034122A1, WO2009/143412A2, WO2011/082368A2, and U.S. Pat. No. 8,618,124B2, which are herewith incorporated by reference in their entirety.

Further embodiments for -L¹- are disclosed in U.S. Pat. No. 8,946,405B2 and U.S. Pat. No. 8,754,190B2, which are herewith incorporated by reference in their entirety. Accordingly, in certain embodiments -L¹- is of formula (III):

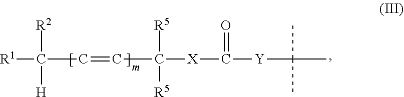

wherein
the dashed line indicates attachment to -D through a functional group of -D selected from the group consisting of —OH, —SH and —NH₂;
m is 0 or 1;
at least one or both of —R¹ and —R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴,
one and only one of —R¹ and —R² is selected from the group consisting of —H, optionally substituted alkyl, optionally substituted arylalkyl, and optionally substituted heteroarylalkyl;
—R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
—R⁴ is selected from the group consisting of optionally substituted alkyl, optionally substituted aryl, optionally substituted arylalkyl, optionally substituted heteroaryl, and optionally substituted heteroarylalkyl;
each —R⁵ is independently selected from the group consisting of —H, optionally substituted alkyl, optionally substituted alkynylalkyl, optionally substituted alkynylalkyl, optionally substituted aryl, optionally substituted arylalkyl, optionally substituted heteroaryl and optionally substituted heteroarylalkyl;
—R⁹ is selected from the group consisting of —H and optionally substituted alkyl;
—Y— is absent and —X— is —O— or —S—; or
—Y— is —N(Q)CH₂— and —X— is —O—;
Q is selected from the group consisting of optionally substituted alkyl, optionally substituted aryl, optionally substituted arylalkyl, optionally substituted heteroaryl and optionally substituted heteroarylalkyl;
optionally, —R¹ and —R² may be joined to form a 3 to 8-membered ring; and
optionally, both —R⁹ together with the nitrogen to which they are attached form a heterocyclic ring; and
wherein -L¹- is substituted with -L²- and wherein -L¹- is optionally further substituted.

Only in the context of formula (III) the terms used have the following meaning: The term “alkyl” as used herein includes linear, branched or cyclic saturated hydrocarbon groups of 1 to 8 carbon atoms, or in some embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, preferably 6 to 10 carbons, including groups such as phenyl, naphthyl, and anthracenyl. The term “heteroaryl” includes aromatic rings comprising 3 to 15 carbons containing at least one N, O or S atom, preferably 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In some instance, alkenyl, alkynyl, aryl or heteroaryl moieties may be coupled to the remainder of the molecule through an alkylene linkage. Under those circumstances, the substituent will be referred to as alkynylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted. Optional substituents on any group, including the above, include halo, nitro, cyano, —OR, —SR, —NR₂, —OCOR, —NRCOR, —COOR, —CONR₂, —SOR, —SO₂R, —SONR₂, —SO₂NR₂, wherein each R is independently alkyl, alkenyl, alkynyl, aryl or heteroaryl, or two R groups taken together with the atoms to which they are attached form a ring.

Another embodiment for -L¹- is disclosed in WO2013/036857A1, which is herewith incorporated by reference in its entirety. Accordingly, in certain embodiments -L¹- is of formula (IV):

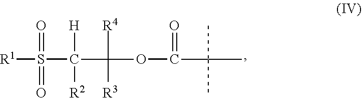

wherein
the dashed line indicates attachment to -D through an amine functional group of -D;
—R¹ is selected from the group consisting of optionally substituted C₁-C₆ linear, branched, or cyclic alkyl; optionally substituted aryl; optionally substituted heteroaryl; alkoxy; and —NR⁵₂;
—R² is selected from the group consisting of —H; optionally substituted C₁-C₆ alkyl; optionally substituted aryl; and optionally substituted heteroaryl;
—R³ is selected from the group consisting of —H; optionally substituted C₁-C₆ alkyl; optionally substituted aryl; and optionally substituted heteroaryl;
—R⁴ is selected from the group consisting of —H; optionally substituted C₁-C₆ alkyl; optionally substituted aryl; and optionally substituted heteroaryl;
each —R⁵ is independently of each other selected from the group consisting of —H; optionally substituted C₁-C₆ alkyl; optionally substituted aryl; and optionally substituted heteroaryl; or when taken together two —R⁵ can be cycloalkyl or cycloheteroalkyl; and
wherein -L¹- is substituted with -L²- and wherein -L¹- is optionally further substituted.

Only in the context of formula (IV) the terms used have the following meaning:

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 C.

“Aryl” includes aromatic hydrocarbon groups of 6-18 carbons, preferably 6-10 carbons, including groups such as phenyl, naphthyl, and anthracene “Heteroaryl” includes aromatic rings comprising 3-15 carbons containing at least one N, O or S atom, preferably 3-7 carbons containing at least one N, O or S atom, including groups such as pyrrolyl, pyridyl, pyrimidinyl, imidazolyl, oxazolyl, isoxazolyl, thiszolyl, isothiazolyl, quinolyl, indolyl, indenyl, and similar.

The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketne; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

A further embodiment for -L¹- is disclosed in U.S. Pat. No. 7,585,837B2, which is herewith incorporated by reference in its entirety. Accordingly, in certain embodiments -L¹- is of formula (V):

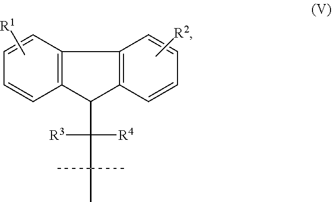

wherein
the dashed line indicates attachment to -D through an amine functional group of -D; R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl; and
wherein -L¹- is substituted with -L²- and wherein -L¹- is optionally further substituted.

Suitable substituents for formulas (V) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Only in the context of formula (V) the terms used have the following meaning:

The terms “alkyl”, “alkoxy”, “alkoxyalkyl”, “aryl”, “alkaryl” and “aralkyl” mean alkyl radicals of 1-8, preferably 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

In certain embodiments -L¹- of formula (V) is not further substituted.

In certain embodiments -L¹- is as disclosed in WO2002/089789A1, which is herewith incorporated by reference in its entirety. Accordingly, in certain embodiments -L¹- is of formula (VI):

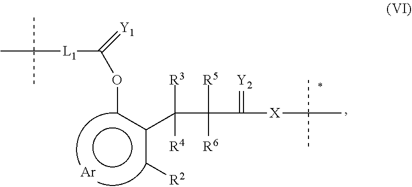

wherein
the dashed line indicates attachment to -D through an amine functional group of -D;
L₁ is a bifunctional linking group,
Y₁ and Y₂ are independently O, S or NR⁷;
R², R³, R⁴, R⁵, R⁶ and R⁷ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
Ar is a moiety which when included in formula (VI) forms a multisubstituted aromatic hydrocarbon or a multi-substituted heterocyclic group;
X is a chemical bond or a moiety that is actively transported into a target cell, a hydrophobic moiety, or a combination thereof,
y is 0 or 1; and
wherein -L¹- is substituted with -L²- and wherein -L¹- is optionally further substituted.

Only in the context of formula (VI) the terms used have the following meaning:

The term “alkyl” shall be understood to include, e.g. straight, branched, substituted C₁₋₁₂ alkyls, including alkoxy, C₃₋₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substituted cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophene; alkoxy includes moeities such as methoxy; and phenoxy includes moieties such as 3-nitrophenoxy. Halo—shall be understood to include fluoro, chloro, iodo and bromo.

In certain embodiments -L¹- of formula (VI) is not further substituted.

In certain embodiments -L¹- comprises a substructure of formula (VII)

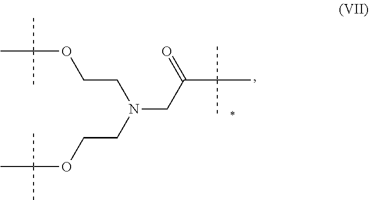

wherein
the dashed line marked with the asterisk indicates attachment to a nitrogen of -D by forming an amide bond;
the unmarked dashed lines indicate attachment to the remainder of -L¹-; and wherein -L¹- is substituted with -L²- and wherein -L¹- is optionally further substituted.

The optional further substituents of -L¹- of formula (VII) are as described elsewhere herein.

In certain embodiments -L¹- of formula (VII) is not further substituted.

In certain embodiments -L¹- comprises a substructure of formula (VIII)

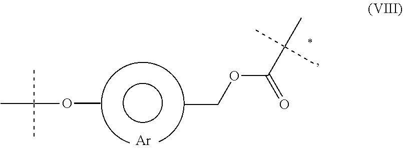

wherein
the dashed line marked with the asterisk indicates attachment to a nitrogen of -D by forming a carbamate bond;
the unmarked dashed lines indicate attachment to the remainder of -L¹-; and wherein -L¹- is substituted with -L²- and wherein -L¹- is optionally further substituted.

The optional further substituents of -L¹- of formula (VIII) are as described above.

In certain embodiments -L¹- of formula (VIII) is not further substituted.

In one embodiment -L¹- is of formula (VIII-a):

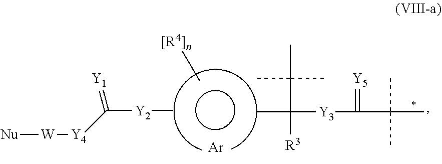

wherein

the dashed line marked with the asterisk indicates attachment to a nitrogen of -D and the unmarked dashed line indicates attachment to -L²-;
n is 0, 1, 2, 3, or 4;
═Y₁, ═Y₅ are independently of each other selected from the group consisting of ═O and ═S;
-Y₂— is selected from the group consisting of —O— and —S—;
-Y₃— is selected from the group consisting of —O— and —S—;
-Y₄— is selected from the group consisting of —O—, —NR⁵— and —C(R⁶R^{6a})—;
—R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
-W- is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
-Nu is a nucleophile selected from the group consisting of —N(R⁷R^{7a}), —N(R⁷OH), —N(R⁷)—N(R^{7a}R^{7b}), —S(R⁷), —COOH,

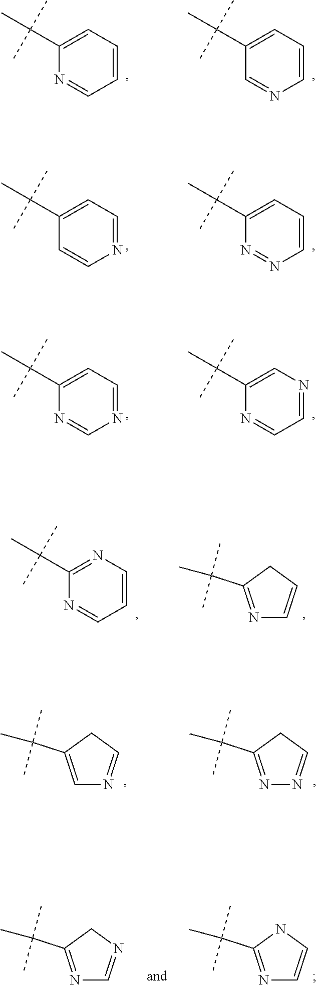

—Ar— is selected from the group consisting of

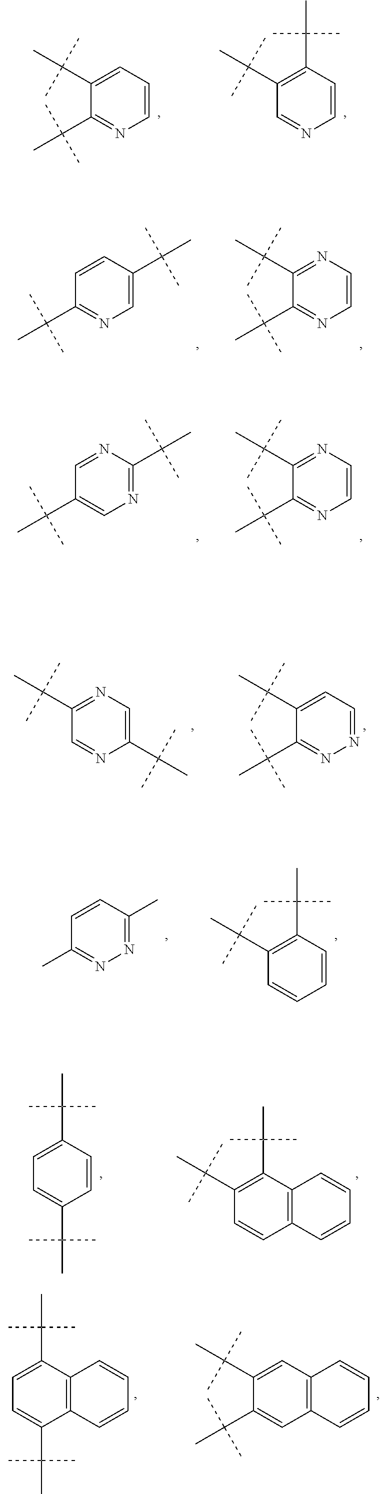

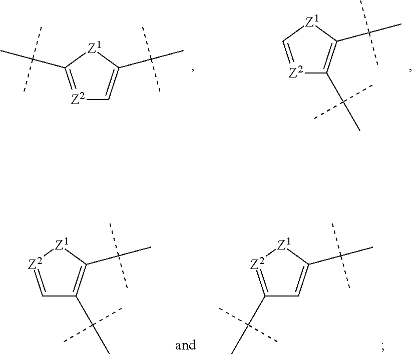

wherein

dashed lines indicate attachment to the remainder of -L¹-,

-Z¹- is selected from the group consisting of —O—, —S— and —N(R⁷)—, and

-Z²— is —N(R⁷)—; and

—R⁷, —R^{7a}, —R^{7b} are independently of each other selected from the group consisting of —H, C₁₋₆ alkyl, C2-6 alkenyl and C2-6 alkynyl;

wherein -L¹- is optionally further substituted.

In one embodiment -L¹- of formula (VIII-a) is not further substituted.

In another embodiment -L¹- is of formula (VIII-b):

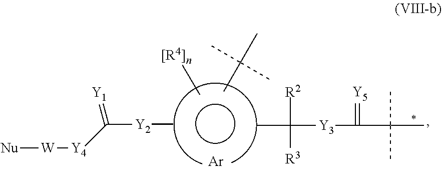

wherein

the dashed line marked with the asterisk indicates attachment to a nitrogen of -D and the unmarked dashed line indicates attachment to -L²-;
n is 0, 1, 2, 3, or 4;
═Y₁, ═Y₅ are independently of each other selected from the group consisting of ═O and ═S;
-Y₂— is selected from the group consisting of —O— and —S—;
-Y₃— is selected from the group consisting of —O— and —S—;
-Y₄— is selected from the group consisting of —O—, —NR⁵— and —C(R⁶R^{6a})—;
—R², —R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
-W- is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
-Nu is a nucleophile selected from the group consisting of —N(R⁷R^{7a}), —N(R⁷OH), —N(R⁷)—N(R^{7a}R^{7b}), —S(R⁷), —COOH,

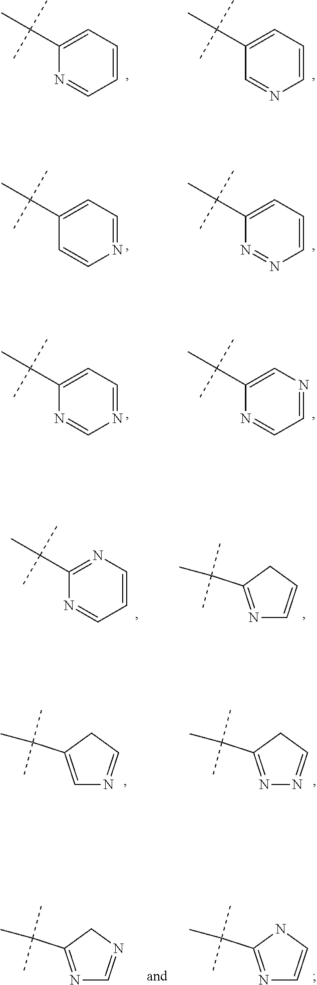

—Ar— is selected from the group consisting of

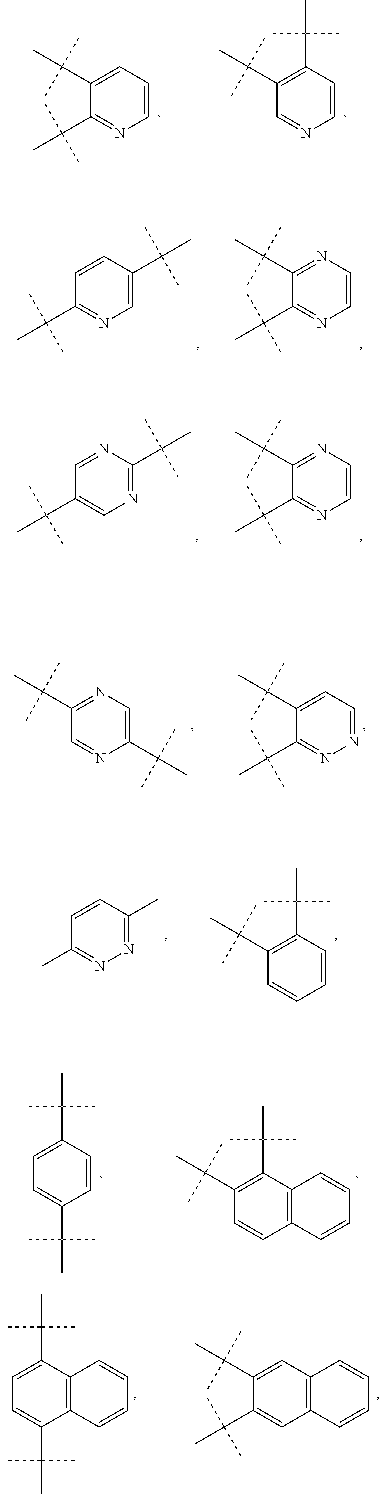

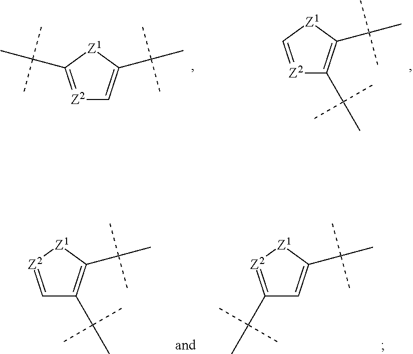

wherein

dashed lines indicate attachment to the remainder of -L¹-,

-Z¹— is selected from the group consisting of —O—, —S— and —N(R⁷)—, and

-Z²— is —N(R⁷)—; and

—R⁷, —R^{7a}, —R^{7b} are independently of each other selected from the group consisting of —H, C₁₋₆ alkyl, C₂₋₆ alkenyl and C₂₋₆ alkynyl;

wherein -L¹- is optionally further substituted.

In one embodiment -L¹- of formula (VIII-b) is not further substituted.

In certain embodiments -L¹- is of formula (IXi)

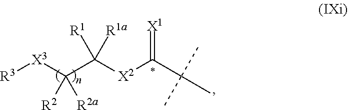

wherein
the dashed line indicates the attachment to the π-electron-pair-donating heteroaromatic N of -D;
n is an integer selected from the group consisting of 0, 1, 2, 3 and 4;
═X¹ is selected from the group consisting of ═O, ═S and ═N(R⁴);
—X²— is selected from the group consisting of —O—, —S—, —N(R⁵)— and —C(R⁶)(R^{6a})—;
—X³- is selected from the group consisting of

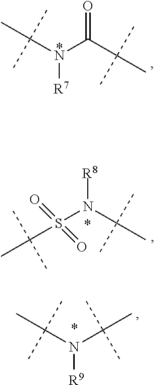

—C(R¹⁰)(R^{10a})—, —C(R¹¹)(R^{11a})—C(R¹²)(R^{12a})—, —O— and —C(O)—;
—R¹, —R^{1a}, —R⁶, —R^{6a}, —R¹⁰, —R^{10a}, —R¹¹, —R^{1a}, —R¹², —R^{12a} and each of —R² and —R^{2a} are independently selected from the group consisting of —H, —C(O)OH, halogen, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
—R³, —R⁴, —R⁵, —R⁷, —R⁸ and —R⁹ are independently selected from the group consisting of —H, -T, —CN, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
   each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
   wherein each T is independently optionally substituted with one or more —R¹³, which are the same or different;
   wherein —R¹³ is selected from the group consisting of —H, —NO₂, —OCH₃, —CN, —N(R¹⁴)(R^{14a}), —OH, —C(O)OH and C₁₋₆ alkyl; wherein C₁₋₆ alkyl is optionally substituted with one or more halogen, which are the same or different;
   wherein —R¹⁴ and —R^{14a} are independently selected from the group consisting of —H and C₁₋₆ alkyl; wherein C₁₋₆ alkyl is optionally substituted with one or more halogen, which are the same or different;
optionally, one or more of the pairs —R¹/—R^{1a}, —R²/—R^{2a}, two adjacent —R², —R⁶/—R^{6a}, —R¹⁰/—R^{10a}, —R¹¹/—R^{11a}, —R¹²/—R^{12a} and —R³/—R⁹ are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁵, —R¹/—R⁶, —R¹/—R⁹, —R¹/—R¹⁰, —R²/—R⁵, —R³/—R^{6a}, —R⁴/—R⁵, —R⁴/—R⁶, —R⁵/—R¹⁰, —R⁶/—R¹⁰ and —R¹¹/—R¹² are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, 3 and 4;
optionally, two adjacent —R² form a carbon-carbon double bond provided that n is selected from the group consisting of 2, 3 and 4;
provided that if —X²— is —N(R⁵)—, —X³— is selected from the group consisting of

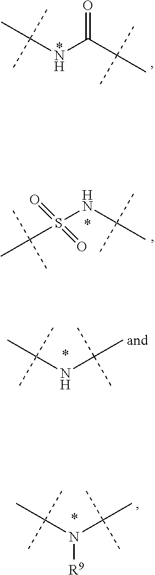

and the distance between the nitrogen atom marked with an asterisk and the carbon atom marked with an asterisk in formula (IXi) is 5, 6 or 7 atoms and if present the carbon-carbon double bond formed between —R¹ and —R² or two adjacent —R² is in a cis configuration; and
wherein -L¹- is substituted with -L²- and wherein -L¹- is optionally further substituted.

In certain embodiments -L¹- is of formula (IX)

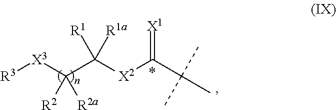

wherein
the dashed line indicates the attachment to a π-electron-pair-donating heteroaromatic N of -D;
n is an integer selected from the group consisting of 0, 1, 2, 3 and 4;
═X¹ is selected from the group consisting of ═O, ═S and ═N(R⁴);
—X²- is selected from the group consisting of —O—, —S—, —N(R⁵)— and —C(R⁶)(R^{6a})—;
—X³- is selected from the group consisting of

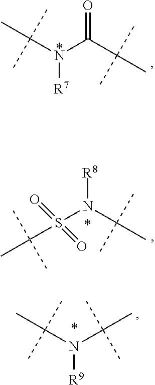

—C(R¹⁰)(R^{10a})—, —C(R¹¹)(R^{11a})—C(R¹²)(R^{12a})—, —O— and —C(O)—;
—R¹, —R^{1a}, —R⁶, —R^{6a}, —R¹⁰, —R^{10a}, —R¹¹, —R^{1a}, —R¹², —R^{12a} and each of —R² and —R^{2a} are independently selected from the group consisting of —H, —C(O)OH, halogen, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
—R³, —R⁴, —R⁵, —R⁷, —R⁸ and —R⁹ are independently selected from the group consisting of —H, -T, —CN, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
   each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R¹³, which are the same or different;
   wherein —R¹³ is selected from the group consisting of —H, —NO₂, —OCH₃, —CN, —N(R¹⁴)(R^{14a}), —OH, —C(O)OH and C₁₋₆ alkyl; wherein C₁₋₆ alkyl is optionally substituted with one or more halogen, which are the same or different;
   wherein —R¹⁴ and —R^{14a} are independently selected from the group consisting of —H and C₁₋₆ alkyl; wherein C₁₋₆ alkyl is optionally substituted with one or more halogen, which are the same or different;
optionally, one or more of the pairs —R¹/—R^{1a}, —R²/—R^{2a}, two adjacent R², —R⁶/—R^{6a}, —R¹⁰/—R^{10a}, —R¹¹/—R^{11a} and —R¹²/—R^{12a} are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁵, —R¹/—R⁶, —R¹/—R⁹, —R¹/—R¹⁰, —R³/—R^{6a}, —R⁴/—R⁵, —R⁴/—R⁶, —R⁵/—R¹⁰, and —R⁶/—R¹⁰ are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, 3 and 4;
optionally, two adjacent —R² form a carbon-carbon double bond provided that n is selected from the group consisting of 2, 3 and 4;
provided that if —X²— is —N(R⁵)—, —X³— is selected from the group consisting of

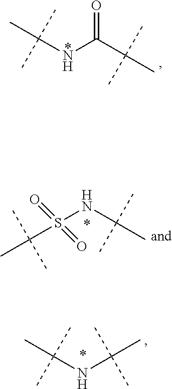

and the distance between the nitrogen atom marked with an asterisk and the carbon atom marked with an asterisk in formula (IX) is 5, 6 or 7 atoms and if present the carbon-carbon double bond formed between —R¹ and —R² or two adjacent —R² is in a cis configuration; and

wherein -L¹- is substituted with -L²- and wherein -L¹- is optionally further substituted.

It is understood that two adjacent —R² in formula (IXi) or (IX) can only exist if n is at least 2.

It is understood that the expression “distance between the nitrogen atom marked with an asterisk and the carbon atom marked with an asterisk” refers to the total number of atoms in the shortest distance between the nitrogen and carbon atoms marked with the asterisk and also includes the nitrogen and carbon atoms marked with the asterisk. For example, in the structure below, n is 1 and the distance between the nitrogen marked with an asterisk and the carbon marked with an asterisk is 5:

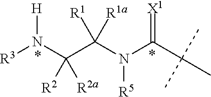

and in the structure below, n is 2, —R¹ and —R^{1a} form a cyclohexyl and the distance between the nitrogen marked with an asterisk and the carbon marked with an asterisk is 6:

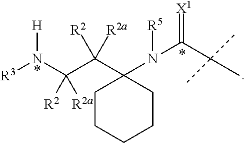

The optional further substituents of -L¹- of formula (IXi) or (IX) are as described elsewhere herein.

In certain embodiments -L¹- of formula (IXi) or (IX) is not further substituted.

In certain embodiments ═X¹ of formula (IXi) or (IX) is ═O. In certain embodiments ═X¹ of formula (IXi) or (IX) is ═S. In certain embodiments ═X¹ of formula (IXi) or (IX) is ═N(R⁴).

In certain embodiments —X²— of formula (IXi) or (IX) is —O—. In certain embodiments —X²— of formula (IXi) or (IX) is —S—. In certain embodiments —X²— of formula (IXi) or (IX) is —N(R⁵)—. In certain embodiments, —X²— of formula (TXi) or (IX) is —C(R⁶)(R^{6a})—.

In certain embodiments —X³— of formula (IXi) or (IX) is

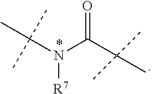

In certain embodiments —X³— of formula (IXi) or (IX) is

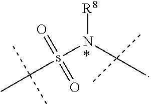

In certain embodiments —X³— of formula (IXi) or (IX) is

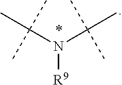

In certain embodiments —X³— of formula (IXi) or (IX) is —C(R¹⁰)(R^{10a})—. In certain embodiments —X³— of formula (IXi) or (IX) is —C(R¹¹)(R^{11a})—C(R¹²)(R^{12a})—. In certain embodiments —X³— of formula (IXi) or (IX) is —O—. In certain embodiments —X³— of formula (IXi) or (IX) is —C(O)—.

In certain embodiments —X²— of formula (IXi) or (IX) is —N(R⁵)—, —X³— is

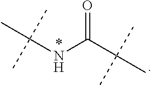

and the distance between the nitrogen atom marked with an asterisk and the carbon atom marked with an asterisk in formula (IXi) or (IX) is 5 atoms.

In certain embodiments —X²— of formula (IXi) or (IX) is —N(R⁵)—, —X³- is

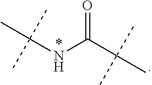

and the distance between the nitrogen atom marked with an asterisk and the carbon atom marked with an asterisk in formula (IXi) or (IX) is 6 atoms.

In certain embodiments —X²— of formula (IXi) or (IX) is —N(R⁵)—, —X³— is

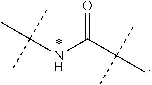

and the distance between the nitrogen atom marked with an asterisk and the carbon atom marked with an asterisk in formula (IXi) or (IX) is 7 atoms.

In certain embodiments —X²— of formula (IXi) or (IX) is —N(R⁵)—, —X³- is

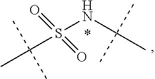

and the distance between the nitrogen atom marked with an asterisk and the carbon atom marked with an asterisk in formula (IXi) or (IX) is 5 atoms.

In certain embodiments —X²— of formula (IXi) or (IX) is —N(R⁵)—, —X³— is

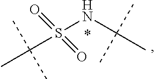

and the distance between the nitrogen atom marked with an asterisk and the carbon atom marked with an asterisk in formula (IXi) or (IX) is 6 atoms.

In certain embodiments —X²— of formula (IXi) or (IX) is —N(R⁵)—, —X³— is

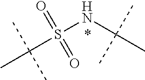

and the distance between the nitrogen atom marked with an asterisk and the carbon atom marked with an asterisk in formula (IXi) or (IX) is 7 atoms.

In certain embodiments —X²— of formula (IXi) or (IX) is —N(R⁵)—, —X³- is

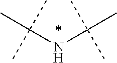

and the distance between the nitrogen atom marked with an asterisk and the carbon atom marked with an asterisk in formula (IXi) or (IX) is 5 atoms.

In certain embodiments —X²— of formula (IXi) or (IX) is —N(R⁵)—, —X³— is

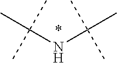

and the distance between the nitrogen atom marked with an asterisk and the carbon atom marked with an asterisk in formula (IXi) or (IX) is 6 atoms.

In certain embodiments —X²— of formula (IXi) or (IX) is —N(R⁵)—, —X³— is

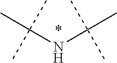

and the distance between the nitrogen atom marked with an asterisk and the carbon atom marked with an asterisk in formula (IXi) or (IX) is 7 atoms.

In certain embodiments, —X²— of formula (IXi) is —N(R⁵)—, —X³— is

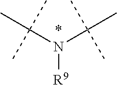

and the distance between the nitrogen atom marked with an asterisk and the carbon atom marked with an asterisk in formula (I) is 5 atoms.

In certain embodiments, —X²— of formula (IXi) is —N(R⁵)—, —X³— is

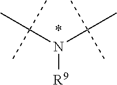

and the distance between the nitrogen atom marked with an asterisk and the carbon atom marked with an asterisk in formula (I) is 6 atoms.

In certain embodiments, —X²— of formula (IXi) is —N(R⁵)—, —X³— is

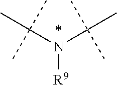

and the distance between the nitrogen atom marked with an asterisk and the carbon atom marked with an asterisk in formula (I) is 7 atoms.

In certain embodiments ═X¹ of formula (IXi) or (IX) is ═O, —X²— of formula (IXi) or (IX) is —C(R⁶)(R^{6a})—, —X³— of formula (IXi) or (IX) is

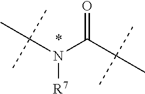

and —R³ of formula (IXi) or (IX) does not comprise an amine.

In certain embodiments —R¹, —R^{1a}, —R⁶, —R¹⁰, —R^{10a}, —R¹¹, —R¹¹, —R¹², —R^{12a} and each of —R² and —R^{2a} of formula (IXi) or (IX) are independently selected from the group consisting of —H, —C(O)OH, halogen, —CN, —OH, C₁₋₆ alkyl, C₂₋₆ alkenyl and C₂₋₆ alkynyl.

In certain embodiments —R¹ of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R¹ of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R¹ of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R¹ of formula (IXi) or (IX) is selected from the group consisting of —H, —C(O)OH, —OH and C₁₋₆ alkyl. In certain embodiments —R¹ of formula (IXi) or (IX) is —H. In certain embodiments —R¹ of formula (IXi) or (IX) is —C(O)OH. In certain embodiments —R¹ of formula (IXi) or (IX) is halogen. In certain embodiments —R¹ of formula (IXi) or (IX) is —F. In certain embodiments —R¹ of formula (IXi) or (IX) is —CN. In certain embodiments —R¹ of formula (IXi) or (IX) is —OH. In certain embodiments —R¹ of formula (TXi) or (IX) is C₁₋₆ alkyl. In certain embodiments —R¹ of formula (TXi) or (IX) is C₂₋₆ alkenyl.

In certain embodiments —R¹ of formula (IXi) or (IX) is C₂₋₆ alkynyl. In certain embodiments —R¹ of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl. In this case it is understood that —R¹/—R^{1a} may optionally be joined together with the atom to which they are attached to form a C₃₋₁₀ cycloalkyl and that one or more of the pairs —R¹/—R², —R¹/—R⁵, —R¹/—R⁶, —R¹/—R⁹ and —R¹/—R¹⁰ may optionally be joined together with the atoms to which they are attached to form a ring -A-, wherein -A- is used as defined for formula (IXi) or (IX).

In certain embodiments —R^{1a} of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R^{1a} of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R^{1a} of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R^{1a} of formula (IXi) or (IX) is selected from the group consisting of —H, —C(O)OH, —OH and C₁₋₆ alkyl. In certain embodiments —R^{1a} of formula (IXi) or (IX) is —H. In certain embodiments —R^{1a} of formula (IXi) or (IX) is —C(O)OH. In certain embodiments, —R^{1a} of formula (IXi) or (IX) is halogen. In certain embodiments —R^{1a} of formula (IXi) or (IX) is —F. In certain embodiments —R^{1a} of formula (IXi) or (IX) is —CN. In certain embodiments —R^{1a} of formula (IXi) or (IX) is —OH. In certain embodiments —R^{1a} of formula (IXi) or (IX) is C₁₋₆ alkyl. In certain embodiments —R^{1a} of formula (IXi) or (IX) is C₂₋₆ alkenyl. In certain embodiments —R^{1a} of formula (IXi) or (IX) is C₂₋₆ alkynyl. In certain embodiments —R^{1a} of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R⁶ of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R⁶ of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R⁶ of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R⁶ of formula (IXi) or (IX) is selected from the group consisting of —H, —C(O)OH, —OH and C₁₋₆ alkyl. In certain embodiments —R⁶ of formula (IXi) or (IX) is —H. In certain embodiments —R⁶ of formula (IXi) or (IX) is —C(O)OH. In certain embodiments —R⁶ of formula (IXi) or (IX) is halogen. In certain embodiments —R⁶ of formula (IXi) or (IX) is —F. In certain embodiments —R⁶ of formula (IXi) or (IX) is —CN. In certain embodiments —R⁶ of formula (IXi) or (IX) is —OH. In certain embodiments —R⁶ of formula (IXi) or (IX) is C₁₋₆ alkyl. In certain embodiments —R⁶ of formula (IXi) or (IX) is C₂₋₆ alkenyl. In certain embodiments —R⁶ of formula (IXi) or (IX) is C₂₋₆ alkynyl. In certain embodiments —R⁶ of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R^{6a} of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R^{6a} of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R^{6a} of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R^{6a} of formula (IXi) or (IX) is selected from the group consisting of —H, —C(O)OH, —OH and C₁₋₆ alkyl. In certain embodiments —R^{6a} of formula (IXi) or (IX) is —H. In certain embodiments —R^{6a} of formula (IXi) or (IX) is —C(O)OH. In certain embodiments, —R^{6a} of formula (IXi) or (IX) is halogen. In certain embodiments —R^{6a} of formula (IXi) or (IX) is —F. In certain embodiments —R^{6a} of formula (IXi) or (IX) is —CN. In certain embodiments —R^{6a} of formula (IXi) or (IX) is —OH. In certain embodiments —R^{6a} of formula (IXi) or (IX) is C₁₋₆ alkyl. In certain embodiments —R^{6a} of formula (IXi) or (IX) is C₂₋₆ alkenyl. In certain embodiments —R^{6a} of formula (IXi) or (IX) is C₂₋₆ alkynyl. In certain embodiments —R^{6a} of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R¹ of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R¹⁰ of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R¹⁰ of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R¹⁰ of formula (IXi) or (IX) is selected from the group consisting of —H, —C(O)OH, —OH and C₁₋₆ alkyl. In certain embodiments —R¹⁰ of formula (IXi) or (IX) is —H. In certain embodiments —R¹⁰ of formula (IXi) or (IX) is —C(O)OH. In certain embodiments —R¹⁰ of formula (IXi) or (IX) is halogen. In certain embodiments —R¹⁰ of formula (IXi) or (IX) is —F. In certain embodiments —R¹⁰ of formula (IXi) or (IX) is —CN. In certain embodiments —R¹⁰ of formula (IXi) or (IX) is —OH. In certain embodiments —R¹⁰ of formula (IXi) or (IX) is C₁₋₆ alkyl. In certain embodiments —R¹⁰ of formula (IXi) or (IX) is C₂₋₆ alkenyl. In certain embodiments —R¹⁰ of formula (IXi) or (IX) is C₂₋₆ alkynyl. In certain embodiments —R¹⁰ of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R^{10a} of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R^{10a} of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R^{10a} of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R^{10a} of formula (IXi) or (IX) is selected from the group consisting of —H, —C(O)OH, —OH and C₁₋₆ alkyl. In certain embodiments —R^{10a} of formula (IXi) or (IX) is —H. In certain embodiments —R^{10a} of formula (IX) is —C(O)OH. In certain embodiments —R^{10a} of formula (IXi) or (IX) is halogen. In certain embodiments —R^{10a} of formula (IXi) or (IX) is —F. In certain embodiments —R^{10a} of formula (IXi) or (IX) is —CN. In certain embodiments —R^{10a} of formula (IXi) or (IX) is —OH. In certain embodiments —R^{10a} of formula (IXi) or (IX) is C₁₋₆ alkyl. In certain embodiments —R^{10a} of formula (IXi) or (IX) is C₂₋₆ alkenyl. In certain embodiments —R^{10a} of formula (IXi) or (IX) is C₂₋₆ alkynyl. In certain embodiments —R^{10a} of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R¹¹ of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R¹¹ of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R¹¹ of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R¹¹ of formula (IXi) or (IX) is selected from the group consisting of —H, —C(O)OH, —OH and C₁₋₆ alkyl. In certain embodiments —R¹¹ of formula (IXi) or (IX) is —H. In certain embodiments —R¹¹ of formula (IXi) or (IX) is —C(O)OH. In certain embodiments —R¹¹ of formula (IXi) or (IX) is halogen. In certain embodiments —R¹¹ of formula (IXi) or (IX) is —F. In certain embodiments —R¹¹ of formula (IXi) or (IX) is —CN. In certain embodiments —R¹¹ of formula (IXi) or (IX) is —OH. In certain embodiments —R¹¹ of formula (IXi) or (IX) is C₁₋₆ alkyl. In certain embodiments —R¹¹ of formula (IX) is C₂₋₆ alkenyl. In certain embodiments —R¹¹ of formula (IXi) or (IX) is C₂₋₆ alkynyl. In certain embodiments —R¹¹ of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R^{11a} of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R^{11a} of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R^{11a} of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R^{11a} of formula (IXi) or (IX) is selected from the group consisting of —H, —C(O)OH, —OH and C₁₋₆ alkyl. In certain embodiments —R^{11a} of formula (IXi) or (IX) is —H. In certain embodiments —R^{11a} of formula (IXi) or (IX) is —C(O)OH. In certain embodiments —R^{11a} of formula (IXi) or (IX) is halogen. In certain embodiments —R^{11a} of formula (IXi) or (IX) is —F. In certain embodiments —R^{11a} of formula (IXi) or (IX) is —CN. In certain embodiments —R^{11a} of formula (IXi) or (IX) is —OH. In certain embodiments —R^{11a} of formula (IXi) or (IX) is C₁₋₆ alkyl. In certain embodiments —R^{11a} of formula (IXi) or (IX) is C₂₋₆ alkenyl. In certain embodiments —R^{11a} of formula (IXi) or (IX) is C₂₋₆ alkynyl. In certain embodiments —R^{11a} of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R¹² of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R¹² of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R¹² of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R¹² of formula (IXi) or (IX) is selected from the group consisting of —H, —C(O)OH, —OH and C₁₋₆ alkyl. In certain embodiments —R¹² of formula (IXi) or (IX) is —H. In certain embodiments —R¹² of formula (IXi) or (IX) is —C(O)OH. In certain embodiments —R¹² of formula (IXi) or (IX) is halogen. In certain embodiments —R¹² of formula (IXi) or (IX) is —F. In certain embodiments —R¹² of formula (IXi) or (IX) is —CN. In certain embodiments —R¹² of formula (IXi) or (IX) is —OH. In certain embodiments —R¹² of formula (IXi) or (IX) is C₁₋₆ alkyl. In certain embodiments —R¹² of formula (IXi) or (IX) is C₂₋₆ alkenyl. In certain embodiments —R¹² of formula (IXi) or (IX) is C₂₋₆ alkynyl. In certain embodiments —R¹² of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R^{12a} of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R^{12a} of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R^{12a} of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R^{12a} of formula (IXi) or (IX) is selected from the group consisting of —H, —C(O)OH, —OH and C₁₋₆ alkyl. In certain embodiments —R^{12a} of formula (IXi) or (IX) is —H. In certain embodiments —R^{12a} of formula (IXi) or (IX) is —C(O)OH. In certain embodiments —R^{12a} of formula (IXi) or (IX) is halogen. In certain embodiments —R^{12a} of formula (IXi) or (IX) is —F. In certain embodiments —R^{12a} of formula (IXi) or (IX) is —CN. In certain embodiments —R^{12a} of formula (IXi) or (IX) is —OH. In certain embodiments —R^{12a} of formula (IXi) or (IX) is C₁₋₆ alkyl. In certain embodiments —R^{12a} of formula (IXi) or (IX) is C₂₋₆ alkenyl. In certain embodiments —R^{12a} of formula (IXi) or (IX) is C₂₋₆ alkynyl. In certain embodiments —R^{12a} of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments each of —R² of formula (IXi) or (IX) is independently selected from the group consisting of —H, —C(O)OH, halogen, —CN, —OH, C₁₋₆ alkyl, C₂₋₆ alkenyl and C₂₋₆ alkynyl. In certain embodiments each of —R² of formula (IXi) or (IX) is independently selected from the group consisting of —H, —C(O)OH, —CN, —OH, C₁₋₆ alkyl, C₂₋₆ alkenyl and C₂₋₆ alkynyl. In certain embodiments each of —R² of formula (IXi) or (IX) is independently selected from the group consisting of —H, —C(O)OH, halogen, —OH, C₁₋₆ alkyl, C₂₋₆ alkenyl and C₂₋₆ alkynyl. In certain embodiments each of —R² of formula (IXi) or (IX) is independently selected from the group consisting of —H, —C(O)OH, —OH and C₁₋₆ alkyl. In certain embodiments each of —R² of formula (IXi) or (IX) is —H. In certain embodiments each of —R² of formula (IXi) or (IX) is —C(O)OH. In certain embodiments each of —R² of formula (IXi) or (IX) is halogen. In certain embodiments each of —R² of formula (IXi) or (IX) is —F. In certain embodiments each of —R² of formula (IXi) or (IX) is —CN. In certain embodiments each of —R² of formula (IXi) or (IX) is —OH. In certain embodiments each of —R² of formula (IXi) or (IX) is C₁₋₆ alkyl. In certain embodiments each of —R² of formula (IXi) or (IX) is C₂₋₆ alkenyl.

In certain embodiments each of —R² of formula (IXi) or (IX) is C₂₋₆ alkynyl. In certain embodiments each of —R² of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl. In this case it is understood that one or more of the pairs —R²/—R^{2a} and two adjacent —R² may optionally be joined with the atom to which they are attached to form a C₃₋₁₀ cycloalkyl and that the pair -R²/—R⁵ may optionally be joined together with the atoms to which they are attached to form a ring -A-, wherein -A- is used as defined in formula (IX) or (IXi).

In certain embodiments each of —R^{2a} of formula (IXi) or (IX) is independently selected from the group consisting of —H, —C(O)OH, halogen, —CN, —OH, C₁₋₆ alkyl, C₂₋₆ alkenyl and C₂₋₆ alkynyl. In certain embodiments each of —R^{2a} of formula (IXi) or (IX) is independently selected from the group consisting of —H, —C(O)OH, —CN, —OH, C₁₋₆ alkyl, C₂₋₆ alkenyl and C₂₋₆ alkynyl. In certain embodiments each of —R^{2a} of formula (IXi) or (IX) is independently selected from the group consisting of —H, —C(O)OH, halogen, —OH, C₁₋₆ alkyl, C₂₋₆ alkenyl and C₂₋₆ alkynyl. In certain embodiments each of —R^{2a} of formula (IXi) or (IX) is independently selected from the group consisting of —H, —C(O)OH, —OH and C₁₋₆ alkyl. In certain embodiments each of —R^{2a} of formula (IXi) or (IX) is —H. In certain embodiments each of —R^{2a} of formula (IXi) or (IX) is —C(O)OH. In certain embodiments each of —R^{2a} of formula (IXi) or (IX) is halogen. In certain embodiments each of —R^{2a} of formula (IXi) or (IX) is —F. In certain embodiments each of —R^{2a} of formula (IXi) or (IX) is —CN. In certain embodiments each of —R^{2a} of formula (IXi) or (IX) is —OH. In certain embodiments each of —R^{2a} of formula (IXi) or (IX) is C₁₋₆ alkyl. In certain embodiments each of —R^{2a} of formula (IXi) or (IX) is C₂₋₆ alkenyl. In certain embodiments each of —R^{2a} of formula (IXi) or (IX) is C₂₋₆ alkynyl. In certain embodiments each of —R^{2a} of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R³, —R⁴, —R⁵, —R⁷, —R⁸ and —R⁹ of formula (IXi) or (IX) are independently selected from the group consisting of —H, -T, —CN, C₁₋₆ alkyl, C₂₋₆ alkenyl and C₂₋₆ alkynyl. In certain embodiments —R³, —R⁴, —R⁵, —R⁷, —R⁸ and —R⁹ of formula (IXi) or (IX) are independently selected from the group consisting of —H, -T, —CN, C₁₋₆ alkyl and C₂₋₆ alkenyl. In certain embodiments —R³, —R⁴, —R⁵, —R⁷, —R⁸ and —R⁹ of formula (IXi) or (IX) are independently selected from the group consisting of —H, -T, —CN and C₁₋₆ alkyl. In certain embodiments —R³, —R⁴, —R⁵, —R⁷, —R⁸ and —R⁹ of formula (IXi) or (IX) are independently selected from the group consisting of —H, -T and C₁₋₆ alkyl. In certain embodiments —R³, —R⁴, —R⁵, —R⁷, —R⁸ and —R⁹ of formula (IXi) or (IX) are independently selected from the group consisting of —H and C₁₋₆ alkyl. In certain embodiments —R³ of formula (IXi) or (IX) is selected from the group consisting of —H, -T, —CN, C₁₋₆ alkyl, C₂₋₆ alkenyl and C₂₋₆ alkynyl. In certain embodiments —R³ of formula (IXi) or (IX) is —H. In certain embodiments —R³ of formula (IXi) or (IX) is -T. In certain embodiments —R³ of formula (IXi) or (IX) is —CN. In certain embodiments —R³ of formula (IXi) or (IX) is C₁₋₆ alkyl. In certain embodiments —R³ of formula (IXi) or (IX) is C₂₋₆ alkenyl. In certain embodiments —R³ of formula (IXi) or (IX) is C₂₋₆ alkynyl.

In certain embodiments —R⁴ of formula (IXi) or (IX) is selected from the group consisting of —H, -T, —CN, C₁₋₆ alkyl, C₂₋₆ alkenyl and C₂₋₆ alkynyl. In certain embodiments —R⁴ of formula (IXi) or (IX) is —H. In certain embodiments —R⁴ of formula (IXi) or (IX) is -T. In certain embodiments —R⁴ of formula (IXi) or (IX) is —CN. In certain embodiments —R⁴ of formula (IXi) or (IX) is C₁₋₆ alkyl. In certain embodiments —R⁴ of formula (IXi) or (IX) is C₂₋₆ alkenyl. In certain embodiments —R⁴ of formula (IXi) or (IX) is C₂₋₆ alkynyl.

In certain embodiments —R⁵ of formula (IXi) or (IX) is selected from the group consisting of —H, -T, —CN, C₁₋₆ alkyl, C₂₋₆ alkenyl and C₂₋₆ alkynyl. In certain embodiments —R⁵ of formula (IXi) or (IX) is —H. In certain embodiments —R⁵ of formula (IXi) or (IX) is -T. In certain embodiments —R⁵ of formula (IXi) or (IX) is —CN. In certain embodiments —R⁵ of formula (IXi) or (IX) is C₁₋₆ alkyl. In certain embodiments —R⁵ of formula (IXi) or (IX) is C₂₋₆ alkenyl. In certain embodiments —R⁵ of formula (IXi) or (IX) is C₂₋₆ alkynyl.

In certain embodiments —R⁷ of formula (IXi) or (IX) is selected from the group consisting of —H, -T, —CN, C₁₋₆ alkyl, C₂₋₆ alkenyl and C₂₋₆ alkynyl. In certain embodiments —R⁷ of formula (IXi) or (IX) is —H. In certain embodiments —R⁷ of formula (IXi) or (IX) is -T. In certain embodiments —R⁷ of formula (IXi) or (IX) is —CN. In certain embodiments —R⁷ of formula (IXi) or (IX) is C₁₋₆ alkyl. In certain embodiments —R⁷ of formula (IXi) or (IX) is C₂₋₆ alkenyl. In certain embodiments —R⁷ of formula (IXi) or (IX) is C₂₋₆ alkynyl.

In certain embodiments —R⁸ of formula (IXi) or (IX) is selected from the group consisting of —H, -T, —CN, C₁₋₆ alkyl, C₂₋₆ alkenyl and C₂₋₆ alkynyl. In certain embodiments —R⁸ of formula (IXi) or (IX) is —H. In certain embodiments —R⁸ of formula (IXi) or (IX) is -T. In certain embodiments —R⁸ of formula (IXi) or (IX) is —CN. In certain embodiments —R⁸ of formula (IXi) or (IX) is C₁₋₆ alkyl. In certain embodiments —R⁸ of formula (IXi) or (IX) is C₂₋₆ alkenyl. In certain embodiments —R⁸ of formula (IXi) or (IX) is C₂₋₆ alkynyl.

In certain embodiments —R⁹ of formula (IXi) or (IX) is selected from the group consisting of —H, -T, —CN, C₁₋₆ alkyl, C₂₋₆ alkenyl and C₂₋₆ alkynyl. In certain embodiments —R⁹ of formula (IXi) or (IX) is —H. In certain embodiments —R⁹ of formula (IXi) or (IX) is -T. In certain embodiments —R⁹ of formula (IXi) or (IX) is —CN. In certain embodiments —R⁹ of formula (IXi) or (IX) is C₁₋₆ alkyl. In certain embodiments —R⁹ of formula (IXi) or (IX) is C₂₋₆ alkenyl. In certain embodiments —R⁹ of formula (IXi) or (IX) is C₂₋₆ alkynyl.

In certain embodiments T of formula (IXi) or (IX) is selected from the group consisting of phenyl, naphthyl, indenyl, indanyl, tetralinyl, C3-10 cycloalkyl, 3- to 10-membered heterocyclyl and 8- to 11-membered heterobicyclyl. In certain embodiments T of formula (IXi) or (IX) is phenyl. In certain embodiments T of formula (IXi) or (IX) is naphthyl. In certain embodiments T of formula (IXi) or (IX) is indenyl. In certain embodiments T of formula (IXi) or (IX) is indanyl. In certain embodiments T of formula (IXi) or (IX) is tetralinyl. In certain embodiments T of formula (TXi) or (IX) is C₃₋₁₀ cycloalkyl. In certain embodiments T of formula (TXi) or (IX) is 3- to 10-membered heterocyclyl. In certain embodiments T of formula (IXi) or (IX) is 8- to 11-membered heterobicyclyl.

In certain embodiments T of formula (IXi) or (IX) is substituted with one or more —R¹³ of formula (IXi) or (IX), which are the same or different.

In certain embodiments T of formula (IXi) or (IX) is substituted with one —R¹³ of formula (IXi) or (IX).

In certain embodiments T of formula (IXi) or (IX) is not substituted with —R¹³ of formula (IXi) or (IX).

In certain embodiments —R¹³ of formula (IXi) or (IX) is selected from the group consisting of —H, —NO₂, —OCH₃, —CN, —N(R¹⁴)(R^{14a}), —OH, —C(O)OH and C₁₋₆ alkyl.

In certain embodiments —R¹³ of formula (IXi) or (IX) is —H. In certain embodiments —R¹³ of formula (IXi) or (IX) is —NO₂. In certain embodiments —R¹³ of formula (IXi) or (IX) is —OCH₃. In certain embodiments —R¹³ of formula (IX) is —CN. In certain embodiments —R¹³ of formula (IXi) or (IX) is —N(R¹⁴)(R^{14a}). In certain embodiments —R¹³ of formula (IXi) or (IX) is —OH. In certain embodiments —R¹³ of formula (IXi) or (IX) is —C(O)OH. In certain embodiments —R¹³ of formula (IXi) or (IX) is C₁₋₆ alkyl.

In certain embodiments —R¹⁴ and —R^{14a} of formula (IXi) or (IX) are independently selected from the group consisting of —H and C₁₋₆ alkyl. In certain embodiments —R¹⁴ of formula (IXi) or (IX) is —H. In certain embodiments —R¹⁴ of formula (IXi) or (IX) is C₁₋₆ alkyl. In certain embodiments —R^{14a} of formula (IXi) or (IX) is —H. In certain embodiments —R^{14a} of formula (IXi) or (IX) is C₁₋₆ alkyl.

In certain embodiments, —R³/—R⁹ of formula (IXi) are joined with the nitrogen atom to which they are attached to form a 3- to 10-membered heterocyclyl or an 8- to 11-membered heterobicyclyl. In certain embodiments, —R³/—R⁹ of formula (IXi) are joined with the nitrogen atom to which they are attached to form a 3- to 10-membered heterocyclyl or an 8- to 11-membered heterobicyclyl, wherein the attachment of the 3- to 10-membered heterocyclyl or 8- to 11-membered heterobicyclyl to the rest of the linker moiety of formula (IXi) takes place via a sp³-hybridized nitrogen.

In certain embodiments, —R³/—R⁹ of formula (IXi) are joined with the nitrogen atom to which they are attached to form a ring selected from the group consisting of aziridine, azetidine, pyrroline, imidazoline, pyrazoline, 4-thiazoline, pyrrolidine, imidazolidine, pyrazolidine, oxazolidine, isoxazolidine, thiazolidine, isothiazolidine, thiadiazolidine, piperazine, piperidine, morpholine, triazolidine, tetrazolidine, diazepane, homopiperazine, indoline, benzimidazoline, dihydroquinazoline, dihydroquinoline, tetrahydroquinoline, decahydroquinoline, decahydroisoquinoline, tetrahydroisoquinoline and dihydroisoquinoline. Each hydrogen atom of such rings may be replaced by a substituent as defined above.

In certain embodiments n of formula (IXi) or (IX) is selected from the group consisting of 0, 1, 2 and 3. In certain embodiments n of formula (IXi) or (IX) is selected from the group consisting of 0, 1 and 2. In certain embodiments n of formula (IXi) or (IX) is selected from the group consisting of 0 and 1. In certain embodiments n of formula (IXi) or (IX) is 0. In certain embodiments n of formula (I) is 1. In certain embodiments n of formula (IXi) or (IX) is 2. In certain embodiments n of formula (I) is 3. In certain embodiments n of formula (IXi) or (IX) is 4.

In certain embodiments -L¹- of formula (IXi) or (IX) is connected to -D through a linkage selected from the group consisting of amide, carbamate, dithiocarbamate, O-thiocarbamate, S-thiocarbamate, urea, thiourea, thioamide, amidine and guanidine. It is understood that some of these linkages may not be reversible per se, but that in the present invention neighboring groups present in -L¹-, such as for example amide, primary amine, secondary amine and tertiary amine, render these linkages reversible.

In certain embodiments -L¹- of formula (IXi) or (IX) is conjugated to -D through an amide linkage, i.e. ═X¹ is ═O and —X²— is —C(R⁶)(R^{6a})—.

In certain embodiments -L¹- of formula (IXi) or (IX) is conjugated to -D through a carbamate linkage, i.e. ═X¹ is ═O and —X²— is —O—.

In certain embodiments -L¹- of formula (IXi) or (IX) is conjugated to -D through a dithiocarbamate linkage, i.e. ═X¹ is ═S and —X²— is —S—.

In certain embodiments -L¹- of formula (IXi) or (IX) is conjugated to -D through an O-thiocarbamate linkage, i.e. ═X¹ is ═S and —X²— is —O—.

In certain embodiments -L¹- of formula (IXi) or (IX) is conjugated to -D through a S-thiocarbamate linkage, i.e. ═X¹ is ═O and —X²— is —S—.

In certain embodiments -L¹- of formula (IXi) or (IX) is conjugated to -D through a urea linkage, i.e. ═X¹ is ═O and —X²— is —N(R⁵)—.

In certain embodiments -L¹- of formula (IXi) or (IX) is conjugated to -D through a thiourea linkage, i.e. ═X¹ is ═S and —X²— is —N(R⁵)—.

In certain embodiments -L¹- of formula (IXi) or (IX) is conjugated to -D through a thioamide linkage, i.e. ═X¹ is ═S and —X²— is —C(R⁶)(R^{6a})—.

In certain embodiments -L¹- of formula (IXi) or (IX) is conjugated to -D through an amidine linkage, i.e. ═X¹ is ═N(R⁴) and —X²— is —C(R⁶)(R^{6a})—.

In certain embodiments -L¹- of formula (IXi) or (IX) is conjugated to -D through a guanidine linkage, i.e. ═X¹ is ═N(R⁴) and —X²— is —N(R⁵)—.

In certain embodiments ═X¹ of formula (IXi) or (IX) is ═O and —X²— of formula (IX) is —N(R⁵) with —R⁵ being ethyl. In certain embodiments ═X¹ of formula (IXi) or (IX) is ═O, —X²— of formula (IXi) or (IX) is —N(R⁵) with —R⁵ being ethyl and both —R¹ and —R^{1a} of formula (IXi) or (IX) are —H. In certain embodiments ═X¹ of formula (IXi) or (IX) is ═O, —X²— of formula (IXi) or (IX) is —N(R⁵) with —R⁵ of formula (IXi) or (IX) being ethyl, both —R¹ and —R^{1a} of formula (IX) are —H and n of formula (IXi) or (IX) is 0. In certain embodiments ═X¹ of formula (IXi) or (IX) is ═O, —X²— of formula (IXi) or (IX) is —N(R⁵) with —R⁵ of formula (IXi) or (IX) being ethyl, both —R¹ and —R^{1a} of formula (IXi) or (IX) are —H, n of formula (IXi) or (IX) is 0 and —X³— of formula (IXi) or (IX) is

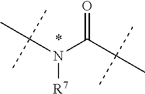

with —R⁷ being —H.

In certain embodiments ═X¹ of formula (IXi) or (IX) is ═O and —X²— of formula (IXi) or (IX) is —N(R⁵) with —R⁵ being ethyl. In certain embodiments ═X¹ of formula (IXi) or (IX) is ═O, —X²— of formula (IXi) or (IX) is —N(R⁵) with —R⁵ being ethyl and both —R¹ and —R^{1a} of formula (IXi) or (IX) are —H. In certain embodiments ═X¹ of formula (IXi) or (IX) is ═O, —X²— of formula (IXi) or (IX) is —N(R⁵) with —R⁵ of formula (IXi) or (IX) being ethyl, both —R¹ and —R^{1a} of formula (IXi) or (IX) are —H and n of formula (IXi) or (IX) is 0. In certain embodiments ═X¹ of formula (IXi) or (IX) is ═O, —X²— of formula (IX) is —N(R⁵) with —R⁵ of formula (IXi) or (IX) being ethyl, both —R¹ and —R^{1a} of formula (IXi) or (IX) are —H, n of formula (IXi) or (IX) is 0 and —X³— of formula (IXi) or (IX) is

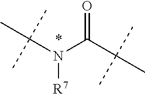

with —R⁷ being —H.

In certain embodiments ═X¹ of formula (IXi) or (IX) is ═O and —X²— of formula (IXi) or (IX) is —N(R⁵) with —R⁵ being methyl. In certain embodiments ═X¹ of formula (IXi) or (IX) is ═O, —X²— of formula (IXi) or (IX) is —N(R⁵) with —R⁵ being methyl, —R¹ of formula (IXi) or (IX) is methyl and —R^{1a} of formula (IXi) or (IX) is —H. In certain embodiments ═X¹ of formula (IXi) or (IX) is ═O, —X²— of formula (IXi) or (IX) is —N(R⁵) with —R⁵ of formula (IXi) or (IX) being methyl, —R¹ of formula (IXi) or (IX) is methyl, —R^{1a} of formula (IXi) or (IX) is —H and n of formula (IX) is 0. In certain embodiments ═X¹ of formula (IXi) or (IX) is ═O, —X²— of formula (IXi) or (IX) is —N(R⁵) with —R⁵ of formula (IXi) or (IX) being methyl, —R¹ of formula (IXi) or (IX) is methyl, —R^{1a} of formula (IXi) or (IX) is —H, n of formula (IXi) or (IX) is 0 and —X³— of formula (IXi) or (IX) is

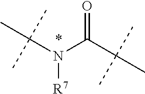

with —R⁷ being —H.

In certain embodiments -L¹- is of formula (IX′):

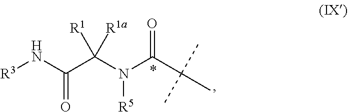

wherein the dashed line indicates the attachment to a π-electron-pair-donating heteroaromatic N of -D;
—R¹, —R^{1a}, —R³ and —R⁵ are used as defined in formula (IXi) or (IX);
optionally, the pair —R¹/—R^{1a} is joined together with the atom to which they are attached to form a C₃₋₁₀ cycloalkyl, 3- to 10-membered heterocyclyl or an 8- to 11-membered heterobicyclyl; and
optionally, the pair —R¹/—R⁵ is joined together with the atoms to which they are attached to form a 3- to 10-membered heterocyclyl or 8- to 11-membered heterobicyclyl.

In certain embodiments, —R¹ and —R^{1a} of formula (IX′) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that —R¹/—R^{1a} may optionally be joined together with the atom to which they are attached to form a C₃₋₁₀ cycloalkyl and that the paired —R¹/—R⁵ may optionally be joined together with the atoms to which they are attached to form a 3- to 10-membered heterocyclyl or 8- to 11-membered heterobicyclyl.

In certain embodiments —R¹ and —R^{1a} of formula (IX′) are both —H. In certain embodiments —R¹ of formula (IX′) is —H and —R^{1a} of formula (IX′) is C₁₋₆ alkyl. In certain embodiments, —R¹ of formula (I′) is —H and —R^{1a} of formula (I′) is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments —R³ of formula (IX′) is C₁₋₆ alkyl. In certain embodiments —R³ is T. In certain embodiments —R³ of formula (IX′) is C₃₋₁₀ cycloalkyl, such as C₅— or C₆-cycloalkyl.

In certain embodiments —R⁵ of formula (IX′) is methyl. In certain embodiments —R⁵ of formula (IX′) is ethyl.

In certain embodiments, —R⁵ of formula (IX′) is —CH₃, —R¹ and —R^{1a} of formula (IX′) are —H and —R³ of formula (IX′) is —H which is replaced by one -L²-Z moiety.

In certain embodiments, —R⁵ of formula (IX′) is —CH₃, —R¹ of formula (IX′) is —H and —R^{1a} of formula (IX′) is —CH₃ and —R³ of formula (IX′) is —H which is replaced by one -L²-Z moiety.

In certain embodiments, —R⁵ of formula (IX′) is ethyl, —R¹ and —R^{1a} of formula (IX′) are —H and —R³ of formula (IX′) is —H which is replaced by one -L²-Z moiety.

In certain embodiments —R¹/—R⁵ of formula (IX′) are joined together with the atoms to which they are attached to form a ring -A- as defined for formula (IXi) or (IX). In certain embodiments —R¹/—R⁵ are joined together with the atoms to which they are attached to form a 3- to 10-membered heterocyclyl, such as a 5-membered heterocyclyl.

In certain embodiments -L¹- is of formula (IX″):

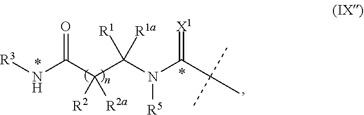

wherein the dashed line indicates the attachment to the π-electron-pair-donating heteroaromatic N of -D;
═X¹, —R¹, —R^{1a}, —R², —R^{2a}, —R³, and —R⁵ and n are used as defined in formula (IXi) or (IX);
optionally, one or more of the pairs —R¹/—R^{1a}, —R²/—R^{2a}, two adjacent —R² are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁵, —R²/—R⁵ and —R⁴/—R⁵ are joined together with the atoms to which they are attached to form a ring -A-;
   wherein -A- is selected from the group consisting of phenyl, naphthyl, indenyl, indanyl, tetralinyl, C3-10 cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, 3 and 4;
optionally, two adjacent —R² form a carbon-carbon double bond provided that n is selected from the group consisting of 2, 3 and 4;
and wherein the distance between the nitrogen atom marked with an asterisk and the carbon atom marked with an asterisk in formula (IX″) is 5, 6 or 7 atoms and if present the carbon-carbon double bond formed between —R¹ and —R² or two adjacent —R² is in a cis configuration.

In certain embodiments, n of formula (IX″) is 0. In certain embodiments, n of formula (IX″) is 1. In certain embodiments, n of formula (IX″) is 2.

In certain embodiments, —R¹ and —R^{1a} of formula (IX″) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that —R¹/—R^{1a} may optionally be joined together with the atom to which they are attached to form a C₃₋₁₀ cycloalkyl and that one or more of the pairs —R¹/—R² and —R¹/—R⁵ may optionally be joined together with the atoms to which they are attached to form a ring -A-, wherein -A- is used as defined for formula (IXi) or (IX).

In certain embodiments, —R² and —R^{2a} of formula (IX″) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that one or more of the pairs —R²/—R^{2a} and two adjacent —R² may optionally be joined with the atom to which they are attached to form a C₃₋₁₀ cycloalkyl and that the pair —R²/—R⁵ may optionally be joined together with the atoms to which they are attached to form a ring -A-, wherein -A- is used as defined in formula (IXi) or (IX).

In certain embodiments, ═X¹ of formula (IX″) is ═O.

In certain embodiments, —R¹ and —R^{1a} of formula (IX″) are both —H.

In certain embodiments, —R¹ of formula (IX″) is —H and —R^{1a} of formula (IX″) is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments, —R³ of formula (IX″) is C₁₋₆ alkyl.

In certain embodiments, —R⁵ of formula (IX″) is —H. In certain embodiments, —R⁵ of formula (IX″) is methyl. In certain embodiments, —R⁵ of formula (IX″) is ethyl.

In certain embodiments, —R⁷ of formula (IX″) is hydrogen. In certain embodiments, —R⁷ of formula (IX″) is methyl. In certain embodiments, —R⁷ of formula (IX″) is ethyl.

In certain embodiments -L¹- is of formula (IX″′).

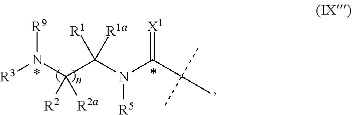

wherein the dashed line indicates the attachment to the π-electron-pair-donating heteroaromatic N of -D;
═X¹, —R¹, —R^{1a}, —R², —R^{2a}, —R³, —R⁵, —R⁹ and n are used as defined in formula (IXi) or (IX);
optionally, one or more of the pairs —R¹/—R^{1a}, —R²/—R^{2a}, two adjacent —R² and —R³/—R⁹ are joined together with the atom to which they are attached to form a C₃-10 cycloalkyl, 3- to 10-membered heterocyclyl or an 8- to 11-membered heterobicyclyl;
optionally, one or more of the pairs —R¹/—R², —R¹/—R⁵, —R²/—R⁵ and —R⁴/—R⁵ are joined together with the atoms to which they are attached to form a ring -A-;
   wherein -A- is selected from the group consisting of phenyl, naphthyl, indenyl, indanyl, tetralinyl, C3-10 cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, and 3;
optionally, two adjacent —R² form a carbon-carbon double bond provided that n is selected from the group consisting of 2, and 3;
and wherein the distance between the nitrogen atom marked with an asterisk and the carbon atom marked with an asterisk in formula (IX″′) is 5, 6 or 7 atoms and if present the carbon-carbon double bond formed between —R¹ and —R² or two adjacent —R² is in a cis configuration.

In certain embodiments, n of formula (IX″′) is 1. In certain embodiments, n of formula (IX″′) is 2. In certain embodiments, n of formula (IX″′) is 3.

In certain embodiments, —R¹ and —R^{1a} of formula (IX″′) are independently selected from the group consisting of —H and C₁₋₆ alkyl. In certain embodiments, —R¹ and —R^{1a} of formula (IX″′) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that —R¹/—R^{1a} may optionally be joined together with the atom to which they are attached to form a C₃₋₁₀ cycloalkyl and that one or more of the pairs —R¹/—R⁵, -R¹/—R⁹ and —R¹/—R¹⁰ may optionally be joined together with the atoms to which they are attached to form a ring -A-, wherein -A- is used as defined for formula (IXi) or (IX).

In certain embodiments, —R¹ and —R^{1a} of formula (IX″′) are both —H.

In certain embodiments, —R² and —R^{2a} of formula (IX″′) are independently selected from the group consisting of —H and C₁₋₆ alkyl. In certain embodiments, —R² and —R^{2a} of formula (IX″′) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that one or more of the pairs —R²/—R^{2a} and two adjacent —R² may optionally be joined with the atom to which they are attached to form a C₃₋₁₀ cycloalkyl and that the pair —R²/—R⁵ may optionally be joined together with the atoms to which they are attached to form a 3- to 10-membered heterocyclyl or 8- to 11-membered heterobicyclyl.

In certain embodiments, —R² and —R^{2a} of formula (IX″′) are both —H.

In certain embodiments, —R³ of formula (IX″′) is H. In certain embodiments, —R³ of formula (IX″′) is methyl.

In certain embodiments, —R⁵ of formula (IX″′) is H. In certain embodiments, —R⁵ of formula (IX″′) is methyl.

In certain embodiments -L¹- is selected from the group consisting of

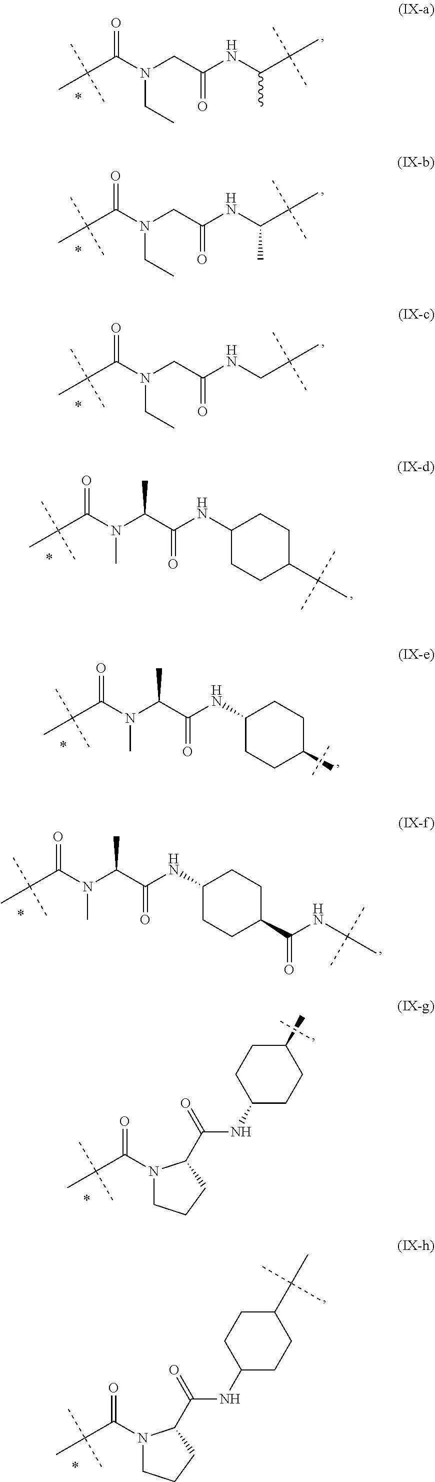

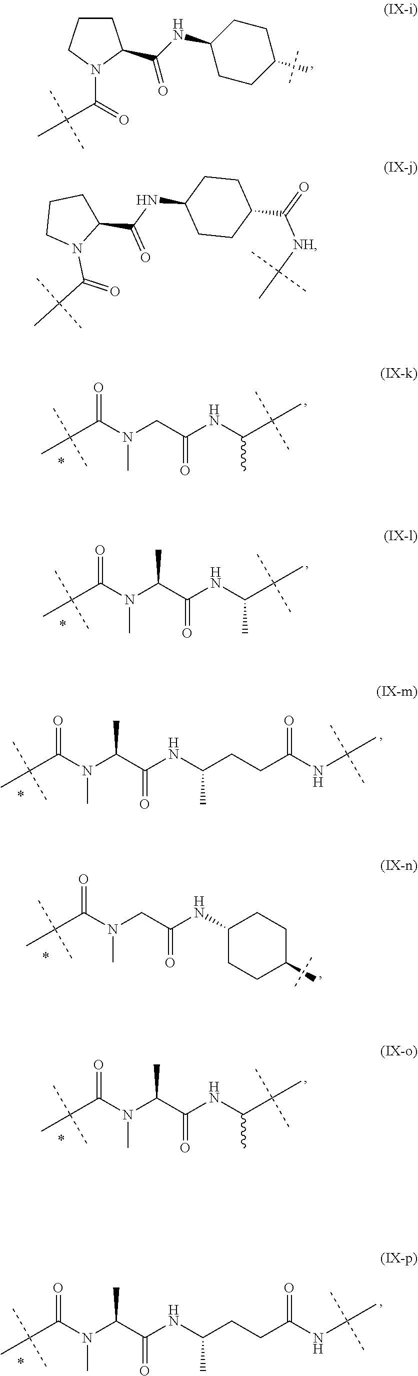

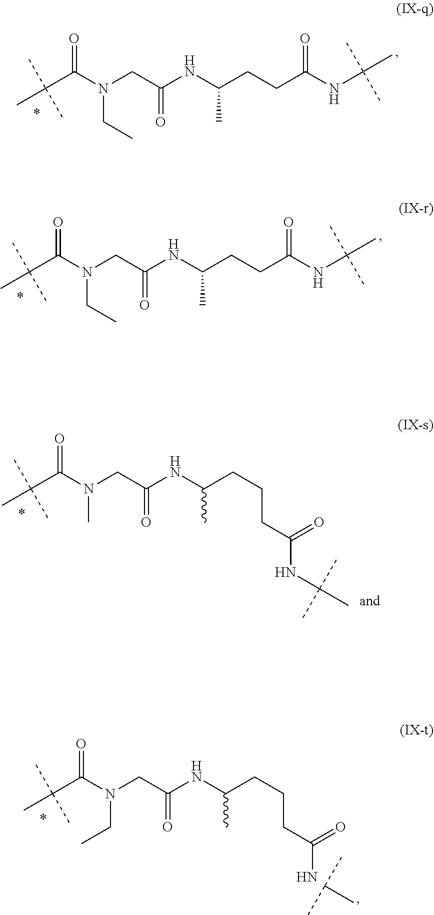

wherein
the dashed line marked with the asterisk indicates attachment to a π-electron-pair-donating heteroaromatic N of -D and the unmarked dashed line indicates attachment to -L²-.

In certain embodiments -L¹- is selected from the group consisting of (IX-a), (IX-k), (IX-m), (IX-q) and (IX-t).

In certain embodiments -L¹- is of formula (IX-a). In certain embodiments -L¹- is of formula (IX-b). In certain embodiments -L¹- is of formula (IX-c). In certain embodiments -L¹- is of formula (IX-d). In certain embodiments -L¹- is of formula (IX-e). In certain embodiments -L¹- is of formula (IX-f). In certain embodiments -L¹- is of formula (IX-g). In certain embodiments -L¹- is of formula (IX-h). In certain embodiments -L¹- is of formula (IX-i). In certain embodiments -L¹- is of formula (IX-j). In certain embodiments -L¹- is of formula (IX-k). In certain embodiments -L¹- is of formula (IX-l). In certain embodiments -L¹- is of formula (IX-m). In certain embodiments -L¹- is of formula (IX-n). In certain embodiments -L¹- is of formula (IX-o). In certain embodiments -L¹- is of formula (IX-p). In certain embodiments -L¹- is of formula (IX-q). In certain embodiments -L¹- is of formula (IX-r). In certain embodiments -L¹- is of formula (IX-s). In certain embodiments -L¹- is of formula (IX-t).

In certain embodiments -L¹- is of formula (X)

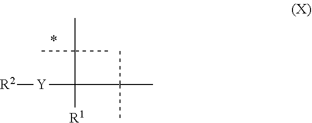

wherein
the dashed line marked with an asterisk indicates the attachment to -L²-;
the unmarked dashed line indicates the attachment to a π-electron-pair-donating heteroaromatic N of -D;
—Y— is selected from the group consisting of —N(R³)—, —O— and —S—;
—R¹, —R² and —R³ are independently selected from the group consisting of —H, -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R⁴, which are the same or different; and
wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
   each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each T is independently optionally substituted with one or more —R⁴, which are the same or different;
   wherein —R⁴, —R⁵ and —R^{5a} are independently selected from the group consisting of —H and C₁₋₆ alkyl; wherein C₁₋₆ alkyl is optionally substituted with one or more halogen, which are the same or different; and
wherein -L¹- is substituted with -L²- and wherein -L¹- is optionally further substituted.

The optional further substituents of -L¹- of formula (X) are as described elsewhere herein.

In certain embodiments -L¹- of formula (X) is not further substituted.

In certain embodiments -Y- of formula (X) is —N(R³)—.

In certain embodiments -Y- of formula (X) is —O—.

In certain embodiments -Y- of formula (X) is —S—.

In certain embodiments —R¹, —R² and —R³ of formula (X) are independently selected from the group consisting of —H, -T, C₁₋₆ alkyl, C₂₋₆ alkenyl and C₂₋₆ alkynyl.

In certain embodiments —R¹ of formula (X) is independently selected from the group consisting of —H, -T, C₁₋₆ alkyl, C₂₋₆ alkenyl and C₂₋₆ alkynyl. In certain embodiments —R¹ of formula (X) is —H. In certain embodiments —R¹ of formula (X) is -T. In certain embodiments —R¹ of formula (X) is C₁₋₆ alkyl. In certain embodiments —R¹ of formula (X) is C₂₋₆ alkenyl. In certain embodiments —R¹ of formula (X) is C₂₋₆ alkynyl.

In certain embodiments —R² of formula (X) is independently selected from the group consisting of —H, -T, C₁₋₆ alkyl, C₂₋₆ alkenyl and C₂₋₆ alkynyl. In certain embodiments —R² of formula (X) is —H. In certain embodiments —R² of formula (X) is -T. In certain embodiments —R² of formula (X) is C₁₋₆ alkyl. In certain embodiments —R² of formula (X) is C₂₋₆ alkenyl. In certain embodiments —R² of formula (X) is C₂₋₆ alkynyl.

In certain embodiments —R³ of formula (X) is independently selected from the group consisting of —H, -T, C₁₋₆ alkyl, C₂₋₆ alkenyl and C₂₋₆ alkynyl. In certain embodiments —R³ of formula (X) is —H. In certain embodiments —R³ of formula (X) is -T. In certain embodiments —R³ of formula (X) is C₁₋₆ alkyl. In certain embodiments —R³ of formula (X) is C₂₋₆ alkenyl. In certain embodiments —R³ of formula (X) is C₂₋₆ alkynyl.

In certain embodiments T of formula (X) is selected from the group consisting of phenyl, naphthyl, indenyl, indanyl, tetralinyl, C₃₋₁₀ cycloalkyl, 3- to 10-membered heterocyclyl and 8- to 11- heterobicyclyl. In certain embodiments T of formula (X) is phenyl. In certain embodiments T of formula (X) is naphthyl. In certain embodiments T of formula (X) is indenyl. In certain embodiments T of formula (X) is indanyl. In certain embodiments T of formula (X) is tetralinyl. In certain embodiments T of formula (X) is C₃₋₁₀ cycloalkyl. In certain embodiments T of formula (X) is 3- to 10-membered heterocyclyl. In certain embodiments T of formula (X) is 8- to 11-heterobicyclyl.

In certain embodiments T of formula (X) is substituted with one or more —R⁴ of formula (X).

In certain embodiments T of formula (X) is substituted with one —R⁴ of formula (X).

In certain embodiments T of formula (X) is not substituted with —R⁴ of formula (X).

In certain embodiments —R⁴, —R⁵ and —R^{5a} of formula (X) are independently selected from the group consisting of —H and C₁₋₆ alkyl.

In certain embodiments —R⁴ of formula (X) is selected from the group consisting of —H and C₁₋₆ alkyl. In certain embodiments —R⁴ of formula (X) is —H. In certain embodiments —R⁴ of formula (X) is C₁₋₆ alkyl.

In certain embodiments —R⁵ of formula (X) is selected from the group consisting of —H and C₁₋₆ alkyl. In certain embodiments —R⁵ of formula (X) is —H. In certain embodiments —R⁵ of formula (X) is C₁₋₆ alkyl.

In certain embodiments —R^{5a} of formula (X) is selected from the group consisting of —H and C₁₋₆ alkyl. In certain embodiments —R^{5a} of formula (X) is —H. In certain embodiments —R^{5a} of formula (X) is C₁₋₆ alkyl.

In certain embodiments -L¹- of formula (X) is connected to -D through a heminal linkage.

In certain embodiments -L¹- of formula (X) is connected to -D through an aminal linkage.

In certain embodiments -L¹- of formula (X) is connected to -D through a hemithioaminal linkage.

In certain embodiments, -Y- of formula (X) is —O— and —R² is C₁₋₆ alkyl. In certain embodiments, -Y- of formula (X) is —O— and —R² is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In certain embodiments, -Y- of formula (X) is —O— and —R² of formula (X) is methyl. In certain embodiments, -Y- of formula (X) is —O— and —R² of formula (X) is ethyl.

In certain embodiments, -Y- of formula (X) is —O— and —R² of formula (X) is C₁₋₆ alkyl, wherein C₁₋₆ alkyl is interrupted by —C(O)—.

In certain embodiments, -Y- of formula (X) is —N(R³)— and —R² of formula (X) is C₁₋₆ alkyl, wherein C₁₋₆ alkyl is interrupted by —C(O)O— and —R³ is as defined in formula (X).

In certain embodiments, —Y— is —N(R³)— and —R² is C₁₋₆ alkyl, wherein C₁₋₆ alkyl is interrupted by —C(O)O— and —R³ is selected from the group consisting of —H, methyl, ethyl and propyl.

In certain embodiments, -L¹- is of formula (Xi)

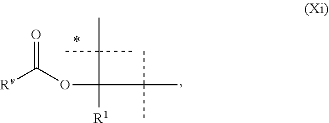

wherein
the dashed line marked with an asterisk indicates the attachment to -L²- and the unmarked dashed line indicates the attachment to the
π-electron-pair-donating heteroaromatic N of -D;
—R^{v} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and —R¹ is used as defined in formula (X).

In certain embodiments, —R^{v} of formula (Xi) is selected from the group consisting of methyl, ethyl and propyl. In certain embodiments, —R^{v} of formula (Xi) is methyl. In certain embodiments, —R^{v} of formula (Xi) is ethyl. In certain embodiments, —R^{v} of formula (Xi) is propyl.

In certain embodiments, -L¹- is of formula (Xii)

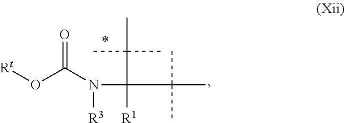

wherein
the dashed line marked with an asterisk indicates the attachment to -L²- and the unmarked dashed line indicates the attachment to the π-electron-pair-donating heteroaromatic N of -D;
—R^{t} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and
—R¹ and —R³ are used as defined in formula (X).

In certain embodiments, —R³ of formula (Xii) is selected from the group consisting of —H, methyl and ethyl. In certain embodiments, —R³ of formula (Xii) is —H. In certain embodiments, —R³ of formula (Xii) is methyl. In certain embodiments, —R³ of formula (Xii) is ethyl.

In certain embodiments, —R^{t} of formula (Xii) is selected from the group consisting of methyl, ethyl and propyl. In certain embodiments, —R^{t} of formula (Xii) is methyl. In certain embodiments, —R^{t} of formula (Xii) is ethyl. In certain embodiments, —R^{t} of formula (Xii) is propyl.

In certain embodiments, -L¹- is of formula (Xiii)

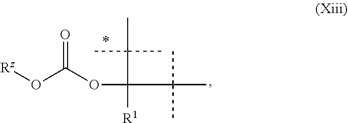

wherein
the dashed line marked with an asterisk indicates the attachment to -L²- and the unmarked dashed line indicates the attachment to the π-electron-pair-donating heteroaromatic N of -D;
—R^{z} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and —R¹ is used as defined in formula (X).

In certain embodiments, —R^{z} of formula (Xiii) is selected from the group consisting of methyl, ethyl and propyl. In certain embodiments, -Rz of formula (Xiii) is methyl. In certain embodiments, —R^{z} of formula (Xiii) is ethyl. In certain embodiments, —R^{z} of formula (Xiii) is propyl.

A moiety -L¹- suitable for drugs D that when bound to -L¹- comprises an electron-donating heteroaromatic N⁺ moiety or a quaternary ammonium cation and becomes a moiety -D⁺ upon linkage with -L¹- is of formula (XI)

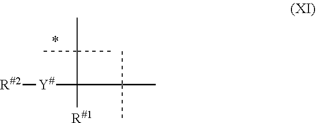

wherein
the dashed line marked with an asterisk indicates the attachment to -L²-, the unmarked dashed line indicates the attachment to the N⁺ of -D⁺;
-Y^{#}- is selected from the group consisting of —N(R^{#3})—, —O— and —S—; —R^{#1}, —R^{#2} and —R^{#3} are independently selected from the group consisting of —H, -T^{#}, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R^{#4}, which are the same or different; and wherein C₁₋₆ alkyl, C₂₋₆ alkenyl and C₂₋₆ alkynyl are optionally interrupted by one or more groups selected from the group consisting of -T^{#}-, —C(O)O—, —O—, —C(O)—, —C(O)N(R^{#5})—, —S(O)₂N(R^{#5})—, —S(O)N(R^{#5})—, —S(O)₂—, —S(O)—, —N(R^{#5})S(O)₂N(R^{#5a})—, —S—, —N(R^{#5})—, —OC(OR^{#5})(R^{#5a})—, —N(R^{#5})C(O)N(R^{#5a})— and —OC(O)N(R^{#5})—;
   each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each T is independently optionally substituted with one or more —R^{#4}, which are the same or different; and
   wherein —R^{#4}, —R^{#5} and —R^{#5a} are independently selected from the group consisting of —H and C₁₋₆ alkyl; wherein C₁₋₆ alkyl is optionally substituted with one or more halogen, which are the same or different; and
each -L¹- is substituted with -L²- and optionally further substituted.

It is understood that in certain embodiments -D⁺ may comprise both an electron-donating heteroaromatic N⁺ and a quaternary ammonium cation and analogously the corresponding D may comprise both an electron-donating heteroaromatic N and a tertiary amine. It is also understood that if D is conjugated to -L¹-, then -D⁺ and -L¹- form a quaternary ammonium cation, for which there may be a counter anion. Examples of counter anions include, but are not limited to, chloride, bromide, acetate, bicarbonate, sulfate, bisulfate, nitrate, carbonate, alkyl sulfonate, aryl sulfonate and phosphate.

Such drug moiety -D⁺ comprises at least one, such as one, two, three, four, five, six, seven, eight, nine or ten electron-donating heteroaromatic N⁺ or quaternary ammonium cations and analogously the corresponding released drug D comprises at least one, such as one, two, three, four, five, six, seven, eight, nine or ten electron-donating heteroaromatic N or tertiary amines. Examples of chemical structures including heteroaromatic nitrogens i.e. N⁺ or N, that donate an electron to the aromatic π-system include, but are not limited to, pyridine, pyridazine, pyrimidine, quinoline, quinazoline, quinoxaline, pyrazole, imidazole, isoindazole, indazole, purine, tetrazole, triazole and triazine. For example, in the imidazole ring below the heteroaromatic nitrogen which donates one electron to the aromatic π-system is marked with “§ ”:

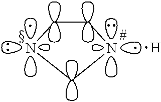

Such electron-donating heteroaromatic nitrogen atoms do not comprise heteroaromatic nitrogen atoms which donate one electron pair (i.e. not one electron) to the aromatic π-system, such as for example the nitrogen that is marked with “#” in the abovementioned imidazole ring structure. The drug D may exist in one or more tautomeric forms, such as with one hydrogen atom moving between at least two heteroaromatic nitrogen atoms. In all such cases, the linker moiety is covalently and reversibly attached at a heteroaromatic nitrogen that donates an electron to the aromatic π-system.

In certain embodiments -Y^{#}- of formula (XI) is —N(R^{#3})—. In certain embodiments -Y^{#}— of formula (XI) is —O—. In certain embodiments -Y^{#}- of formula (XI) is —S—.

In certain embodiments —R^{#1}, —R^{#2} and —R^{#3} of formula (XI) are independently selected from the group consisting of —H, -T^{#}, C₁₋₆ alkyl, C₂₋₆ alkenyl and C₂₋₆ alkynyl.

In certain embodiments —R^{#1} of formula (XI) is independently selected from the group consisting of —H, -T^{#}, C₁₋₆ alkyl, C₂₋₆ alkenyl and C₂₋₆ alkynyl. In certain embodiments —R^{#1} of formula (XI) is —H. In certain embodiments —R^{#1} of formula (XI) is -T^{#}. In certain embodiments —R^{#1} of formula (XI) is C₁₋₆ alkyl. In certain embodiments —R^{#1} of formula (XI) is C₂₋₆ alkenyl. In certain embodiments —R^{#1} of formula (XI) is C₂₋₆ alkynyl.

In certain embodiments —R^{#2} of formula (XI) is independently selected from the group consisting of —H, -T^{#}, C₁₋₆ alkyl, C₂₋₆ alkenyl and C₂₋₆ alkynyl. In certain embodiments —R^{#2} of formula (XI) is —H. In certain embodiments —R² of formula (XI) is -T^{#}. In certain embodiments —R^{#2} of formula (XI) is C₁₋₆ alkyl. In certain embodiments —R^{#2} of formula (XI) is C₂₋₆ alkenyl. In certain embodiments —R^{#2} of formula (XI) is C₂₋₆ alkynyl.

In certain embodiments, —R^{#3} of formula (XI) is independently selected from the group consisting of —H, -T^{#}, C₁₋₆ alkyl, C₂₋₆ alkenyl and C₂₋₆ alkynyl. In certain embodiments —R^{#3} of formula (XI) is —H. In certain embodiments —R^{#3} of formula (XI) is -T^{#}. In certain embodiments, —R^{#3} is C₁₋₆ alkyl. In certain embodiments —R^{#3} of formula (XI) is C₂₋₆ alkenyl. In certain embodiments —R^{#3} of formula (XI) is C₂₋₆ alkynyl.

In certain embodiments T^{#} of formula (XI) is selected from the group consisting of phenyl, naphthyl, indenyl, indanyl, tetralinyl, C₃₋₁₀ cycloalkyl, 3- to 10-membered heterocyclyl and 8- to 11- heterobicyclyl. In certain embodiments T^{#} of formula (XI) is phenyl. In certain embodiments T^{#} of formula (XI) is naphthyl. In certain embodiments T^{#} of formula (XI) is indenyl. In certain embodiments T^{#} of formula (XI) is indanyl. In certain embodiments T^{#} of formula (XI) is tetralinyl. In certain embodiments T^{#} of formula (XI) is C₃₋₁₀ cycloalkyl. In certain embodiments T^{#} of formula (XI) is 3- to 10-membered heterocyclyl. In certain embodiments T^{#} of formula (XI) is 8- to 11-heterobicyclyl. In certain embodiments T^{#} of formula (XI) is substituted with one or more —R⁴ of formula (XI).

In certain embodiments T^{#} of formula (XI) is substituted with one —R⁴ of formula (XI).

In certain embodiments T^{#} of formula (XI) is not substituted with —R⁴ of formula (XI).

In certain embodiments —R^{#4}, —R^{#5} and —R^{#5a} of formula (XI) are independently selected from the group consisting of —H and C₁₋₆ alkyl.

In certain embodiments —R^{#4} of formula (XI) is selected from the group consisting of —H and C₁₋₆ alkyl. In certain embodiments —R^{#4} of formula (XI) is —H. In certain embodiments —R^{#4} of formula (XI) is C₁₋₆ alkyl.

In certain embodiments —R^{#5} of formula (XI) is selected from the group consisting of —H and C₁₋₆ alkyl. In certain embodiments —R⁵ of formula (XI) is —H. In certain embodiments —R^{#5} of formula (XI) is C₁₋₆ alkyl.

In certain embodiments —R^{#5a} of formula (XI) is selected from the group consisting of —H and C₁₋₆ alkyl. In certain embodiments —R^{#5a} of formula (XI) is —H. In certain embodiments —R^{#5a} of formula (XI) is C₁₋₆ alkyl.

In certain embodiments, -Y^{#}- of formula (XI) is —O— and —R^{#2} of formula (XI) is C₁₋₆ alkyl. In certain embodiments, -Y^{#}- of formula (XI) is —O— and —R^{#2} of formula (XI) is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In certain embodiments, -Y^{#}- of formula (XI) is —O— and —R^{#2} of formula (XI) is methyl. In certain embodiments, -Y^{#}- of formula (XI) is —O— and —R^{#2} of formula (XI) is ethyl.

In certain embodiments, -Y^{#}- of formula (XI) is —O— and —R^{#2} of formula (XI) is C₁₋₆ alkyl, wherein C₁₋₆ alkyl is interrupted by —C(O)—.

In certain embodiments, -Y^{#}- of formula (XI) is —N(R³)— and —R^{#2} of formula (XI) is C₁₋₆ alkyl, wherein C₁₋₆ alkyl is interrupted by —C(O)O— and —R^{#3} is as defined in formula (XI).

In certain embodiments, -Y^{#}- of formula (XI) is —N(R³)— and —R^{#2} of formula (XI) is C₁₋₆ alkyl, wherein C₁₋₆ alkyl is interrupted by —C(O)O— and —R^{#3} of formula (XI) is selected from the group consisting of —H, methyl, ethyl and propyl.

In certain embodiments, -L¹- is of formula (XIi)

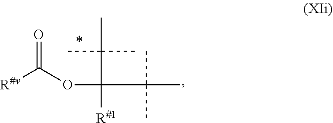

wherein
the dashed line marked with an asterisk indicates the attachment to -L²- and the unmarked dashed line indicates the attachment to the π-electron-pair-donating heteroaromatic N of -D;
—R^{#v} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and
—R^{#1} is used as defined in formula (XI).

In certain embodiments, —R^{#v} of formula (XIi) is selected from the group consisting of methyl, ethyl and propyl. In certain embodiments, —R^{#v} of formula (XIi) is methyl. In certain embodiments, —R^{#v} of formula (XIi) is ethyl. In certain embodiments, —R^{#v} of formula (XIi) is propyl.

In certain embodiments, -L¹- of formula (XIii)

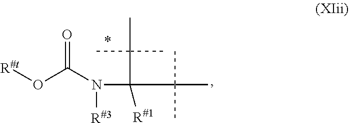

wherein
the dashed line marked with an asterisk indicates the attachment to -L²- and the unmarked dashed line indicates the attachment to the π-electron-pair-donating heteroaromatic N of -D;
—R^{#t} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and
—R^{#1} and —R^{#3} are used as defined in formula (XI).

In certain embodiments, —R^{#3} of formula (XIii) is selected from the group consisting of —H, methyl and ethyl. In certain embodiments, —R^{#3} of formula (XIii) is —H. In certain embodiments, —R^{#3} of formula (XIii) is methyl. In certain embodiments, —R^{#3} of formula (XIii) is ethyl.

In certain embodiments, —R^{#t} of formula (XIii) is selected from the group consisting of methyl, ethyl and propyl. In certain embodiments, —R^{#t} of formula (XIii) is methyl. In certain embodiments, —R^{#t} of formula (XIii) is ethyl. In certain embodiments, —R^{#t} of formula (XIii) is propyl.

In certain embodiments, -L¹- is of formula (XIiii)

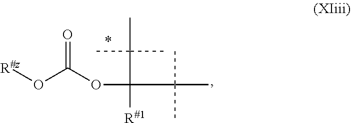

wherein
the dashed line marked with an asterisk indicates the attachment to -L²- and the unmarked dashed line indicates the attachment to the π-electron-pair-donating heteroaromatic N of -D;
—R^{#z} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and
—R^{#1} is used as defined in formula (XI).

In certain embodiments, —R^{#z} of formula (XIiii) is selected from the group consisting of methyl, ethyl and propyl. In certain embodiments, —R^{#z} of formula (XIiii) is methyl. In certain embodiments, —R^{#z} of formula (XIiii) is ethyl. In certain embodiments, —R^{#z} of formula (XIiii) is propyl.

A moiety -L¹- suitable for drugs D that when bound to -L¹- comprise an electron-donating heteroaromatic N⁺ moiety or a quaternary ammonium cation and becomes a moiety -D⁺ upon linkage with -L¹- is of formula (XII)

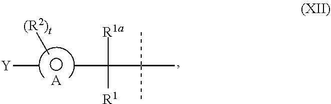

wherein
the dashed line indicates the attachment to the N⁺ of -D⁴;
t is selected from the group consisting of 0, 1, 2, 3, 4, 5 and 6;
-A- is a ring selected from the group consisting of monocyclic or bicyclic aryl and heteroaryl, provided that -A- is connected to -Y and —C(R¹)(R^{1a})— via carbon atoms; wherein said monocyclic or bicyclic aryl and heteroaryl are optionally substituted with one or more —R², which are the same or different;
—R¹, —R^{1a} and each —R² are independently selected from the group consisting of —H, —C(O)OH, -halogen, —NO₂, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})— and —OC(O)N(R⁴)—;
   each -T- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each -T- is independently optionally substituted with one or more —R³, which are the same or different;
   wherein —R³ is selected from the group consisting of —H, —NO₂, —OCH₃, —CN, —N(R⁴)(R^{4a}), —OH, —C(O)OH and C₁₋₆ alkyl; wherein C₁₋₆ alkyl is optionally substituted with one or more halogen, which are the same or different;
   wherein —R⁴ and —R^{4a} are independently selected from the group consisting of —H and C₁₋₆ alkyl; wherein C₁₋₆ alkyl is optionally substituted with one or more halogen, which are the same or different;
—Y is selected from the group consisting of:

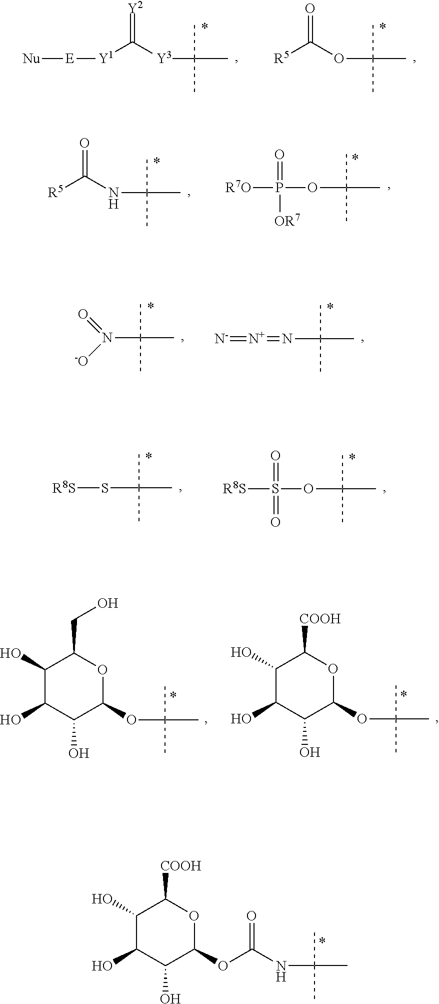

and a peptidyl moiety;
wherein
the dashed line marked with an asterisk indicates the attachment to -A-;
-Nu is a nucleophile;
-Y¹- is selected from the group consisting of —O—, —C(R¹⁰)(R^{10a})—, —N(R¹¹)— and —S—;
═Y² is selected from the group consisting of ═O, ═S and ═N(R¹²);
-Y³- is selected from the group consisting of —O—, —S— and —N(R¹³)—;
-E- is selected from the group consisting of C₁₋₆ alkyl, C₂₋₆ alkenyl, C₂₋₆ alkynyl and -Q-; wherein C₁₋₆ alkyl, C₂₋₆ alkenyl, C₂₋₆ alkynyl are optionally substituted with one or more —R¹⁴, which are the same or different;
—R⁵, —R⁶, each —R⁷, —R⁸, —R⁹, —R¹⁰, —R^{10a}, —R¹¹, —R¹² and —R¹³ are independently selected from the group consisting of C₁₋₂₀ alkyl, C₂₋₂₀ alkenyl, C₂₋₂₀ alkynyl and -Q; wherein C₁₋₂₀ alkyl, C₂₋₂₀ alkenyl and C₂₋₂₀ alkynyl are optionally substituted with one or more —R¹⁴, which are the same or different; and wherein C₁₋₂₀ alkyl, C₂₋₂₀ alkenyl and C₂₋₂₀ alkynyl are optionally interrupted by one or more groups selected from the group consisting of -Q-, —C(O)O—, —O—, —C(O)—, —C(O)N(R¹⁵)—, —S(O)₂N(R¹⁵)—, —S(O)N(R¹⁵)—, —S(O)₂—, —S(O)—, —N(R¹⁵)S(O)₂N(R^{15a})—, —S—, —N(R¹⁵)—, —OC(OR¹⁵)R^{15a}—, —N(R¹⁵)C(O)N(R^{15a})— and —OC(O)N(R¹⁵)—;
each Q is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q is independently optionally substituted with one or more —R¹⁴, which are the same or different; wherein —R¹⁴, —R¹⁵ and —R^{15a} are independently selected from the group consisting of —H and C₁₋₆ alkyl; wherein C₁₋₆ alkyl is optionally substituted with one or more halogen, which are the same or different; and
each -L¹- is substituted with -L²- and optionally further substituted.

It is understood that in certain embodiments -D⁺ may comprise both an electron-donating heteroaromatic N⁺ and a quaternary ammonium cation and analogously the corresponding D may comprise both an electron-donating heteroaromatic N and a tertiary amine. It is also understood that if D is conjugated to -L¹-, then -D⁺ and -L¹- form a quaternary ammonium cation, for which there may be a counter anion. Examples of counter anions include, but are not limited to, chloride, bromide, acetate, bicarbonate, sulfate, bisulfate, nitrate, carbonate, alkyl sulfonate, aryl sulfonate and phosphate.

The optional further substituents of -L¹- of formula (XII) are as described elsewhere herein.

In certain embodiments -L¹- of formula (XII) is not further substituted.

Such drug moiety -D⁺ comprises at least one, such as one, two, three, four, five, six, seven, eight, nine or ten electron-donating heteroaromatic N⁺ or quaternary ammonium cations and analogously the corresponding released drug D comprises at least one, such as one, two, three, four, five, six, seven, eight, nine or ten electron-donating heteroaromatic N or tertiary amines. Examples of chemical structures including heteroaromatic nitrogens i.e. N⁺ or N, that donate an electron to the aromatic n-system include, but are not limited to, pyridine, pyridazine, pyrimidine, quinoline, quinazoline, quinoxaline, pyrazole, imidazole, isoindazole, indazole, purine, tetrazole, triazole and triazine. For example, in the imidazole ring below the heteroaromatic nitrogen which donates one electron to the aromatic n-system is marked with

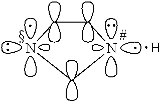

Such electron-donating heteroaromatic nitrogen atoms do not comprise heteroaromatic nitrogen atoms which donate one electron pair (i.e. not one electron) to the aromatic π-system, such as for example the nitrogen that is marked with “#” in the abovementioned imidazole ring structure. The drug D may exist in one or more tautomeric forms, such as with one hydrogen atom moving between at least two heteroaromatic nitrogen atoms. In all such cases, the linker moiety is covalently and reversibly attached at a heteroaromatic nitrogen that donates an electron to the aromatic π-system.

As used herein, the term “monocyclic or bicyclic aryl” means an aromatic hydrocarbon ring system which may be monocyclic or bicyclic, wherein the monocyclic aryl ring consists of at least 5 ring carbon atoms and may comprise up to 10 ring carbon atoms and wherein the bicyclic aryl ring consists of at least 8 ring carbon atoms and may comprise up to 12 ring carbon atoms. Each hydrogen atom of a monocyclic or bicyclic aryl may be replaced by a substituent as defined below.

As used herein, the term “monocyclic or bicyclic heteroaryl” means a monocyclic aromatic ring system that may comprise 2 to 6 ring carbon atoms and 1 to 3 ring heteroatoms or a bicyclic aromatic ring system that may comprise 3 to 9 ring carbon atoms and 1 to 5 ring heteroatoms, such as nitrogen, oxygen and sulfur. Examples for monocyclic or bicyclic heteroaryl groups include, but are not limited to, benzofuranyl, benzothiophenyl, furanyl, imidazolyl, indolyl, azaindolyl, azabenzimidazolyl, benzoxazolyl, benzthiazolyl, benzthiadiazolyl, benzotriazolyl, tetrazinyl, tetrazolyl, isothiazolyl, oxazolyl, isoxazolyl, pyrazinyl, pyrazolyl, pyridazinyl, pyridinyl, pyrimidinyl, pyrrolyl, quinolinyl, quinazolinyl, quinoxalinyl, triazolyl, thiazolyl and thiophenyl. Each hydrogen atom of a monocyclic or bicyclic heteroaryl may be replaced by a substituent as defined below.

As used herein, the term “nucleophile” refers to a reagent or functional group that forms a bond to its reaction partner, i.e. the electrophile by donating both bonding electrons.

In certain embodiments t of formula (XII) is 0. In certain embodiments t of formula (XII) is 1.

In certain embodiments t of formula (XII) is 2. In certain embodiments t of formula (XII) is 3.

In certain embodiments t of formula (XII) is 4. In certain embodiments t of formula (XII) is 5.

In certain embodiments t of formula (XII) is 6.

In certain embodiments -A- of formula (XII) is a ring selected from the group consisting of monocyclic or bicyclic aryl and heteroaryl, provided that -A- is connected to -Y and —C(R¹)(R^{1a})— via carbon atoms. In certain embodiments -A- of formula (XII) is substituted with one or more —R² of formula (XII) which are the same or different. In certain embodiments -A- of formula (XII) is not substituted with —R² of formula (XII). In certain embodiments -A- of formula (XII) is selected from the group consisting of:

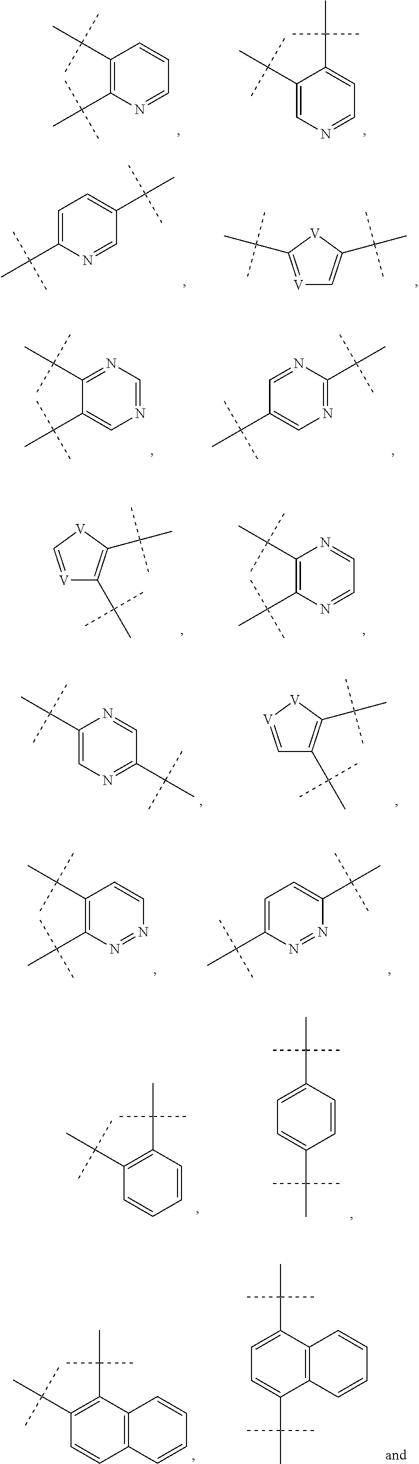

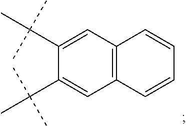

wherein each V is independently selected from the group consisting of O, S and N.

In certain embodiments —R¹, —R^{1a} and each —R² of formula (XII) are independently selected from the group consisting of —H, —C(O)OH, -halogen, —CN, —NO₂, —OH, C₁₋₆ alkyl, C₂₋₆ alkenyl and C₂₋₆ alkynyl. In certain embodiments —R¹, —R^{1a} and each —R² of formula (XII) are independently selected from the group consisting of —H, —C(O)OH, —CN, C₁₋₆ alkyl, C₂₋₆ alkenyl and C₂₋₆ alkynyl.

In certain embodiments —R¹ of formula (XII) is —H. In certain embodiments —R¹ of formula (XII) is —C(O)OH. In certain embodiments —R¹ of formula (XII) is -halogen. In certain embodiments —R¹ of formula (XII) is —F. In certain embodiments —R¹ of formula (XII) is —CN. In certain embodiments —R¹ of formula (XII) is —NO₂. In certain embodiments —R¹ of formula (XII) is —OH. In certain embodiments —R¹ of formula (XII) is C₁₋₆ alkyl. In certain embodiments —R¹ of formula (XII) is C₂₋₆ alkenyl. In certain embodiments —R¹ of formula (XII) is C₂₋₆ alkynyl. In certain embodiments —R^{1a} of formula (XII) is —H. In certain embodiments —R^{1a} of formula (XII) is —C(O)OH. In certain embodiments —R^{1a} of formula (XII) is -halogen. In certain embodiments —R^{1a} of formula (XII) is —F. In certain embodiments —R^{1a} of formula (XII) is —CN. In certain embodiments —R^{1a} of formula (XII) is —NO₂. In certain embodiments —R^{1a} of formula (XII) is —OH. In certain embodiments —R^{1a} of formula (XII) is C₁₋₆ alkyl. In certain embodiments —R^{1a} of formula (XII) is C₂₋₆ alkenyl. In certain embodiments —R^{1a} of formula (XII) is C₂₋₆ alkynyl.

In certain embodiments each of —R² of formula (XII) is independently selected from the group consisting of —H, —C(O)OH, -halogen, —CN, —NO₂, —OH, C₁₋₆ alkyl, C₂₋₆ alkenyl and C₂₋₆ alkynyl. In certain embodiments each of —R² of formula (XII) is —H. In certain embodiments each of —R² of formula (XII) is —C(O)OH. In certain embodiments each of —R² of formula (XII) is -halogen. In certain embodiments each of —R² of formula (XII) is —F. In certain embodiments each of —R² of formula (XII) is —CN. In certain embodiments each of —R² of formula (XII) is —NO₂. In certain embodiments each of —R² of formula (XII) is —OH. In certain embodiments each of —R² of formula (XII) is C₁₋₆ alkyl. In certain embodiments each of —R² of formula (XII) is C₂₋₆ alkenyl. In certain embodiments each of —R² of formula (XII) is C₂₋₆ alkynyl.

In certain embodiments T of formula (XII) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments T of formula (XII) is phenyl. In certain embodiments T of formula (XII) is naphthyl. In certain embodiments T of formula (XII) is indenyl. In certain embodiments T of formula (XII) is indanyl. In certain embodiments T of formula (XII) is tetralinyl. In certain embodiments T of formula (XII) is C₃₋₁₀ cycloalkyl. In certain embodiments T of formula (XII) is 3- to 10-membered heterocyclyl. In certain embodiments T of formula (XII) is 8- to 11-membered heterobicyclyl.

In certain embodiments T of formula (XII) is substituted with one or more —R³ of formula (XII), which are the same or different. In certain embodiments T of formula (XII) is substituted with one —R³ of formula (XII). In certain embodiments T of formula (XII) is not substituted with —R³ of formula (XII).

In certain embodiments —R³ of formula (XII) is selected from the group consisting of —H, —NO₂, —OCH₃, —CN, —N(R⁴)(R^{4a}), —OH, —C(O)OH and C₁₋₆ alkyl. In certain embodiments —R³ of formula (XII) is —H. In certain embodiments —R³ of formula (XII) is —NO₂. In certain embodiments —R³ of formula (XII) is —OCH₃. In certain embodiments —R³ of formula (XII) is —CN. In certain embodiments —R³ of formula (XII) is —N(R⁴)(R^{4a}). In certain embodiments —R³ of formula (XII) is —OH. In certain embodiments —R³ of formula (XII) is —C(O)OH. In certain embodiments —R³ of formula (XII) is C₁₋₆ alkyl. In certain embodiments —R⁴ and —R^{4a} of formula (XII) are independently selected from the group consisting of —H and C₁₋₆ alkyl. In certain embodiments —R⁴ of formula (XII) is —H. In certain embodiments —R⁴ is C₁₋₆ alkyl. In certain embodiments —R^{4a} of formula (XII) is —H. In certain embodiments —R^{4a} of formula (XII) is C₁₋₆ alkyl.

In certain embodiments, -Y of formula (XII) is selected from the group consisting of

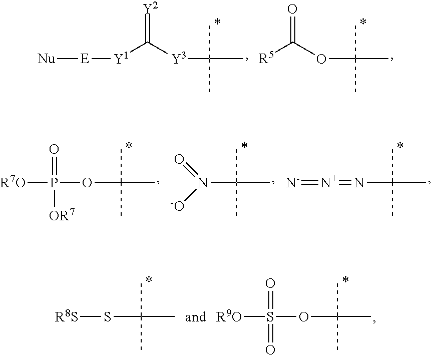

wherein -Nu, -E-, —Y¹—, ═Y², —Y³—, —R⁵, —R⁷, —R⁸ and —R⁹ are defined as above.

In certain embodiments -Y of formula (XII) is

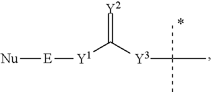

wherein -Nu, -E, —Y¹—, ═Y² and -Y³- are as defined elsewhere herein and the dashed line marked with an asterisk indicates the attachment to -A- of formula (XII). It is understood that in this instance the release of the drug D is not triggered by an enzyme, and that the drug is released in its unmodified, pharmacologically fully active form in the absence of an enzyme.

In certain embodiments -Nu of formula (XII) is a nucleophile selected from the group consisting of primary, secondary, or tertiary amine and amide. In certain embodiments -Nu of formula (XII) is a primary amine. In certain embodiments -Nu of formula (XII) is a secondary amine. In certain embodiments -Nu of formula (XII) is a tertiary amine. In certain embodiments -Nu of formula (XII) is an amide.

In certain embodiments -Y¹- of formula (XII) is selected from the group consisting of —O—, —C(R¹⁰)(R^{10a})—, —N(R¹¹)— and —S—. In certain embodiments -Y¹- of formula (XII) is —O—. In certain embodiments -Y¹- of formula (XII) is —C(R¹⁰)(R^{10a})—. In certain embodiments -Y¹- of formula (XII) is —N(R¹¹)—. In certain embodiments -Y¹- of formula (XII) is —S—.

In certain embodiments ═Y² of formula (XII) is selected from the group consisting of ═O, ═S and ═N(R¹²). In certain embodiments ═Y² of formula (XII) is ═O. In certain embodiments ═Y² of formula (XII) is ═S. In certain embodiments ═Y² of formula (XII) is ═N(R¹²).

In certain embodiments -Y³- of formula (XII) is selected from the group consisting of —O—, —S— and —N(R¹³). In certain embodiments -Y³- of formula (XII) is —O—. In certain embodiments -Y³- of formula (XII) is —S—. In certain embodiments -Y³- of formula (XII) is —N(R¹³)—.

In certain embodiments -Y¹- of formula (XII) is —N(R¹¹)—, ═Y² of formula (XII) is ═O and -Y³— is —O—.

In certain embodiments -Y¹- of formula (XII) is —N(R¹¹)—, ═Y² of formula (XII) is ═O, -Y³- of formula (XII) is —O— and -Nu of formula (XII) is —N(CH₃)₂.

In certain embodiments -E- of formula (XII) is selected from the group consisting of C₁₋₆ alkyl, C₂₋₆ alkenyl, C₂₋₆ alkynyl and -Q-. In certain embodiments -E- of formula (XII) is C₁₋₆ alkyl. In certain embodiments -E- of formula (XII) is C₂₋₆ alkenyl. In certain embodiments -E- of formula (XII) is C₂₋₆ alkynyl. In certain embodiments -E- of formula (XII) is -Q-.

In certain embodiments Q of formula (XII) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments Q of formula (XII) is phenyl. In certain embodiments Q of formula (XII) is naphthyl. In certain embodiments Q of formula (XII) is indenyl. In certain embodiments Q of formula (XII) is indanyl. In certain embodiments Q of formula (XII) is tetralinyl. In certain embodiments Q of formula (XII) is C₃₋₁₀ cycloalkyl. In certain embodiments Q of formula (XII) is 3- to 10-membered heterocyclyl. In certain embodiments Q of formula (XII) is 8- to 11-membered heterobicyclyl. In certain embodiments Q of formula (XII) is substituted with one or more —R¹⁴. In certain embodiments Q of formula (XII) is not substituted with —R¹⁴.

In certain embodiments —R⁵, —R⁶, each —R⁷, —R⁸, —R⁹, —R¹⁰, —R^{10a}, —R¹¹, —R¹² and —R¹³ of formula (XII) are independently selected from the group consisting of C₁₋₂₀ alkyl, C₂₋₂₀ alkenyl, C₂₋₂₀ alkynyl and -Q.

In certain embodiments —R⁵ of formula (XII) is C₁₋₂₀ alkyl. In certain embodiments —R⁵ of formula (XII) is C₂₋₂₀ alkenyl. In certain embodiments —R⁵ of formula (XII) is C₂₋₂₀ alkynyl. In certain embodiments —R⁵ of formula (XII) is -Q.

In certain embodiments —R⁶ of formula (XII) is C₁₋₂₀ alkyl. In certain embodiments —R⁶ of formula (XII) is C₂₋₂₀ alkenyl. In certain embodiments —R⁶ of formula (XII) is C₂₋₂₀ alkynyl. In certain embodiments —R⁶ is -Q.

In certain embodiments each of —R⁷ of formula (XII) is independently selected from the group consisting of C₁₋₂₀ alkyl, C₂₋₂₀ alkenyl, C₂₋₂₀ alkynyl and -Q. In certain embodiments each of —R⁷ of formula (XII) is C₁₋₂₀ alkyl. In certain embodiments each of —R⁷ of formula (XII) is C₂₋₂₀ alkenyl. In certain embodiments each of —R⁷ of formula (XII) is C₂₋₂₀ alkynyl. In certain embodiments each of —R⁷ of formula (XII) is -Q.

In certain embodiments —R⁸ of formula (XII) is C₁₋₂₀ alkyl. In certain embodiments —R⁸ of formula (XII) is C₂₋₂₀ alkenyl. In certain embodiments —R⁸ of formula (XII) is C₂₋₂₀ alkynyl. In certain embodiments —R⁸ of formula (XII) is -Q.

In certain embodiments —R⁹ of formula (XII) is C₁₋₂₀ alkyl. In certain embodiments —R⁹ of formula (XII) is C₂₋₂₀ alkenyl. In certain embodiments —R⁹ of formula (XII) is C₂₋₂₀ alkynyl. In certain embodiments —R⁹ of formula (XII) is -Q.

In certain embodiments —R¹⁰ of formula (XII) is C₁₋₂₀ alkyl. In certain embodiments —R¹⁰ of formula (XII) is C₂₋₂₀ alkenyl. In certain embodiments —R¹⁰ of formula (XII) is C₂₋₂₀ alkynyl. In certain embodiments —R¹⁰ of formula (XII) is -Q.

In certain embodiments —R^{10a} of formula (XII) is C₁₋₂₀ alkyl. In certain embodiments —R^{10a} of formula (XII) is C₂₋₂₀ alkenyl. In certain embodiments —R^{10a} of formula (XII) is C₂₋₂₀ alkynyl. In certain embodiments —R^{10a} of formula (XII) is -Q.

In certain embodiments —R¹¹ of formula (XII) is C₁₋₂₀ alkyl. In certain embodiments —R¹¹ of formula (XII) is C₂₋₂₀ alkenyl. In certain embodiments —R¹¹ of formula (XII) is C₂₋₂₀ alkynyl. In certain embodiments —R¹¹ of formula (XII) is -Q.

In certain embodiments —R¹² of formula (XII) is C₁₋₂₀ alkyl. In certain embodiments —R¹² of formula (XII) is C₂₋₂₀ alkenyl. In certain embodiments —R¹² of formula (XII) is C₂₋₂₀ alkynyl. In certain embodiments —R¹² of formula (XII) is -Q.

In certain embodiments —R¹³ of formula (XII) is C₁₋₂₀ alkyl. In certain embodiments —R¹³ of formula (XII) is C₂₋₂₀ alkenyl. In certain embodiments —R¹³ of formula (XII) is C₂₋₂₀ alkynyl. In certain embodiments —R¹³ of formula (XII) is -Q.

In certain embodiments —R¹⁴, —R¹⁵ and —R^{15a} of formula (XII) are selected from the group consisting of —H and C₁₋₆ alkyl.

In certain embodiments —R¹⁴ of formula (XII) is —H. In certain embodiments —R¹⁴ of formula (XII) is C₁₋₆ alkyl.

In certain embodiments —R¹⁵ of formula (XII) is —H. In certain embodiments —R¹⁵ of formula (XII) is C₁₋₆ alkyl.

In certain embodiments —R^{15a} of formula (XII) is —H. In certain embodiments —R^{15a} of formula (XII) is C₁₋₆ alkyl.

In certain embodiments -Y of formula (XII) is

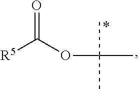

wherein —R⁵ is as defined above and the dashed line marked with an asterisk indicates the attachment to -A-.

In certain embodiments -Y of formula (XII) is

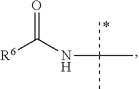

wherein —R⁶ is as defined above and the dashed line marked with an asterisk indicates the attachment to -A-.

In certain embodiments —R⁶ of formula (XII) is of formula (XIIa):

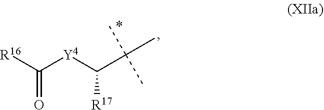

wherein -Y⁴— is selected from the group consisting of C₃₋₁₀ cycloalkyl, 3- to 10-membered heterocyclyl and 8- to 11-membered heterobicyclyl, which are optionally substituted with one or more —R¹⁸ which are the same or different;
—R¹⁶ and —R¹⁷ are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R¹⁸ which are the same or different; and wherein C₁₋₁₀ alkyl, C₂₋₁₀ alkenyl and C₂₋₁₀ alkynyl are optionally interrupted by one or more groups selected from the group consisting of -A′-, —C(O)O—, —O—, —C(O)—, —C(O)N(R¹⁹)—, —S(O)₂N(R¹⁹), —S(O)N(R¹⁹)—, —S(O)₂—, —S(O)—, —N(R¹⁹)S(O)₂N(R^{19a})—, —S—, —N(R¹⁹)—, —OC(OR¹⁹)R^{19a}—, —N(R¹⁹)C(O)N(R^{19a})—, —OC(O)N(R¹⁹)— and —N(R¹⁹)C(NH₂)N(R^{19a})—;
   each A′ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each A′ is independently optionally substituted with one or more —R¹⁸ which are the same or different;
   wherein —R¹⁸, —R¹⁹ and —R^{19a} are independently selected from the group consisting of —H and C₁₋₆ alkyl; wherein C₁₋₆ alkyl is optionally substituted with one or more halogen, which are the same or different; and
wherein the dashed line marked with an asterisk indicates the attachment to the rest of -Y.

In certain embodiments -Y⁴- of formula (XIIa) is selected from the group consisting of C₃₋₁₀ cycloalkyl, 3- to 10-membered heterocyclyl and 8- to 11-membered heterobicyclyl. In certain embodiments -Y⁴- of formula (XIIa) is C₃₋₁₀ cycloalkyl. In certain embodiments -Y⁴- of formula (XIIa) is 3- to 10-membered heterocyclyl. In certain embodiments -Y⁴- of formula (XIIa) is 8- to 11-membered heterobicyclyl. In certain embodiments -Y⁴- of formula (XIIa) is substituted with one or more —R¹⁸ which are the same or different. In certain embodiments -Y⁴- of formula (XIIa) is not substituted with —R¹⁸.

In certain embodiments —R¹⁶ and —R¹⁷ of formula (XIIa) are selected from the group consisting of C₁₋₁₀ alkyl, C₂₋₁₀ alkenyl and C₂₋₁₀ alkynyl. In certain embodiments —R¹⁶ of formula (XIIa) is C₁₋₁₀ alkyl. In certain embodiments —R¹⁶ of formula (XIIa) is C₂₋₁₀ alkenyl. In certain embodiments —R¹⁶ of formula (XIIa) is C₂₋₁₀ alkynyl. In certain embodiments —R¹⁷ of formula (XIIa) is C₁₋₁₀ alkyl. In certain embodiments —R¹⁷ of formula (XIIa) is C₂₋₁₀ alkenyl. In certain embodiments —R¹⁷ of formula (XIIa) is C₂₋₁₀ alkynyl.

In certain embodiments A′ of formula (XIIa) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments A′ of formula (XIIa) is phenyl. In certain embodiments A′ of formula (XIIa) is naphthyl. In certain embodiments A′ of formula (XIIa) is indenyl. In certain embodiments A′ of formula (XIIa) is indanyl. In certain embodiments A′ of formula (XIIa) is tetralinyl. In certain embodiments A′ of formula (XIIa) is C₃₋₁₀ cycloalkyl. In certain embodiments A′ of formula (XIIa) is 3- to 10-membered heterocyclyl. In certain embodiments A′ of formula (XIIa) is 8- to 11-membered heterobicyclyl.

In certain embodiments A′ of formula (XIIa) is substituted with one or more —R¹⁸, which are the same or different. In certain embodiments A′ of formula (XIIa) is not substituted with —R¹⁸.

In certain embodiments —R¹⁸, —R¹⁹ and —R^{19a} of formula (XIIa) are selected from the group consisting of —H and C₁₋₆ alkyl.

In certain embodiments —R¹⁸ of formula (XIIa) is —H. In certain embodiments —R¹⁸ of formula (XIIa) is C₁₋₆ alkyl. In certain embodiments —R¹⁹ of formula (XIIa) is —H. In certain embodiments —R¹⁹ of formula (XIIa) is C₁₋₆ alkyl. In certain embodiments —R^{19a} of formula (XIIa) is —H. In certain embodiments —R^{19a} of formula (XIIa) is C₁₋₆ alkyl.

In certain embodiments —R⁶ of formula (XII) is of formula (XIIb):

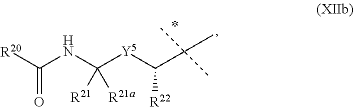

wherein -Y⁵— is selected from the group consisting of -Q′-, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²³, which are the same or different; and wherein C₁₋₁₀ alkyl, C₂₋₁₀ alkenyl and C₂₋₁₀ alkynyl are optionally interrupted by one or more groups selected from the group consisting of -Q′-, —C(O)O—, —O—, —C(O)—, —C(O)N(R²⁴)—, —S(O)₂N(R²⁴)—, —S(O)N(R²⁴)—, —S(O)₂—, —S(O)—, —N(R²⁴)S(O)₂N(R^{24a})—, —S—, —N(R²⁴)—, —OC(OR²⁴)R^{24a}—, —N(R²⁴)C(O)N(R^{24a})—, —OC(O)N(R²⁴)— and —N(R²⁴)C(NH₂)N(R^{24a})—; —R²⁰, —R²¹, —R^{21a} and —R²² are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²³ which are the same or different; and wherein C₁₋₁₀ alkyl, C₂₋₁₀ alkenyl and C₂₋₁₀ alkynyl are optionally interrupted by one or more groups selected from the group consisting of -Q′-, —C(O)O—, —O—, —C(O)—, —C(O)N(R²⁴)—, —S(O)₂N(R²⁴)—, —S(O)N(R²⁴)—, —S(O)₂—, —S(O)—, —N(R²⁴)S(O)₂N(R^{24a})—, —S—, —N(R²⁴)—, —OC(OR²⁴)R^{24a}—, —N(R²⁴)C(O)N(R^{24a})—, —OC(O)N(R²⁴)— and —N(R²⁴)C(NH₂)N(R^{24a})—;
   each Q′ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q′ is independently optionally substituted with one or more —R²³, which are the same or different;
   wherein —R²³, —R²⁴ and —R^{24a} are independently selected from the group consisting of —H and C₁₋₆ alkyl; wherein C₁₋₆ alkyl is optionally substituted with one or more halogen, which are the same or different;
optionally, the pair —R²¹/—R^{21a} is joined together with the atoms to which is attached to form a C₃₋₁₀ cycloalkyl, 3- to 10-membered heterocyclyl or an 8- to 11-membered heterobicyclyl; and
wherein the dashed line marked with an asterisk indicates the attachment to the rest of -Y.

In certain embodiments -Y⁵- of formula (XIIb) is selected from the group consisting of -Q′-, C₁₋₁₀ alkyl, C₂₋₁₀ alkenyl and C₂₋₁₀ alkynyl. In certain embodiments -Y⁵- of formula (XIIb) is -Q′-. In certain embodiments -Y⁵- of formula (XIIb) is C₁₋₁₀ alkyl. In certain embodiments -Y⁵- of formula (XIIb) is C₂₋₁₀ alkenyl. In certain embodiments -Y⁵- of formula (XIIb) is C₂₋₁₀ alkynyl.

In certain embodiments Q′ of formula (XIIb) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments Q′ of formula (XIIb) is phenyl. In certain embodiments Q′ of formula (XIIb) is naphthyl. In certain embodiments Q′ of formula (XIIb) is indenyl. In certain embodiments Q′ of formula (XIIb) is indanyl. In certain embodiments Q′ of formula (XIIb) is C₃₋₁₀ cycloalkyl. In certain embodiments Q′ of formula (XIIb) is 3- to 10-membered heterocyclyl. In certain embodiments Q′ of formula (XIIb) is 8- to 11-membered heterobicyclyl. In certain embodiments Q′ of formula (XIIb) is substituted with one or more —R²³ which are the same or different. In certain embodiments Q′ of formula (XIIb) is not substituted with —R²³.

In certain embodiments —R²⁰, —R²¹, —R^{21a} and —R²² of formula (XIIb) are selected from the group consisting of —H, C₁₋₁₀ alkyl, C₂₋₁₀ alkenyl and C₂₋₁₀ alkynyl. In certain embodiments —R²⁰ of formula (XIIb) is —H. In certain embodiments —R²⁰ of formula (XIIb) is C₁₋₁₀ alkyl. In certain embodiments —R²⁰ of formula (XIIb) is C₂₋₁₀ alkenyl. In certain embodiments —R²⁰ of formula (XIIb) is C₂₋₁₀ alkynyl. In certain embodiments —R²¹ of formula (XIIb) is —H. In certain embodiments —R²¹ of formula (XIIb) is C₁₋₁₀ alkyl. In certain embodiments —R²¹ of formula (XIIb) is C₂₋₁₀ alkenyl. In certain embodiments —R²¹ of formula (XIIb) is C₂₋₁₀ alkynyl. In certain embodiments —R^{21a} of formula (XIIb) is —H. In certain embodiments —R^{21a} of formula (XIIb) is C₁₋₁₀ alkyl. In certain embodiments —R^{21a} of formula (XIIb) is C₂₋₁₀ alkenyl. In certain embodiments —R^{21a} of formula (XIIb) is C₂₋₁₀ alkynyl. In certain embodiments —R²² of formula (XIIb) is —H. In certain embodiments —R²² of formula (XIIb) is C₁₋₁₀ alkyl. In certain embodiments —R²² of formula (XIIb) is C₂₋₁₀ alkenyl. In certain embodiments —R²² of formula (XIIb) is C₂₋₁₀ alkynyl.

In certain embodiments —R²³, —R²⁴ and —R^{24a} of formula (XIIb) are selected from the group consisting of —H and C₁₋₆ alkyl. In certain embodiments —R²³ of formula (XIIb) is —H. In certain embodiments —R²³ of formula (XIIb) is C₁₋₆ alkyl. In certain embodiments —R²⁴ of formula (XIIb) is —H. In certain embodiments —R²⁴ of formula (XIIb) is C₁₋₆ alkyl. In certain embodiments —R^{24a} of formula (XIIb) is —H. In certain embodiments —R^{24a} of formula (XIIb) is C₁₋₆ alkyl.

In certain embodiments the pair —R²¹/—R^{21a} of formula (XIIb) is joined together with the atoms to which is attached to form a C₃₋₁₀ cycloalkyl.

In certain embodiments —R⁶ of formula (XIIb) is of formula (XIIc):

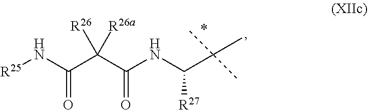

wherein
—R²⁵, —R²⁶, —R^{26a} and —R²⁷ are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²⁸ which are the same or different; and wherein C₁₋₁₀ alkyl, C₂₋₁₀ alkenyl and C₂₋₁₀ alkynyl are optionally interrupted by one or more groups selected from the group consisting of -Q*-, —C(O)O—, —O—, —C(O)—, —C(O)N(R²⁹)—, —S(O)₂N(R²⁹)—, —S(O)N(R²⁹)—, —S(O)₂—, —S(O)—, —N(R²⁹)S(O)₂N(R^{29a})—, —S—, —N(R²⁹)—, —OC(OR²⁹)R^{29a}—, —N(R²⁹)C(O)N(R^{29a})—, —OC(O)N(R²⁹)— and —N(R²⁹)C(NH₂)N(R^{29a})_;
   each Q* is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q* is independently optionally substituted with one or more —R²⁸, which are the same or different;
   wherein —R²⁸, —R²⁹ and —R^{29a} are independently selected from the group consisting of —H and C₁₋₆ alkyl; wherein C₁₋₆ alkyl is optionally substituted with one or more halogen, which are the same or different;
optionally, the pair —R²⁶/—R^{26a} is joined together with the atoms to which is attached to form a C₃₋₁₀ cycloalkyl, 3- to 10-membered heterocyclyl or an 8- to 11-membered heterobicyclyl; and
wherein the dashed line marked with an asterisk indicates the attachment to the rest of -Y.

In certain embodiments —R²⁵, —R²⁶, —R^{26a} and —R²⁷ of formula (XIIc) are selected from the group consisting of —H, C₁₋₁₀ alkyl, C₂₋₁₀ alkenyl and C₂₋₁₀ alkynyl. In certain embodiments —R²⁵ of formula (XIIc) is —H. In certain embodiments —R²⁵ of formula (XIIc) is C₁₋₁₀ alkyl. In certain embodiments —R²⁵ of formula (XIIc) is C₂₋₁₀ alkenyl. In certain embodiments —R²⁵ of formula (XIIc) is C₂₋₁₀ alkynyl. In certain embodiments —R²⁶ of formula (XIIc) is —H. In certain embodiments —R²⁶ of formula (XIIc) is C₁₋₁₀ alkyl. In certain embodiments —R²⁶ of formula (XIIc) is C₂₋₁₀ alkenyl. In certain embodiments —R²⁶ of formula (XIIc) is C₂₋₁₀ alkynyl. In certain embodiments —R^{26a} of formula (XIIc) is —H. In certain embodiments —R^{26a} of formula (XIIc) is C₁₋₁₀ alkyl. In certain embodiments —R^{26a} of formula (XIIc) is C₂₋₁₀ alkenyl. In certain embodiments —R^{26a} of formula (XIIc) is C₂₋₁₀ alkynyl. In certain embodiments —R²⁷ of formula (XIIc) is —H. In certain embodiments —R²⁷ of formula (XIIc) is C₁₋₁₀ alkyl. In certain embodiments —R²⁷ of formula (XIIc) is C₂₋₁₀ alkenyl. In certain embodiments —R²⁷ of formula (XIIc) is C₂₋₁₀ alkynyl.

In certain embodiments Q* of formula (XIIc) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments Q* of formula (XIIc) is phenyl. In certain embodiments Q* of formula (XIIc) is naphthyl. In certain embodiments Q* of formula (XIIc) is indenyl. In certain embodiments Q* of formula (XIIc) is indanyl. In certain embodiments Q* of formula (XIIc) is tetralinyl. In certain embodiments Q* of formula (XIIc) is C₃₋₁₀ cycloalkyl. In certain embodiments Q* of formula (XIIc) is 3- to 10-membered heterocyclyl. In certain embodiments Q* of formula (XIIc) is 8- to 11-membered heterobicyclyl. In certain embodiments Q* of formula (XIIc) is substituted with one or more —R²⁸, which are the same or different. In certain embodiments Q* of formula (XIIc) is not substituted with —R²⁸.

In certain embodiments —R²⁸, —R²⁹ and —R^{29a} of formula (XIIc) are selected from the group consisting of —H and C₁₋₆ alkyl. In certain embodiments —R²⁸ of formula (XIIc) is —H. In certain embodiments —R²⁸ of formula (XIIc) is C₁₋₆ alkyl. In certain embodiments —R²⁹ of formula (XIIc) is —H. In certain embodiments —R²⁹ of formula (XIIc) is C₁₋₆ alkyl. In certain embodiments —R^{29a} of formula (XIIc) is —H. In certain embodiments —R^{29a} of formula (XIIc) is C₁₋₆ alkyl.

In certain embodiments the pair —R²⁶/—R^{26a} of formula (XIIc) is joined together with the atoms to which is attached to form a C₃₋₁₀ cycloalkyl. In certain embodiments the pair —R²⁶/—R^{26a} of formula (XIIc) is joined together with the atoms to which is attached to form a cyclobutyl.

In certain embodiments -Y of formula (XII) is

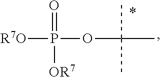

wherein each —R⁷ is as defined above and the dashed line marked with an asterisk indicates the attachment to -A-. It is understood that in this instance the release of the drug D may be triggered by an enzyme, such as phosphatase.

In certain embodiments -Y of formula (XII) is

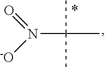

wherein the dashed line marked with an asterisk indicates the attachment to -A-.

In certain embodiments -Y of formula (XII) is

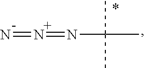

wherein the dashed line marked with an asterisk indicates the attachment to -A-.

In certain embodiments -Y of formula (XII) is

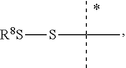

wherein —R⁸ is as defined above and the dashed line marked with an asterisk indicates the attachment to -A-.

In certain embodiments -Y of formula (XII) is

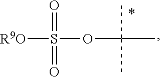

wherein —R⁹ is as defined above and the dashed line marked with an asterisk indicates the attachment to -A-. It is understood that in this instance the release of the drug D may be triggered by an enzyme, such as sulfatase.

In certain embodiments -Y of formula (XII) is

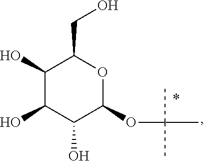

wherein the dashed line marked with an asterisk indicates the attachment to -A-. It is understood that in this instance the release of the drug D may be triggered by an enzyme, such as a-galactosidase.

In certain embodiments -Y of formula (XII) is

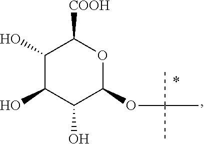

wherein the dashed line marked with an asterisk indicates the attachment to -A-. It is understood that in this instance the release of the drug D may be triggered by an enzyme, such as β-glucuronidase.

In certain embodiments -Y of formula (XII) is

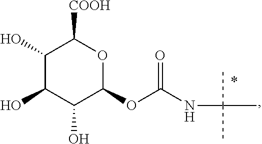

wherein the dashed line marked with an asterisk indicates the attachment to -A-. It is understood that in this instance the release of the drug D may be triggered by an enzyme, such as β-glucuronidase.

In certain embodiments -Y of formula (XII) is a peptidyl moiety.

It is understood that if -Y of formula (XII) is a peptidyl moiety, then the release of the drug D may be triggered by an enzyme, such as protease. In certain embodiments the protease is selected from the group consisting of cathepsin B and cathepsin K. In certain embodiments the protease is cathepsin B. In certain embodiments the protease is cathepsin K.

In certain embodiments -Y of formula (XII) is a peptidyl moiety, such as a dipeptidyl, tripeptidyl, tetrapeptidyl, pentapeptidyl or hexapeptidyl moiety. In certain embodiments -Y of formula (XII) is a dipeptidyl moiety. In certain embodiments -Y of formula (XII) is a tripeptidyl moiety. In certain embodiments -Y of formula (XII) is a tetrapeptidyl moiety. In certain embodiments -Y of formula (XII) is a pentapeptidyl moiety. In certain embodiments -Y of formula (XII) is a hexapeptidyl moiety.

In certain embodiments -Y of formula (XII) is a peptidyl moiety selected from the group consisting of

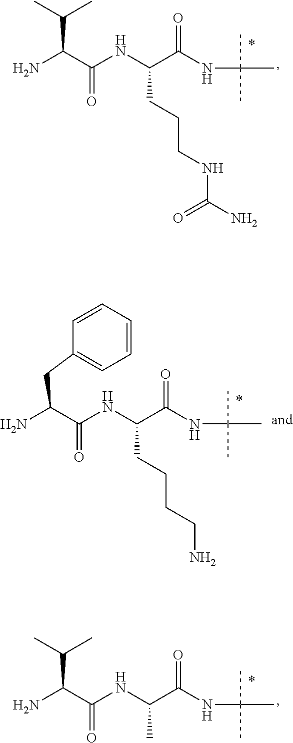

wherein the dashed line marked with an asterisk indicates the attachment to -A-.

In certain embodiments -Y of formula (XII) is

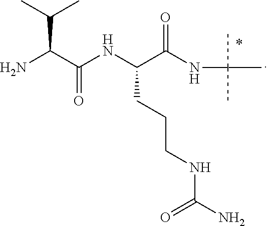

In certain embodiments -Y of formula (XII) is

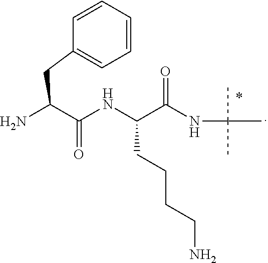

In certain embodiments -Y of formula (XII) is

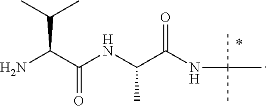

In certain embodiments one hydrogen given by —R^{1a} of formula (XII) is replaced by -L²- and -L¹- is of formula (XII′):

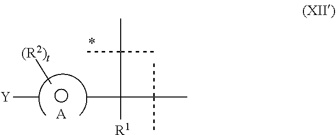

wherein
the unmarked dashed line indicates the attachment to the N⁺ of -D⁺, the dashed line marked with an asterisk indicates the attachment to -L²-; and
—R¹, -A-, —Y, R² and t are defined as in formula (XII).

In certain embodiments one hydrogen given by —R² of formula (XII) is replaced by -L²- and -L¹- is of formula (XII″):

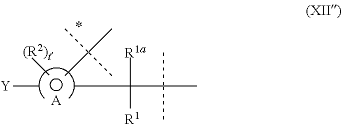

wherein
the unmarked dashed line indicates the attachment to the N⁺ of -D⁺, the dashed line marked with an asterisk indicates the attachment to -L²-;
—R¹, —R^{1a}—, -A-, —Y and R² are defined as in formula (XII); and
t′ is selected from the group consisting of 0, 1, 2, 3, 4 and 5.

In certain embodiments t′ of formula (XII″) is 0. In certain embodiments t′ of formula (XII″) is 1. In certain embodiments t′ of formula (XII″) is 2. In certain embodiments t′ of formula (XII″) is 3. In certain embodiments t′ of formula (XII″) is 4. In certain embodiments t′ of formula (XII″) is 5.

In certain embodiments -L¹- is of formula (XIII):

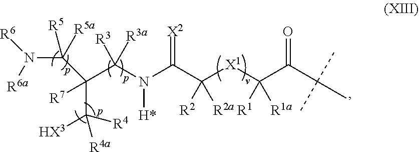

wherein
the dashed line indicates the attachment to the nitrogen of the primary or secondary amine of -D;
v is selected from the group consisting of 0 or 1;
—X¹- is selected from the group consisting of —C(R⁵)(R^{8a})—, —N(R⁹)— and —O—;
═X² is selected from the group consisting of ═O and ═N(R¹⁰);
—X³ is selected from the group consisting of —O, —S and —Se;
each p is independently selected from the group consisting of 0 or 1, provided that at most one p is 0;
—R⁶, —R^{6a}, —R¹⁰ are independently selected from the group consisting of —H, —C(R¹¹)(R^{11a})(R^{11b}) and -T;
—R⁹ is selected from the group consisting of —C(R¹¹)(R^{11a})(R^{11b}) and -T;
—R¹, —R^{1a}, —R², —R^{2a}, —R³, —R^{3a}, —R⁴, —R^{4a}, —R⁵, —R^{5a}, —R⁷, —R⁸, —R^{8a}, —R¹¹, —R^{11a} and —R^{11b} are independently selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
   —R¹², —R^{12a}, —R^{12b} are independently selected from the group consisting of —H, -T, C₁₋₆ alkyl, C₂₋₆ alkenyl and C₂₋₆ alkynyl; wherein -T, C₁₋₆ alkyl, C₂₋₆ alkenyl and C₂₋₆ alkynyl are optionally substituted with one or more —R¹³, which are the same or different and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
      wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
      wherein each T is independently optionally substituted with one or more —R¹³, which are the same or different;
   —R¹³ is selected from the group consisting of halogen, —CN, oxo, —C(O)OR ¹⁵, —OR¹⁵, —C(O)R¹⁵, —C(O)N(R¹⁵)(R^{15a}), —S(O)₂N(R¹⁵)(R^{15a}), —S(O) N(R¹⁵)(R^{15a}), —S(O)₂R¹⁵, —S(O)R¹⁵, —N(R¹⁵)S(O)₂N(R^{15a})(R^{15b}), —SR¹⁵, —N(R¹⁵)(R^{15a}), —NO₂, —OC(O)R¹⁵, —N(R¹⁵)C(O)R^{15a}, —N(R¹⁵)S(O)₂R^{15a}, —N(R¹⁵)S(O)R^{15a}, —N(R¹⁵)C(O)OR^{15a}, —N(R¹⁵)C(O)N(R^{15a})(R^{15b}), —OC(O)N(R¹⁵)(R^{15a}) and C₁₋₆ alkyl; wherein C₁₋₆ alkyl is optionally substituted with one or more halogen, which are the same or different;
      wherein —R¹⁴, —R^{14a}, —R¹⁵, —R^{15a} and —R^{15b} are independently selected from the group consisting of —H and C₁₋₆ alkyl; wherein C₁₋₆ alkyl is optionally substituted with one or more halogen, which are the same or different;
   optionally, one or more of the pairs —R¹/—R^{11a}, —R²/—R^{2a}, —R³/—R^{3a}, —R⁴/—R^{4a}, —R⁵/—R^{5a} or —R⁵/—R^{5a} are joined together with the atom to which they are attached to form a C₃₋₁₀ cycloalkyl, 3- to 10-membered heterocyclyl or an 8- to 11-membered heterobicyclyl;
   optionally, one or more of the pairs —R¹/—R², —R¹/—R⁸, —R¹/—R⁹, —R²/—R⁹ or —R²/—R¹⁰ are joined together with the atoms to which they are attached to form a ring -A-;
      wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
   optionally, one or more of the pairs —R³/—R⁶, —R⁴/—R⁶, —R⁵/—R⁶, —R⁶/—R^{6a} or —R⁶/—R⁷ form together with the atoms to which they are attached a ring -A′-;
      wherein -A′- is selected from the group consisting of 3- to
      10-membered heterocyclyl and 8- to 11-membered heterobicyclyl; and each -L¹- is substituted with at least one -L²- and optionally further substituted provided that the hydrogen marked with the asterisk in formula (XIII) is not replaced by a substituent.

It is understood that a moiety -L²-L¹-D is connected to Z through covalent attachment of -L²- to -Z. In certain embodiments -L²- is connected to Z through a stable covalent linkage. In certain embodiments -L¹- is connected to -L²- through a stable covalent linkage.

In certain embodiments all moieties -L²- of the conjugate of the present invention are identical. In certain embodiments a conjugate of the present invention comprises more than one type of -L²-, such as two, three or four different types of -L²-.

In the conjugate of the present invention -L²- is a chemical bond or a spacer moiety. In certain embodiments -L²- does not comprise a reversible linkage, i.e. all linkages in -L²- are stable linkages. In certain embodiments -L²- is connected to Z via a stable linkage.

In certain embodiments -L²- is chemical bond.

In certain embodiments -L²- is a spacer moiety.

In certain embodiments -L²- is a spacer moiety selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

In certain embodiments -L²- is a spacer moiety selected from -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{Y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

—R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C1-6 alkyl is optionally substituted with one or more halogen, which are the same or different.

In certain embodiments -L²- is a spacer moiety selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently selected from the group consisting of —H, -T, C₁₋₁₀ alkyl, C₂₋₁₀ alkenyl, and C₂₋₁₀ alkynyl;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;

each —R^{y2} is independently selected from the group consisting of halogen, and C₁₋₆ alkyl; and

each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is a C₁₋₂₀ alkyl chain, which is optionally interrupted by one or more groups independently selected from —O—, -T- and —C(O)N(R^{y1})—; and which C₁₋₂₀ alkyl chain is optionally substituted with one or more groups independently selected from —OH, -T and —C(O)N(R^{y6}R^{y6a}); wherein —R^{y1}, —R^{y6}, —R^{y6a} are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments -L²- has a molecular weight ranging from 14 g/mol to 750 g/mol.

In certain embodiments -L²- comprises a moiety selected from

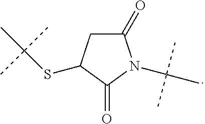

In certain embodiments -L²- has a chain length of 1 to 20 atoms.

As used herein the term “chain length” with regard to the moiety -L²- refers to the number of atoms of -L²- present in the shortest connection between -L¹- and -Z.

In certain embodiments a moiety -L¹-L²- is selected from the group consisting of

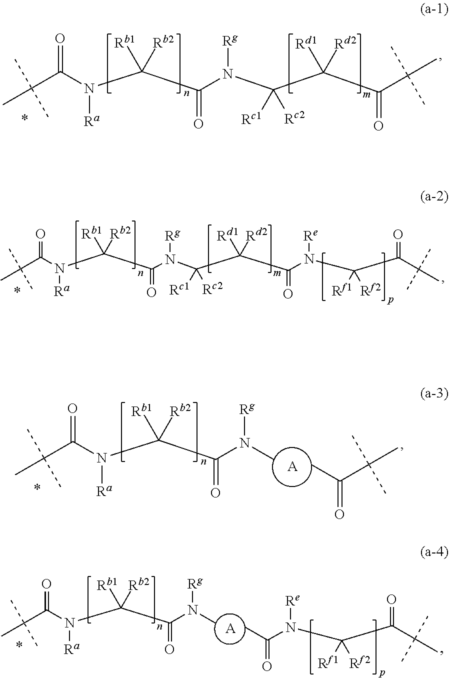

wherein
the dashed line marked with the asterisk indicates attachment to a π-electron-pair-donating heteroaromatic N of -D and the unmarked dashed line indicates attachment to Z, in particular to a nitrogen of an amine of Z;
—R^{a} each —R^{b1}, each —R^{b2}, —R^{c1}, —R^{c2}, each —R^{d1}, each —R^{d2}, —Re each —R^{f1}, each —R^{f2} and —R^{g} are independently selected from the group consisting of —H and C₁₋₆ alkyl;
n is an integer selected from the group consisting of 1, 2 and 3;
m is an integer selected from the group consisting of 0, 1, 2, 3, 4, 5, 6, 7, 8, 9 and 10;
p is an integer selected from the group consisting of 1, 2, 3, 4, 5, 6, 7, 8, 9, 10, 11, 12, 13, 14, 15, 16, 17, 18, 19 and 20;
A is a C₃₋₁₀ cycloalkyl; and
optionally, —R^{a} and the adjacent —R^{b1} are joined together with the atoms to which they are attached to form a ring -A*-, wherein -A*- is selected from the group consisting of 3- to 10-membered heterocyclyl and 8- to 11-membered heterobicyclyl.

In certain embodiments a moiety -L¹-L²- is of formula (a-1). In certain embodiments —R^{a} of formula (a-1) is selected from the group consisting of —H, methyl and ethyl. In certain embodiments —R^{a} of formula (a-1) is —H. In certain embodiments —R^{a} of formula (a-1) is methyl. In certain embodiments —R^{a} of formula (a-1) is ethyl. In certain embodiments n of formula (a-1) is selected from the group consisting of 1, 2 and 3. In certain embodiments n of formula (a-1) is selected from the group consisting of 1 and 2. In certain embodiments n of formula (a-1) is 1. In certain embodiments n of formula (a-1) is 2. In certain embodiments —R^{b1} is selected from the group consisting of —H, methyl and ethyl. In certain embodiments —R^{b1} of formula (a-1) is —H. In certain embodiments —R^{b1} of formula (a-1) is methyl. In certain embodiments —R^{b1} of formula (a-1) is ethyl. In certain embodiments —R^{b2} is selected from the group consisting of —H, methyl and ethyl. In certain embodiments —R^{b2} of formula (a-1) is —H. In certain embodiments —R^{b2} of formula (a-1) is methyl. In certain embodiments —R^{b2} of formula (a-1) is ethyl. In certain embodiments —R^{a} and —R^{b1} of formula (a-1) form a C₅ cycloalkyl. In certain embodiments n of formula (a-1) is 1 and —R^{a} and —R^{b1} of formula (a-1) form a C₅ cycloalkyl. In certain embodiments n of formula (a-1) is 1, —R^{a} and —R^{b1} of formula (a-1) form a C₅ cycloalkyl and —R^{b2} is —H. In certain embodiments —R^{g} is selected from the group consisting of —H, methyl and ethyl. In certain embodiments —R^{g} of formula (a-1) is —H. In certain embodiments —R^{g} of formula (a-1) is methyl. In certain embodiments —R^{c1} is selected from the group consisting of —H, methyl and ethyl. In certain embodiments —R^{c1} of formula (a-1) is —H. In certain embodiments —R^{c1} of formula (a-1) is methyl. In certain embodiments —R^{c1} of formula (a-1) is ethyl. In certain embodiments —R^{c2} is selected from the group consisting of —H, methyl and ethyl. In certain embodiments —R^{c2} of formula (a-1) is —H. In certain embodiments —R^{c2} of formula (a-1) is methyl. In certain embodiments —R^{c2} of formula (a-1) is ethyl. In certain embodiments —R^{d1} is selected from the group consisting of —H, methyl and ethyl. In certain embodiments —R^{d1} of formula (a-1) is —H. In certain embodiments —R^{d1} of formula (a-1) is methyl. In certain embodiments —R^{d1} of formula (a-1) is ethyl. In certain embodiments —R^{d2} is selected from the group consisting of —H, methyl and ethyl. In certain embodiments —R^{d2} of formula (a-1) is —H. In certain embodiments —R^{d2} of formula (a-1) is methyl. In certain embodiments —R^{d2} of formula (a-1) is ethyl. In certain embodiments m of formula (a-1) is selected from the group consisting of 0, 1, 2, 3, 4, 5 and 6. In certain embodiments m of formula (a-1) is 0. In certain embodiments m of formula (a-1) is 1. In certain embodiments m of formula (a-1) is 2. In certain embodiments m of formula (a-1) is 4. In certain embodiments m of formula (a-1) is 5. In certain embodiments m of formula (a-1) is 6.

In certain embodiments a moiety -L¹-L²- is of formula (a-2). In certain embodiments —R^{a} of formula (a-2) is selected from the group consisting of —H, methyl and ethyl. In certain embodiments —R^{a} of formula (a-2) is —H. In certain embodiments —R^{a} of formula (a-2) is methyl. In certain embodiments —R^{a} of formula (a-2) is ethyl. In certain embodiments n of formula (a-2) is selected from the group consisting of 1, 2 and 3. In certain embodiments n of formula (a-2) is selected from the group consisting of 1 and 2. In certain embodiments n of formula (a-2) is 1. In certain embodiments n of formula (a-2) is 2. In certain embodiments —R^{b1} is selected from the group consisting of —H, methyl and ethyl. In certain embodiments —R^{b1} of formula (a-2) is —H. In certain embodiments —R^{b1} of formula (a-2) is methyl. In certain embodiments —R^{b1} of formula (a-2) is ethyl. In certain embodiments —R^{b2} is selected from the group consisting of —H, methyl and ethyl. In certain embodiments —R^{b2} of formula (a-2) is —H. In certain embodiments —R^{b2} of formula (a-2) is methyl. In certain embodiments —R^{b2} of formula (a-2) is ethyl. In certain embodiments —R^{a} and —R^{b1} of formula (a-2) form a C₅ cycloalkyl. In certain embodiments n of formula (a-2) is 1 and —R^{a} and —R^{b1} of formula (a-2) form a C₅ cycloalkyl. In certain embodiments n of formula (a-2) is 1, —R^{a} and —R^{b1} of formula (a-2) form a C₅ cycloalkyl and —R^{b2} is —H. In certain embodiments —R^{g} is selected from the group consisting of —H, methyl and ethyl. In certain embodiments —R^{g} of formula (a-2) is —H. In certain embodiments —R^{g} of formula (a-2) is methyl. In certain embodiments —R^{c1} is selected from the group consisting of —H, methyl and ethyl. In certain embodiments —R¹ of formula (a-2) is —H. In certain embodiments —R^{c1} of formula (a-2) is methyl. In certain embodiments —R^{c1} of formula (a-2) is ethyl. In certain embodiments —R^{c2} is selected from the group consisting of —H, methyl and ethyl. In certain embodiments —R^{c2} of formula (a-2) is —H. In certain embodiments —R^{c2} of formula (a-2) is methyl. In certain embodiments —R^{c2} of formula (a-2) is ethyl. In certain embodiments —R^{d1} is selected from the group consisting of —H, methyl and ethyl. In certain embodiments —R^{d1} of formula (a-2) is —H. In certain embodiments —R^{d1} of formula (a-2) is methyl. In certain embodiments —R^{d1} of formula (a-2) is ethyl. In certain embodiments —R^{d2} is selected from the group consisting of —H, methyl and ethyl. In certain embodiments —R^{d2} of formula (a-2) is —H. In certain embodiments —R^{d2} of formula (a-2) is methyl. In certain embodiments —R^{d2} of formula (a-2) is ethyl. In certain embodiments m of formula (a-2) is selected from the group consisting of 0, 1, 2, 3, 4, 5 and 6. In certain embodiments m of formula (a-2) is 0. In certain embodiments m of formula (a-2) is 1. In certain embodiments m of formula (a-2) is 2. In certain embodiments m of formula (a-2) is 4. In certain embodiments m of formula (a-2) is 5. In certain embodiments m of formula (a-2) is 6. In certain embodiments —R^{e} is selected from the group consisting of —H, methyl and ethyl. In certain embodiments —R^{e} of formula (a-2) is —H. In certain embodiments —R^{e} of formula (a-2) is methyl. In certain embodiments —R^{e} of formula (a-2) is ethyl. In certain embodiments p of formula (a-2) is selected from the group consisting of 0, 1, 2, 3, 4, 5 and 6. In certain embodiments p of formula (a-2) is 0. In certain embodiments p of formula (a-2) is 1. In certain embodiments p of formula (a-2) is 2. In certain embodiments p of formula (a-2) is 4. In certain embodiments p of formula (a-2) is 5. In certain embodiments p of formula (a-2) is 6. In certain embodiments —R^{f1} is selected from the group consisting of —H, methyl and ethyl. In certain embodiments —R^{f1} of formula (a-2) is —H. In certain embodiments —R^{f1} of formula (a-2) is methyl. In certain embodiments —R¹ of formula (a-2) is ethyl. In certain embodiments —R^{f} is selected from the group consisting of —H, methyl and ethyl. In certain embodiments —R^{f2} of formula (a-2) is —H. In certain embodiments —R^{f} of formula (a-2) is methyl. In certain embodiments —R^{f} of formula (a-2) is ethyl.

In certain embodiments a moiety -L¹-L²- is of formula (a-3). In certain embodiments —R^{a} of formula (a-3) is selected from the group consisting of —H, methyl and ethyl. In certain embodiments —R^{a} of formula (a-3) is —H. In certain embodiments —R^{a} of formula (a-3) is methyl. In certain embodiments —R^{a} of formula (a-3) is ethyl. In certain embodiments n of formula (a-3) is selected from the group consisting of 1, 2 and 3. In certain embodiments n of formula (a-3) is selected from the group consisting of 1 and 2. In certain embodiments n of formula (a-3) is 1. In certain embodiments n of formula (a-3) is 2. In certain embodiments —R^{b1} is selected from the group consisting of —H, methyl and ethyl. In certain embodiments —R^{b1} of formula (a-3) is —H. In certain embodiments —R^{b1} of formula (a-3) is methyl. In certain embodiments —R^{b1} of formula (a-3) is ethyl. In certain embodiments —R^{b2} is selected from the group consisting of —H, methyl and ethyl. In certain embodiments —R^{b2} of formula (a-3) is —H. In certain embodiments —R^{b2} of formula (a-3) is methyl. In certain embodiments —R^{b2} of formula (a-3) is ethyl. In certain embodiments —R^{a} and —R^{b1} of formula (a-3) form a C₅ cycloalkyl. In certain embodiments n of formula (a-3) is 1 and —R^{a} and —R^{b1} of formula (a-3) form a C₅ cycloalkyl. In certain embodiments n of formula (a-3) is 1, —R^{a} and —R^{b1} of formula (a-3) form a C₅ cycloalkyl and —R^{b2} is —H. In certain embodiments —R^{g} is selected from the group consisting of —H, methyl and ethyl. In certain embodiments —R^{g} of formula (a-3) is —H. In certain embodiments —R^{g} of formula (a-3) is methyl. In certain embodiments A of formula (a-3) is C₅ cycloalkyl. In certain embodiments A of formula (a-3) is C₆ cycloalkyl.

In certain embodiments a moiety -L¹-L²- is of formula (a-4). In certain embodiments —R^{a} of formula (a-4) is selected from the group consisting of —H, methyl and ethyl. In certain embodiments —R^{a} of formula (a-4) is —H. In certain embodiments —R^{a} of formula (a-4) is methyl. In certain embodiments —R^{a} of formula (a-4) is ethyl. In certain embodiments n of formula (a-4) is selected from the group consisting of 1, 2 and 3. In certain embodiments n of formula (a-4) is selected from the group consisting of 1 and 2. In certain embodiments n of formula (a-4) is 1. In certain embodiments n of formula (a-4) is 2. In certain embodiments —R^{b1} is selected from the group consisting of —H, methyl and ethyl. In certain embodiments —R^{b1} of formula (a-4) is —H. In certain embodiments —R^{b1} of formula (a-4) is methyl. In certain embodiments —R^{b1} of formula (a-4) is ethyl. In certain embodiments —R^{b2} is selected from the group consisting of —H, methyl and ethyl. In certain embodiments —R^{b2} of formula (a-4) is —H. In certain embodiments —R^{b2} of formula (a-4) is methyl. In certain embodiments —R^{b2} of formula (a-4) is ethyl. In certain embodiments —R^{a} and —R^{b1} of formula (a-4) form a C₅ cycloalkyl. In certain embodiments n of formula (a-4) is 1 and —R^{a} and —R^{b1} of formula (a-4) form a C₅ cycloalkyl. In certain embodiments n of formula (a-4) is 1, —R^{a} and —R^{b1} of formula (a-4) form a C₅ cycloalkyl and —R^{b2} is —H. In certain embodiments —R^{g} is selected from the group consisting of —H, methyl and ethyl. In certain embodiments —R^{g} of formula (a-4) is —H. In certain embodiments —R⁹ of formula (a-4) is methyl. In certain embodiments A of formula (a-4) is C₅ cycloalkyl. In certain embodiments A of formula (a-4) is C₆ cycloalkyl. In certain embodiments —R^{e} is selected from the group consisting of —H, methyl and ethyl. In certain embodiments —R^{e} of formula (a-4) is —H. In certain embodiments —R^{e} of formula (a-4) is methyl. In certain embodiments —R^{e} of formula (a-4) is ethyl. In certain embodiments p of formula (a-4) is selected from the group consisting of 0, 1, 2, 3, 4, 5 and 6. In certain embodiments p of formula (a-4) is 0. In certain embodiments p of formula (a-4) is 1. In certain embodiments p of formula (a-4) is 2. In certain embodiments p of formula (a-4) is 4. In certain embodiments p of formula (a-4) is 5. In certain embodiments p of formula (a-4) is 6. In certain embodiments —R^{f} is selected from the group consisting of —H, methyl and ethyl. In certain embodiments —R^{f1} of formula (a-4) is —H. In certain embodiments —R^{f1} of formula (a-4) is methyl. In certain embodiments —R^{f1} of formula (a-4) is ethyl. In certain embodiments —R² is selected from the group consisting of —H, methyl and ethyl. In certain embodiments —R² of formula (a-4) is —H. In certain embodiments —R¹ of formula (a-4) is methyl. In certain embodiments —R^{f} of formula (a-4) is ethyl.

In certain embodiments a moiety -L¹-L²- is selected from the group consisting of

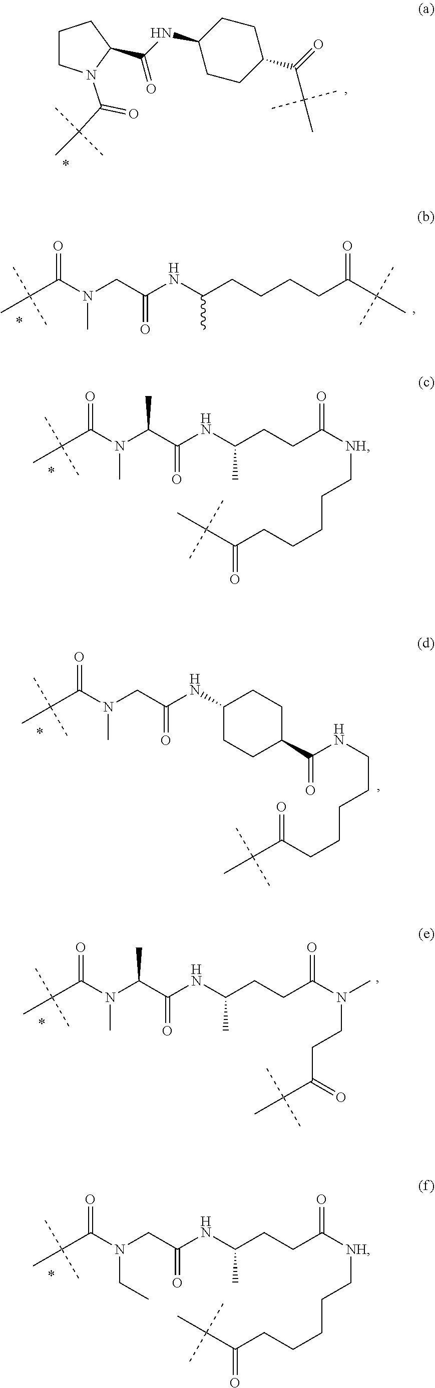

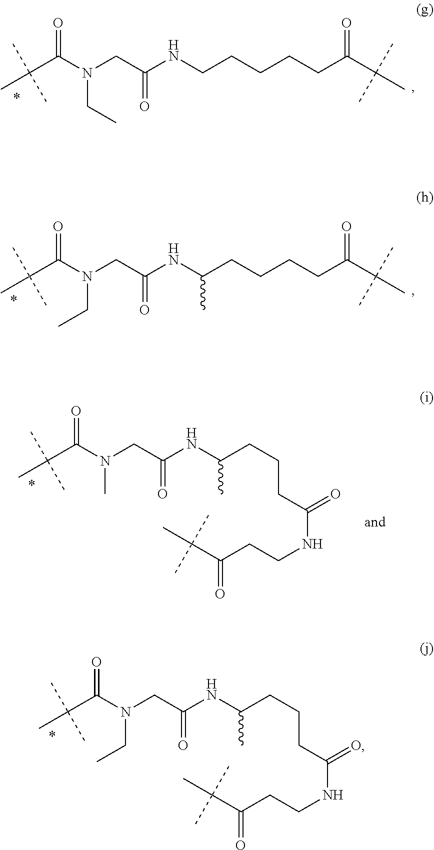

wherein
the dashed line marked with the asterisk indicates attachment to a π-electron-pair-donating heteroaromatic N of -D and the unmarked dashed line indicates attachment to Z, in particular to a nitrogen of an amine of Z.

In certain embodiments the moiety -L¹-L²- has the structure of formula (a). In certain embodiments the moiety -L¹-L²- has the structure of formula (b). In certain embodiments the moiety -L¹-L²- has the structure of formula (c). In certain embodiments the moiety -L¹-L²- has the structure of formula (d). In certain embodiments the moiety -L¹-L²- has the structure of formula (e). In certain embodiments the moiety -L¹-L²- has the structure of formula (f. In certain embodiments the moiety -L¹-L²- has the structure of formula (g). In certain embodiments the moiety -L¹-L²- has the structure of formula (h). In certain embodiments the moiety -L¹-L²- has the structure of formula (i). In certain embodiments the moiety -L¹-L²- has the structure of formula (j).

In certain embodiments the dashed line marked with the asterisk in formula (a), (b), (c), (d), (e), (f), (g), (h), (i) and (j) indicates attachment to a π-electron-pair-donating heteroaromatic N of axitinib. In certain embodiments the unmarked dashed line in formula (a), (b), (c), (d), (e), (f), (g), (h), (i) and (j) indicates attachment to a hydrogel, in particular to a PEG-based hydrogel.

In certain embodiments Z comprises a polymer.

In certain embodiments Z is not degradable. In certain embodiments Z is degradable. A degradable moiety Z has the effect that the carrier moiety degrades over time which may be advantageous in certain applications.

In certain embodiments Z is a hydrogel. Such hydrogel may be degradable or non-degradable, i.e. stable. In certain embodiments such hydrogel is degradable. In certain embodiments such hydrogel is non-degradable.

In certain embodiments such hydrogel Z comprises a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(alkylene glycols), such as poly(ethylene glycols) and poly(propylene glycol), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments Z is a poly(alkylene glycol)-based hydrogel, such as a poly(propylene glycol)-based hydrogel or a poly(ethylene glycol)-based (PEG-based) hydrogel, or a hyaluronic acid-based hydrogel. In certain embodiments such hydrogel is degradable. In certain embodiments such hydrogel is non-degradable, i.e. stable.

In certain embodiments Z is a PEG-based hydrogel. Suitable hydrogels are known in the art. Examples are WO2006/003014, WO2011/012715 and WO2014/056926, which are herewith incorporated by reference.

In certain embodiments such PEG-based hydrogel comprises a plurality of backbone moieties that are crosslinked via crosslinker moieties -CL^{p}-. Optionally, there is a spacer moiety -SP¹- between a backbone moiety and a crosslinker moiety. In certain embodiments such spacer -SP¹- is defined as described above for -L²-.

In certain embodiments a backbone moiety has a molecular weight ranging from 1 kDa to 20 kDa.

In certain embodiments a backbone moiety is of formula (A)

B*-(A-Hyp)ₓ (A),
wherein
B* is a branching core,
A is a PEG-based polymer,
Hyp is a branched moiety,
x is an integer of from 3 to 16;
and wherein each backbone moiety is connected to one or more crosslinker moieties and to one or more moieties -L²-, which crosslinker moieties and moieties -L²- are connected to Hyp, either directly or through a spacer moiety -SP¹-.

In certain embodiments B* of formula (A) is selected from the group consisting of polyalcohol moieties and polyamine moieties. In certain embodiments B* of formula (A) is a polyalcohol moiety. In certain embodiments B* of formula (A) is a polyamine moiety.

In certain embodiments the polyalcohol moieties for B* of formula (A) are selected from the group consisting of a pentaerythritol moiety, tripentaerythritol moiety, hexaglycerine moiety, sucrose moiety, sorbitol moiety, fructose moiety, mannitol moiety and glucose moiety. In certain embodiments B* of formula (A) is a pentaerythritol moiety, i.e. a moiety of formula

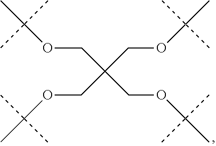

wherein dashed lines indicate attachment to -A-.

In certain embodiments the polyamine moieties for B* of formula (A) is selected from the group consisting of an ornithine moiety, diaminobutyric acid moiety, trilysine moiety, tetralysine moiety, pentalysine moiety, hexalysine moiety, heptalysine moiety, octalysine moiety, nonalysine moiety, decalysine moiety, undecalysine moiety, dodecalysine moiety, tridecalysine moiety, tetradecalysine moiety and pentadecalysine moiety. In certain embodiments B* of formula (A) is selected from the group consisting of an ornithine moiety, diaminobutyric acid moiety and a trilysine moiety.

A backbone moiety of formula (A) may consist of the same or different PEG-based moieties -A- and each moiety -A- may be chosen independently. In certain embodiments all moieties -A- present in a backbone moiety of formula (A) have the same structure. It is understood that the phrase “have the same structure” with regard to polymeric moieties, such as with regard to the PEG-based polymer -A-, means that the number of monomers of the polymer, such as the number of ethylene glycol monomers, may vary due to the polydisperse nature of polymers. In certain embodiments the number of monomer units does not vary by more than a factor of 2 between all moieties -A- of a hydrogel.

In certain embodiments each -A- of formula (A) has a molecular weight ranging from 0.3 kDa to 40 kDa; e.g. from 0.4 to 30 kDa, from 0.4 to 25 kDa, from 0.4 to 20 kDa, from 0.4 to 15 kDa, from 0.4 to 10 kDa or from 0.4 to 5 kDa. In certain embodiments each -A- has a molecular weight from 0.4 to 5 kDa. In certain embodiments -A- has a molecular weight of about 0.5 kDa. In certain embodiments -A- has a molecular weight of about 1 kDa. In certain embodiments -A- has a molecular weight of about 2 kDa. In certain embodiments -A- has a molecular weight of about 3 kDa. In certain embodiments -A- has a molecular weight of about 5 kDa.

In certain embodiments -A- of formula (A) is of formula (A-i)

—(CH₂)ₙ₁(OCH₂CH₂)ₙX— (A-i),
wherein
n1 is 1 or 2;
n is an integer ranging from 3 to 250, such as from 5 to 200, such as from 8 to 150 or from 10 to 100; and
X is a chemical bond or a linkage covalently linking A and Hyp.

In certain embodiments -A- of formula (A) is of formula (A-i)

—(CH₂)ₙ₁(OCH₂CH₂)ₙ—(CH₂)ₙ₂X— (A-i),
wherein
n1 is 1 or 2;
n is an integer ranging from 3 to 250, such as from 5 to 200, such as from 8 to 150 or from 10 to 100;
n2 is 0 or 1; and
X is a chemical bond or a linkage covalently linking A and Hyp.

In certain embodiments -A- of formula (A) is of formula (A-iii)

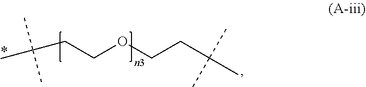

wherein
the dashed line marked with the asterisk indicates attachment to B*,
the unmarked dashed line indicates attachment to -Hyp; and
n3 is an integer ranging from 10 to 50.

In certain embodiments n3 of formula (A-iii) is 25. In certain embodiments n3 of formula (A-iii) is 26. In certain embodiments n3 of formula (A-iii) is 27. In certain embodiments n3 of formula (A-iii) is 28. In certain embodiments n3 of formula (A-iii) is 29. In certain embodiments n3 of formula (A-iii) is 30.

In certain embodiments a moiety B*-(A)₄ is of formula (A-iv)

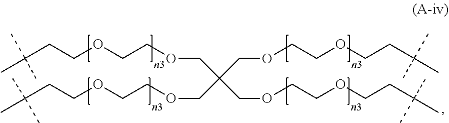

wherein
dashed lines indicate attachment to Hyp; and
each n3 is independently an integer selected from 10 to 50.

In certain embodiments n3 of formula (A-iv) is 25. In certain embodiments n3 of formula (A-iv) is 26. In certain embodiments n3 of formula (A-iv) is 27. In certain embodiments n3 of formula (B-a) is 28. In certain embodiments n3 of formula (A-iv) is 29. In certain embodiments n3 of formula (A-iv) is 30.

A backbone moiety of formula (A) may consist of the same or different dendritic moieties -Hyp and that each -Hyp can be chosen independently. In certain embodiments all moieties -Hyp present in a backbone moiety of formula (A) have the same structure.

In certain embodiments each -Hyp of formula (A) has a molecular weight ranging from 0.3 kDa to 5 kDa.

In certain embodiments -Hyp is selected from the group consisting of a moiety of formula (A-va)

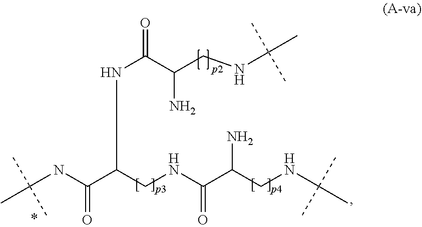

wherein
the dashed line marked with the asterisk indicates attachment to -A-,
the unmarked dashed lines indicate attachment to a spacer moiety -SP¹, a crosslinker moiety -CL^{p}- or to -L²-; and
p2, p3 and p4 are identical or different and each is independently of the others an integer from 1 to 5;

a moiety of formula (A-vb)

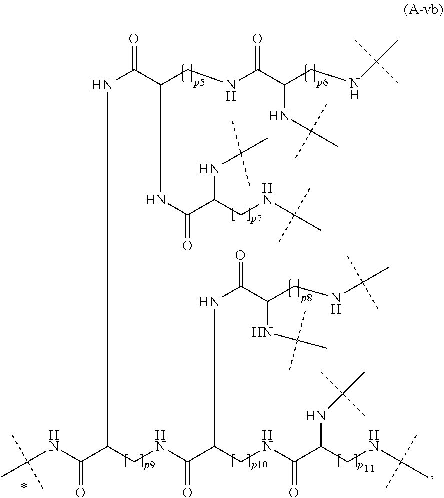

wherein
the dashed line marked with the asterisk indicates attachment to -A-,
the unmarked dashed lines indicate attachment to a spacer moiety -SP¹, a crosslinker moiety -CL^{p}- or to -L²-; and
p5 to p11 are identical or different and each is independently of the others an integer from 1 to 5;

a moiety of formula (A-vc)

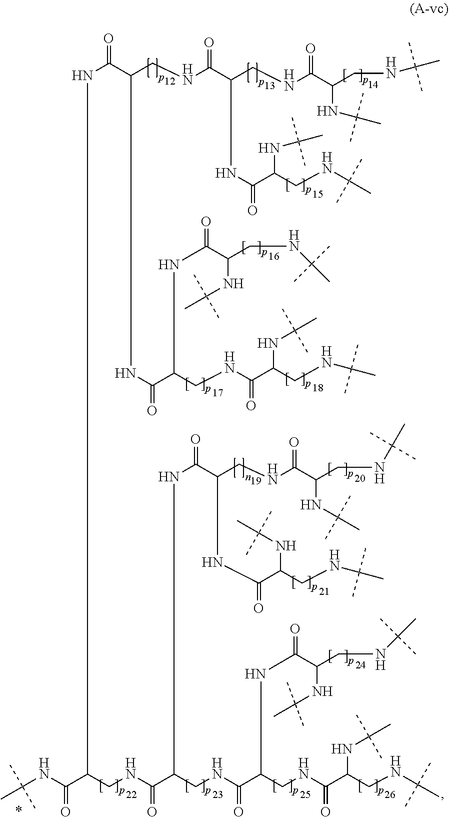

wherein
the dashed line marked with the asterisk indicates attachment to -A-, the unmarked dashed lines indicate attachment to a spacer moiety -SP¹-, a crosslinker moiety -CL^{p}- or to -L²-; and
p12 to p26 are identical or different and each is independently of the others an integer from 1 to 5; and

a moiety of formula (A-vd)

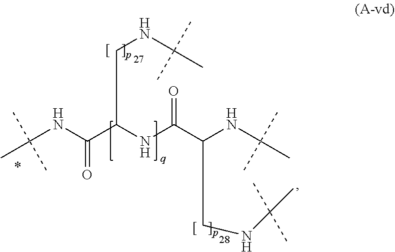

wherein
the dashed line marked with the asterisk indicates attachment to -A-,
the unmarked dashed lines indicate attachment to a spacer moiety -SP¹, a crosslinker moiety -CL^{p}- or to -L²-;
p27 and p28 are identical or different and each is independently of the other an integer from 1 to 5; and
q is an integer from 1 to 8;

wherein the moieties (A-va) to (A-vd) may at each chiral center be in either R- or S-configuration.

In certain embodiments all chiral centers of a moiety (A-va), (A-vb), (A-vc) or (A-vd) are in the same configuration. In certain embodiments all chiral centers of a moiety (A-va), (A-vb), (A-vc) or (A-vd) are in R-configuration. In certain embodiments all chiral centers of a moiety (A-va), (A-vb), (A-vc) or (A-vd) are in S-configuration.

In certain embodiments p2, p3 and p4 of formula (A-va) are 4.

In certain embodiments p5 to p11 of formula (A-vb) are 4.

In certain embodiments p12 to p26 of formula (A-vc) are 4.

In certain embodiments q of formula (A-vd) is 2 or 6. In certain embodiments q of formula (A-vd) q is 6.

In certain embodiments p27 and p28 of formula (A-vd) are 4.

In certain embodiments -Hyp of formula (A) comprises a branched polypeptide moiety.

In certain embodiments -Hyp of formula (A) comprises a lysine moiety. In certain embodiments each -Hyp of formula (A) is independently selected from the group consisting of a trilysine moiety, tetralysine moiety, pentalysine moiety, hexalysine moiety, heptalysine moiety, octalysine moiety, nonalysine moiety, decalysine moiety, undecalysine moiety, dodecalysine moiety, tridecalysine moiety, tetradecalysine moiety, pentadecalysine moiety, hexadecalysine moiety, heptadecalysine moiety, octadecalysine moiety and nonadecalysine moiety.

In certain embodiments -Hyp comprises 3 lysine moieties. In certain embodiments -Hyp comprises 7 lysine moieties. In certain embodiments -Hyp comprises 15 lysine moieties. In certain embodiments -Hyp comprises heptalysinyl.

In certain embodiments x of formula (A) is 3. In certain embodiments x of formula (A) is 4. In certain embodiments x of formula (A) is 6. In certain embodiments x of formula (A) is 8.

In certain embodiments the backbone moiety is of formula (A-vi)

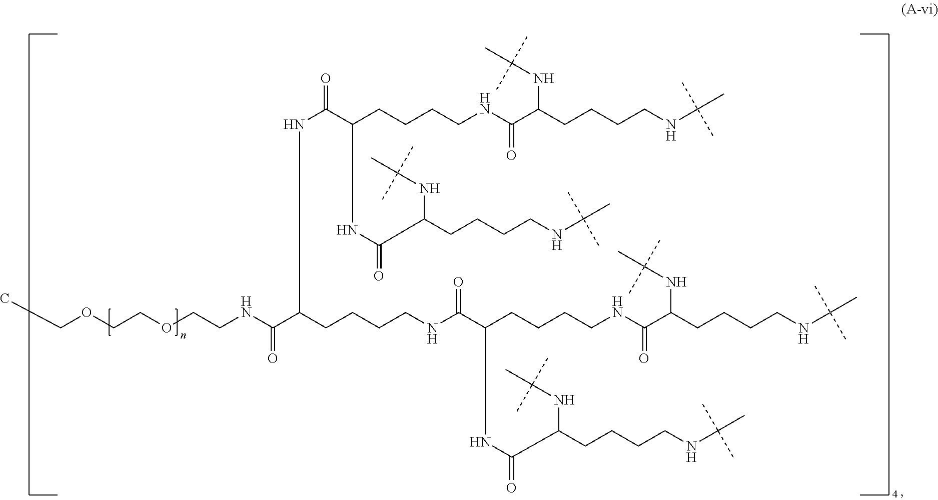

wherein
dashed lines indicate attachment to a spacer moiety -SP¹-, a crosslinker moiety -CL^{p}- or to -L²-; and
n ranges from 10 to 40.

In certain embodiments n of formula (A-vi) is about 28.

In certain embodiments the backbone moiety is of formula (A-vi), wherein n ranges from 100 to 140 and is about 113 and wherein the lysine moieties may be in either D- or L-conformation.

In certain embodiments the backbone moiety is of formula (A-vii)

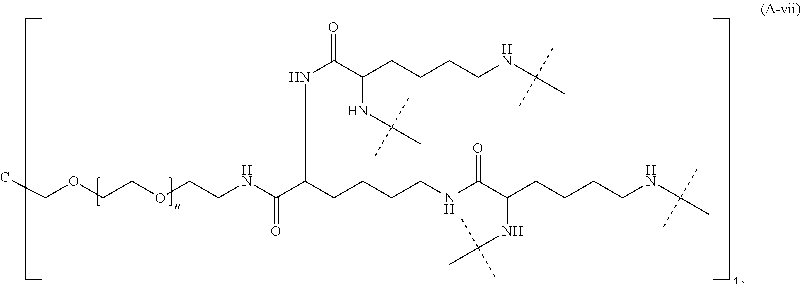

wherein
dashed lines indicate attachment to a spacer moiety -SP¹, a crosslinker moiety -CL^{p}- or to -L²-; and
n ranges from 10 to 40.

In certain embodiments there is no spacer moiety -SP¹- between a backbone moiety and a crosslinker moiety -CL^{p}-, i.e. -CL^{p}- is directly linked to -Hyp.

The crosslinker -CL^{p}- of the PEG-based hydrogel is in certain embodiments poly(alkylene glycol) (PAG)-based. In certain embodiments the crosslinker is poly(propylene glycol)-based. In certain embodiments the crosslinker -CL^{p}- is PEG-based.

In certain embodiments such PAG-based crosslinker moiety -CL^{p}- is of formula (A-viii)

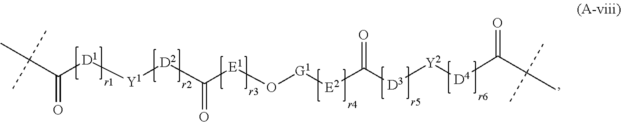

wherein
dashed lines indicate attachment to a backbone moiety or to a spacer moiety -SP¹;
-Y¹— is of formula

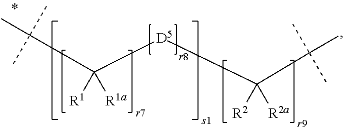

wherein the dashed line marked with the asterisk indicates attachment to -D¹- and the unmarked dashed line indicates attachment to -D²-;
-Y²- is of formula

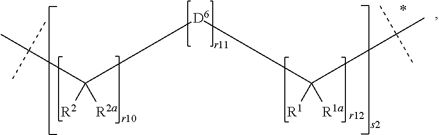

wherein the dashed line marked with the asterisk indicates attachment to -D⁴- and the unmarked dashed line indicates attachment to -D³-;
-E¹- is of formula

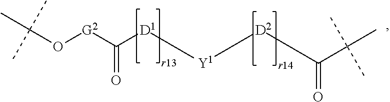

wherein the dashed line marked with the asterisk indicates attachment to —(C═O)— and the unmarked dashed line indicates attachment to —O—;
-E²- is of formula

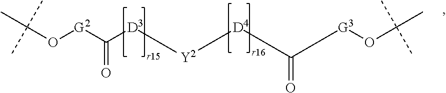

wherein the dashed line marked with the asterisk indicates attachment to -G¹- and the unmarked dashed line indicates attachment to —(C═O)—;
-G¹- is of formula

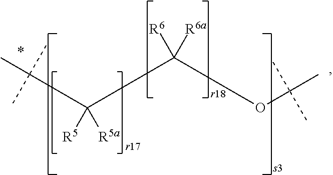

wherein the dashed line marked with the asterisk indicates attachment to —O— and the unmarked dashed line indicates attachment to -E²-;
-G²- is of formula

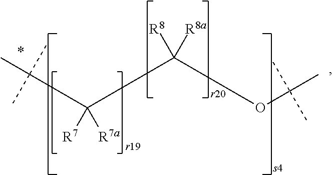

wherein the dashed line marked with the asterisk indicates attachment to —O— and the unmarked dashed line indicates attachment to —(C═O)—;
-G³- is of formula

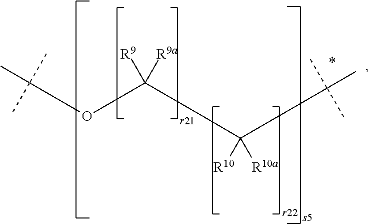

wherein the dashed line marked with the asterisk indicates attachment to —O— and the unmarked dashed line indicates attachment to —(C═O)—;
D¹-, -D²-, -D³-, -D⁴-, -D⁵- and -D⁶- are identical or different and each is independently of the others selected from the group comprising —O—, —NR¹¹—, —N⁺R¹²R^{12a}—, —S—, —(S═O)—, —(S(O)₂)—, —C(O)—, —P(O)R¹³—, —P(O)(OR¹³) and —CR¹⁴R^{14a}—;
—R¹, —R^{1a}, —R², —R^{2a}, —R³, —R^{3a}, —R⁴, —R^{4a}, —R⁵, —R^{5a}, —R⁶, —R^{6a}, —R⁷, —R^{7a}, —R⁸, —R^{8a}, —R⁹, —R^{9a}, —R¹⁰, —R^{10a}, —R¹¹, —R¹, —R^{12a}, —R¹³, —R¹⁴ and —R^{14a} are identical or different and each is independently of the others selected from the group consisting of —H and C₁₋₆ alkyl;
optionally, one or more of the pairs —R¹/—R^{1a}, —R²/—R^{2a}, —R³/—R^{3a}, —R⁴/—R^{4a}, —R¹/—R², —R³/—R⁴, —R^{1a}/—R^{2a}, —R^{3a}/—R^{4a}, —R²/—R^{12a}, and —R¹⁴/—R^{14a} form a chemical bond or are joined together with the atom to which they are attached to form a C₃₋₈ cycloalkyl or to form a ring A or are joined together with the atom to which they are attached to form a 4- to 7-membered heterocyclyl or 8- to 11-membered heterobicyclyl or adamantyl;
A is selected from the group consisting of phenyl, naphthyl, indenyl, indanyl and tetralinyl;
r1, r2, r5, r6, r13, r14, r15 and r16 are independently 0 or 1;
r3, r4, r7, r8, r9, r10, r11, r12 are independently 0, 1, 2, 3, or 4;
r17, r18, r19, r20, r21 and r22 are independently 1, 2, 3, 4, 5, 6, 7, 8, 9 or 10;
s1, s2, s4, s5 are independently 1, 2, 3, 4, 5 or 6; and
s3 ranges from 1 to 900.

In certain embodiments s3 ranges from 1 to 500. In certain embodiments s3 ranges from 1 to 200.

In certain embodiments r1 of formula (A-viii) is 0. In certain embodiments r1 of formula (A-viii) is 1. In certain embodiments r2 of formula (A-viii) is 0. In certain embodiments r2 of formula (A-viii) is 1. In certain embodiments r5 of formula (A-viii) is 0. In certain embodiments r5 of formula (A-viii) is 1.

In certain embodiments r1, r2, r5 and r6 of formula (A-viii) are 0.

In certain embodiments r6 of formula (A-viii) is 0. In certain embodiments r6 of formula (A-viii) is 1. In certain embodiments r13 of formula (A-viii) is 0. In certain embodiments r13 of formula (A-viii) is 1. In certain embodiments r14 of formula (A-viii) is 0. In certain embodiments r14 of formula (A-viii) is 1. In certain embodiments r15 of formula (A-viii) is 0. In certain embodiments r15 of formula (A-viii) is 1. In certain embodiments r16 of formula (A-viii) is 0. In certain embodiments r16 of formula (A-viii) is 1.

In certain embodiments r3 of formula (A-viii) is 1. In certain embodiments r3 of formula (A-viii) is 2. In certain embodiments r4 of formula (A-viii) is 1. In certain embodiments r4 of formula (A-viii) is 2. In certain embodiments r3 and r4 of formula (A-viii) are both 1. In certain embodiments r3 and r4 of formula (A-viii) are both 2. In certain embodiments r3 and r4 of formula (A-viii) are both 3.

In certain embodiments r7 of formula (A-viii) is 0. In certain embodiments r7 of formula (A-viii) is 1. In certain embodiments r7 of formula (A-viii) is 2. In certain embodiments r8 of formula (A-viii) is 0. In certain embodiments r8 of formula (A-viii) is 1. In certain embodiments r8 of formula (A-viii) is 2. In certain embodiments r9 of formula (A-viii) is 0. In certain embodiments r9 of formula (A-viii) is 1. In certain embodiments r9 of formula (A-viii) is 2. In certain embodiments r10 of formula (A-viii) is 0. In certain embodiments r10 of formula (A-viii) is 1. In certain embodiments r10 of formula (A-viii) is 2. In certain embodiments r11 of formula (A-viii) is 0. In certain embodiments r11 of formula (A-viii) is 1. In certain embodiments r11 of formula (A-viii) is 2. In certain embodiments r12 of formula (A-viii) is 0. In certain embodiments r12 of formula (A-viii) is 1. In certain embodiments r12 of formula (A-viii) is 2.

In certain embodiments r17 of formula (A-viii) is 1. In certain embodiments r18 of formula (A-viii) is 1. In certain embodiments r19 of formula (A-viii) is 1. In certain embodiments r20 of formula (A-viii) is 1. In certain embodiments r21 of formula (A-viii) is 1.

In certain embodiments s1 of formula (A-viii) is 1. In certain embodiments s1 of formula (A-viii) is 2. In certain embodiments s2 of formula (A-viii) is 1. In certain embodiments s2 of formula (A-viii) is 2. In certain embodiments s4 of formula (A-viii) is 1. In certain embodiments s4 of formula (A-viii) is 2.

In certain embodiments s3 of formula (A-viii) ranges from 5 to 500. In certain embodiments s3 of formula (A-viii) ranges from 10 to 250. In certain embodiments s3 of formula (A-viii) ranges from 12 to 150. In certain embodiments s3 of formula (A-viii) ranges from 15 to 100. In certain embodiments s3 of formula (A-viii) ranges from 18 to 75. In certain embodiments s3 of formula (A-viii) ranges from 20 to 50.

In certain embodiments —R¹ of formula (A-viii) is —H. In certain embodiments —R¹ of formula (A-viii) is methyl. In certain embodiments —R¹ of formula (A-viii) is ethyl. In certain embodiments —R^{1a} of formula (A-viii) is —H. In certain embodiments —R^{1a} of formula (A-viii) is methyl. In certain embodiments —R^{1a} of formula (A-viii) is ethyl. In certain embodiments —R² of formula (A-viii) is —H. In certain embodiments —R² of formula (A-viii) is methyl. In certain embodiments —R² of formula (A-viii) is ethyl. In certain embodiments —R^{2a} of formula (A-viii) is —H. In certain embodiments —R^{2a} of formula (A-viii) is methyl. In certain embodiments —R^{2a} of formula (A-viii) is ethyl. In certain embodiments —R³ of formula (A-viii) is —H. In certain embodiments —R³ of formula (A-viii) is methyl. In certain embodiments —R³ of formula (A-viii) is ethyl. In certain embodiments —R^{3a} of formula (A-viii) is —H. In certain embodiments —R^{3a} of formula (A-viii) is methyl. In certain embodiments —R^{3a} of formula (A-viii) is ethyl. In certain embodiments —R⁴ of formula (A-viii) is —H. In certain embodiments —R⁴ of formula (A-viii) is methyl. In certain embodiments —R⁴ of formula (A-viii) is methyl. In certain embodiments —R^{4a} of formula (A-viii) is —H. In certain embodiments —R^{4a} of formula (A-viii) is methyl. In certain embodiments —R^{4a} of formula (A-viii) is ethyl. In certain embodiments —R⁵ of formula (A-viii) is —H. In certain embodiments —R⁵ of formula (A-viii) is methyl. In certain embodiments —R⁵ of formula (A-viii) is ethyl. In certain embodiments —R^{5a} of formula (A-viii) is —H. In certain embodiments —R^{5a} of formula (A-viii) is methyl. In certain embodiments —R^{5a} of formula (A-viii) is ethyl. In certain embodiments —R⁶ of formula (A-viii) is —H. In certain embodiments —R⁶ of formula (A-viii) is methyl. In certain embodiments —R⁶ of formula (A-viii) is ethyl. In certain embodiments —R^{6a} of formula (A-viii) is —H. In certain embodiments —R^{6a} of formula (A-viii) is methyl. In certain embodiments —R^{6a} of formula (A-viii) is ethyl. In certain embodiments —R⁷ of formula (A-viii) is —H. In certain embodiments —R⁷ of formula (A-viii) is methyl. In certain embodiments —R⁷ of formula (A-viii) is ethyl. In certain embodiments —R⁸ of formula (A-viii) is —H. In certain embodiments —R⁸ of formula (A-viii) is methyl. In certain embodiments —R⁸ of formula (A-viii) is ethyl. In certain embodiments —R^{8a} of formula (A-viii) is —H. In certain embodiments —R^{8a} of formula (A-viii) is methyl. In certain embodiments —R^{8a} of formula (A-viii) is ethyl. In certain embodiments —R⁹ of formula (A-viii) is —H. In certain embodiments —R⁹ of formula (A-viii) is methyl. In certain embodiments —R⁹ of formula (A-viii) is ethyl. In certain embodiments —R^{9a} of formula (A-viii) is —H. In certain embodiments —R^{9a} of formula (A-viii) is methyl. In certain embodiments —R^{9a} of formula (A-viii) is ethyl. In certain embodiments —R^{9a} of formula (A-viii) is —H. In certain embodiments —R^{9a} of formula (A-viii) is methyl. In certain embodiments —R^{9a} of formula (A-viii) is ethyl. In certain embodiments —R¹⁰ of formula (A-viii) is —H. In certain embodiments —R¹⁰ of formula (A-viii) is methyl. In certain embodiments —R¹⁰ of formula (A-viii) is ethyl. In certain embodiments —R^{10a} of formula (A-viii) is —H. In certain embodiments —R^{10a} of formula (A-viii) is methyl. In certain embodiments —R^{10a} of formula (A-viii) is ethyl. In certain embodiments —R¹¹ of formula (A-viii) is —H. In certain embodiments —R¹¹ of formula (A-viii) is methyl. In certain embodiments —R¹¹ of formula (A-viii) is ethyl. In certain embodiments —R¹² of formula (A-viii) is —H. In certain embodiments —R¹² of formula (A-viii) is methyl. In certain embodiments —R¹² of formula (A-viii) is ethyl. In certain embodiments —R^{12a} of formula (A-viii) is —H. In certain embodiments —R^{12a} of formula (A-viii) is methyl. In certain embodiments —R^{12a} of formula (A-viii) is ethyl. In certain embodiments —R¹³ of formula (A-viii) is —H. In certain embodiments —R¹³ of formula (A-viii) is methyl. In certain embodiments —R¹³ of formula (A-viii) is ethyl. In certain embodiments —R¹⁴ of formula (A-viii) is —H. In certain embodiments —R¹⁴ of formula (A-viii) is methyl. In certain embodiments —R¹⁴ of formula (A-viii) is ethyl. In certain embodiments —R^{14a} of formula (A-viii) is —H. In certain embodiments —R^{14a} of formula (A-viii) is methyl. In certain embodiments —R^{14a} of formula (A-viii) is ethyl.

In certain embodiments -D¹- of formula (A-viii) is —O—. In certain embodiments -D¹- of formula (A-viii) is —NR¹¹—. In certain embodiments -D¹- of formula (A-viii) is —N⁺R¹²R^{12a}—. In certain embodiments -D¹- of formula (A-viii) is —S—. In certain embodiments -D¹- of formula (A-viii) is —(S═O). In certain embodiments -D¹- of formula (A-viii) is —(S(O)₂)—. In certain embodiments -D¹- of formula (A-viii) is —C(O)—. In certain embodiments -D¹- of formula (A-viii) is —P(O)R¹³—. In certain embodiments -D¹- of formula (A-viii) is —P(O)(OR¹³)—. In certain embodiments -D¹- of formula (A-viii) is —CR¹⁴R^{14a}—.

In certain embodiments -D²- of formula (A-viii) is —O—. In certain embodiments -D²- of formula (A-viii) is —NR¹¹—. In certain embodiments -D²- of formula (A-viii) is —N⁺R¹²R^{12a}—. In certain embodiments -D²- of formula (A-viii) is —S—. In certain embodiments -D²- of formula (A-viii) is —(S═O). In certain embodiments -D²- of formula (A-viii) is —(S(O)₂)—. In certain embodiments -D²- of formula (A-viii) is —C(O)—. In certain embodiments -D²- of formula (A-viii) is —P(O)R¹³—. In certain embodiments -D²- of formula (A-viii) is —P(O)(OR¹³)—. In certain embodiments -D²- of formula (A-viii) is —CR¹⁴R^{14a}—.

In certain embodiments -D³- of formula (A-viii) is —O—. In certain embodiments -D³- of formula (A-viii) is —NR¹¹—. In certain embodiments -D³- of formula (A-viii) is —N⁺R¹²R^{12a}—. In certain embodiments -D³- of formula (A-viii) is —S—. In certain embodiments -D³- of formula (A-viii) is —(S═O). In certain embodiments -D³- of formula (A-viii) is —(S(O)₂)—. In certain embodiments -D³- of formula (A-viii) is —C(O)—. In certain embodiments -D³- of formula (A-viii) is —P(O)R¹³—. In certain embodiments -D³- of formula (A-viii) is —P(O)(OR¹³)—. In certain embodiments -D³- of formula (A-viii) is —CR¹⁴R^{14a}—.

In certain embodiments -D⁴- of formula (A-viii) is —O—. In certain embodiments -D⁴- of formula (A-viii) is —NR¹¹—. In certain embodiments -D⁴- of formula (A-viii) is —N⁺R¹²R^{12a}—. In certain embodiments -D⁴- of formula (A-viii) is —S—. In certain embodiments -D⁴- of formula (A-viii) is —(S═O). In certain embodiments -D⁴- of formula (A-viii) is —(S(O)₂)—. In certain embodiments -D⁴- of formula (A-viii) is —C(O)—. In certain embodiments -D⁴- of formula (A-viii) is —P(O)R¹³—. In certain embodiments -D⁴- of formula (A-viii) is —P(O)(OR¹³)—. In certain embodiments -D⁴- of formula (A-viii) is —CR¹⁴R^{14a}—.

In certain embodiments -D⁵- of formula (A-viii) is —O—. In certain embodiments -D⁵- of formula (A-viii) is —NR¹¹—. In certain embodiments -D⁵- of formula (A-viii) is —N⁺R¹²R^{12a}—. In certain embodiments -D⁵- of formula (A-viii) is —S—. In certain embodiments -D⁵- of formula (A-viii) is —(S═O)—. In certain embodiments -D⁵- of formula (A-viii) is —(S(O)₂)—. In certain embodiments -D⁵- of formula (A-viii) is —C(O)—. In certain embodiments -D⁵- of formula (A-viii) is —P(O)R¹³—. In certain embodiments -D⁵- of formula (A-viii) is —P(O)(OR¹³)—. In certain embodiments -D⁵- of formula (A-viii) is —CR¹⁴R^{14a}—.

In certain embodiments -D⁶- of formula (A-viii) is —O—. In certain embodiments -D⁶- of formula (A-viii) is —NR¹¹—. In certain embodiments -D⁶- of formula (A-viii) is —N⁺R¹²R^{12a}—. In certain embodiments -D⁶- of formula (A-viii) is —S—. In certain embodiments -D⁶- of formula (A-viii) is —(S═O). In certain embodiments -D⁶- of formula (A-viii) is —(S(O)₂)—. In certain embodiments -D⁶- of formula (A-viii) is —C(O)—. In certain embodiments -D⁶- of formula (A-viii) is —P(O)R¹³—. In certain embodiments -D⁶- of formula (A-viii) is —P(O)(OR¹³)—. In certain embodiments -D⁶- of formula (A-viii) is —CR¹⁴R^{14a}—.

In one embodiment -CL^{p}- is of formula (A-ix)

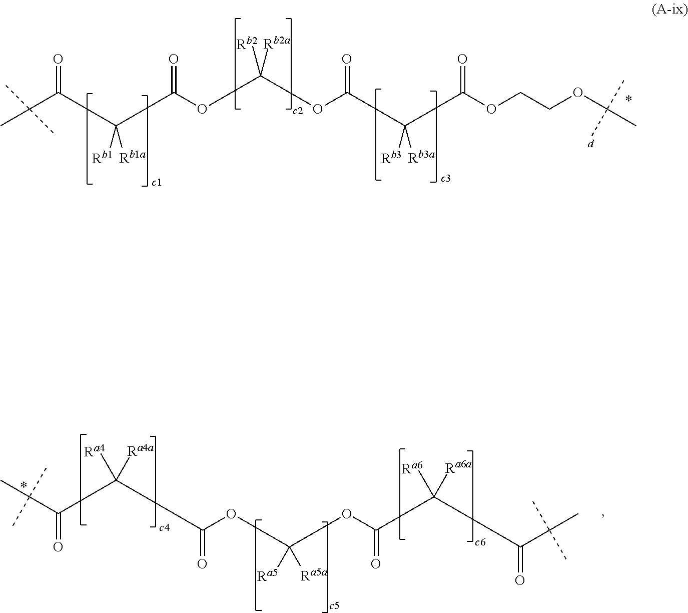

wherein
dashed lines marked with an asterisk indicate the connection point between the upper and the lower substructure,
unmarked dashed lines indicate attachment to a backbone moiety or to a spacer moiety -SP¹;
—R^{b1}, —R^{b1a}, —R^{b2}, —R^{b2a}, —R^{b3}, —R^{b3a}, —R^{b4}, —R^{b4a}, —R^{b5}, —R^{b5a}, —R^{b6} and —R^{b6} are independently selected from the group consisting of —H and C₁₋₆ alkyl;
c1, c2, c3, c4, c5 and c6 are independently selected from the group consisting of 1, 2, 3, 4, 5 and 6;
d is an integer ranging from 2 to 250.

In certain embodiments d of formula (A-ix) ranges from 3 to 200. In certain embodiments d of formula (A-ix) ranges from 4 to 150. In certain embodiments d of formula (A-ix) ranges from 5 to 100. In certain embodiments d of formula (A-ix) ranges from 10 to 50. In certain embodiments d of formula (A-ix) ranges from 15 to 30. In certain embodiments d of formula (A-ix) is about 23.

In certain embodiments —R^{b1} and —R^{b1a} of formula (A-ix) are —H. In certain embodiments —R^{b1} and —R^{b1a} of formula (A-ix) are —H. In certain embodiments —R^{b2} and —R^{b2a} of formula (A-ix) are —H. In certain embodiments —R^{b3} and —R^{b3a} of formula (A-ix) are —H. In certain embodiments —R^{b4} and —R^{b4a} of formula (A-ix) are —H. In certain embodiments —R^{b}s and —R^{b5a} of formula (A-ix) are —H. In certain embodiments —R^{b6} and —R^{b6a} of formula (A-ix) are —H.

In certain embodiments —Rb¹, —R^{b1a}, R^{b2}, —R^{b2a}, —R^{b3}, —R^{b3a}, —R^{b4}, —R^{b4a}, —R^{b5}, —R^{b5a}, —R^{b6} and —R^{b6} of formula (A-ix) are all —H.

In certain embodiments c1 of formula (A-ix) is 1. In certain embodiments c1 of formula (A-ix) is 2. In certain embodiments c1 of formula (A-ix) is 3. In certain embodiments c1 of formula (A-ix) is 4. In certain embodiments c1 of formula (A-ix) is 5. In certain embodiments c1 of formula (A-ix) is 6.

In certain embodiments c2 of formula (A-ix) is 1. In certain embodiments c2 of formula (A-ix) is 2. In certain embodiments c2 of formula (A-ix) is 3. In certain embodiments c2 of formula (A-ix) is 4. In certain embodiments c2 of formula (A-ix) is 5. In certain embodiments c2 of formula (A-ix) is 6.

In certain embodiments c3 of formula (A-ix) is 1. In certain embodiments c3 of formula (A-ix) is 2. In certain embodiments c3 of formula (A-ix) is 3. In certain embodiments c3 of formula (A-ix) is 4. In certain embodiments c3 of formula (A-ix) is 5. In certain embodiments c3 of formula (A-ix) is 6.

In certain embodiments c4 of formula (A-ix) is 1. In certain embodiments c4 of formula (A-ix) is 2. In certain embodiments c4 of formula (A-ix) is 3. In certain embodiments c4 of formula (A-ix) is 4. In certain embodiments c4 of formula (A-ix) is 5. In certain embodiments c4 of formula (A-ix) is 6.

In certain embodiments c5 of formula (A-ix) is 1. In certain embodiments c5 of formula (A-ix) is 2. In certain embodiments c5 of formula (A-ix) is 3. In certain embodiments c5 of formula (A-ix) is 4. In certain embodiments c5 of formula (A-ix) is 5. In certain embodiments c5 of formula (A-ix) is 6.

In certain embodiments c6 of formula (A-ix) is 1. In certain embodiments c6 of formula (A-ix) is 2. In certain embodiments c6 of formula (A-ix) is 3. In certain embodiments c6 of formula (A-ix) is 4. In certain embodiments c6 of formula (A-ix) is 5. In certain embodiments c6 of formula (A-ix) is 6.

In certain embodiments a crosslinker moiety -CL^{P}- is of formula (A-x)

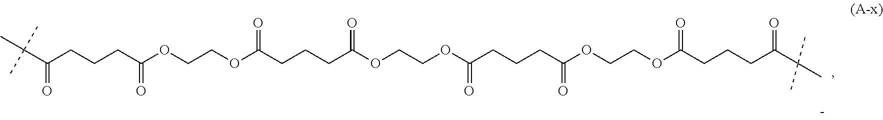

wherein

dashed lines indicate attachment to a backbone moiety or to a spacer moiety -SP¹-.

In certain embodiments -CL^{p}- is a C₆, C₈ or C₉ diacid comprising a PEG moiety of 2 to 3.3 kDa.

In certain embodiments -Z is a hyaluronic acid-based hydrogel. Such hyaluronic acid-based hydrogels are known in the art, such as for example from WO2018/175788, which is incorporated herewith by reference.

In certain embodiments the hyaluronic acid-based hydrogel is formed from linear hyaluronic acid strands having a molecular weight ranging from 100 to 150 kDa which are crosslinked with crosslinker moieties.

If -Z is a hyaluronic acid-based hydrogel, a conjugate of the present invention is in certain embodiments a conjugate comprising crosslinked hyaluronic acid strands to which a plurality of drug moieties is covalently and reversibly conjugated, wherein the conjugate comprises a plurality of connected units selected from the group consisting of

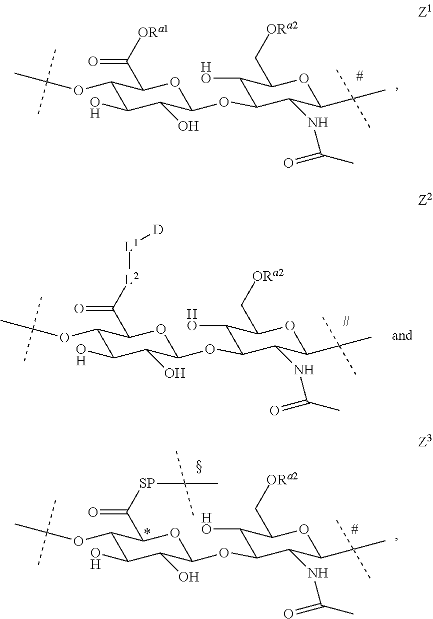

wherein
an unmarked dashed line indicates a point of attachment to an adjacent unit at a dashed line marked with # or to a hydrogen;
a dashed line marked with # indicates a point of attachment to an adjacent unit at an unmarked dashed line or to a hydroxyl;
a dashed line marked with § indicates a point of connection between at least two units Z³ via a moiety -CL-;
each -D, -L¹-, and -L² are used as defined above;
each -CL- is independently a moiety connecting at least two units Z³ and wherein there is at least one degradable bond in the direct connection between any two carbon atoms marked with the * connected by a moiety -CL-;
each -SP- is independently absent or a spacer moiety;
each —R^{a1} is independently selected from the group consisting of —H, C₁₋₄ alkyl, an ammonium ion, a tetrabutylammonium ion, a cetyl methylammonium ion, an alkali metal ion and an alkaline earth metal ion;
each —R^{a2} is independently selected from the group consisting of —H and C₁₋₁₀ alkyl;
wherein
all units Z¹ present in the conjugate may be the same or different;
all units Z² present in the conjugate may be the same or different;
all units Z³ present in the conjugate may be the same or different;
at least one unit Z³ is present per hyaluronic acid strand which is connected to at least one unit Z³ on a different hyaluronic acid strand; and
the conjugate comprises at least one moiety -L²-L¹-D.

The presence of at least one degradable bond between the carbon atom marked with the * of a first moiety Z³ and the direct connection to the carbon atom marked with the * of a second moiety Z³ ensures that after cleavage of all such degradable bonds the hyaluronic acid strands present in said conjugate are no longer crosslinked, which allows clearance of the hyaluronic acid network

It is understood that in case a degradable bond is located in a ring structure present in the direct connection of the carbon atom marked with the * of a first moiety Z³ and the carbon atom marked with the * of a second moiety Z³ such degradable bond is not sufficient to allow complete cleavage and accordingly one or more additional degradable bonds are present in the direct connection of the carbon atom marked with the * of a first moiety Z³ and the carbon atom marked with the * of a second moiety Z³.

It is understood that the phrase “a dashed line marked with § indicates a point of connection between at least two units Z³ via a moiety -CL-” refers to the following structure

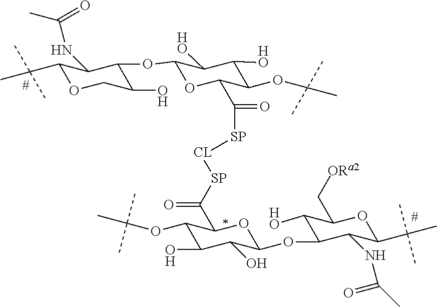

if -CL- is for example connected to two units Z³, which two moieties Z³ are connected at the position indicated with § via a moiety -CL-.

It is understood that no three-dimensionally crosslinked hydrogel can be formed if all hyaluronic acid strands of the present conjugate comprise only one unit Z³, which is connected to only one unit Z³ on a different hyaluronic acid strand. However, if a first unit Z³ is connected to more than one unit Z³ on a different strand, i.e. if -CL- is branched, such first unit Z³ may be crosslinked to two or more other units Z³ on two or more different hyaluronic acid strands. Accordingly, the number of units Z³ per hyaluronic acid strand required for a crosslinked hyaluronic acid hydrogel depends on the degree of branching of -CL-. In certain embodiments at least 30% of all hyaluronic acid strands present in the conjugate are connected to at least two other hyaluronic acid strands. It is understood that it is sufficient if the remaining hyaluronic acid strands are connected to only one other hyaluronic acid strand.

It is understood that such hydrogel also comprises partly reacted or unreacted units and that the presence of such moieties cannot be avoided. In certain embodiments the sum of such partly reacted or unreacted units is no more than 25% of the total number of units present in the conjugate, such as no more than 10%, such as no more than 15% or such as no more than 10%.

Furthermore, it is understood that in addition to units Z¹, Z² and Z³, partly reacted and unreacted units a conjugate may also comprise units that are the result of cleavage of the reversible bond between -D and -L¹- or of one or more of the degradable bonds present in the direct connection between any two carbon atoms marked with the * connected by a moiety -CL-, i.e. units resulting from degradation of the conjugate.

In certain embodiments each strand present in the conjugates of the present invention comprises at least 20 units, such as from 20 to 2500 units, from 25 to 2200 units, from 50 to 2000 units, from 75 to 100 units, from 75 to 100 units, from 80 to 560 units, from 100 to 250 units, from 200 to 800 units, from 20 to 1000, from 60 to 1000, from 60 to 400 or from 200 to 600 units.

In certain embodiments the moieties -CL- present in Z have different structures. In certain embodiments the moieties -CL- present in Z have the same structure.

In general, any moiety that connects at least two other moieties is suitable for use as a moiety -CL-, which may also be referred to as a “crosslinker moiety”.

The at least two units Z³ that are connected via a moiety -CL- may either be located on the same hyaluronic acid strand or on different hyaluronic acid strands.

The moiety -CL- may be linear or branched. In certain embodiments -CL- is linear. In certain embodiments -CL- is branched.

In certain embodiments -CL- connects two units Z³. In certain embodiments -CL- connects three units Z³. In certain embodiments -CL- connects four units Z³. In certain embodiments -CL- connects five units Z³. In certain embodiments -CL- connects six units Z³. In certain embodiments -CL- connects seven units Z³. In certain embodiments -CL- connects eight units Z³. In certain embodiments -CL- connects nine units Z³.

If -CL- connects two units Z³-CL- may be linear or branched. If -CL- connects more than two units Z³-CL- is branched.

A branched moiety -CL- comprises at least one branching point from which at least three branches extend, which branches may also be referred to as “arms”. Such branching point may be selected from the group consisting of

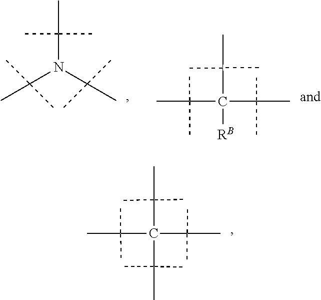

wherein
dashed lines indicate attachment to an arm; and
—R^{B} is selected from the group consisting of —H, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R^{B1}, which are the same or different, and wherein C₁₋₆ alkyl, C₂₋₆ alkenyl and C₂₋₆ alkynyl are optionally interrupted with —C(O)O—, —O—, —C(O)—, —C(O)N(R^{B2})—, —S(O)₂N(R^{B2})—, —S(O)N(R^{B2})—, —S(O)₂—, —S(O)—, —N(R^{B2})S(O)₂N(R^{B2a})—, —S—, —N(R^{B2})—, —OC(OR^{B2})(R^{B2a})—, —N(R^{B2})C(O)N(R^{B2a})—, and —OC(O)N(R^{B2})—; wherein —R^{B1}, —R^{B2} and —R^{B2a} are selected from —H, C₁₋₆ alkyl, C₂₋₆ alkenyl and C₂₋₆ alkynyl.

In certain embodiments —R^{B} is selected from the group consisting of —H, methyl and ethyl.

A branched moiety -CL- may comprise a plurality of branching points, such as 1, 2, 3, 4, 5, 6, 7 or more branching points, which may be the same or different.

If a moiety -CL- connects three units Z³, such moiety -CL- comprises at least one branching point from which at least three arms extend.

If a moiety -CL- connects four units Z³, such moiety -CL- may comprise one branching point from which four arms extend. However, alternative geometries are possible, such as at least two branching points from which at least three arms each extend. The larger the number of connected units Z³, the larger the number of possible geometries is.

In a first embodiment at least 70%, such as at least 75%, such as at least 80%, such as at least 85%, such as at least 90% or such as at least 95% of the number of hyaluronic acid strands of the conjugate of the present invention comprise at least one moiety Z² and at least one moiety Z³. In such embodiment units Z² and Z³ can be found in essentially all hyaluronic acid strands present in the conjugates of the present invention.

Accordingly, a conjugate of this first embodiment comprises crosslinked hyaluronic acid strands to which a plurality of drug moieties are covalently and reversibly conjugated, wherein the conjugate of the present invention comprises a plurality of connected units selected from the group consisting of

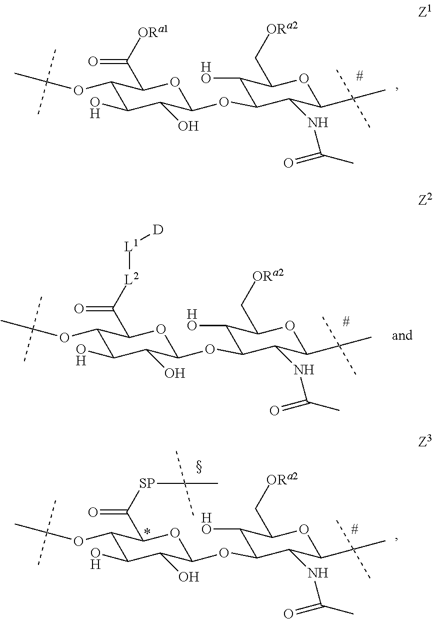

wherein
an unmarked dashed line indicates a point of attachment to an adjacent unit at a dashed line marked with # or to a hydrogen;
a dashed line marked with # indicates a point of attachment to an adjacent unit at an unmarked dashed line or to a hydroxyl;
a dashed line marked with § indicates a point of connection between at least two units Z³ via a moiety -CL-;
D, -L¹-, -L²-, are used as defined above;
wherein
all units Z¹ present in the conjugate may be the same or different;
all units Z² present in the conjugate may be the same or different;
all units Z³ present in the conjugate may be the same or different;
the number of Z¹ units ranges from 1% to 98% of the total number of units present in the conjugate of the present invention;
the number of Z² units ranges from 1% to 98% of the total number of units present in the conjugate, provided at least one unit Z² is present in the conjugate of the present invention;
the number of Z³ units ranges from 1% to 97% of the total number of units present in the conjugate of the present invention, provided that at least one unit Z³ is present per strand; and
wherein at least 70% of all hyaluronic acid strands comprise at least one moiety Z² and at least one moiety Z³.

In a conjugate of the present invention according to this first embodiment the number of units Z² ranges from 1 to 70% of all units present in the conjugate of the present invention, such as from 2 to 15%, from 2 to 10%, from 16 to 39, from 40 to 65%, or from 50 to 60% of all units present in the conjugate of the present invention.

In a conjugate of the present invention according to this first embodiment the number of units Z³ ranges from 1 to 30% of all units present in conjugate of the present invention, such as from 2 to 5%, from 5 to 20%, from 10 to 18%, or from 14 to 18% of all units present in the conjugate of the present invention.

In a conjugate of the present invention according to this first embodiment the number of units Z¹ ranges from 10 to 97% of all units present in the conjugate of the present invention, such as from 20 to 40%, such as from 25 to 35%, such as from 41 to 95%, such as from 45 to 90%, such as from 50 to 70% of all units present in the conjugate of the present invention.

Each degradable bond present in the direct connection between any two carbon atoms marked with the * connected by a moiety -CL- may be different or all such degradable bonds present in the conjugate of the present invention may be the same.

Each direct connection between two carbon atoms marked with the * connected by a moiety -CL- may have the same or a different number of degradable bonds.

In certain embodiments the number of degradable bonds present in the conjugate of the present invention between all combinations of two carbon atoms marked with the * connected by a moiety -CL- is the same and all such degradable bonds have the same structure.

In the first embodiment the at least one degradable bond present in the direct connection between any two carbon atoms marked with the * connected by a moiety -CL- may be selected from the group consisting of ester, carbonate, sulfate, phosphate bonds, carbamate and amide bonds. It is understood that carbamates and amides are not reversible per se, and that in this context neighboring groups render these bonds reversible. In certain embodiments there is one degradable bond selected from the group consisting of ester, carbonate, sulfate, phosphate bonds, carbamate and amide bonds in the direct connection between any two carbon atoms marked with the * connected by a moiety -CL-. In certain embodiments there are two degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. In certain embodiments there are three degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. In certain embodiments there are four degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. In certain embodiments there are five degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. In certain embodiments there are six degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. It is understood that if more than two units Z³ are connected by -CL- there are more than two carbons marked with * that are connected and thus there is more than one shortest connection with at least one degradable bond present. Each shortest connection may have the same or different number of degradable bonds.

In certain embodiments the at least one degradable bond, such as one, two, three, four, five, six degradable bonds, are located within -CL-.

In certain embodiments the at least one degradable bond present in the direct connection between any two carbon atoms marked with * connected by a moiety -CL- is one ester bond. In other embodiments the at least one degradable bond are two ester bonds. In other embodiments the at least one degradable bond are three ester bonds. In other embodiments the at least one degradable bond are four ester bonds. In other embodiments the at least one degradable bond are five ester bonds. In other embodiments the at least one degradable bond are six ester bonds.

In certain embodiments the at least one degradable bond present in the direct connection between any two carbon atoms marked with * connected by a moiety -CL- is one carbonate bond. In other embodiments the at least one degradable bond are two carbonate bonds. In other embodiments the at least one degradable bond are three carbonate bonds. In other embodiments the at least one degradable bond are four carbonate bonds. In other embodiments the at least one degradable bond are five carbonate bonds. In other embodiments the at least one degradable bond are six carbonate bonds.

In certain embodiments the at least one degradable bond present in the direct connection between any two carbon atoms marked with * connected by a moiety -CL- is one phosphate bond. In other embodiments the at least one degradable bond are two phosphate bonds. In other embodiments the at least one degradable bond are three phosphate bonds. In other embodiments the at least one degradable bond are four phosphate bonds. In other embodiments the at least one degradable bond are five phosphate bonds. In other embodiments the at least one degradable bond are six phosphate bonds.

In certain embodiments the at least one degradable bond present in the direct connection between any two carbon atoms marked with * connected by a moiety -CL- is one sulfate bond. In other embodiments the at least one degradable bond are two sulfate bonds. In other embodiments the at least one degradable bond are three sulfate bonds. In other embodiments the at least one degradable bond are four sulfate bonds. In other embodiments the at least one degradable bond are five sulfate bonds. In other embodiments the at least one degradable bond are six sulfate bonds.

In certain embodiments the at least one degradable bond present in the direct connection between any two carbon atoms marked with * connected by a moiety -CL- is one carbamate bond. In other embodiments the at least one degradable bond are two carbamate bonds. In other embodiments the at least one degradable bond are three carbamate bonds. In other embodiments the at least one degradable bond are four carbamate bonds. In other embodiments the at least one degradable bond are five carbamate bonds. In other embodiments the at least one degradable bond are six carbamate bonds.

In certain embodiments the at least one degradable bond present in the direct connection between any two carbon atoms marked with * connected by a moiety -CL- is one amide bond. In other embodiments the at least one degradable bond are two amide bonds. In other embodiments the at least one degradable bond are three amide bonds. In other embodiments the at least one degradable bond are four amide bonds. In other embodiments the at least one degradable bond are five amide bonds. In other embodiments the at least one degradable bond are six amide bonds.

In some embodiments -CL- is C₁₋₅₀ alkyl, which is optionally interrupted by one or more atoms or groups selected from the group consisting of -T-, —C(O)O—, —O—, —C(O)—, —C(O)N(R^{c1})—, —S(O)₂—, —S(O)—, —S—, —N(R^{c1})—, —OC(OR^{c1})(R^{c1a})— and —OC(O)N(R^{c1})—;
wherein -T- is selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; and
—R^{c1} and —R^{c1a} are selected from the group consisting of —H and C₁₋₆ alkyl.

In certain embodiments -CL- is a moiety of formula (B)

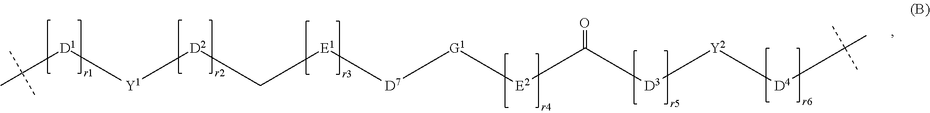

wherein
-Y¹- is of formula

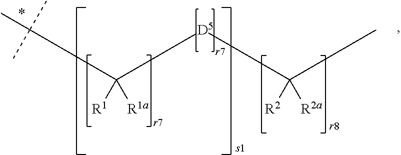

wherein the dashed line marked with the asterisk indicates attachment to -D¹- and the unmarked dashed line indicates attachment to -D²-;
-Y²- is of formula

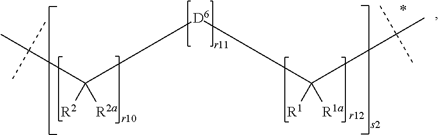

wherein the dashed line marked with the asterisk indicates attachment to -D⁴- and the unmarked dashed line indicates attachment to -D³-;
-E¹- is of formula

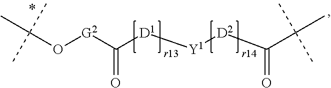

wherein the dashed line marked with the asterisk indicates attachment to —(C═O)- and the unmarked dashed line indicates attachment to —O—;
-E²- is of formula

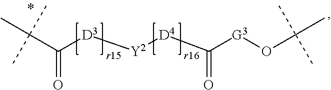

wherein the dashed line marked with the asterisk indicates attachment to -G¹- and the unmarked dashed line indicates attachment to —(C═O)—;
-G¹- is of formula

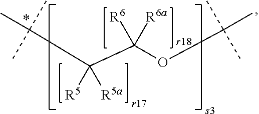

wherein the dashed line marked with the asterisk indicates attachment to —O— and the unmarked dashed line indicates attachment to -E²-;
-G²- is of formula

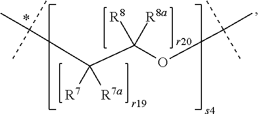

wherein the dashed line marked with the asterisk indicates attachment to —O— and the unmarked dashed line indicates attachment to —(C═O)—;
-G³- is of formula (C-vii),

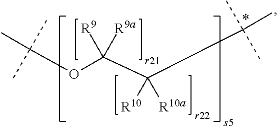

wherein the dashed line marked with the asterisk indicates attachment to —O— and the unmarked dashed line indicates attachment to —(C═O)—;
-D¹-, -D²-, -D³-, -D⁴-, -D⁵-, -D⁶- and -D⁷- are identical or different and each is independently of the others selected from the group comprising —O—, —NR¹¹—, —N⁺R¹², R^{12a}—, —S—, —(S═O)—, —(S(O)₂), —C(O)—, —P(O)R¹³ and —CR¹⁴R^{14a}—.
—R¹, —R^{1a}, —R², —R^{2a}, —R³, —R^{3a}, —R⁴, —R^{4a}, —R⁵, —R^{5a}, —R⁶, —R^{6a}, —R⁷, —R^{7a}, -R⁸, —R^{8a}, —R⁹, —R^{9a}, —R¹⁰, —R^{10a}, —R¹¹, —R¹², —R^{12a}, —R¹³, —R¹⁴ and —R^{14a} are identical or different and each is independently of the others selected from the group comprising —H and C₁₋₆ alkyl; optionally, one or more of the pairs —R¹/—R^{1a}, —R²/—R^{2a}, —R³/—R^{3a}, —R⁴/—R^{4a}, —R¹/—R², —R³/—R⁴, —R^{1a}/—R^{2a}, —R^{3a}/—R^{4a}, —R²/—R^{12a}, and —R¹⁴/—R^{14a} form a chemical bond or are joined together with the atom to which they are attached to form a C₃₋₈ cycloalkyl or to form a ring A or are joined together with the atom to which they are attached to form a 4- to 7-membered heterocyclyl or 8- to 11-membered heterobicyclyl or adamantyl; A is selected from the group consisting of phenyl, naphthyl, indenyl, indanyl and tetralinyl;
r1, r2, r5, r6, r13, r14, r15 and r16 are independently 0 or 1;
r3, r4, r7, r8, r9, r10, r11, r12 are independently 0, 1, 2, 3, or 4;
r17, r18, r19, r20, r21 and r22 are independently 1, 2, 3, 4, 5, 6, 7, 8, 9 or 10; and
s1, s2, s4, s5 are independently 1, 2, 3, 4, 5 or 6.
s3 ranges from 1 to 200, preferably from 1 to 100 and more preferably from 1 to 50.

In certain embodiments r1 of formula (B) is 0. In certain embodiments r1 of formula (B) is 1. In certain embodiments r2 of formula (B) is 0. In certain embodiments r2 of formula (B) is 1. In certain embodiments r5 of formula (B) is 0. In certain embodiments r5 of formula (B) is 1. In certain embodiments r6 of formula (B) is 0. In certain embodiments r6 of formula (B) is 1. In certain embodiments r13 of formula (B) is 0. In certain embodiments r13 of formula (B) is 1. In certain embodiments r14 of formula (B) is 0. In certain embodiments r14 of formula (B) is 1. In certain embodiments r15 of formula (B) is 0. In certain embodiments r15 of formula (B) is 1. In certain embodiments r16 of formula (B) is 0. In certain embodiments r16 of formula (B) is 1.

In certain embodiments r3 of formula (B) is 0. In certain embodiments r3 of formula (B) is 1. In certain embodiments r4 of formula (B) is 0. In certain embodiments r4 of formula (B) is 1. In certain embodiments r3 of formula (B) and r4 of formula (B) are both 0.

In certain embodiments r7 of formula (B) is 0. In certain embodiments r7 of formula (B) is 1. In certain embodiments r7 of formula (B) is 2. In certain embodiments r8 of formula (B) is 0. In certain embodiments r8 of formula (B) is 1. In certain embodiments r8 of formula (B) of formula (B) is 2. In certain embodiments r9 of formula (B) is 0. In certain embodiments r9 of formula (B) is 1. In certain embodiments r9 of formula (B) is 2. In certain embodiments r10 of formula (B) is 0. In certain embodiments r10 of formula (B) is 1. In certain embodiments r10 of formula (B) is 2. In certain embodiments r11 of formula (B) is 0. In certain embodiments r11 of formula (B) is 1. In certain embodiments r11 of formula (B) is 2. In certain embodiments r12 of formula (B) is 0. In certain embodiments r12 of formula (B) is 1. In certain embodiments r12 of formula (B) is 2.

In certain embodiments r17 of formula (B) is 1. In certain embodiments r18 of formula (B) is 1. In certain embodiments r19 of formula (B) is 1. In certain embodiments r20 of formula (B) is 1. In certain embodiments r21 of formula (B) is 1.

In certain embodiments s1 of formula (B) is 1. In certain embodiments s1 of formula (B) is 2. In certain embodiments s2 of formula (B) is 1. In certain embodiments s2 of formula (B) is 2. In certain embodiments s4 of formula (B) is 1. In certain embodiments s4 of formula (B) is 2.

In certain embodiments s3 of formula (B) ranges from 1 to 100. In certain embodiments s3 of formula (B) ranges from 1 to 75. In certain embodiments s3 of formula (B) ranges from 2 to 50. In certain embodiments s3 of formula (B) ranges from 2 to 40. In certain embodiments s3 of formula (B) ranges from 3 to 30. In certain embodiments s3 of formula (B) is about 3.

In certain embodiments —R¹ of formula (B) is —H. In certain embodiments —R¹ of formula (B) is methyl. In certain embodiments —R¹ of formula (B) is ethyl. In certain embodiments —R^{1a} of formula (B) is —H. In certain embodiments —R^{1a} of formula (B) is methyl. In certain embodiments —R^{1a} of formula (B) is ethyl. In certain embodiments —R² of formula (B) is —H. In certain embodiments —R² of formula (B) is methyl. In certain embodiments —R² of formula (B) is ethyl. In certain embodiments —R^{2a} of formula (B) is —H. In certain embodiments —R^{2a} of formula (B) is methyl. In certain embodiments —R^{2a} of formula (B) is ethyl. In certain embodiments —R³ of formula (B) is —H. In certain embodiments —R³ of formula (B) is methyl. In certain embodiments —R³ of formula (B) is ethyl. In certain embodiments —R^{3a} of formula (B) is —H. In certain embodiments —R^{3a} of formula (B) is methyl. In certain embodiments —R^{3a} of formula (B) is ethyl. In certain embodiments —R⁴ of formula (B) is —H. In certain embodiments —R⁴ of formula (B) is methyl. In certain embodiments —R⁴ of formula (B) is methyl. In certain embodiments —R^{4a} of formula (B) is —H. In certain embodiments —R^{4a} of formula (B) is methyl. In certain embodiments —R^{4a} of formula (B) is ethyl. In certain embodiments —R⁵ of formula (B) is —H. In certain embodiments —R⁵ of formula (B) is methyl. In certain embodiments —R⁵ of formula (B) is ethyl. In certain embodiments —R^{5a} of formula (B) is —H. In certain embodiments —R^{5a} of formula (B) is methyl. In certain embodiments —R^{5a} of formula (B) is ethyl. In certain embodiments —R⁶ of formula (B) is —H. In certain embodiments —R⁶ of formula (B) is methyl. In certain embodiments —R⁶ of formula (B) is ethyl. In certain embodiments —R^{6a} of formula (B) is —H. In certain embodiments —R^{6a} of formula (B) is methyl. In certain embodiments —R^{6a} of formula (B) is ethyl. In certain embodiments —R⁷ of formula (B) is —H. In certain embodiments —R⁷ of formula (B) is methyl. In certain embodiments —R⁷ of formula (B) is ethyl. In certain embodiments —R⁸ of formula (B) is —H. In certain embodiments —R⁸ of formula (B) is methyl. In certain embodiments —R⁸ of formula (B) is ethyl. In certain embodiments —R^{8a} of formula (B) is —H. In certain embodiments —R^{8a} of formula (B) is methyl. In certain embodiments —R^{8a} of formula (B) is ethyl. In certain embodiments —R⁹ of formula (B) is —H. In certain embodiments —R⁹ of formula (B) is methyl. In certain embodiments —R⁹ of formula (B) is ethyl. In certain embodiments —R^{9a} of formula (B) is —H. In certain embodiments —R^{9a} of formula (B) is methyl. In certain embodiments —R^{9a} of formula (B) is ethyl. In certain embodiments —R^{9a} of formula (B) is —H. In certain embodiments —R^{9a} of formula (B) is methyl. In certain embodiments —R^{9a} of formula (B) is ethyl. In certain embodiments —R¹⁰ of formula (B) is —H. In certain embodiments —R¹⁰ of formula (B) is methyl. In certain embodiments —R¹⁰ of formula (B) is ethyl. In certain embodiments —R^{10a} of formula (B) is —H. In certain embodiments —R^{10a} of formula (B) is methyl. In certain embodiments —R^{10a} of formula (B) is ethyl. In certain embodiments —R¹¹ of formula (B) is —H. In certain embodiments —R¹¹ of formula (B) is methyl. In certain embodiments —R¹¹ of formula (B) is ethyl. In certain embodiments —R¹² of formula (B) is —H. In certain embodiments —R¹² of formula (B) is methyl. In certain embodiments —R¹² of formula (B) is ethyl. In certain embodiments —R^{12a} of formula (B) is —H. In certain embodiments —R^{12a} of formula (B) is methyl. In certain embodiments —R^{12a} of formula (B) is ethyl. In certain embodiments —R¹³ of formula (B) is —H. In certain embodiments —R¹³ of formula (B) is methyl. In certain embodiments —R¹³ of formula (B) is ethyl In certain embodiments —R¹⁴ of formula (B) is —H. In certain embodiments —R¹⁴ of formula (B) is methyl. In certain embodiments —R¹⁴ of formula (B) is ethyl. In certain embodiments —R^{14a} of formula (B) is —H. In certain embodiments —R¹4^{a} of formula (B) is methyl. In certain embodiments —R^{14a} of formula (B) is ethyl.

In certain embodiments -D¹- of formula (B) is —O—. In certain embodiments -D¹- of formula (B) is —NR¹¹—. In certain embodiments -D¹- of formula (B) is —N⁺R¹²R^{12a}—. In certain embodiments -D¹- of formula (B) is —S—. In certain embodiments -D¹- of formula (B) is —(S═O). In certain embodiments -D¹- of formula (B) is —(S(O)₂)—. In certain embodiments -D¹- of formula (B) is —C(O)—. In certain embodiments -D¹- of formula (B) is —P(O)R¹³—. In certain embodiments -D¹- of formula (B) is —P(O)(OR¹³)—. In certain embodiments -D¹- of formula (B) is —CR¹⁴R^{14a}—,

In certain embodiments -D²- of formula (B) is —O—. In certain embodiments -D²- of formula (B) is —NR¹¹—. In certain embodiments -D²- of formula (B) is —N⁺R¹²R^{12a}—. In certain embodiments -D²- of formula (B) is —S—. In certain embodiments -D²- of formula (B) is —(S═O). In certain embodiments -D²- of formula (B) is —(S(O)₂)—. In certain embodiments -D²- of formula (B) is —C(O)—. In certain embodiments -D²- of formula (B) is —P(O)R¹³—. In certain embodiments -D²- of formula (B) is —P(O)(OR¹³)—. In certain embodiments -D²- of formula (B) is —CR¹⁴R^{14a}—.

In certain embodiments -D³- of formula (B) is —O—. In certain embodiments -D³- of formula (B) is —NR¹¹—. In certain embodiments -D³- of formula (B) is —N⁺R¹²R^{12a}—. In certain embodiments -D³- of formula (B) is —S—. In certain embodiments -D³- of formula (B) is —(S═O). In certain embodiments -D³- of formula (B) is —(S(O)₂)—. In certain embodiments -D³- of formula (B) is —C(O)—. In certain embodiments -D³- of formula (B) is —P(O)R¹³—. In certain embodiments -D³- of formula (B) is —P(O)(OR¹³)—. In certain embodiments -D³- of formula (B) is —CR¹⁴R^{14a}—.

In certain embodiments -D⁴- of formula (B) is —O—. In certain embodiments -D⁴- of formula (B) is —NR¹¹—. In certain embodiments -D⁴- of formula (B) is —N⁺R¹²R^{12a}—. In certain embodiments -D⁴- of formula (B) is —S—. In certain embodiments -D⁴- of formula (B) is —(S═O). In certain embodiments -D⁴- of formula (B) is —(S(O)₂)—. In certain embodiments -D⁴- of formula (B) is —C(O)—. In certain embodiments -D⁴- of formula (B) is —P(O)R¹³—. In certain embodiments -D⁴- of formula (B) is —P(O)(OR¹³)—. In certain embodiments -D⁴- of formula (B) is —CR¹⁴R^{14a}—.

In certain embodiments -D⁵- of formula (B) is —O—. In certain embodiments -D⁵- of formula (B) is —NR¹¹—. In certain embodiments -D⁵- of formula (B) is -N⁺R¹²R^{12a}—. In certain embodiments -D⁵- of formula (B) is —S—. In certain embodiments -D⁵- of formula (B) is —(S═O)—. In certain embodiments -D⁵- of formula (B) is —(S(O)₂)—. In certain embodiments -D⁵- of formula (B) is —C(O)—. In certain embodiments -D⁵- of formula (B) is —P(O)R¹³—. In certain embodiments -D⁵- of formula (B) is —P(O)(OR¹³)—. In certain embodiments -D⁵- of formula (B) is —CR¹⁴R^{14a}—.

In certain embodiments -D⁶- of formula (B) is —O—. In certain embodiments -D⁶- of formula (B) is —NR¹¹—. In certain embodiments -D⁶- of formula (B) is —N⁺R¹²R^{12a}—. In certain embodiments -D⁶- of formula (B) is —S—. In certain embodiments -D⁶- of formula (B) is —(S═O). In certain embodiments -D⁶- of formula (B) is —(S(O)₂)—. In certain embodiments -D⁶- of formula (B) is —C(O)—. In certain embodiments -D⁶- of formula (B) is —P(O)R¹³—. In certain embodiments -D⁶- of formula (B) is —P(O)(OR¹³)—. In certain embodiments -D⁶- of formula (B) is —CR¹⁴R^{14a}—.

In certain embodiments -D⁷- of formula (B) is —O—. In certain embodiments -D⁷- of formula (B) is —NR¹¹—. In certain embodiments -D⁷- of formula (B) is -N⁺R¹²R^{12a}—. In certain embodiments -D⁷- of formula (B) is —S—. In certain embodiments -D⁷- of formula (B) is —(S═O). In certain embodiments -D⁷- of formula (B) is —(S(O)₂)—. In certain embodiments -D⁷- of formula (B) is —C(O)—. In certain embodiments -D⁷- of formula (B) is —P(O)R¹³—. In certain embodiments -D⁷- of formula (B) is —P(O)(OR¹³)—. In certain embodiments -D⁷- of formula (B) is —CR¹⁴R^{14a}—.

In certain embodiments -CL- is of formula (B-i)

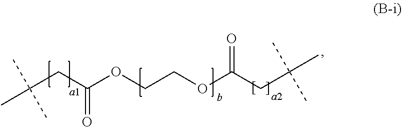

wherein
a1 and a2 are independently selected from the group consisting of a1 and a2 are independently selected from the group consisting of 1, 2, 3, 4, 5, 6, 7, 8, 9, 10, 11, 12, 13 and 14; and
b is an integer ranging from 1 to 50.

In certain embodiments a1 and a2 of formula (B-i) are different. In certain embodiments a1 and a2 of formula (B-i) are the same.

In certain embodiments a1 of formula (B-i) is 1. In certain embodiments a1 of formula (B-i) is 2. In certain embodiments a1 of formula (B-i) is 3. In certain embodiments a1 of formula (B-i) is 4. In certain embodiments a1 of formula (B-i) is 5. In certain embodiments a1 of formula (B-i) is 6. In certain embodiments a1 of formula (B-i) is 7. In certain embodiments a1 of formula (B-i) is 8. In certain embodiments a1 of formula (B-i) is 9. In certain embodiments a1 of formula (B-i) is 10.

In certain embodiments a2 of formula (B-i) is 1. In certain embodiments a2 of formula (B-i) is 2. In certain embodiments a2 of formula (B-i) is 3. In certain embodiments a2 of formula (B-i) is 4. In certain embodiments a2 of formula (B-i) is 5. In certain embodiments a2 of formula (B-i) is 6. In certain embodiments a2 of formula (B-i) is 7. In certain embodiments a2 of formula (B-i) is 8. In certain embodiments a2 of formula (B-i) is 9. In certain embodiments a2 of formula (B-i) is 10.

In certain embodiments b of formula (B-i) ranges from 1 to 500. In certain embodiments b of formula (B-i) ranges from 2 to 250. In certain embodiments b of formula (B-i) ranges from 3 to 100. In certain embodiments b of formula (B-i) ranges from 3 to 50. In certain embodiments b of formula (B-i) ranges from 3 to 25. In certain embodiments b of formula (B-i) is 3. In certain embodiments b of formula (B-i) is 25.

In certain embodiments -CL- is of formula (B-i)

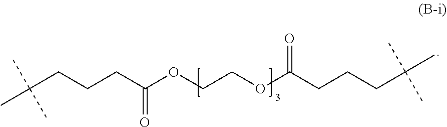

In certain embodiments -CL- is of formula (B-ii)

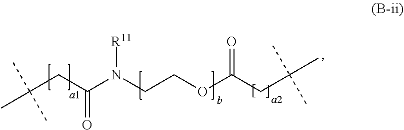

wherein
a1 and a2 are independently selected from the group consisting of 1, 2, 3, 4, 5, 6, 7, 8, 9, 10, 11, 12, 13 and 14;
b is an integer ranging from 1 to 50; and
—R¹¹ is selected from the group comprising —H and C₁₋₆ alkyl.

In certain embodiments a1 and a2 of formula (B-ii) are different. In certain embodiments a1 and a2 of formula (B) are the same.

In certain embodiments a1 of formula (B-ii) is 1. In certain embodiments a1 of formula (B-ii) is 2. In certain embodiments a1 of formula (B-ii) is 3. In certain embodiments a1 of formula (B-ii) is 4. In certain embodiments a1 of formula (B-ii) is 5. In certain embodiments a1 of formula (B-ii) is 6. In certain embodiments a1 of formula (B-ii) is 7. In certain embodiments a1 of formula (B-ii) is 8. In certain embodiments a1 of formula (B-ii) is 9. In certain embodiments a1 of formula (B-ii) is 10.

In certain embodiments a2 of formula (B-ii) is 1. In certain embodiments a2 of formula (B-ii) is 2. In certain embodiments a2 of formula (B-ii) is 3. In certain embodiments a2 of formula (B-ii) is 4. In certain embodiments a2 of formula (B-ii) is 5. In certain embodiments a2 of formula (B-ii) is 6. In certain embodiments a2 of formula (B-ii) is 7. In certain embodiments a2 of formula (B-ii) is 8. In certain embodiments a2 of formula (B-ii) is 9. In certain embodiments a2 of formula (B-ii) is 10.

In certain embodiments b of formula (B-ii) ranges from 1 to 500. In certain embodiments b of formula (B-ii) ranges from 2 to 250. In certain embodiments b of formula (B-ii) ranges from 3 to 100. In certain embodiments b of formula (B-ii) ranges from 3 to 50. In certain embodiments b of formula (B-ii) ranges from 3 to 25. In certain embodiments b of formula (B-ii) is 3. In certain embodiments b of formula (B-ii) is 25.

In certain embodiments —R¹¹ of formula (B-ii) is —H. In certain embodiments —R¹¹ of formula (B-ii) is methyl. In certain embodiments —R¹¹ of formula (B-ii) is ethyl. In certain embodiments —R¹¹ of formula (B-ii) is n-propyl. In certain embodiments —R¹¹ of formula (B-ii) is isopropyl. In certain embodiments —R¹¹ of formula (B-ii) is n-butyl. In certain embodiments —R¹¹ of formula (B-ii) is isobutyl. In certain embodiments —R¹¹ of formula (B-ii) is sec-butyl. In certain embodiments —R¹¹ of formula (B-ii) is tert-butyl. In certain embodiments —R¹¹ of formula (B-ii) is n-pentyl. In certain embodiments —R¹¹ of formula (B-ii) is 2-methylbutyl. In certain embodiments —R¹¹ of formula (B-ii) is 2,2-dimethylpropyl. In certain embodiments —R¹¹ of formula (B-ii) is n-hexyl. In certain embodiments —R¹¹ of formula (B-ii) is 2-methylpentyl. In certain embodiments —R¹¹ of formula (B-ii) is 3-methylpentyl. In certain embodiments —R¹¹ of formula (B-ii) is 2,2-dimethylbutyl. In certain embodiments —R¹¹ of formula (B-ii) is 2,3-dimethylbutyl. In certain embodiments —R¹¹ of formula (B-ii) is 3,3-dimethylpropyl.

In certain embodiments -CL- is of formula (B-iii)

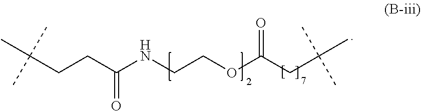

In a second embodiment the moiety -CL- is selected from the group consisting of

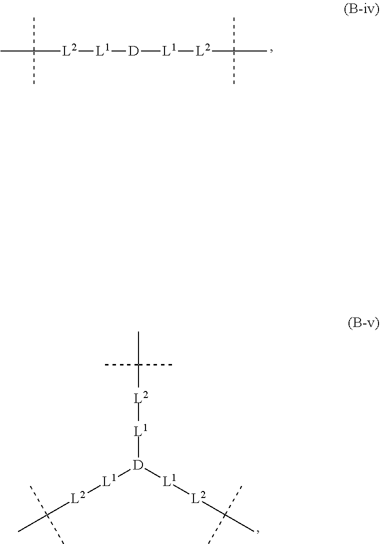

wherein
each dashed line indicates attachment to a unit Z³; and
L¹-, -L²- and -D are used as defined for Z.

It is understood that in formula (B-iv) two functional groups of the drug are conjugated to one moiety -L¹- each and that in formula (B-v) three functional groups of the drug are conjugated to one moiety -L¹- each. The moiety -CL- of formula (B-iv) connects two moieties Z³ and the moiety -CL- of formula (B-v) connects three moieties Z³, which may be on the same or different hyaluronic acid strand. In this embodiment -CL- comprises at least two degradable bonds, if -CL- is of formula (B-iv) or at least three degradable bonds, if -CL- is of formula (B-v), namely the degradable bonds that connect D with a moiety -L¹-. A conjugate of the present invention may only comprise moieties -CL- of formula (B-iv), may only comprise moieties -CL- of formula (B-v) or may comprise moieties -CL- of formula (B-iv) and formula (B-v).

Accordingly, a conjugate of the present invention of this second embodiment comprises crosslinked hyaluronic acid strands to which a plurality of drug moieties are covalently and reversibly conjugated, wherein the conjugate of the present invention comprises a plurality of connected units selected from the group consisting of

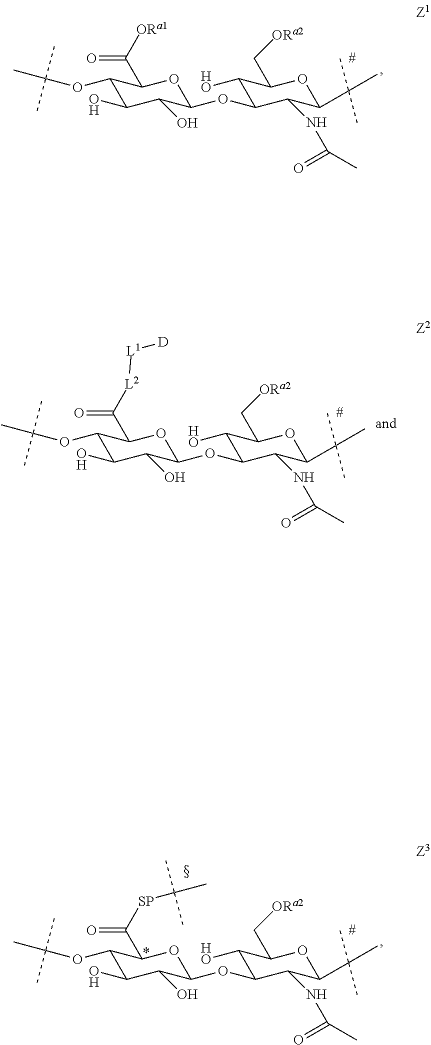

wherein
an unmarked dashed line indicates a point of attachment to an adjacent unit at a dashed line marked with # or to a hydrogen;
a dashed line marked with # indicates a point of attachment to an adjacent unit at an unmarked dashed line or to a hydroxyl;
a dashed line marked with § indicates a point of connection between at least two units Z³ via a moiety -CL-;
each -CL- comprises at least one degradable bond between the two carbon atoms marked with the * connected by a moiety -CL- and each -CL- is independently selected from the group consisting of formula (B-iv) and (B-v)

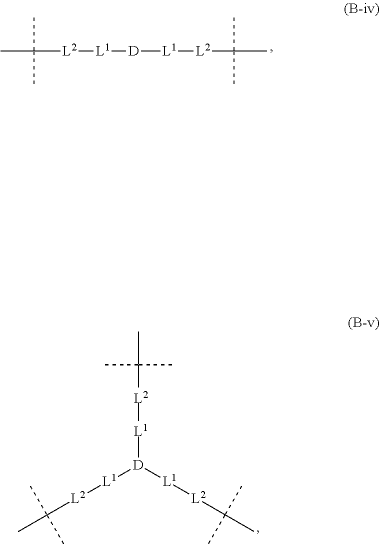

wherein
dashed lines indicate attachment to a unit Z³;
-D, -L¹-, -L²-, -SP-, —R^{a1} and —R^{a2} are used as defined for Z¹, Z² and Z³;
wherein
all units Z¹ present in the conjugate may be the same or different;
all units Z² present in the conjugate may be the same or different;
all units Z³ present in the conjugate may be the same or different;
the number of Z¹ units ranges from 1% to 98% of the total number of units present in the conjugate of the present invention;
the number of Z² units ranges from 0% to 98% of the total number of units present in the conjugate of the present invention;
the number of Z³ units ranges from 1% to 97% of the total number of units present in the conjugate of the present invention, provided that at least one unit Z³ is present per strand which is connected to at least one unit Z³ on a different hyaluronic acid strand.

It is understood that such hydrogel according to the second embodiment also comprises partly reacted or unreacted units and that the presence of such moieties cannot be avoided. In certain embodiments the sum of such partly reacted or unreacted units is no more than 25% of the total number of units present in the conjugate, such as no more than 10%, such as no more than 15% or such as no more than 10%.

In a conjugate of the present invention according to this second embodiment the number of units Z² ranges from 0 to 70% of all units present in the conjugate of the present invention, such as from 2 to 15%, from 2 to 10%, from 16 to 39, from 40 to 65%, or from 50 to 60% of all units present in the conjugate of the present invention.

In a conjugate of the present invention according to this second embodiment the number of units Z³ ranges from 1 to 30% of all units present in the conjugate of the present invention, such as from 2 to 5%, from 5 to 20%, from 10 to 18%, or from 14 to 18% of all units present in the conjugate of the present invention.

In a conjugate of the present invention according to this second embodiment the number of units Z¹ ranges from 10 to 97% of all units present in the conjugate of the present invention, such as from 20 to 40%, such as from 25 to 35%, such as from 41 to 95%, such as from 45 to 90%, such as from 50 to 70% of all units present in the conjugate of the present invention.

More specific embodiments for -D, -L¹-, -L²-, -SP-, —R^{a1} and —R^{a2} of the second embodiment are as described elsewhere herein.

In a third embodiment the moiety -CL- is a moiety

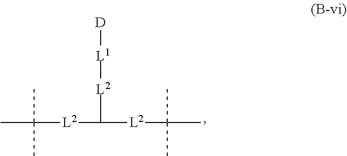

wherein
each dashed line indicates attachment to a unit Z³.

It is understood that a moiety -CL- of formula (B-vi) comprises at least one branching point, which branching point may be selected from the group consisting of

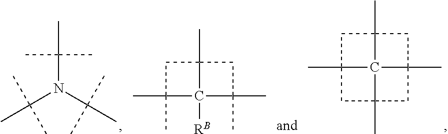

wherein
dashed lines indicate attachment to an arm; and
—R^{B} is selected from the group consisting of —H, C₁₋₆ alkyl, C₂₋₆ alkenyl and C₂₋₆ alkynyl;
wherein C₁₋₆ alkyl, C₂₋₆ alkenyl and C₂₋₆ alkynyl are optionally substituted with one or more —R^{B1}, which are the same or different, and wherein C₁₋₆ alkyl, C₂₋₆ alkenyl and C₂₋₆ alkynyl are optionally interrupted with —C(O)O—, —O—, —C(O)—, —C(O)N(R^{B2})—, —S(O)₂N(R^{B2})—, —S(O)N(R^{B2})—, —S(O)₂—, —S(O)—, —N(R^{B2})S(O)₂N(R^{B2a})—, —S—, —N(R^{B2})—, —OC(OR^{B2})(R^{B2a})—, —N(R^{B2})C(O)N(R^{B2a})—, and —OC(O)N(R^{B2})—;
wherein —R^{B1}, —R^{B2} and —R^{B2a} are selected from —H, C₁₋₆ alkyl, C₂₋₆ alkenyl and C₂₋₆ alkynyl.

In certain embodiments —R^{B} is selected from the group consisting of —H, methyl and ethyl.

Accordingly, a conjugate of the present invention of the third embodiment comprises crosslinked hyaluronic acid strands to which a plurality of drug moieties are covalently and reversibly conjugated, wherein the conjugate of the present invention comprises a plurality of connected units selected from the group consisting of

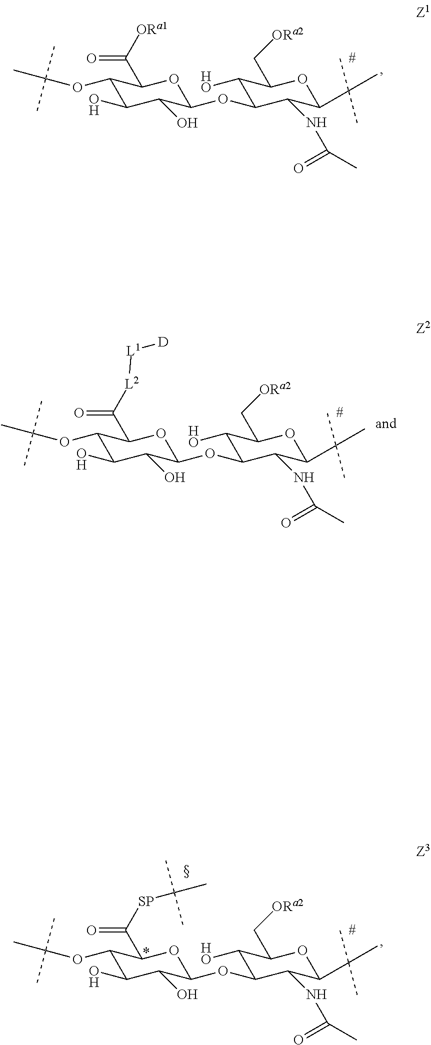

wherein
an unmarked dashed line indicates a point of attachment to an adjacent unit at a dashed line marked with # or to a hydrogen;
a dashed line marked with # indicates a point of attachment to an adjacent unit at an unmarked dashed line or to a hydroxyl;
a dashed line marked with § indicates a point of connection between two units Z³ via a moiety -CL-;
each -CL- comprises at least one degradable bond between the two carbon atoms marked with the * connected by a moiety -CL- and each -CL- is independently of formula (B-vi)

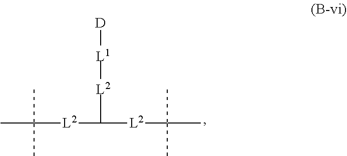

wherein
dashed lines indicate attachment to a unit Z³;
-D, -L¹-, -L²-, -SP-, —R^{a1} and —R^{a2} are used as defined for Z¹, Z² and Z³;
wherein
all units Z¹ present in the conjugate may be the same or different;
all units Z² present in the conjugate may be the same or different;
all units Z³ present in the conjugate may be the same or different;
the number of units Z¹ ranges from 1% to 99% of the total number of units present in the conjugate of the present invention;
the number of units Z² ranges from 0% to 98% of the total number of units present in the conjugate of the present invention; and
the number of units Z³ ranges from 1% to 97% of the total number of units present in the conjugate of the present invention, provided that at least one unit Z³ is present per strand.

It is understood that such hydrogel according to the third embodiment also comprises partly reacted or unreacted units and that the presence of such moieties cannot be avoided. In certain embodiments the sum of such partly reacted or unreacted units is no more than 25% of the total number of units present in the conjugate of the present invention, such as no more than 10%, such as no more than 15% or such as no more than 10%.

In a conjugate of the present invention according to this third embodiment the number of units Z² ranges from 0 to 70% of all units present in the conjugate of the present invention, such as from 2 to 15%, from 2 to 10%, from 16 to 39, from 40 to 65%, or from 50 to 60% of all units present in the conjugate of the present invention.

In a conjugate of the present invention according to this third embodiment the number of units Z³ ranges from 1 to 30% of all units present in the conjugate of the present invention, such as from 2 to 5%, from 5 to 20%, from 10 to 18%, or from 14 to 18% of all units present in the conjugate of the present invention.

In a conjugate of the present invention according to this third embodiment the number of units Z¹ ranges from 10 to 97% of all units present in the conjugate of the present invention, such as from 20 to 40%, such as from 25 to 35%, such as from 41 to 95%, such as from 45 to 90%, such as from 50 to 70% of all units present in the conjugate of the present invention.

In this third embodiment -CL- comprises a moiety -L²- L¹-D, so the presence of units Z² is optional in this embodiment. In certain embodiment no units Z² are present in the third embodiment. In certain embodiments the conjugate of the present invention according to the third embodiment also comprises units Z². The presence of units Z² may have the effect that in case of a high drug loading is desired, which in this embodiment also means a high degree of crosslinking, an undesired high degree of crosslinking can be avoided by the presence of units Z².

More specific embodiments for -D, -L¹-, -L²-, -SP-, —R^{a1} and —R^{a2} of the second embodiment are as described elsewhere herein.

-SP- is absent or a spacer moiety. In certain embodiments -SP- does not comprise a reversible linkage, i.e. all linkages in -SP- are stable linkages.

In certain embodiments -SP- is absent.

In certain embodiments -SP- is a spacer moiety.

In certain embodiments -SP- does not comprise a degradable bond, i.e. all bonds of -SP- are stable bonds. In certain embodiments at least one of the at least one degradable bond in the direct connection between two carbon atoms marked with the * connected by a moiety -CL- is provided by -SP-.

In certain embodiments -SP- is a spacer moiety selected from the group consisting of -T-, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

each —R^{y2} is independently selected from the group consisting of halogen, —CN, oxo (═O), —COOR^{y5}, -OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

In certain embodiments -SP- is a spacer moiety selected from the group consisting of -T-, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

—R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -SP- is a spacer moiety selected from the group consisting of -T-, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently selected from the group consisting of —H, -T, C₁₋₁₀ alkyl, C₂₋₁₀ alkenyl, and C₂₋₁₀ alkynyl;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;

each —R^{y2} is independently selected from the group consisting of halogen and C₁₋₆ alkyl; and

each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -SP- is a C₁₋₂₀ alkyl chain, which is optionally interrupted by one or more groups independently selected from —O—, -T-, —N(R^{y3})— and —C(O)N(R^{y1})—; and which C₁₋₂₀ alkyl chain is optionally substituted with one or more groups independently selected from —OH, -T, —N(R^{y3})— and —C(O)N(R^{y6}R^{y6a}); wherein —R^{y1}, —R^{y6}, —R^{y6a} are independently selected from the group consisting of H and C₁₋₄ alkyl, wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments -SP- has a molecular weight ranging from 14 g/mol to 750 g/mol.

In certain embodiments -SP- has a chain length ranging from 1 to 20 atoms.

In certain embodiments all moieties -SP- of a conjugate are identical.

In certain embodiments -SP- is a C₁₋₁₀ alkyl. In certain embodiments -SP- is a C₁ alkyl. In certain embodiments -SP- is a C₂ alkyl. In certain embodiments -SP- is a C₃ alkyl. In certain embodiments -SP- is a C₄ alkyl. In certain embodiments -SP- is a C₅ alkyl. In certain embodiments -SP- is a C₆ alkyl. In certain embodiments -SP- is a C₇ alkyl. In certain embodiments -SP- is a C₈ alkyl. In certain embodiments -SP- is a C₉ alkyl. In certain embodiments -SP- is a C₁₀ alkyl.

In certain embodiments the TKT conjugate is selected from the group consisting of

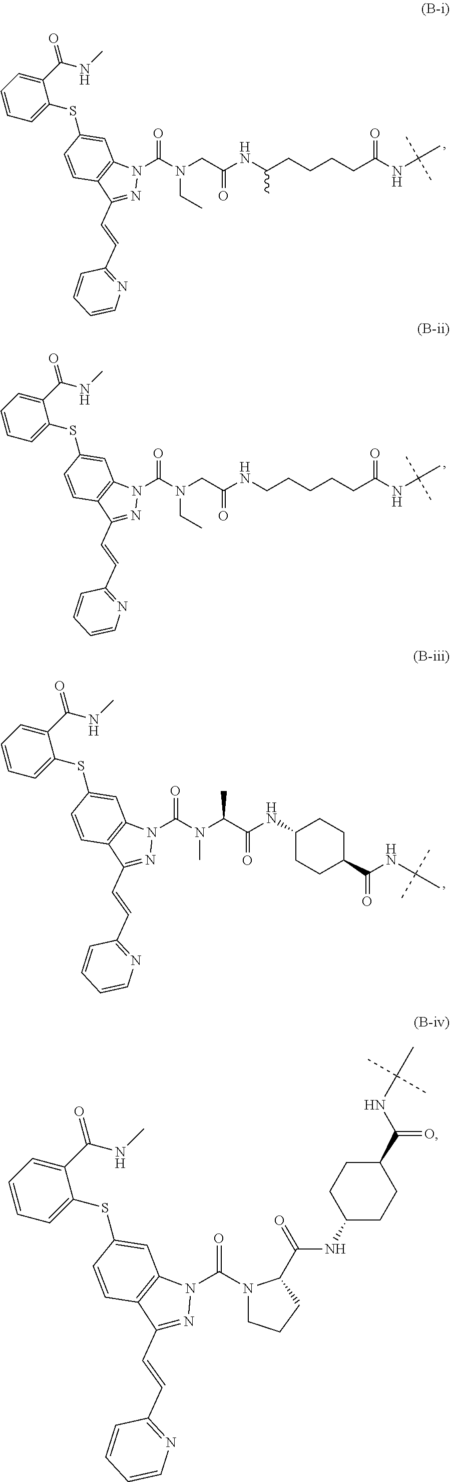

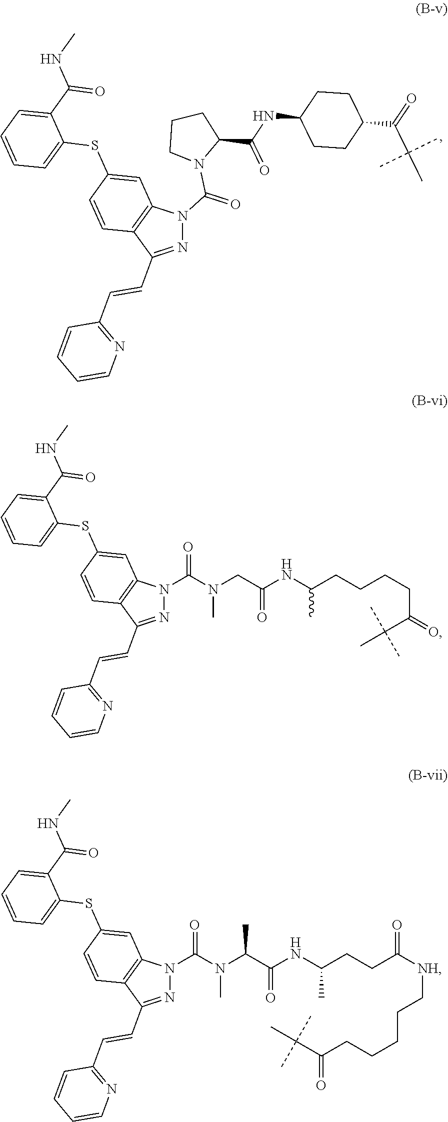

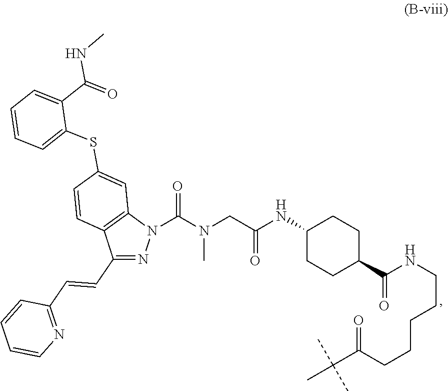

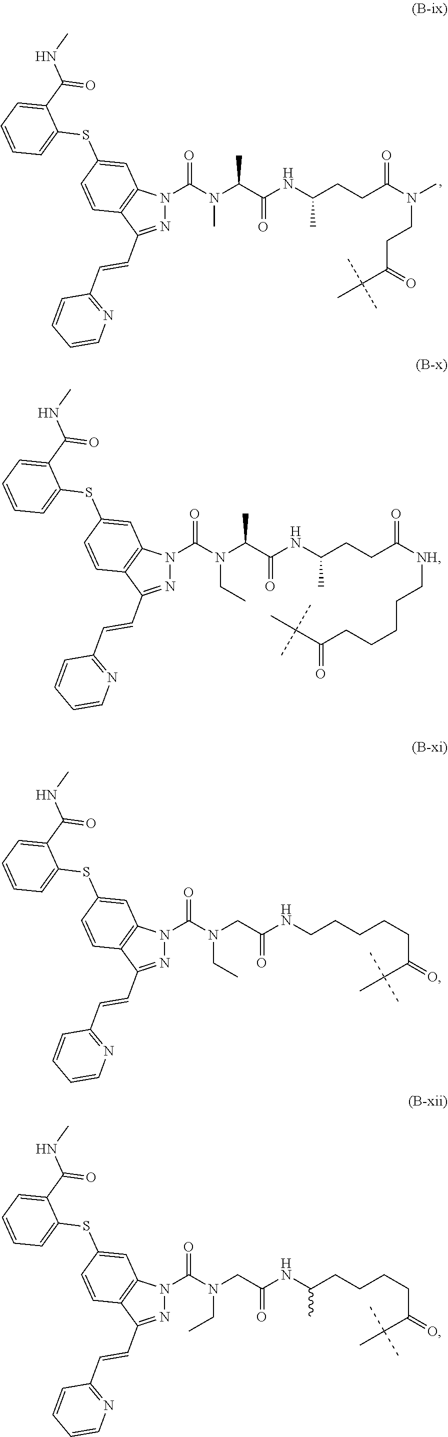

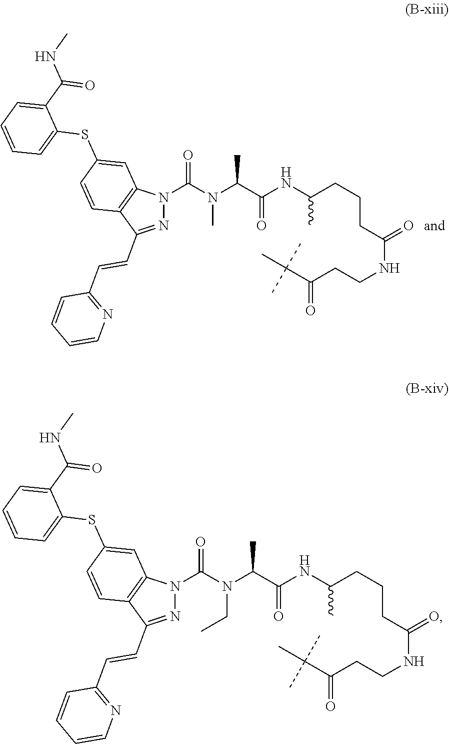

wherein
the dashed line indicates attachment to Z, such as to a PEG-based hydrogel or a hyaluronic acid-based hydrogel.

In certain embodiments the dashed line of formula (B-i), (B-ii), (B-iii), (B-iv), (B-v), (B-vi), (B-vii), (B-viii), (B-ix), (B-x), (B-xi), (B-xii), (B-xiii) and (B-xiv) indicates attachment to a PEG-based hydrogel, in particular to a nitrogen of a PEG-based hydrogel.

In certain embodiments the dashed line of formula (B-i), (B-ii), (B-iii), (B-iv), (B-v), (B-vi), (B-vii), (B-viii), (B-ix), (B-x), (B-xi), (B-xii), (B-xiii) and (B-xiv) indicates attachment to a hyaluronic acid-based hydrogel, in particular to a nitrogen of a hyaluronic acid-based hydrogel.

In certain embodiments the TKI conjugate is of formula (B-i). In certain embodiments the TKI conjugate is of formula (B-ii). In certain embodiments the TKI conjugate is of formula (B-iii). In certain embodiments the TKI conjugate is of formula (B-iv). In certain embodiments the TKI conjugate is of formula (B-v). In certain embodiments the TKI conjugate is of formula (B-vi). In certain embodiments the TKI conjugate is of formula (B-vii). In certain embodiments the TKI conjugate is of formula (B-viii). In certain embodiments the TKI conjugate is of formula (B-ix). In certain embodiments the TKI conjugate is of formula (B-x). In certain embodiments the TKI conjugate is of formula (B-xi). In certain embodiments the TKI conjugate is of formula (B-xii). In certain embodiments the TKI conjugate is of formula (B-xiii). In certain embodiments the TKI conjugate is of formula (B-xiv).

In certain embodiments the TKI conjugate is selected from the group consisting of crystals, nanoparticles, microparticles, nanospheres, microspheres, particles with a diameter larger than about 1 mm and continuous gels. In certain embodiments the TKI conjugate is a crystal. In certain embodiments the TKI conjugate is a nanoparticle, such as a nanoparticle with an average diameter ranging from 5 to 800 nm, a nanoparticle with an average diameter ranging from 10 to 600 nm or a nanoparticle with an average diameter ranging from 20 to 500 nm. In certain embodiments the TKI conjugate is a microparticle, such as microparticle with an average diameter ranging from 10 to 950 μm, such as a microparticle with an average diameter ranging from 20 to 500 μm, such as a microparticle with an average diameter ranging from 25 to 250 μm, such as a microparticle with an average diameter ranging from 30 to 250 μm or a microparticle with an average diameter ranging from 35 to 150 μm. In certain embodiments the TKI conjugate is a nanosphere, such as a nanosphere with an average diameter ranging from 5 to 800 nm, a nanosphere with an average diameter ranging from 10 to 600 nm or a nanosphere with an average diameter ranging from 20 to 500 nm. In certain embodiments the TKI conjugate is a microsphere, such as microsphere with an average diameter ranging from 10 to 700 μm, such as a microsphere with an average diameter ranging from 20 to 500 μm, such as a microsphere with an average diameter ranging from 25 to 250 μm, such as a microsphere with an average diameter ranging from 30 to 250 μm or a microsphere with a n average diameter ranging from 35 to 150 μm. In certain embodiments the TKI conjugate is a particle with an average diameter larger than about 1 mm, such as with an average diameter of at least 2 mm, with an average diameter of at least 4 mm or with an average diameter of at least 5 mm. In certain embodiments the TKI conjugate is a continuous gel.

In certain embodiments upon intra-tissue administration of a single dose of the conjugate of the present invention anti-tumor activity is observed between 7 and 21 days following administration of the conjugate, and wherein the change in mean arterial blood pressure as measured in mmHg is less than 50% of the change in mean arterial blood pressure observed in the same animal species treated with a daily equimolar dose of the corresponding free TKI drug.

The change in mean arterial blood pressure, as measured in mmHg, is less than 50% of the change in mean arterial blood pressure, such as no more than 40%, no more than 30% or no more than 25%.

It is understood that the amount of drug present in a single dose depends on a number of parameters, such as the specific drug for example. In general, it is dose that achieves an anti-tumor activity between 7 and 21 days after administration.

In certain embodiments intra-tissue administration of the conjugate of the present invention results in local inhibition of angiogenesis.

Local inhibition of angiogenesis may be measured in several ways, such as for example by taking a local tissue sample and measuring certain markers, such as protein or mRNA markers. In certain embodiments expression levels 24 hours after intra-tissue administration of at least five mRNAs selected from the group consisting of Hif1a, Vegfa, Vegfb, Vegfc, Mmp19, Plau, Ptgs2, Bcl2l1, Nos3, Egr3, Egr1, Adamts1, Ackr3, Rnd1, Hbegf, Cxcl8, Jag1, Dkk1, Ccl2, Amot, Bmp10, Rcan1, Vcam1, Rcan2, Mmp10, Mmp14, Cyp2c8, Sod2, Icam1, Sele, Mef2c, Notch4, Dll4, Ctnnb1, Ccnd1, Dnajb9, Herpud1, Bcl2, Pecam1, Il10, Fos, Zfp36, Dusp1, Fosb, Nedd9, Atf3, Trib1, Junb, Bhlhb2, Dusp5, Nr4a1, Kif10, K3, Cebpd, Nr4a3, Cxcl2, Il8, Njkbiz, Cxcl1, Cxcl3, Il1a, Dscr1, Hbegf, Maff, Klf9, Map3k8, Bmp2, Stc1, Apold1, Kcnj2, Itgav, Cnksr3, Kitlg, Hivep2, Creb5, Nab2, Klf4, Rgs2, Nr4a2, Per1, Igfbp3, Dnajb9, Ndrg1, Hlx1, Crem, Cited2, Mycn, Ccrl1, Mef2c, and Thbd vary by at least 50% compared to baseline tissue. In certain embodiments the at least five mRNAs are selected from the group consisting of Actb, Aggf1, Angpt1, Angpt2, Angptl1, Angptl3, Angptl4, Anpep, B2m, Bai1, Btg1, Ccl11, Cd55, Cd59b, Cdh5, Cga, Chga, Cited1, Col18a1, Col4a3, Crhr2, Csf3, Ctgf, Cxcl10, Cxcl5, Edil3, Efna1, Efnb2, Egf, Egfl7, Eng, Epas1, Ephb4, Erap1, Erbb2, Ereg, F2, Fgf1, Fgf2, Fgf6, Fgfr3, Figf Flt1, Fn1, Foxf1a, Foxm1, Foxo4, Fst, Fzd5, Gapdh, Glmn, Gna13, Grn, Gusb, Hand2, Hey1, Hey2, Hgf, Hpse, Ifnb1, Ifng, Igf1, Il2a, Il2b, Il1b, Il6, Ipo8, Itgb3, Kdr, Lama5, Lect1, Lep, Mapk14, Mdk, Mmp2, Mmp9, Myocd, Npr1, Nrp1, Nrp2, Ntrk2, Pdgfa, Pdgfb, Pf4, Pgf Pgk1, Plg, Plxdc1, Polr2a, Ppia, Prl, Prl2c2, Prl7d1, Ptn, Ptprj, Qk, Rasa1, Rhob, Rnase4, Rnh1, Rplp2, Runx1, S1pr1, Serpinc1, Serpine1, Serpinf1, Shh, Smad5, Smo, Sphk1, Stab1, Tbx1, Tbx4, Tdgf1, Tek, Tgfa, Tgfb1, Tgfb2, Tgfb3, Tgfbr1, Thbs1, Thbs2, Tie1, Timp1, Timp2, Timp3, Timp4, Tmprss6, Tnf, Tnfaip2, Tnfsfl2, Tnni2, Tnni3, Wars2 and Wt1. In certain embodiments the at least five mRNAs are selected from the group consisting of Hif1a, Vegfa, Vegfb, Vegfc, Mmp19, Plau, Ptgs2, Bcl2l1, Nos3, Egr3, Egr1, Adamts1, Ackr3, Rnd1, Hbegf Cxcl8, Jag1, Dkk1, Ccl2, Amot, Bmp10, Rcan1, Vcam1, Rcan2, Mmp10, Mmp14, Cyp2c8, Sod2, Icam1, Sele, Mej2c, Notch4, Dll4, Ctnnb1, Ccnd1, Dnajb9, Herpud1, Bcl2, Pecam1, Il10, Fos, Zfp36, Dusp1, Fosb, Nedd9, Atf3, Trib1, Junb, Bhlhb2, Dusp5, Nr4a1, Kif10, K3, Cebpd, Nr4a3, Cxcl2, Il8, Njkbiz, Cxcl1, Cxcl3, Il1a, Dscr1, Hbegf, Maff, Klf9, Map3k8, Bmp2, Stc1, Apold1, Kcnj2, Itgav, Cnksr3, Kitlg, Hivep2, Creb5, Nab2, Klf4, Rgs2, Nr4a2, Per1, Igfbp3, Dnajb9, Ndrg1, Hlx1, Crem, Cited2, Mycn, Ccrl1, Mef2c, Thbd, Actb, Aggf1, Angpt1, Angpt2, Angptl1, Angptl3, Angptl4, Anpep, B2m, Bai1, Btg1, Ccl11, Cd55, Cd59b, Cdh5, Cga, Chga, Cited1, Col18a1, Col4a3, Crhr2, Csf3, Ctgf, Cxcl10, Cxcl5, Edil3, Efna1, Efnb2, Egf, Egfl7, Eng, Epas1, Ephb4, Erap1, Erbb2, Ereg, F2, Fgf1, Fgf2, Fgf6, Fgfr3, Figf, Flt1, Fn1, Foxf1a, Foxm1, Foxo4, Fst, Fzd5, Gapdh, Glmn, Gna13, Grn, Gusb, Hand2, Hey1, Hey2, Hgf Hpse, Ifnb1, Ifng, Igf1, Il12a, Il12b, Il1b, Il6, Ipo8, Itgb3, Kdr, Lama5, Lect1, Lep, Mapk14, Mdk, Mmp2, Mmp9, Myocd, Npr1, Nrp1, Nrp2, Ntrk2, Pdgfa, Pdgfb, Pf4, Pgf Pgk1, Plg, Plxdc1, Polr2a, Ppia, Prl, Prl2c2, Prl7d1, Ptn, Ptprj, Qk, Rasa1, Rhob, Rnase4, Rnh1, Rplp2, Runx1, S1pr1, Serpinc1, Serpine1, Serpinf1, Shh, Smad5, Smo, Sphk1, Stab1, Tbx1, Tbx4, Tdgf1, Tek, Tgfa, Tgfb1, Tgfb2, Tgfb3, Tgfbr1, Thbs1, Thbs2, Tie1, Timp1, Timp2, Timp3, Timp4, Tmprss6, Tnf Tnfaip2, Tnfsfl2, Tnni2, Tnni3, Wars2 and Wt1. In certain embodiments expression levels 24 hours after intra-tissue administration of at least five mRNAs selected from the group consisting of Angpt2, Apold1, Dll4, Hey2, Ifnb1, Igfbp3, Il12a, Kcnj2, Kdr, Lep, Mycn, Notch4, Stc1, Tgfa and Timp4 are upregulated by at least 50% compared to baseline tissue. In certain embodiments expression levels 24 hours after intra-tissue administration of at least five mRNAs selected from the group consisting of Angptl3, Bmp10, Cga, Chga, Csf3, Cxcl5, Dkk1, F2, Fgf6, Hand2, Il1a, Il1b, 116, Myocd, Plg, Ptgs2, Rcan2, Sele, Tbx4, Tdgf1, Thbs1, Tmprss6 and Wt1 are downregulated by at least 50% compared to baseline tissue.

It is understood that the term “vary by at least 50%” means that each of the mRNAs may independently of the other mRNAs either be upregulated by at least 50% compared to baseline tissue or may be downregulated by at least 50% compared to baseline tissue.

At least five mRNA from the above list of mRNAs vary by at least 50% compared to baseline tissue, such as 5 mRNA, 6 mRNA, 7 mRNAs, 8 mRNAs, 9 mRNAs or 10 mRNAs.

In certain embodiments the at least five mRNAs from the above list vary by at least 50%, such as by at least 60%, by at least 70%, by at least 80%, by at least 90% or by at least 100%.

In certain embodiments the total amount of TKI moieties and TKI drug molecules remaining locally 3 days after intra-tissue administration of the conjugate of the present invention is at least 25% of the amount of TKI moieties administered by intra-tissue administration.

It is understood that the total amount of TKI moieties and TKI drug molecules remaining locally in such tissue includes both the TKI drug molecules released from the conjugate of the present invention (but remaining in the local tissue) and the TKI moieties not yet released from the conjugate of the present invention and that the determination of the total amount of TKI moieties and TKI drug molecules remaining locally is made 3 days after said intra-tissue administration.

This total amount of TKI moieties and TKI drug molecules may be measured by subjecting a sample to conditions under which unreleased TKI moieties are released from the conjugate (in certain embodiments with an accelerated release half-life) and subsequently determining the amount of TKI drug in said sample, measured in g TKI drug per g tissue.

The amount of TKI moieties and TKI drug molecules remaining locally after 3 days is at least 25% of the amount of administered TKI moieties, such as at least 30%, at least 35%, at least 40%, at least 45%, at least 50%, at least 55% or at least 60%.

In certain embodiments the maximum systemic molar concentration in plasma of TKI drug released from the conjugate of the present invention within 24 hours after intra-tissue administration is at least 50% lower than the maximum systemic molar concentration in plasma of TKI drug within 24 hours after intra-tissue administration of an equimolar dose of the corresponding free TKI drug.

The maximum systemic molar concentration of TKI drug in serum within 24 hours after administration may be determined by taking multiple serum samples within a time period ranging from 0 to 24 hours, determining the TKI drug content in each of them, plotting the TKI drug concentrations as a function of time and determining the maximum concentration using suitable mathematical models. Exemplary time points for taking of the samples may be 1 hour, 3 hours, 6 hours, 12 hours and 24 hours after intra-tissue administration.

The maximum systemic molar concentration of TKI drug released from the conjugate of the present invention in plasma within 24 hours after said intra-tissue administration is at least 50% lower than the maximum systemic molar concentration of TKI drug in plasma within 24 hours after intra-tissue administration of an equimolar dose of the corresponding free TKI drug, such as at least 55% lower, at least 60% lower, at least 65% lower or at least 70% lower.

In certain embodiments anti-tumor activity is present 7 to 21 days post intra-tissue administration and the maximum systemic concentration of TKI drug measured in plasma within 24 hours after said intra-tissue administration that is less than 50%, such as no more than 45%, no more than 40%, no more than 35%, no more than 30%, no more than 25%, no more than 20%, no more than 15% or no more than 10%, than the maximum systemic concentrations of TKI drug measured in plasma within 24 h following systemic administration of a dose of the corresponding free TKI drug required to achieve the same level of anti-tumor activity 7 to 21 days post administration.

In certain embodiments systemic administration of the dose of the corresponding free TKI drug is via oral or intravenous route. In certain said systemic administration is via oral administration. In certain embodiments said systemic administration is via intravenous administration.

In another aspect the present invention relates to a pharmaceutical composition comprising at least one conjugate or a pharmaceutically acceptable salt thereof of the present invention and at least one excipient. In certain embodiments such pharmaceutical composition has a pH ranging from and including pH 3 to pH 8. In certain embodiments such pharmaceutical composition is a suspension formulation. In certain embodiments such pharmaceutical composition is a dry formulation.

Such suspension or dry pharmaceutical composition comprises at least one excipient. Excipients used in parenteral formulations may be categorized as, for example, buffering agents, isotonicity modifiers, preservatives, stabilizers, anti-adsorption agents, oxidation protection agents, viscosifiers/viscosity enhancing agents, or other auxiliary agents. However, in some cases, one excipient may have dual or triple functions. In certain embodiments the at least one excipient comprised in the pharmaceutical composition of the present invention is selected from the group consisting of
(i) Buffering agents: physiologically tolerated buffers to maintain pH in a desired range, such as sodium phosphate, bicarbonate, succinate, histidine, citrate and acetate, sulphate, nitrate, chloride, pyruvate; antacids such as Mg(OH)₂ or ZnCO₃ may be also used;
(ii) Isotonicity modifiers: to minimize pain that can result from cell damage due to osmotic pressure differences at the injection depot; glycerin and sodium chloride are examples; effective concentrations can be determined by osmometry using an assumed osmolality of 285-315 mOsmol/kg for serum;
(iii) Preservatives and/or antimicrobials: multidose parenteral formulations require the addition of preservatives at a sufficient concentration to minimize risk of patients becoming infected upon injection and corresponding regulatory requirements have been established; typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol, and benzalkonium chloride;
(iv) Stabilizers: Stabilisation is achieved by strengthening of the protein-stabilising forces, by destabilisation of the denatured state, or by direct binding of excipients to the protein; stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives; in addition, oligomers or polymers such as cyclodextrins, dextran, dendrimers, PEG or PVP or protamine or HSA may be used;
(v) Anti-adsorption agents: Mainly ionic or non-ionic surfactants or other proteins or soluble polymers are used to coat or adsorb competitively to the inner surface of the formulation's container; e.g., poloxamer (Pluronic F-68), PEG dodecyl ether (Brij 35), polysorbate 20 and 80, dextran, polyethylene glycol, PEG-polyhistidine, BSA and HSA and gelatins; chosen concentration and type of excipient depends on the effect to be avoided but typically a monolayer of surfactant is formed at the interface just above the CMC value;
(vi) Oxidation protection agents: antioxidants such as ascorbic acid, ectoine, methionine, glutathione, monothioglycerol, morin, polyethylenimine (PEI), propyl gallate, and vitamin E; chelating agents such as citric acid, EDTA, hexaphosphate, and thioglycolic acid may also be used;
(vii) Viscosifiers or viscosity enhancers: retard settling of the particles in the vial and syringe and are used in order to facilitate mixing and resuspension of the particles and to make the suspension easier to inject (i.e., low force on the syringe plunger); suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxypropylene)-poly(oxyethylene) (e.g. Pluronic®), polyetherester copolymer, such as a polyethylene glycol terephthalate/polybutylene terephthalate copolymer, sucrose acetate isobutyrate (SAIB), dextran or derivatives thereof, combinations of dextrans and PEG, polydimethylsiloxane, collagen, chitosan, polyvinyl alcohol (PVA) and derivatives, polyalkylimides, poly (acrylamide-co-diallyldimethyl ammonium (DADMA)), polyvinylpyrrolidone (PVP), gly cosaminogly cans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone; such block copolymers as well as the abovementioned poloxamers may exhibit reverse thermal gelation behavior (fluid state at room temperature to facilitate administration and gel state above sol-gel transition temperature at body temperature after injection);
(viii) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue; a spreading agent such as but not limited to hyaluronidase temporarily decreases the viscosity of the extracellular matrix and promotes diffusion of injected drugs; and
(ix) Other auxiliary agents: such as wetting agents, viscosity modifiers, antibiotics, hyaluronidase; acids and bases such as hydrochloric acid and sodium hydroxide are auxiliary agents necessary for pH adjustment during manufacture.

In another aspect the present invention relates to the TKI conjugate for use as a medicament, such as a medicament for the treatment of a cell-proliferation disorder.

In another aspect the present invention relates to the TKI conjugate for use in the manufacture of a medicament, such as for the manufacture of a medicament for the treatment of a cell-proliferation disorder.

In another aspect the present invention relates to the TKI conjugate of the present invention for use in the treatment a cell-proliferation disorder.

In another aspect the present invention relates to a method of treating in a mammalian patient in need of the treatment of one or more diseases which can be treated with a TKI drug, comprising the step of administering to said patient in need thereof a therapeutically effective amount of the TKI conjugate or a pharmaceutically acceptable salt thereof or a pharmaceutical composition comprising the TKI conjugate of the present invention.

In certain embodiments the treatment of the cell-proliferation disorder is in a patient undergoing treatment with at least one additional drug or therapy selected from the group consisting of anti-PD1 and anti-PDL1 compounds, other immune checkpoint antagonist therapies, pattern recognition receptor agonist compounds, immune agonist therapy, oncolytic viral therapy, anti-cancer vaccination, immunostimulatory cytokines, kinase inhibitors, transcription factor inhibitors, DNA repair inhibitors, cellular therapy, chemotherapy, radiotherapy and surgery.

Such at least one additional drug may be administered to the patient prior to, simultaneously with or after administration of the TKI conjugate of the present invention. In certain embodiments at least one additional drug may be administered to the patient prior to administration of the TKI conjugate of the present invention. In certain embodiments at least one additional drug may be administered to the patient simultaneously with administration of the TKI conjugate of the present invention. In certain embodiments at least one additional drug may be administered to the patient after administration of the TKI conjugate of the present invention.

In certain embodiments the treatment of a cell-proliferation disorder is administered to a mammalian patient together with one or more further drug molecules or treatments. It is understood that the one or more further drug molecules may be administered in the form of a pharmaceutically acceptable salt or as a pharmaceutical composition comprising such one or more further drug molecules or their pharmaceutically acceptable salts. In certain embodiments the mammalian patient is selected from mouse, rat, non-human primate and human. In certain embodiments the mammalian patient is a human patient.

In certain embodiments the treatment with the TKI conjugate, its pharmacologically acceptable salt or the pharmaceutical composition of the present invention may be initiated prior to, concomitant with, or following surgical removal of a tumor or radiation therapy. In addition, such treatment may optionally be combined with at least one other cancer therapeutic, such as systemic immunotherapy. Examples for the at least one cancer therapeutic, such as systemic immunotherapy, are as provided elsewhere herein for the one or more further drug molecules. In certain embodiments the TKI conjugate, its pharmacologically acceptable salt or the pharmaceutical composition of the present invention is administered intratumorally prior to, concomitant with, or following combination with at least one systemic immunotherapy, prior to radiation therapy or surgical removal of the injected tumor. In certain embodiments the TKI conjugate, its pharmacologically acceptable salt or the pharmaceutical composition of the present invention is administered intratumorally prior to, concomitant with, or following combination with at least one systemic immunotherapy, following radiation therapy or surgical removal of a tumor. In certain embodiments the TKI conjugate, its pharmacologically acceptable salt or the pharmaceutical composition of the present invention is administered into tumor draining lymph nodes prior to, concomitant with, or following surgical removal of a tumor or radiation therapy. In certain embodiments the TKI conjugate, its pharmacologically acceptable salt or the pharmaceutical composition of the present invention is administered into tumor draining lymph nodes prior to, concomitant with, or following combination with at least one systemic immunotherapy, and prior to, concomitant with, or following surgical removal of a tumor or radiation therapy. In certain embodiments the TKI conjugate, its pharmacologically acceptable salt or the pharmaceutical composition of the present invention is administered intratumorally into metastatic tumors that may arise prior to or following surgical removal or radiation therapy of primary tumor. In certain embodiments the TKI conjugate, its pharmacologically acceptable salt or the pharmaceutical composition of the present invention is administered intratumorally into metastatic tumors that may arise prior to, concomitant with, or following combination with at least one systemic immunotherapy, and prior to, concomitant with, or following surgical removal or radiation therapy of primary tumor. In certain embodiments at least one systemic therapy is administered prior to surgical removal of a tumor or radiation therapy, followed by intratumoral administration of the TKI conjugate, its pharmacologically acceptable salt or the pharmaceutical composition of the present invention. In certain embodiments intratumoral administration of the TKI conjugate, its pharmacologically acceptable salt or the pharmaceutical composition of the present invention is administered first, followed by subsequent treatment in combination with at least one systemic therapy. In certain embodiments at least one systemic therapy is administered prior to surgical removal of a tumor, followed by administration of the TKI conjugate, its pharmacologically acceptable salt or the pharmaceutical composition of the present invention to the tumor bed following surgery or by intratumoral administration in tumor not removed by surgery.

Said one or more further drug molecules may be administered to said patient prior to, together with or after administration of the conjugate of the present invention or the pharmaceutically acceptable salt thereof or the pharmaceutical composition comprising the conjugate of the present invention. If the one or more further drug molecules are administered together with the conjugate of the present invention or a pharmaceutically acceptable salt thereof or the pharmaceutical composition comprising the conjugate said one or more further drug molecules may be either present in the same preparation, such as the same pharmaceutical composition, may be present in the conjugate of the present invention or may be present in a different preparation.

In certain embodiments such one or more further drug molecules are selected from the group cytotoxic/chemotherapeutic agents, immune checkpoint inhibitors or antagonists, immune agonists, multi-specific drugs, antibody-drug conjugates (ADC), radionuclides or targeted radionuclide therapeutics, DNA damage repair inhibitors, tumor metabolism inhibitors, pattern recognition receptor agonists, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, and hormones including hormone peptides and synthetic hormones.

In certain embodiments the one or more further drug is a cytotoxic/chemotherapeutic agent. In certain embodiments the one or more further drug is an immune checkpoint inhibitor or antagonist. In certain embodiments the one or more further drug is a multi-specific drug. In certain embodiments the one or more further drug is an antibody-drug conjugate (ADC). In certain embodiments the one or more further drug is a radionuclide or a targeted radionuclide therapeutic. In certain embodiments the one or more further drug is DNA damage repair inhibitor. In certain embodiments the one or more further drug is a tumor metabolism inhibitor. In certain embodiments the one or more further drug is a pattern recognition receptor agonist. In certain embodiments the one or more further drug is a chemokine and chemoattractant receptor agonist. In certain embodiments the one or more further drug is a chemokine or chemokine receptor antagonist. In certain embodiments the one or more further drug is a cytokine receptor agonist. In certain embodiments the one or more further drug is a death receptor agonist. In certain embodiments the one or more further drug is a CD47 antagonist. In certain embodiments the one or more further drug is a SIRPα antagonist. In certain embodiments the one or more further drug is an oncolytic drug. In certain embodiments the one or more further drug is a signal converter protein. In certain embodiments the one or more further drug is an epigenetic modifier. In certain embodiments the one or more further drug is a tumor peptide or tumor vaccine. In certain embodiments the one or more further drug is a heat shock protein (HSP) inhibitor. In certain embodiments the one or more further drug is a proteolytic enzyme. In certain embodiments the one or more further drug is a ubiquitin and proteasome inhibitor. In certain embodiments the one or more further drug is an adhesion molecule antagonist. In certain embodiments the one or more further drug is a hormone including hormone peptides and synthetic hormones.

In certain embodiments said one or more further drug is an inhibitor of PD-1. In certain embodiments said one or more further drug is an inhibitor of PD-L1.

Examples for cytotoxic/chemotherapeutic agents, immune checkpoint inhibitors or antagonists, immune agonists, multi-specific drugs, antibody-drug conjugates (ADC), radionuclides or targeted radionuclide therapeutics, DNA damage repair inhibitors, tumor metabolism inhibitors, pattern recognition receptor agonists, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, and hormones including hormone peptides and synthetic hormones are as described elsewhere herein.

In certain embodiments the cell-proliferation disorder is cancer. Such cancer may be selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma.

Examples for lung cancer are non-small cell lung cancer and small cell lung cancer. In certain embodiments the cancer is a non-small cell lung cancer. In certain embodiment the cancer is a small cell lung cancer.

Example for lymphomas are AIDS-related lymphoma, primary central nervous system lymphoma, T-cell lymphoma, cutaneous T-cell lymphoma, Hodgkin's lymphoma, Hodgkin's lymphoma during pregnancy, non-Hodgkin's lymphoma, follicular lymphoma, marginal zone lymphoma, diffuse large B-cell lymphoma, non-Hodgkin's lymphoma during pregnancy and angioimmunoblastic lymphoma. In certain embodiments the cancer is a cutaneous T-cell lymphoma.

Examples for skin cancer are melanoma and Merkel cell carcinoma. In certain embodiments the cancer is a skin cancer. In certain embodiments the cancer is a Merkel cell carcinoma.

An ovarian cancer may for example be an epithelial cancer, a germ cell tumor or a low malignant potential tumor. In certain embodiments the cancer is an epithelial cancer. In certain embodiments the cancer is a germ cell tumor. In certain embodiments the cancer is a low malignant potential tumor.

A pancreatic cancer may for example be an exocrine tumor/adenocarcinoma, pancreatic endocrine tumor (PET) or neuroendocrine tumor (NET). In certain embodiments the cancer is an exocrine tumor/adenocarcinoma. In certain embodiments the tumor is a pancreatic endocrine tumor. In certain embodiments the cancer is a neuroendocrine tumor.

A brain and nervous system cancer may be for example be a medulloblastoma, such as a childhood medulloblastoma, astrocytoma, ependymoma, neuroectodermal tumors, schwannoma, meningioma, pituitary adenoma and glioma. In certain embodiment the cancer is a medullablastoma. In certain embodiments the cancer is a childhood medullablastoma. In certain embodiments the cancer is an astrocytoma. In certain embodiments the cancer is an ependymoma. In certain embodiments the cancer is a neuroectodermal tumor. In certain embodiments the tumor is a schwannoma. In certain embodiments the cancer is a meningioma. In certain embodiments the cancer is a pituitary adenoma. In certain embodiments the cancer is a glioma.

An astrocytoma may be selected from the group consisting of giant cell glioblastoma, glioblastoma, secondary glioblastoma, primary adult glioblastoma, primary pediatric glioblastoma, oligodendroglial tumor, oligodendroglioma, anaplastic oligodendroglioma, oligoastrocytic tumor, oligoastrocytoma, anaplastic oligodendroglioma, oligoastrocytic tumor, oligoastrocytoma, anaplastic oligoastrocytoma, anaplastic astrocytoma, pilocytic astrocytoma, subependymal giant-cell astrocytoma, diffuse astrocytoma, pleomorphic xanthoastrocytoma and cerebellar astrocytoma.

Examples for a neuroectodermal tumor are a pineal primitive neuroectodermal tumor and a supratentorial primitive neuroectodermal tumor.

An ependymoma may be selected from the group consisting of subependymoma, ependymoma, myxopapillary ependymoma and anaplastic ependymoma.

A meningioma may be an atypical meningioma or an anaplastic meningioma.

A glioma may be selected from the group consisting of glioblastoma multiforme, paraganglioma, suprantentorial primordial neuroectodermal tumor (sPNET), brain stem glioma, childhood brain stem glioma, hypothalamic and visual pathway glioma, childhood hypothalamic and visual pathway glioma and malignant glioma.

Examples for breast cancer are breast cancer during pregnancy, triple negative breast cancer, ductal carcinoma in situ (DCIS), invasive ductal carcinoma (IDC), tubular carcinoma of the breast, medullary carcinoma of the breast, mucinous carcinoma of the breast, papillary carcinoma of the breast, cribriform carcinoma of the breast, invasive lobular carcinoma (ILC), inflammatory breast cancer, lobular carcinoma in situ (LCIS), male breast cancer, Paget's disease of the nipple, phyllodes tumors of the breast and metastasic breast cancer. In certain embodiments the cancer is a breast cancer during pregnancy. In certain embodiments the cancer is a triple negative breast cancer. In certain embodiments the cancer is a ductal carcinoma in situ. In certain embodiments the cancer is an invasive ductal carcinoma. In certain embodiments the cancer is a tubular carcinoma of the breast. In certain embodiments the cancer is a medullary carcinoma of the breast. In certain embodiments the cancer is a mucinous carcinoma of the breast. In certain embodiments the cancer is a papillary carcinoma of the breast. In certain embodiments the cancer is a cribriform carcinoma of the breast. In certain embodiments the cancer is an invasive lobular carcinoma. In certain embodiments the cancer is an inflammatory breast cancer. In certain embodiments the cancer is a lobular carcinoma in situ. In certain embodiments the cancer is a male breast cancer. In certain embodiments the cancer is a Paget's disease of the nipple. In certain embodiments the cancer is a phyllodes tumor of the breast. In certain embodiments the cancer is a metastatic breast cancer.

Examples for a carcinoma are neuroendocrine carcinoma, adrenocortical carcinoma and Islet cell carcinoma. In certain embodiments the cancer is a neuroendocrine carcinoma. In certain embodiments the cancer is an adrenocortical carcinoma. In certain embodiments the cancer is an Islet cell carcinoma.

Examples for a colorectal cancer are colon cancer and rectal cancer. In certain embodiments the cancer is a colon cancer. In certain embodiments the cancer is a rectal cancer.

A sarcoma may be selected from the group consisting of Kaposi's sarcoma, osteosarcoma/malignant fibrous histiocytoma of bone, soft tissue sarcoma, Ewing's family of tumors/sarcomas, rhabdomyosarcoma, clear cell sarcoma of tendon sheaths, central chondrosarcoma, central and periosteal chondroma, fibrosarcoma and uterine sarcoma. In certain embodiments the cancer may be a Kaposi's sarcoma. In certain embodiments the cancer may be an osteosarcoma/malignant fibrous histiocytoma of bone. In certain embodiments the cancer may be a soft tissue sarcoma. In certain embodiments the cancer may be an Ewing's family of tumors/sarcomas. In certain embodiments the cancer may be a rhabdomyosarcoma. In certain embodiments the cancer may be a clear cell sarcoma of tendon sheaths. In certain embodiments the cancer may be a central chondrosarcoma. In certain embodiments the cancer may be a central and periosteal chondroma. In certain embodiments the cancer may be a fibrosarcoma. In certain embodiments the cancer may be a uterine sarcoma.

Examples for a genitourinary cancer are testicular cancer, urethral cancer, vaginal cancer, cervical cancer, penile cancer and vulvar cancer. In certain embodiments the cancer may be a testicular cancer. In certain embodiments the cancer may be a urethral cancer. In certain embodiments the cancer may be a vaginal cancer. In certain embodiments the cancer may be a cervical cancer. In certain embodiments the cancer may be a penile cancer. In certain embodiments the cancer may be a vaginal cancer.

In certain embodiments the cell-proliferation disorder is a glioblastoma. Especially with brain tumors intra-tumoral administration has the advantage of bypassing the blood-brain-barrier and the TKI conjugate allows treatment of these hard-to-inject tumors that otherwise cannot be injected frequently enough with the corresponding free drug molecules.

In certain embodiments the cell-proliferation disorder is an inoperable or surgically challenging cancer of the lung, liver or pancreas.

In certain embodiments the TKI conjugate is administered to a patient via intra-tissue administration, which in certain embodiments is intra-tumoral administration or an administration into one or more tumor-associated draining lymph nodes. In certain embodiments the intra-tissue administration is an intra-tumoral administration. In certain embodiments the intra-tissue administration is an administration into one or more tumor-associated draining lymph nodes.

In certain embodiments an intra-tumoral administration is an administration into a solid tumor.

In certain embodiments the tumor for intra-tumoral administration or the tumor of the tumor-associated draining lymph nodes is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma. Examples for these types of tumors and cancers are as described elsewhere herein.

In certain embodiments the tumor for intra-tumoral administration or the tumor of the tumor-associated draining lymph nodes is a glioblastoma. Especially with brain tumors intra-tumoral administration has the advantage of bypassing the blood-brain-barrier and the TKI conjugate of the present invention allows treatment of these hard-to-inject tumors that otherwise cannot be injected frequently enough with the corresponding free drug molecules.

In certain embodiments the tumor for intra-tumoral administration or the tumor of the tumor-associated draining lymph nodes is an inoperable or surgically challenging cancer of the lung, liver or pancreas.

Materials and Methods

Chemicals

All materials were obtained from commercial vendors except where stated otherwise.

Amino Hydrogels

PEG based amino hydrogels were synthesized as described in example 3 of WO2011/012715A1 using different crosslinkers and crosslinking degrees to give different levels of amine content. All crosslinkers were based on 2 kDa PEG and were synthesized as described in example 2 of WO2011/012715A1 using adipic acid (C6), suberic acid (C8), or azelaic acid (C9). The choice of crosslinker is in brackets and the hydrogels were characterized by their free amine content:

HG-1: 0.309 mmol/g, HG-2: 0.300 mmol/g (C6), HG-3: 0.134 mmol/g (C6); HG-4: 0.668 mmol/g (C9); HG-5: 0.303 mmol/g (C6); HG-6: 0.668 mmol/g (C9); HG-7: 0.331 mmol/g (C6); HG-8: 0.686 mmol/g (C9); HG-9: 0.393 mmol/g (C9); HG-10: 0.474 mmol/g (C8); HG-16: 0.483 mmol/g (C9)

The following hydrogels were prepared by modification of amine hydrogels with lysine as described in example 5 of WO2011/042450A1, and were characterised by their free amine content:

HG-11: 0564 mmol/g (from HG-5); HG-12: 0.614 mmol/g (from HG-7), HG-13: 0.691 mmol/g (from HG-9), HG-14: 0.934 mmol/g (from HG-10), HG-15: 0.621 mmol/g (from HG-7), HG-17: 0.864 mmol/g (from HG-16)

Reactions

Reactions were performed with dry solvents (CH₂Cl₂, DMF, THF) stored over molecular sieves purchased from Sigma-Aldrich Chemie GmbH, Munich, Germany. Generally, reactions were stirred at room temperature and monitored by LCMS.

Solid Phase Synthesis

Solid phase synthesis was performed in syringe reactors with frit. A standard Fmoc protocol was used. 2-Chlorotrityl chloride resin (100-200 mesh), 1% DVB (Merck, Darmstadt, Germany) was loaded with the first amino acid using DIPEA in DCM. Fmoc deprotection was performed using 2:2:96 piperidine/DBU/DMF. Coupling of the next amino acid was performed using PyBOP/DIPEA or HATU/DIPEA in DMF. Cleavage from the resin was effected by HFIP or TFA/TES/water/DCM 48:2:2:48. Products were concentrated in vacuo.

RP-HPLC Purification

Preparative RP-HPLC purifications were performed with a Waters 600 controller with a 2487 Dual Absorbance Detector or an Agilent Infinity 1260 preparative system using a Waters XBridge BEH300 Prep C18 10 μm, 150×30 mm column as stationary phase. Products were detected at 215 nm, 320 nm or 360 nm. Linear gradients of solvent system A (water containing 0.1% TFA v/v) and solvent system B (acetonitrile containing 0.1% TFA v/v) were used. HPLC fractions containing product were pooled and lyophilized if not stated otherwise.

Flash Chromatography

Flash chromatography purifications were performed on an Isolera One system or an Isolera Four system from Biotage AB, Sweden, using Biotage KP-Sil silica cartridges. Products were detected at 254 nm, 280 nm, or 360 nm.

RP-LPLC Purification

Low pressure RP chromatography purifications were performed on an Isolera One system or an Isolera Four system from Biotage AB, Sweden, using Biotage SNAP C18 cartridges. Products were detected at 215 nm and 360 nm. Linear gradients of solvent system A (water containing 0.1% TFA v/v) and solvent system B (acetonitrile containing 0.1% TFA v/v). Fractions containing product were pooled and lyophilized if not stated otherwise.

UPLC-MS Analysis

Analytical ultra-performance LC (UPLC)-MS was performed on a Waters Acquity system or an Agilent 1290 Infinity II equipped with a Waters BEH300 C18 column (2.1×50 mm, 1.7 μm particle size or 2.1×100 mm, 1.7 μm particle size); solvent A: water containing 0.04% TFA (v/v), solvent B: acetonitrile containing 0.05% TFA (v/v) coupled to a Waters Micromass ZQ or coupled to an Agilent Single Quad MS system.

Drug Moiety Content Determination from Hydrogels

Drug moiety contents of hydrogels were determined by total release of the drug after basic incubation and LCMS quantification (UV based).

### Example 1

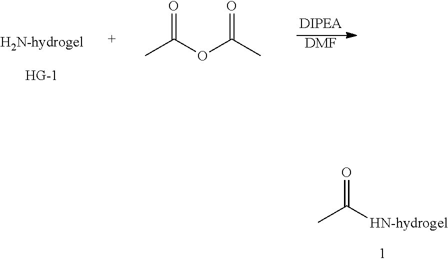

Hydrogel HG-1 (1.13 g, 0.350 mmol) was split between two 20 mL syringes, each equipped with a PE frit, and each washed 3× with a 1% (v/v) solution of DIPEA in anhydrous DMF. Into each syringe was drawn a solution of acetic anhydride (0.33 mL; 3.51 mmol; 20.0 eq.) and DIPEA, (0.61 mL; 3.51 mmol; 20.00 eq.) in anhydrous DMF (6.81 mL). The syringes were closed with a sterile cap and shaken for 1.5 h at 1000 rpm at r.t. The solvent was expelled, and each syringe was washed 10× with anhydrous DMF, and 10× with ethanol. The resulting hydrogel was dried in vacuo.

Under sterile conditions, the dried hydrogel (1.09 g; 1.00 eq.) was transferred into a 50 ml Falcon tube. To this was added pH 5.5 aq. 20 mM sodium succinate, 77 g/l trehalose dihydrate, 0.2% Pluronic F-68 buffer (14 mL), and the Falcon tube was agitated for 30 min on a shaker until a homogenous suspension had formed. A 7% w/w suspension of hydrogel 1 in pH 5.5 buffer was obtained.

### Example 2

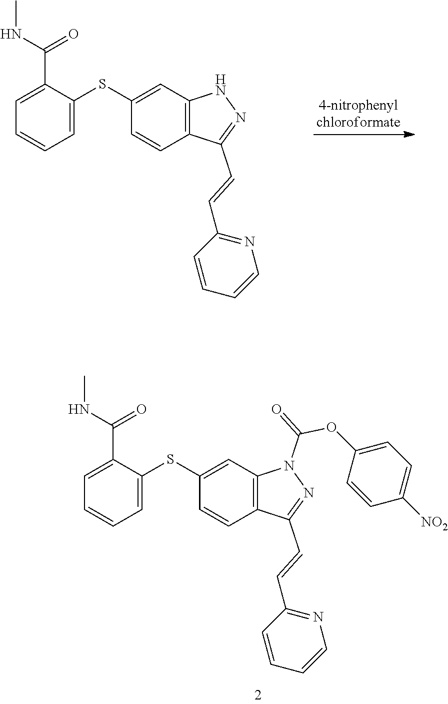 [SMILES: CNC(=O)c1ccccc1Sc1ccc2c(/C=C/c3ccccn3)n[nH]c2c1 ; CNC(=O)c1ccccc1Sc1ccc2c(/C=C/c3ccccn3)nn(C(=O)Oc3ccc([N+](=O)[O-])cc3)c2c1]

4-nitrophenyl chloroformate (188 mg, 0.93 mmol) was dissolved in THF (8 mL). This solution was added to axitinib (100 mg, 0.26 mmol) and the reaction heated at 80° C. for 7 h with stirring (yellow suspension). The reaction suspension was left standing at rt overnight. The suspension was centrifuged, the supernatant removed, and the precipitate washed with ethyl acetate (2 times 6 ml). The precipitate was dried in high vacuum.

Yield: 139 mg (92%, HCl salt)

MS: m/z 552.11=[M+H]⁺, (calculated=552.14).

### Example 3

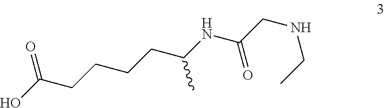 [SMILES: CCNCC(=O)NC(C)CCCCC(=O)O]

Methyl 6-oxo-heptanoate (2 g, 12.64 mmol) was dissolved in methanol (13 mL) and ammonium acetate (9.75 g, 126.43 mmol) and sodium cyanoborohydride (1.19 g, 18.96 mmol) was added with stirring. The resulting suspension turned into a solution and stirring was continued overnight. The mixture was diluted with water (70 ml) and ethyl acetate was added (80 ml). The pH of the water phase was adjusted to ca pH 11 with 25 ml 4 M NaOH. The water phase was extracted 3 more times with 70 ml ethyl acetate. The combined organic phases were dried (MgSO₄), filtered and concentrated in vacuo. The crude from the first step was dissolved in DMF (20 mL) and N-Boc-N-ethylglycine (2.55 g, 12.56 mmol), PyBOP (7.19 g, 13.82 mmol) and DIPEA (6.56 mL, 37.68 mmol) were added with stirring. After 1 h the reaction was diluted with 60 ml ethyl acetate and washed with 0.1 M HCl (3 times 80 ml), 0.5 M NaOH (3 times 50 ml) and brine (50 ml). The organic phase was dried (MgSO₄), filtered and concentrated in vacuo. The residue was purified using flash chromatography (heptane/ethyl acetate). The product from the former step was dissolved in THF (10 mL) and LiOH (0.46 g, 19.21 mmol) was dissolved in water (4 mL). The solutions were combined and stirred vigorously. After 3 h the reaction was diluted with 80 ml ethyl acetate and 60 ml 1 M HCl was added. The pH of the aqueous phase was below 2. The organic phase was collected, and the aqueous phase extracted with ethyl acetate (2 times 50 ml). The combined organic solution was dried (MgSO₄), filtered and concentrated in vacuo. The residue was dissolved in DCM (10 mL) and TFA (5 mL) was added with vigorous stirring in an open flask. After 30 min the reaction was concentrated in vacuo and co-evaporated once with 5 ml DCM. The crude was dissolved in water (40 ml) and lyophilized.

Yield: 2.54 g (59%, TFA salt)

MS: m/z 230.94=[M+H]⁺, (calculated=231.17).

### Example 4

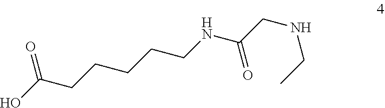

4 was synthesized using solid phase synthesis following the general protocol using Fmoc-Ahx-OH and Boc-N-ethyl glycine as building blocks. Upon cleavage from the resin the BOC protecting group was removed concurrently using the TFA cleavage cocktail.

The cleavage solution was concentrated in vacuo, and the residue was dissolved in acetonitrile/water and lyophilized.

Yield: 1.01 g (quant., TFA salt)

MS: m/z 216.92=[M+H]⁺, (calculated=217.16).

### Example 5

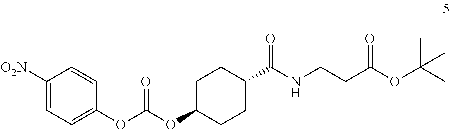 [SMILES: CC(C)(C)OC(=O)CCNC(=O)[C@H]1CC[C@H](OC(=O)Oc2ccc([N+](=O)[O-])cc2)CC1]

To a suspension of trans-4-hydroxycyclohexanoic acid (61 mg, 0.43 mmol) in DCM (0.8 ml) was added HOBt (63 mg, 0.47 mmol) then DIC (73 μL, 0.47 mmol). To the suspension was added DMF (0.2 ml). H-beta-Ala-OtBu hydrochloride (86 mg, 0.47 mmol) in DCM (0.2 ml). After 4.5 h DIPEA (60 μl) was added. After 5 h the reaction was diluted with DCM (ca. 10 mL) and filtered. The filtrate was washed with aq. 0.1 M HCl, then brine. The organic phase was dried over MgSO₄, filtered and concentrated in vacuo. The product was purified by RP-HPLC. The product of the former step (57.00 mg, 0.21 mmol) was dissolved in DCM (2.5 mL) and DMAP (26 mg, 0.21 mmol) was added. 4-nitrophenyl chloroformate (85 mg, 0.42 mmol) in DCM (0.5 mL) and DIPEA (110 μL, 0.63 mmol) were added. After 1 h 0.1 M aq. HCl (15 mL) was added and the mixture diluted with ethyl acetate (30 mL). The organic phase was washed with 0.1 M aq. HCl (2 times 10 mL). The aq phase was re-extracted with ethyl acetate (3 times 10 mL). The organic phases were combined, dried over MgSO₄, filtered, and concentrated in vacuo.

Yield: 119 mg (65%)

MS: m/z 437.21=[M+H]⁺, (calculated=437.19).

### Example 6

Synthesis of 6a and 6b

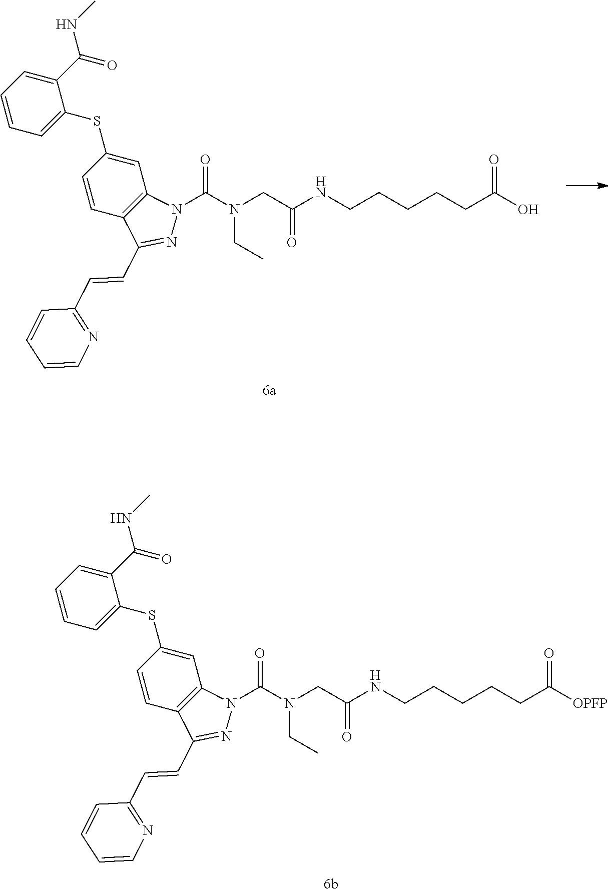

4 (689 mg, 2.09 mmol) was dissolved in 4 mL of DMF and DIPEA (0.9 mL, 5.2 mmol) was added. A suspension of 2 (0.61 g, 0.98 mmol) in DMF (8.2 mL) was added. After 30 min the reaction was added to a solution of 2.6 mL 4 N HCl in dioxane and 237 mL of ethyl acetate. The precipitate was centrifuged, the supernatant decanted and the residue washed once with 180 ml ethyl acetate. The residue was purified by RP-LPLC to obtain 6a.

Yield: 0.34 g (46%, TFA salt)

MS: m/z 629.34=[M+H]⁺, (calculated=629.26).

6a (0.34 gg; 0.45 mmol) was dissolved in DMF (6.76 mL) and bis(pentafluorophenyl) carbonate (0.21 mg, 0.54 mmol) was added. DIPEA (0.48 mL, 2.73 mmol) was added. After 45 min acetic acid (0.48 ml) was added and the product purified by RP-LPLC to obtain 6b.

Yield: 0.40 g (98%, TFA salt)

MS: m/z 795.39=[M+H]⁺, (calculated=795.24).

Synthesis of 6c

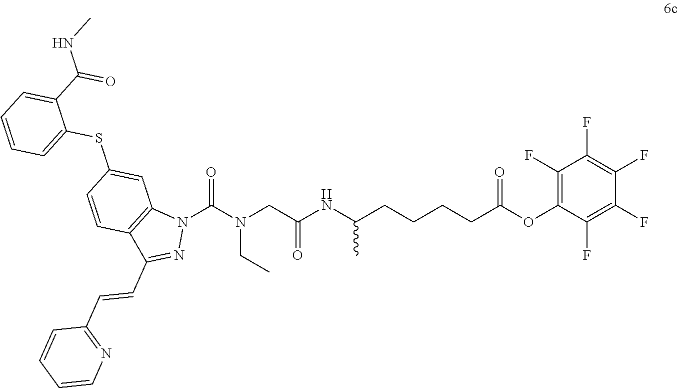 [SMILES: CCN(CC(=O)NC(C)CCCCC(=O)Oc1c(F)c(F)c(F)c(F)c1F)C(=O)n1nc(/C=C/c2ccccn2)c2ccc(Sc3ccccc3C(=O)NC)cc21]

A suspension of 2 (8.50 mL, 0.13 mol/L; 1.04 mmol) in DMF was added to 3 (0.72 g, 2.08 mmol) and DIPEA (0.91 mL, 5.21 mmol) was added. After 45 min the reaction was added to a solution of 2.6 mL 4 N HCl in dioxane and 160 mL of ethyl acetate. The precipitate was centrifuged, the supernatant decanted and the residue purified by RP-LPLC. The product from the former step (0.42 g, 0.55 mmol) was dissolved in DMF (8.40 mL) and bis(pentafluorophenyl) carbonate (0.27 mg, 0.67 mmol) was added. DIPEA (0.58 mL, 3.33 mmol) was added. After 1 h acetic acid (0.48 ml) was added and the product purified by RP-LPLC.

Yield: 0.27 g (29%, TFA salt)

MS: m/z 809.36=[M+H]⁺, (calculated=809.26).

Synthesis of 6d

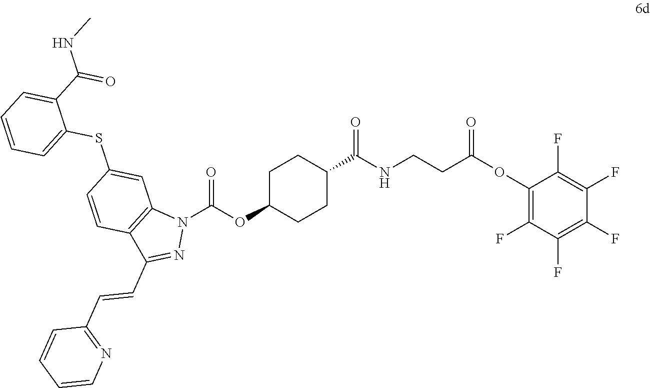 [SMILES: CNC(=O)c1ccccc1Sc1ccc2c(/C=C/c3ccccn3)nn(C(=O)O[C@H]3CC[C@H](C(=O)NCCC(=O)Oc4c(F)c(F)c(F)c(F)c4F)CC3)c2c1]

A solution of 5 (49 mg, 0.11 mmol) in THF (1.60 mL) was added to axitinib (22 mg, 56 μmol). DIPEA (49 μL, 0.28 mmol) was added. The reaction was heated to 60° C. for 6 h and stirred overnight at RT. DMF (0.5 ml) was added. suspension became a solution. Heated again at 60° C. The reaction was heated to 60° C. for 6.5 h and stirred for 3 days at RT. DMAP (>1 eq) was added and the reaction stirred at RT for 1 day. TFA (25 μl) was added and the product purified by RP-HPLC. The product from the former step (16 mg, 20 μmol) was dissolved in a mixture of DCM (1 mL) and TFA (1 ml). After 2 h the volatiles were removed in vacuo and the residue dissolved in 3 ml acetonitrile/water/TFA 1:1:0.002 and lyophilized. The product from the former step (15 mg, 20 μmol) was dissolved in DMF (0.29 mL) and bis(pentafluorophenyl) carbonate (9.4 mg 24 μmol) was added. DIPEA (21 μL; 0.12 mmol) was added. After 1.5 h TFA (10 μl) was added and the reaction purified by RP-HPLC.

Yield: 13 mg (25%, TFA salt)

MS: m/z 794.25=[M+H]⁺, (calculated=794.21).

### Example 7

Synthesis of 7a-c

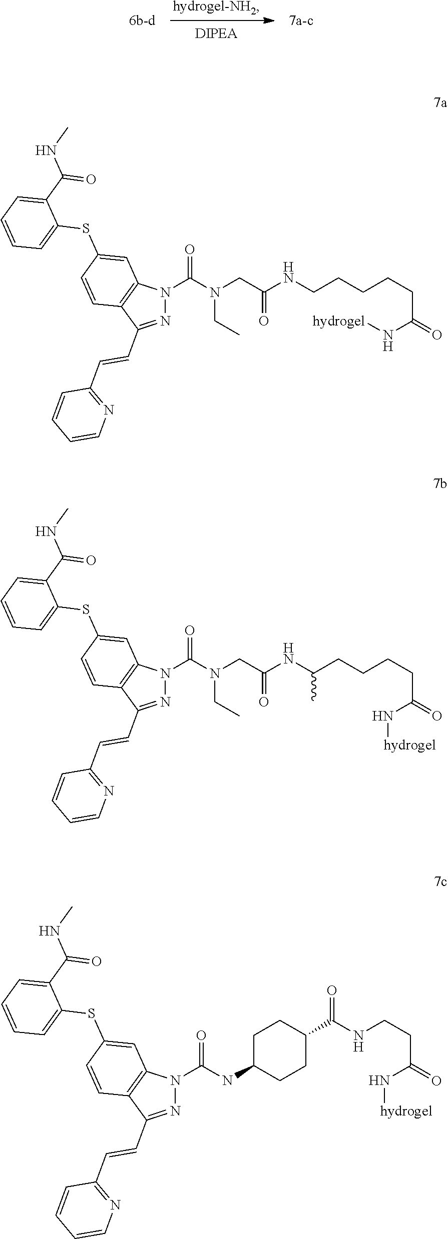

The hydrogel was swollen in 1% DIPEA in DMF in a syringe reactor containing a PE frit. The syringe reactor was 3 times filled, shaken for 1 min and drained. 6 was dissolved in DMF and DIPEA was added. The solution was drawn into the syringe containing the hydrogel. The syringe was shaken for longer than 16 h at RT. The syringe was drained, and the hydrogel was washed several times with DMF, water and pH 5.5 aq. 20 mM sodium succinate, 77 g/l trehalose dihydrate, 0.2% Pluronic F-68 buffer and a hydrogel suspension in buffer was obtained.

7a: Materials: HG-2: 0.82 g, DIPEA: 0.21 ml, 6b: 0.40 g yield: suspension, 7.55 mg/ml axitinib in hydrogel suspension

7b: Materials: HG-3: 30 mg, DIPEA: 3.5 μl, 6c: 6.7 mg, yield: suspension, 2.93 mg/ml axitinib in hydrogel suspension

7c: Materials: HG-3: 30 mg, DIPEA: 3.5 μl, 16 μmol, 6d: 13 mg yield: suspension, 3.65 mg/ml axitinib in hydrogel suspension

### Example 8

Synthesis of 8a and 8b

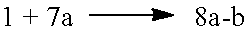

Under sterile conditions, the formulated acylated hydrogel 1 and axtinib loaded hydrogel 7a were combined in a Falcon tube and the mixture vortexed to give a homogenized hydrogel formulated in pH 5.5 20 mM sodium succinate, 77 g/l trehalose dihydrate, 0.2% Pluronic F-68 buffer.

8a: Materials: 1: 4.365 mL, 7a: 0.485 mL

yield: suspension, 0.925 mg/ml axitinib in hydrogel suspension

8b: Materials: 1: 3.235 mL, 7a: 1.615 mL

yield: suspension, 2.72 mg/ml axitinib in hydrogel suspension

### Example 9

In Vitro Release Kinetics

The cleavage rate of the reversible bond from conjugates 7a-c was monitored at pH 7.4 and 37° C. in aqueous buffer (pH 7.4 48 mM sodium phosphate, 0.1% Pluronic F68, 20% acetonitrile). The increase in released axitinib in the supernatant was determined by LCMS (UV detection) and used as input for the curve fitting software to obtain the preliminary half-life of the release.

| Compound | t_{1/2} (pH 7.4) | Released product |
|---|---|---|
| 7a | 5.4 d | axitinib |
| 7b | 17.4 d | axitinib |
| 7c | 62 d | axitinib |

### Example 10

In Vivo Anti-Tumor Efficacy and Combination Efficacy Activity with Anti-PD1

The study was conducted in female C57BL/6 mice with an age of 6-8 weeks at the day of tumor inoculation. Mice were implanted with 1×10⁶ MC-38 tumor cells into the left and right flanks. When right flank tumors were grown to a mean tumor volume of ˜104 mm³, mice were randomized into treatment cohorts. Animals were treated by group as shown in the table below. For animals that received intratumoral injections, the right tumor was selected for treatment.

| | | | Dosing | | | |
|---|---|---|---|---|---|---|
| | | Dose | solution | Route of | Volume, | |
| Group | Treatment | level | (mg/mL) | exposure | Dose | Frequency |
| 1 | Vehicle (0.5% | — | — | Oral | 100 μL | Twice daily |
| | Carboxymethyl- | | | | | |
| | cellulose) | | | | | |
| 2 | Axitinib | 25 mg/kg | 5.0 | Oral | 100 μL | Twice daily |
| 3 | 1 | — | — | IT | 50 μL | Once on Day 0 |
| | Rat IgG2a | 10 mg/kg | 2.0 | IP | 100 μL | Every Monday |
| | | | | | | and Friday for 2 |
| | | | | | | weeks |
| 4 | 1 | — | | IT | 50 μL | Once on Day 0 |
| | Anti-PD1 | 10 mg/kg | 2.0 | IP | 100 μL | Every Monday |
| | (RMP1-14) | | | | | and Friday for 2 |
| | | | | | | weeks |
| 5 | 7a | 378 μg | 378 μg | IT | 50 μL | Once on Day 0 |
| | | eq. axitinib | | | | |
| | Rat IgG2a | 10 mg/kg | 2.0 | IP | 100 μL | Every Monday |
| | | | | | | and Friday for 2 |
| | | | | | | weeks |
| 6 | 7a | 378 μg | 378 μg | IT | 50 μL | Once on Day 0 |
| | | eq. axitinib | | | | |
| | Anti-PD1 | 10 mg/kg | 2.0 | IP | 100 μL | Every Monday |
| | (RMP1-14) | | | | | and Friday for 2 |
| | | | | | | weeks |
| 7 | 8a | 136 μg | 136 μg | IT | 50 μL | Once on Day 0 |
| | | eq axitinib | | | | |
| | Rat IgG2a | 10 mg/kg | 2.0 | IP | 100 μL | Every Monday |
| | | | | | | and Friday for 2 |
| | | | | | | weeks |
| 8 | 8b | 47 μg | 47 μg | IT | 50 μL | Once on Day 0 |
| | | eq. axitinib | | | | |
| | Rat IgG2a | 10 mg/kg | 2.0 | IP | 100 μL | Every Monday |
| | | | | | | and Friday for 2 |
| | | | | | | weeks |

*oral = oral gavage, IT = intratumorally injected, IP = intraperitoneally injected
**For orally dosed axitinib, axitinib was suspended in 0.5% carboxymethylcellulose.
***Hydrogels were administered as suspensions in pH 5.5 20 mM sodium succinate, 77 g/l trehalose dihydrate, 0.2% Pluronic F-68 buffer
****D 0 = day when mice were randomized into treatment cohorts

Following treatment initiation, anti-tumor efficacy was assessed by determination of tumor volumes at various time points from tumor size measurements with a caliper. Tumor volumes were calculated according to the formula:

Tumor volume=(L×W²)×0.5

where L is the length of the tumor and W the width (both in mm).

Results:

Absolute Tumor Volumes (Mm³) of Right Flank Tumors

| | | Days post-treatment | | | | | |
|---|---|---|---|---|---|---|---|
| Group | | 0 | 3 | 5 | 7 | 10 | 12 |
| Vehicle | Mean | 104.12 | 335.30 | 524.19 | 699.70 | 1108.34 | 1508.65 |
| | (mm³) | | | | | | |
| | SEM | 3.86 | 41.30 | 48.54 | 63.79 | 93.66 | 151.35 |
| | (mm³) | | | | | | |
| | n | 8 | 8 | 8 | 8 | 8 | 8 |
| Axitinib | Mean | 104.22 | 304.55 | 419.99 | 543.06 | 763.99 | 994.16† |
| | (mm³) | | | | | | |
| | SEM | 3.76 | 26.75 | 38.95 | 57.70 | 85.96 | 96.75 |
| | (mm³) | | | | | | |
| | n | 8 | 8 | 8 | 8 | 8 | 8 |
| 1 + | Mean | 104.28 | 315.83 | 495.70 | 731.61 | 1127.21 | 1606.63†† |
| rIgG2a | (mm³) | | | | | | |
| | SEM | 3.69 | 42.49 | 70.13 | 115.39 | 214.36 | 267.56 |
| | (mm³) | | | | | | |
| | n | 8 | 8 | 8 | 8 | 8 | 8 |
| 1 + | Mean | 104.28 | 283.62 | 459.16 | 667.35 | 973.48 | 1200.68‡ |
| Anti- | (mm³) | | | | | | |
| PD1 | | | | | | | |
| | SEM | 3.63 | 16.98 | 25.03 | 56.27 | 96.03 | 127.10 |
| | (mm³) | | | | | | |
| | n | 8 | 8 | 8 | 8 | 8 | 8 |
| 7a + Rat | Mean | 104.29 | 218.73 | 348.18 | 471.03 | 632.50†, ‡ | 842.49†, ‡ , ‡‡ |
| IgG2a | (mm³) | | | | | | |
| | SEM | 3.65 | 28.56 | 47.53 | 45.60 | 66.22 | 96.64 |
| | (mm³) | | | | | | |
| | n | 8 | 8 | 8 | 8 | 8 | 8 |
| 7a + | Mean | 104.26 | 304.29 | 422.70 | 539.59 | 661.11†, ‡ | 833.24†, ‡, ‡‡ |
| Anti- | (mm³) | | | | | | |
| PD1 | | | | | | | |
| | SEM | 3.63 | 29.36 | 41.25 | 64.38 | 84.86 | 120.24 |
| | (mm³) | | | | | | |
| | n | 8 | 8 | 8 | 8 | 8 | 8 |
| 8a + Rat | Mean | 104.19 | 246.70 | 402.30 | 561.01 | 813.64 | 1204.69‡ |
| IgG2a | (mm³) | | | | | | |
| | SEM | 3.62 | 17.54 | 19.44 | 45.03 | 77.26 | 133.04 |
| | (mm³) | | | | | | |
| | n | 8 | 8 | 8 | 8 | 8 | 8 |
| 8b + | Mean | 104.30 | 351.52 | 455.93 | 573.02 | 929.74 | 1313.79 |
| Rat | (mm³) | | | | | | |
| IgG2a | | | | | | | |
| | SEM | 3.66 | 73.88 | 90.09 | 110.03 | 176.42 | 230.74 |
| | (mm³) | | | | | | |
| | n | 8 | 8 | 8 | 8 | 8 | 7 |

SEM = standard error of the mean,
n = sample size;
†p < 0.02 vs Vehicle,
‡p < 0.05 vs 1 + rIgG2a,
††p < 0.0001 vs Axitinib,
‡‡p < 0.02 vs 8b + Rat IgG2a.
Significance was determined by Two-way ANOVA followed by multiple comparisons using Tukey's Honest Significant Differences (HSD) post-hoc test. These data indicate treating animals with 7a + Rat IgG2a or 7a + Anti-PD1 leads to tumor growth inhibition compared to control animals treated with 1 + rIgG2a over the course of the study.

Absolute Tumor Volumes (Mm³) of Left Flank Tumors

| | | Days post-treatment | | | | | |
|---|---|---|---|---|---|---|---|
| Group | | 0 | 3 | 5 | 7 | 10 | 12 |
| Vehicle | Mean | 112.31 | 362.33 | 505.16 | 729.37 | 1063.48 | 1487.75 |
| | (mm³) | | | | | | |
| | SEM | 2.88 | 49.51 | 44.14 | 65.12 | 96.45 | 154.68 |
| | (mm³) | | | | | | |
| | n | 8 | 8 | 8 | 8 | 8 | 8 |
| Axitinib | Mean | 112.18 | 321.44 | 432.85 | 601.11 | 741.81† | 967.56†, †† |
| | (mm³) | | | | | | |
| | SEM | 4.70 | 34.00 | 49.77 | 66.32 | 87.30 | 109.58 |
| | (mm³) | | | | | | |
| | n | 8 | 8 | 8 | 8 | 8 | 8 |
| 1 + | Mean | 121.14 | 327.39 | 504.59 | 781.75 | 1271.27 | 1747.38 |
| rIgG2a | (mm³) | | | | | | |
| | SEM | 2.34 | 41.86 | 32.87 | 52.79 | 81.19 | 128.25 |
| | (mm³) | | | | | | |
| | n | 8 | 8 | 8 | 8 | 8 | 8 |
| 1 + Anti- | Mean | 109.55 | 309.76 | 446.38 | 630.89 | 921.18 | 1434.99‡‡ |
| PD1 | (mm³) | | | | | | |
| | SEM | 1.68 | 30.85 | 46.83 | 59.36 | 109.07 | 145.90 |
| | (mm³) | | | | | | |
| | n | 8 | 8 | 8 | 8 | 8 | 8 |
| 7a + Rat | Mean | 103.38 | 347.57 | 544.40 | 724.09 | 985.40 | 1376.25‡‡ |
| IgG2a | (mm³) | | | | | | |
| | SEM | 3.94 | 45.46 | 64.97 | 70.66 | 71.66 | 129.56 |
| | (mm³) | | | | | | |
| | n | 8 | 8 | 8 | 8 | 8 | 8 |
| | Mean | 109.74 | 275.82 | 398.52 | 534.67 | 695.94†, ‡ | 913.27†, ††, ‡, *, **, *** |
| | (mm³) | | | | | | |
| 7a + | SEM | 4.46 | 29.51 | 42.82 | 63.15 | 77.53 | 133.71 |
| Anti- | (mm³) | | | | | | |
| PD1 | | | | | | | |
| | n | 8 | 8 | 8 | 8 | 8 | 8 |
| 8a + Rat | Mean | 103.86 | 243.52 | 452.20 | 730.36 | 1072.16 | 1669.22‡‡ |
| IgG2a | (mm³) | | | | | | |
| | SEM | 3.29 | 22.45 | 58.76 | 127.57 | 209.95 | 316.35 |
| | (mm³) | | | | | | |
| | n | 8 | 8 | 8 | 8 | 8 | 8 |
| 8b + Rat | Mean | 113.80 | 340.55 | 556.26 | 757.01 | 1138.57 | 1555.92‡‡ |
| IgG2a | (mm³) | | | | | | |
| | SEM | 5.19 | 26.46 | 62.39 | 91.22 | 162.54 | 176.04 |
| | (mm³) | | | | | | |
| | n | 8 | 8 | 8 | 8 | 8 | 7 |

SEM = standard error of the mean,
n = sample size;
†p < 0.003 vs 1 + rIgG2a,
‡p < 0.03 vs 8b + Rat IgG2a,
††p < 0.0001 vs Vehicle,
‡‡p < 0.05 vs Axitinib,
*p < 0.003 vs 1 + Anti-PD1,
**p < 0.02 vs 7a + Rat IgG2a,
***p < 0.0002 vs 8a + Rat IgG2a.
Significance was determined by Two-way ANOVA followed by multiple comparisons using Tukey's Honest Significant Differences (HSD) post-hoc test. These data indicate treating animals with 7a + Anti-PD1 leads to significant tumor growth inhibition in tumors that were not injected with 7a sooner than mice treated with 1 + Anti-PD1 compared to control animals treated with 1 + rIgG2a over the course of the study.

### Example 11

Synthesis of 9a, 9b, and 9c

As described in example 8, acylated hydrogel 1 and axtinib loaded hydrogel 7a were combined in the appropriate ratio yielding hydrogel suspensions with the following axitinib content:

9a: suspension, 7.38 mg/ml axitinib in hydrogel suspension

9b: suspension, 0.76 mg/ml axitinib in hydrogel suspension

9c: suspension, 2.46 mg/ml axitinib in hydrogel suspension

### Example 12

In Vivo PK Study of Plasma and Tumor Axitinib Concentration

The study was conducted in female C57BL/6 mice with an age of 6-8 weeks at the day of tumor inoculation. Mice were implanted with 1×10⁶ of MC-38/C11G tumor cells into the right flank.

When tumors were grown to a mean tumor volume of ˜94 mm³, mice were randomized into treatment cohorts (day 0). Following randomization, animals received either a single intratumoral injection of hydrogel 1 or hydrogel 9c as a single intratumoral dose in an injection volume of 50 μL, or with orally administered Axitinib suspended in 0.5% carboxymethylcellulose at 3 mg/kg in a 100 μl dose volume twice a day for 14 days. Hydrogels were administered as suspensions in pH 5.5 20 mM sodium succinate, 77 g/l trehalose dihydrate, 0.2% Pluronic F-68 buffer.

Following treatment initiation, anti-tumor efficacy was assessed by determination of tumor volumes at various time points from tumor size measurements with a caliper. Tumor volumes were calculated according to the formula:

Tumor volume=(L×W²)×0.5

where L is the length of the tumor and W the width (both in mm).

After 0.5, 1, 4 12, and 24 hours following treatment, 4 mice from the hydrogel 9c and Axitinib treated groups were sacrificed and terminal blood was collected in EDTA. Plasma was prepared after blood withdrawal. After 72 hours following treatment, 4 mice from the hydrogel 9c treated group were sacrificed and tumors were excised and snap frozen. Plasma samples underwent further processing by solid-phase extraction prior to Axitinib concentration determination by LCMS/MS.

All solvents (UPLC grade) as well as formic acid (ULC/MS quality) were purchased from Biosolve BV, Valkenswaard, The Netherlands. Axitinib concentrations in plasma were determined after solid phase extraction via liquid chromatography separation and detection by mass spectrometry (LCMS). As internal standard [¹³C, D₃]-axitinib was used. LCMS analysis was carried out by using an Agilent Infinity ultra high performance liquid chromatography (UHPLC) system coupled to an AbSciex 6500+ QTrap triple quadrupole mass spectrometer (QQQ) via an ESI probe. Chromatography was performed on a C18 analytical UHPLC column (YMC-Triart, 2.1×50 mm, 1.9 μm particle). Water containing 0.1% formic acid (v/v) was used as mobile phase A and acetonitrile with 0.1% formic acid as mobile phase B. The gradient system comprised a linear increase from 15% B to 30% B in 2.7 min. Mass analysis was performed in multiple reaction monitoring (MRM) mode with the selected transitions for axitinib and the internal standard ([¹³C, D₃]-axitinib). Calibration standards of axitinib in blank plasma were prepared as follows: thawed Li-Heparin C57BL/6 mouse plasma (Biotrend, Köln, Germany) was homogenized. The axitinib formulation was spiked into blank plasma at concentrations between 5,000 pg/mL and 50 pg/mL. These solutions were used for the generation of a calibration curve. Calibration curves were weighted 1/x². For quality control, three quality control samples were prepared accordingly with contents of 4,000 pg/mL (high QC), 400 pg/mL (mid QC) and 90 pg/mL (low QC). For sample preparation, 25 μL of sample were spiked with 175 μL of internal standard solution in 2.25% H₃PO₄ (prepared from 85% H₃PO₄, ACS quality, Merck KGaA, Darmstadt, Germany). Solid phase extraction was performed in 96-well HLB μ-elution plates (Waters Corporation, Milford, Mass., USA, 186001828BA) with standard procedures and an elution mixture of methanol/acetonitrile/water/formic acid (v/v/v/v 24.5/24.5/50/1). 3 μL were injected into the LCMS system.

The excised tumor samples (with weights between 150 and 500 mg) were thawed and transferred into homogenizer tubes containing beads for cell lysis (MP Biomedicals, Eschwege, Germany, Lysing Matrix D, order number: 6913-100). 500 μL NMP, 224 μL deuterated internal standard in NMP, and 300 μL KOH (90%, Sigma-Aldrich Chemie GmbH, Munich, Germany) in water (1/1, m/m) were added to the tumor and the cells lysed with a FastPrep-24 5G homogenizer (MP Biomedicals, Eschwege, Germany) (3 times for 40 seconds with a speed of 6 m/s). The resulting mixture was further incubated at 50° C. and 1,800 rpm for 2 h. After incubation, 300 μL formic acid were added and the dissolved samples were vortexed, centrifuged and diluted 1:10,000 in plasma. The diluted samples were quantified via a LCMS/MS method with selected MRM transitions using a solid-phase extraction method for sample preparation (see above). The amount of axitinib in the tumor sample was back-calculated using the dilution factor and the determined tumor weights.

Results:

Absolute Tumor Volumes (Mm³)

| | | Days post-treatment | | | | |
|---|---|---|---|---|---|---|
| Group | | 0 | 4 | 7 | 11 | 14 |
| 1 | Mean | 95.772 | 318.024 | 539.657 | 1045.452 | 2623.041 |
| | (mm³) | | | | | |
| | SEM | 7.906 | 40.431 | 65.598 | 157.601 | 401.580 |
| | (mm³) | | | | | |
| | n | 10 | 10 | 10 | 10 | 10 |
| Axitinib | Mean | 93.862 | 243.647 | 418.787 | 710.390 | 977.619* |
| | (mm³) | | | | | |
| | SEM | 6.789 | 21.056 | 43.028 | 99.336 | 100.520 |
| | (mm³) | | | | | |
| | n | 10 | 10 | 10 | 9 | 9 |
| 9c | Mean | 95.409 | 251.745 | 427.795 | 656.775 | 1276.250* |
| | (mm³) | | | | | |
| | SEM | 3.231 | 25.014 | 48.843 | 66.012 | 139.650 |
| | (mm³) | | | | | |
| | n | 36 | 12 | 8 | 4 | 4 |

SEM = standard error of the mean,
n = sample size;
*p < 0.05 vs hydrogel 1.
Significance was determined by One-way ANOVA followed by multiple comparisons using Tukey's post-hoc test. These data show both Axitinib and hydrogel 9c treated groups displayed significant anti-tumor activity compared to group 1 treated animals by 14 days post-treatment initiation. Anti-tumor activity observed between Axitinib and hydrogel 9c treated groups was not significantly different than one another.

Axitinib Concentration in Plasma Samples

| | Time (h) | | | | |
|---|---|---|---|---|---|
| Treatment | 0.5 | 1 | 4 | 12 | 24 |
| 9c | 3.23 | 4.12 | 2.34 | 2.98 | 2.19 |
| Axitinib | 101 | 76.8 | — | 0.797 | — |

Data is represented as determined mean axitinib concentrations in ng/mL per time point and group (n=4 for the hydrogel 9c treated group and n=3 for the Axitinib treated group); method LLOQ at 50.0 pg/mL; ,,-” denotes sample not taken. Values in italics denote the maximum axitinib concentration measured in the plasma in the respective treatment group over the course of the study. Specifically, axitinib concentrations in the hydrogel 9c treated group led to a maximum systemic concentration of axitinib within 24 h after said intratumoral injection that was less than 50% than the maximum systemic axitinib concentration of the Axitinib treated group within 24 h following systemic administration.

Total Tumor Drug Levels

The nominal amount of axitinib in the tumor samples directly after dosing was 123 μg. The mean determined amount of Axitinib in the tumor samples 72 h after dosing was 66.3±23.2 μg (N=4). Therefore, 72 h after dosing at least 25% of the injected amount was still present in the injected tumor tissue.

### Example 13

In Vivo Xenograft Anti-Tumor Efficacy and Tumor Vessel Density Assessment

The study was conducted in female Balb/c nude mice with an age of 6-8 weeks at the day of tumor inoculation. Mice were implanted with 5×10⁶ of MIA-PaCa-2 tumor cells into the right flank. When tumors were grown to a mean tumor volume of ˜91 mm³, mice were randomized into treatment cohorts (day 0). Following randomization, animals received either a single intratumoral injection of 1, 9a, or 9b as a single intratumoral dose in an injection volume of 50 μL. Hydrogels were administered as suspensions in pH 5.5 20 mM sodium succinate, 77 g/l trehalose dihydrate, 0.2% Pluronic F-68 buffer. After 3, 7, and 14 days following treatment, 3 mice were sacrificed from each treatment group and their tumors were harvested and were placed in 10% NBF and fixed for 24 hours at room temperature, then transferred to the Vacuum Tissue Processor (HistoCore PEARL, Leica) for dehydration, and embedded into FFPE blocks using a Tissue embedding center (EG1150, Leica). The blocks were sectioned at 4 um (RM2235, Leica). The resulting sections were used for IHC analysis. The IHC staining was performed on a Leica Bond RX autostainer. The sections were blocked with Peroxide Block (Leica) after pretreatment with EDTA solution (pH9.0) (Bond™ Epitope Retrieval Solution 2, Leica) at 100° C. for 20 minutes, then incubated with rabbit anti-human CD31 (1:200, Abcam, ab28364) at room temperature for 1 hour. This was followed by secondary antibody (Bond Polymer Refine Detection, Leica) for 20 minutes. Color was developed with DAB solution (Leica). Sections were counterstained with hematoxylin. All stained sections were scanned with NanoZoomer-HT 2.0 Image system, and the whole slide image was analysed with HALO™ (version 3.0.311.363) platform. Areas of necrosis were excluded. Vessel Density was analysed by the module Indica Labs-Object Colocalization v1.3.

Absolute Tumor Volumes (Mm³)

| | | Days post-treatment | | | | |
|---|---|---|---|---|---|---|
| Group | | 0 | 3 | 7 | 10 | 14 |
| 1 | Mean | 91.033 | 180.499 | 322.505 | 620.297 | 848.223 |
| | (mm³) | | | | | |
| | SEM | 4.673 | 14.290 | 48.233 | 94.484 | 183.341 |
| | (mm³) | | | | | |
| | n | 9 | 9 | 6 | 3 | 3 |
| 9a | Mean | 91.190 | 129.422* | 180.297 | 340.033 | 431.853 |
| | (mm³) | | | | | |
| | SEM | 5.422 | 8.686 | 38.714 | 163.662 | 241.120 |
| | (mm³) | | | | | |
| | n | 9 | 9 | 6 | 3 | 3 |
| 9b | Mean | 91.012 | 146.969 | 263.207 | 348.097 | 471.920 |
| | (mm³) | | | | | |
| | SEM | 4.825 | 6.189 | 27.199 | 40.648 | 74.720 |
| | (mm³) | | | | | |
| | n | 9 | 9 | 6 | 3 | 3 |

SEM = standard error of the mean,
n = sample size;
*p < 0.05 vs 1.
Significance was determined by One-way ANOVA followed by multiple comparisons using Tukey's post-hoc test. These data show 9a treated animals displayed significant anti-tumor activity compared to group 1 treated animals at 3 days post-treatment initiation.

Vessel Density (Count/Mm²)

| | | Days post-treatment | | |
|---|---|---|---|---|
| Group | | 3 | 7 | 14 |
| 1 | Mean (count/mm²) | 143.630 | 96.253 | 81.427 |
| | SEM (mm³) | 6.509 | 4.031 | 4.260 |
| | N | 3 | 3 | 3 |
| | Fold change of 1 | 1 | 1 | 1 |
| 9a | Mean (count/mm²) | 60.793* | 80.653 | 53.735* |
| | SEM (mm³) | 16.806 | 39.656 | 0.705 |
| | N | 3 | 3 | 2 |
| | Fold change of 1 | −2.36 | −1.19 | −1.52 |
| 9b | Mean (count/mm²) | 100.187 | 79.533 | 76.910 |
| | SEM (mm³) | 19.907 | 6.095 | 6.167 |
| | N | 3 | 3 | 3 |
| | Fold change of 1 | −1.43 | −1.21 | −1.06 |

SEM = standard error of the mean,
N = sample size;
*p < 0.05 vs 1.
Significance was determined by One-way ANOVA followed by multiple comparisons using Tukey's post-hoc test. These data show 9a treated animals displayed significant anti-angiogenic activity compared to group 1 treated animals during the course of the study.

### Example 14

24—Hour RNA-Seq Analysis of Gene Expression

The study was conducted in female C57BL/6 mice with an age of 6-8 weeks at the day of tumor inoculation. Mice were implanted with 1×10⁶ MC-38 tumor cells into the right flank. When tumors were grown to a mean tumor volume of ˜102 mm³, mice were randomized into treatment cohorts. Animals were treated by group as shown in the table below.

| | | Dosing | | | |
|---|---|---|---|---|---|
| | Dose | solution | Route of | Volume, | |
| Treatment | level | (mg/mL) | exposure | Dose | Frequency |
| 1 | — | — | IT | 50 μL | Once on Day 0 |
| 9a | 369 μg | 369 μg | IT | 50 μL | Once on Day 0 |
| | eq. | | | | |
| | axitinib | | | | |

Tumors were harvested 24-hours post-dosing (N=3 for each group). Total RNA from tumor tissue was purified using RNeasy Mini Kit (QIAGEN, Cat. 74106, CA) according to the manufacturer's instructions. The integrity of the total RNA was determined by 2100 Bioanalyser (Agilent) and quantified using the NanoDrop (Thermo Scientific). One aliquot of high-quality RNA sample (OD260/280=1.8-2.2, OD260/230>2.0, RIN>8.0, >1 μg) was used to for RNAseq assay. PolyA mRNA was purified from total RNA using oligo-dT-attached magnetic beads and then fragmented by fragmentation buffer. Taking these short fragments as templates, first stranded cDNA was synthesized using reverse transcriptase and random primers, followed by second stranded cDNA synthesis. Then the synthesized cDNA was subjected to end-repair, phosphorylation and ‘A’ base addition according to library construction protocol. Sequencing adapters were added to both sides of the cDNA fragments. After PCR amplification for cDNA fragments, the targets of 250-350 bp were cleaned up. After library construction, Qubit 3.0 fluorometer dsDNA HS Assay (Thermo Fisher Scientific) was used to quantify concentration of the resulting sequencing libraries, while the size distribution was analyzed using Agilent BioAnalyzer 2100 (Agilent). After library validation, Illumina cBOT cluster generation system with HiSeq PE Cluster Kits (Illumina) was used to generate clusters. Paired-end sequencing was performed using an Illumina system following Illumina-provided protocols for 2×150 paired-end sequencing. The quality of RNAseq raw data was checked by FastQC software. The adapter and low quality sequences were trimmed by Trimmomatic software. The clean data after trimming were used for analysis. The reads were mapped to reference genes (ENSEMBL GRCh37.66) by Bowtie software, and the gene expression was calculated by MMSEQ software. The expression values are log_2(FPKM). Log_2 fold changes were converted to standard fold change. Genes exhibiting changes of more than or equal to 1.5 fold are listed in the tables below.

| | |
|---|---|
| Upregulated Genes | Downregulated Genes |
| 24-Hours Post-Dose | 24-Hours Post-Dose |

| | Fold | | Fold |
|---|---|---|---|
| Gene | Change | Gene | Change |
| Angpt2 | 1.79 | Angptl3 | −1.75 |
| Apold1 | 1.56 | Bmp10 | −5.43 |
| Dll4 | 2.02 | Cga | −2.01 |
| Hey2 | 2.38 | Chga | −1.74 |
| Ifnb1 | 1.50 | Csf3 | −3.00 |
| Igfbp3 | 1.98 | Cxcl5 | −1.70 |
| Il12a | 2.25 | Dkk1 | −1.94 |
| Kcnj2 | 1.76 | F2 | −2.79 |
| Kdr | 1.55 | Fgf6 | −1.94 |
| Lep | 1.55 | Hand2 | −1.94 |
| Mycn | 2.88 | Il1a | −1.93 |
| Notch4 | 1.60 | Il1b | −1.65 |
| Stc1 | 1.69 | Il6 | −1.88 |
| Tgfa | 1.65 | Myocd | −2.25 |
| Timp4 | 2.91 | Plg | −1.94 |
| | | Ptgs2 | −1.54 |
| | | Rcan2 | −1.54 |
| | | Sele | −1.76 |
| | | Tbx4 | −1.66 |
| | | Tdgf1 | −4.99 |
| | | Thbs1 | −1.61 |
| | | Tmprss6 | −1.63 |
| | | Wt1 | −2.62 |

These data show 9a treated animals displayed differentially expressed genes associated with angiogenesis compared to animals treated with 1 during the course of the study.

### Example 15 Synthesis of Compound 10a

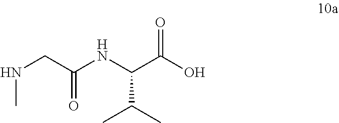

Boc-Sar-OH (99 mg, 0.52 mmol) was dissolved in DCM (1 mL). L-Valine tert-butyl ester hydrochloride (111 mg, 0.53 mmol), EDC HCl (109 mg, 0.57 mmol) and DIPEA (276 μL, 1.59 mmol) were added with stirring. After 3 h the reaction was diluted with 30 mL of DCM and was washed 3 times with 30 mL of 0.1 N HCl, 2 times with sat. NaHCO₃ and once with brine. The organic phase was dried over Na₂SO₄, filtered and evaporated. The product was purified by RP-HPLC. The product was dissolved in 0.5 ml of DCM. 0.5 ml of TFA were added with stirring in an open flask. After 5 h the reaction was concentrated in a stream of nitrogen and the product co-evaporated 3 times with DCM.

Yield: 44 mg (28%, TFA salt)

MS: m/z 188.88=[M+H]⁺, (calculated=189.13).

### Example 16: Synthesis of Compound 10b

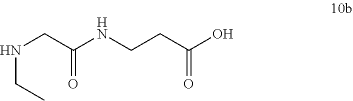

N-Boc-N-ethylglycine (100 mg, 0.49 mmol) and HOBt (66 mg, 0.49 mmol) were suspended in DCM (1 mL). H-beta-Ala-OtBu hydrochloride (107 mg, 0.59 mmol) was added and a solution was obtained. EDC HCl (99 mg, 0.52 mmol) was added and the reaction was stirred for 1.5 h. The volatiles were removed in vacuo and the product purified by RP-HPLC. The product was dissolved in 0.5 mL of DCM. 0.5 mL of TFA were added with stirring in an open flask. After 30 min the reaction was concentrated in vacuo and the product co-evaporated 2 times with DCM. The residue was dissolved in acetonitrile/water 1:1 (2 mL) and lyophilized.

Yield: 125 mg (88%, TFA salt)

MS: m/z 174.98=[M+H]⁺, (calculated=175.11).

### Example 17: Synthesis of Compound 10c

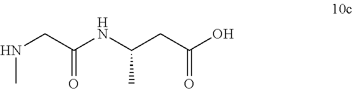

Boc-Sar-OH (103 mg, 0.54 mmol) was dissolved in DCM (1 mL). tert-Butyl-(3S)-3-aminobutanoate (84 mg, 0.53 mmol), EDC HCl (113 mg, 0.59 mmol) and DIPEA (0.28 mL, 1.58 mmol) were added. After 3 h the reaction mixture was diluted with 30 mL of DCM and was washed 3 times with 30 mL of 0.1 N HCl, 2 times with sat. NaHCO₃ and once with brine. The organic phase was dried over Na₂SO₄, filtered and evaporated. The product was purified by RP-HPLC. The product was dissolved in 0.5 mL of DCM. 0.5 ml of TFA were added with stirring in an open flask. After 3 h the reaction was concentrated in vacuo and the product co-evaporated 3 times with DCM (5 mL).

Yield: 73 mg (47%)

MS: m/z 437.21=[M+H]⁺, (calculated=437.19).

### Example 18: Synthesis of Compound 11a

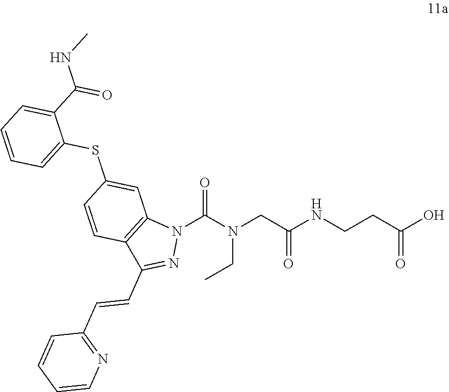 [SMILES: CCN(CC(=O)NCCC(=O)O)C(=O)n1nc(/C=C/c2ccccn2)c2ccc(Sc3ccccc3C(=O)NC)cc21]

10b (29 mg, 82 μmol) was dissolved in 100 μL of DMF and DIPEA (48 μL, 0.27 mmol) was added. A suspension of 3 (40 mg, 68 μmol) (0.79 mL in DMF) was added. After 3.5 h 10b (14 mg, 41 μmol) in 50 μL of DMF was added. After 4.75 h TFA (21 μL) were added and the reaction purified by RP-HPLC.

Yield: 22 mg (45%, TFA salt)

MS: m/z 350.06=[M+H]⁺, (calculated=350.10).

### Example 19: Synthesis of Compound 11b

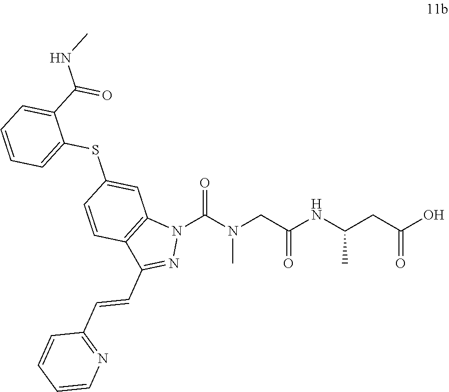 [SMILES: CNC(=O)c1ccccc1Sc1ccc2c(/C=C/c3ccccn3)nn(C(=O)N(C)CC(=O)N[C@@H](C)CC(=O)O)c2c1]

10c (28 mg, 88 μmol) was dissolved in 100 μL of DMF and DIPEA (38 μL, 0.22 mmol) was added. A suspension of 2 (26 mg, 44 μmol) (508 μL in DMF) was added. After 30 min TFA (6.7 μL) was added and the product purified by RP-HPLC.

Yield: 31 mg (quant, TFA salt)

MS: m/z 587.16=[M+H]⁺, (calculated=587.21).

### Example 20: Synthesis of Compound 11c

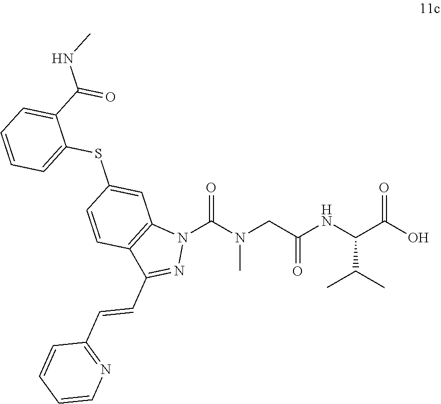 [SMILES: CNC(=O)c1ccccc1Sc1ccc2c(/C=C/c3ccccn3)nn(C(=O)N(C)CC(=O)N[C@H](C(=O)O)C(C)C)c2c1]

10a (22 mg, 68 μmol) was dissolved in 100 μL of DMF and DIPEA (30 μL, 0.17 mmol) was added. A suspension of 2 (20 mg, 34 μmol) (393 μL in DMF) was added. After 1 h TFA (5.2 μl) was added and the product purified by RP-HPLC.

Yield: 26 mg (quant, TFA salt)

MS: m/z 601.10=[M+H]⁺, (calculated=601.23).

### Example 21: Synthesis of Compounds 12a-d

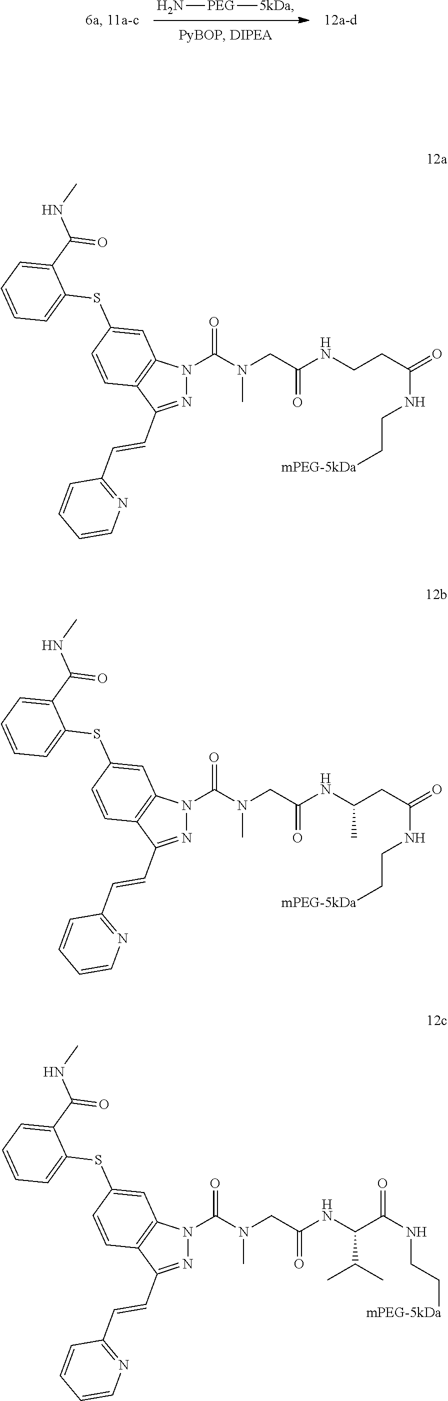

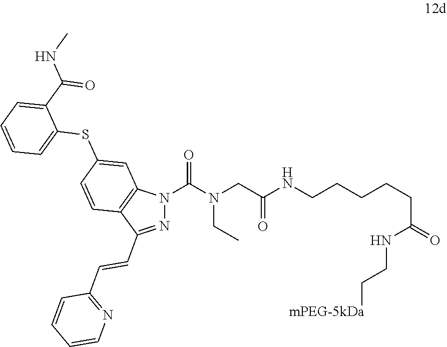

Methoxy polyethylene glycol amine-5 kDa PEG, PyBOP, DIPEA and an acid selected from 6a or 11a-c were stirred at RT. After the reaction was finished, acetic acid was added, and the product purified by RP-HPLC.

12a: PEG: 33 mg, 6.0 μmol, PyBOP: 3.6 mg, 6.9 μmol, DIPEA: 3.1 μl, 18 μmol, 11a: 4.2 mg, 6 μmol, yield: 21 mg (58%, TFA salt).

12b: PEG: 21 mg, 3.8 μmol, PyBOP: 3.5 mg, 6.7 μmol, DIPEA: 1.9 μl, 11 μmol, 11b: 2.6 mg, 3.7 μmol, yield: 18 mg (77%, TFA salt).

12c: PEG: 47 mg, 8.5 μmol, PyBOP: 4.9 mg, 9.4 μmol, DIPEA: 4.4 μl, 25 μmol, 11c: 6 mg, 8.4 μmol, yield: 31 mg (60%, TFA salt).

12d: PEG: 31 mg, 5.6 μmol, PyBOP: 3.5 mg, 6.8 μmol, DIPEA: 2.8 μL, 16 μmol, 6a: 4 mg, 5.4 μmol, yield: 34 mg (quant, TFA salt).

### Example 22: Synthesis of Compound 13a

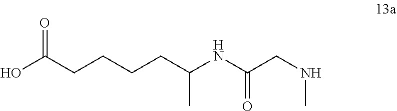

Methyl 6-oxo-heptanoate (2 g, 12.6 mmol) was dissolved in methanol (13 mL) and ammonium acetate (9.75 g, 126 mmol), and sodium cyanoborohydride (1.19 g, 19.0 mmol) was added with stirring. The resulting suspension turned into a solution and stirring was continued overnight. The mixture was diluted with water (70 mL) and ethyl acetate was added (80 mL). The pH of the water phase was adjusted to circa pH 11 with 25 mL 4 M NaOH. The aqueous phase was extracted with ethyl acetate (three times 70 mL). The combined organic phases were dried (MgSO₄), filtered and concentrated in vacuo to give a yellow oil (1.83 g). A portion of the crude oil (200 mg) from the first step was dissolved in DMF (2 mL) and N-Boc-Sar-OH (238 mg, 1.26 mmol), PyBOP (719 mg, 1.38 mmol) and DIPEA (656 μL, 3.77 mmol) were added with stirring. The reaction was stirred at RT for 2 h. The mixture was diluted with 25 mL ethyl acetate and washed with 0.1 N HCl (3 times 15 mL), 0.5 M NaOH (3 times 15 mL) and brine (15 mL). The organic phase was dried (MgSO₄), filtered and concentrated in vacuo. The residue was purified using flash chromatography (heptane/ethyl acetate). The product (235 mg) was dissolved in THF (1 mL), and LiOH (51 mg, 2.13 mmol) was dissolved in water (0.4 mL). The solutions were combined and stirred vigorously at RT. After 5 h the mixture was diluted with 80 mL ethyl acetate, and 60 mL 1 N HCl was added. The pH of the aqueous phase was below 2. The organic phase was collected, and the aqueous phase extracted with ethyl acetate (three times 20 mL). The combined organics were dried (MgSO₄), filtered, and concentrated in vacuo. The residue was dissolved in DCM (1.0 mL) and TFA (0.5 mL) was added with vigorous stirring in an open flask. After 75 min the reaction was concentrated in vacuo and co-evaporated once with 5 mL DCM. The crude was dissolved in 1:2 acetonitrile/H₂O+0.1% TFA (20 mL) and lyophilized.

Yield: 213 mg (47%, TFA salt)

MS: m/z 217.05=[M+H]⁺, (calculated=217.15).

### Example 23: Synthesis of Compound 13b

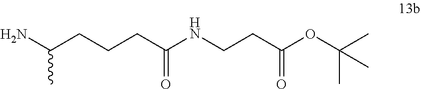 [SMILES: CC(N)CCCC(=O)NCCC(=O)OC(C)(C)C]

Methyl 5-oxohexanoate (2.00 g, 13.9 mmol) was dissolved in THF (60 mL) and LiOH (1.00 g, 41.6 mmol) and water (20 mL) were added. The mixture was stirred at RT for 5 h before dilution with ethyl acetate (300 mL). 1 N aq.HCl (80 mL) was added, and the aqueous phase extracted with ethyl acetate (2 times 100 mL). The combined organics were dried (MgSO₄) and concentrated in vacuo. The resulting colorless oil (1.6 g) was dissolved in DMF (32 mL), and PyBOP (7.68 g, 14.8 mmol) then DIPEA (10.7 mL, 61.5 mmol) were added to the mixture. After stirring for 5 min tert-butyl 3-aminopropanoate hydrochloride (2.69 g, 14.8 mmol) was added and the mixture stirred at RT for 105 min. The mixture was diluted with ethyl acetate (400 mL) and washed with 0.55 M aq. HCl (100 mL), 0.1 M aq. HCl (2 times 100 mL), sat. NaHCO₃ (3 times 100 mL), and brine (100 mL). The organics were dried (MgSO₄) and concentrated in vacuo before being purified by flash chromatography (ethyl acetate/heptane). The purified material was then dissolved in MeOH (14.2 mL) and ammonium acetate (6.60 g, 85.6 mmol) and sodium cyanoborohydride (801 mg; 12.8 mmol) were added. The mixture was stirred overnight at RT. The mixture was diluted with water (70 ml) and ethyl acetate (80 ml). Using 4 M NaOH (15 mL) the pH of the aq. phase was adjusted to ca. pH 2. The aq. phase was extracted with ethyl acetate (3 times 70 mL), the organics combined and TFA (648 μL) added. To the aq. phase was added further 4 M NaOH (5 mL) and again it was extracted with ethyl acetate (3 times 70 mL), these organics were combined and TFA (400 μL) added. The organics were dried (MgSO₄), filtered, and the volatiles removed in vacuo.

Yield: 3.40 g (66%, TFA salt)

MS: m/z 259.12=[M+H]⁺, (calculated=259.20).

### Example 24: Synthesis of Compound 13c

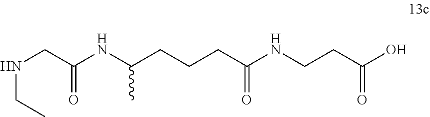 [SMILES: CCNCC(=O)NC(C)CCCC(=O)NCCC(=O)O]

Compound 13b (249 mg, 0.62 mmol) was dissolved in DMF (2.30 mL) and N-Boc-N-ethylglycine (132 mg, 0.65 mmol) and PyBOP (353 mg; 0.68 mmol) were added followed by DIPEA (326 μL, 1.87 mmol) to form a light yellow solution. After stirring at RT for 90 min, the mixture was diluted with ethyl acetate (50 mL) and washed with 0.1 M HCl (3 times 25 mL), sat. aq. NaHCO₃ (25 mL), a 3:5 v/v mixture of brine and sat. aq. NaHCO₃ (2 times 40 mL), and brine (30 mL). The organics were dried (MgSO₄) and the volatiles removed in vacuo. The intermediate was purified by flash chromatography (methanol/DCM) and then purified by RP-HPLC to give a colourless oil. The oil was dissolved in DCM (0.5 mL) and TFA (0.5 mL) was added. After stirring at RT for 55 min, the volatiles were removed under a stream of nitrogen. The residues were diluted with acetonitrile/H₂O 1:1+0.1% TFA (2 mL)+0.1% TFA and water (4 mL). The mixture was lyophilized to give a colourless oil.

Yield: 136 mg (52%, TFA salt)

MS: m/z 288.19=[M+H]⁺, (calculated=288.19).

### Example 25: Synthesis of Compound 13d

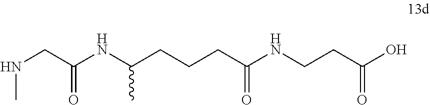 [SMILES: CNCC(=O)NC(C)CCCC(=O)NCCC(=O)O]

Compound 13b (251 mg, 0.63 mmol) was dissolved in DMF (2.30 mL) and N-Boc-Sar-OH (121 mg, 0.64 mmol) and PyBOP (358 mg; 0.69 mmol) were added followed by DIPEA (326 μL, 1.87 mmol) to form a light-yellow solution. After stirring at RT for 90 min, the mixture was diluted with ethyl acetate (50 mL) and washed with 0.1 M HCl (3 times 25 mL), sat. aq. NaHCO₃ (25 mL), a 3:5 v/v mixture of brine and sat. aq. NaHCO₃ (2 times 40 mL), and brine (30 mL). The organics were dried over MgSO₄ and the volatiles removed in vacuo. The intermediate was purified by flash chromatography (methanol/DCM) to give a colourless oil. The oil was dissolved in DCM (0.5 mL) and the solution treated with TFA (0.5 mL). After stirring at RT for 55 min, the volatiles were removed under a stream of nitrogen. The residues were diluted with 1:1 acetonitrile/H₂O+0.1% TFA (2 mL)+0.1% TFA and water (4 mL). The mixture lyophilized to give a colourless oil.

Yield: 129 mg (51%, TFA salt)

MS: m/z 274.18=[M+H]⁺, (calculated=274.17).

### Example 26: Synthesis of Compound 13e

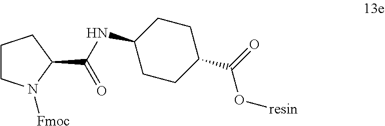

13e was synthesized using solid-phase synthesis following the general protocol using Fmoc-trans-1,4-ACHC-OH and Fmoc-Pro-OH as building blocks.

### Example 27: Synthesis of Compound 13f

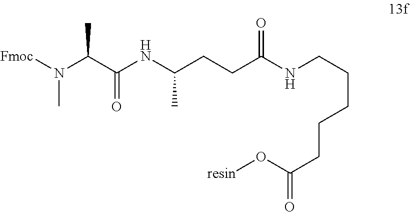

13f was synthesized using solid-phase synthesis following the general protocol using Fmoc-Ahx-OH, (S)-Fmoc-4-aminopentanoic acid, and Fmoc-N-Methyl-Ala-OH as building blocks.

### Example 28 Synthesis of Compound 13g

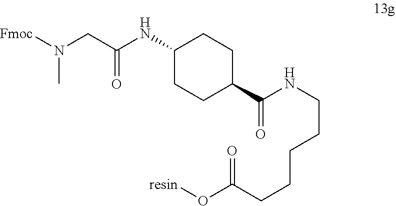

13g was synthesized using solid-phase synthesis following the general protocol using Fmoc-Ahx-OH, Fmoc-trans-1,4-ACHC-OH, and Fmoc-Sar-OH as building blocks.

### Example 29 Synthesis of Compound 13h

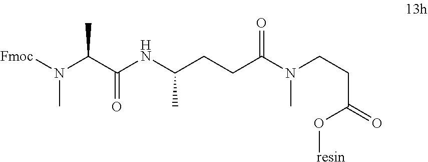

13h was synthesized using solid-phase synthesis following the general protocol using Fmoc-N-Methyl-P-Ala-OH (S)-Fmoc-4-aminopentanoic acid, and Fmoc-N-Methyl-Ala-OH as building blocks.

### Example 30 Synthesis of Compound 13i

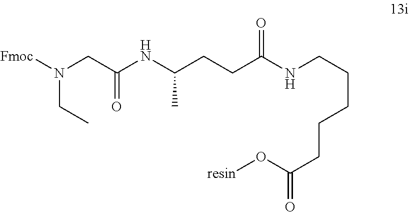

13i was synthesized using solid-phase synthesis following the general protocol using Fmoc-Ahx-OH, (S)-Fmoc-4-aminopentanoic acid, and Fmoc-N-Ethyl-Gly-OH as building blocks.

### Example 31: Synthesis of 14a

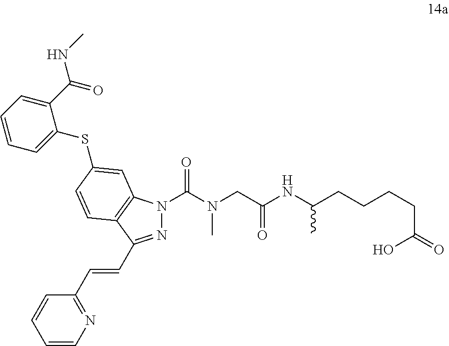 [SMILES: CNC(=O)c1ccccc1Sc1ccc2c(/C=C/c3ccccn3)nn(C(=O)N(C)CC(=O)NC(C)CCCCC(=O)O)c2c1]

13a (213 mg, 0.64 mmol) was dissolved in 500 μL of DMF and DIPEA (247 μL, 1.42 mmol) was added. A suspension of 2 (162 mg, 0.28 mmol, in 2.0 mL DMF) was added. After 2 h TFA (110 μl, 1.44 mmol) was added and the product purified by RP-HPLC.

Yield: 111 mg (54%, TFA salt)

MS: m/z 629.20=[M+H]⁺, (calculated=629.25).

### Example 32: Synthesis of 14b

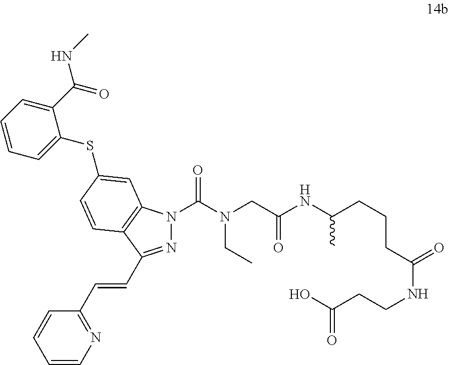 [SMILES: CCN(CC(=O)NC(C)CCCC(=O)NCCC(=O)O)C(=O)n1nc(/C=C/c2ccccn2)c2ccc(Sc3ccccc3C(=O)NC)cc21]

Compound 13c (136 mg, 0.338 mmol) was dissolved in DMF (0.25 mL) and DIPEA (147 μL, 0.845 mmol) was added. To the stirred colourless solution was added 2 (100 mg, 0.169 mmol) in DMF (1.23 mL) and the mixture immediately turned clear yellow. The mixture was stirred at RT for 105 min then TFA (65 μL, 0.845 mmol) was added. The product was purified by RP-HPLC to give a yellow solid.

Yield: 100 mg (72%, TFA salt)

MS: m/z 700.24=[M+H]⁺, (calculated=700.29).

### Example 33: Synthesis of 14c

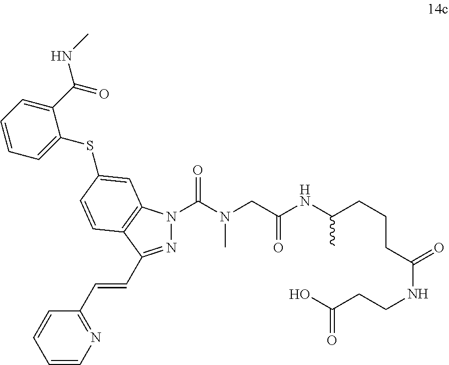 [SMILES: CNC(=O)c1ccccc1Sc1ccc2c(/C=C/c3ccccn3)nn(C(=O)N(C)CC(=O)NC(C)CCCC(=O)NCCC(=O)O)c2c1]

Compound 13d (129 mg, 0.333 mmol) was dissolved in DMF (0.25 mL) and DIPEA (145 μL, 0.83 mmol) was added. To the stirred colourless solution was added 2 (98 mg, 0.169 mmol) in DMF (1.21 mL) and the mixture immediately turned clear yellow. The mixture was stirred at RT for 105 min then TFA (65 μL, 0.845 mmol) was added. The product was purified by RP-HPLC to give a yellow solid.

Yield: 101 mg (75%, TFA salt)

MS: m/z 686.19=[M+H]⁺, (calculated=686.27).

### Example 34: Synthesis of 14d-h

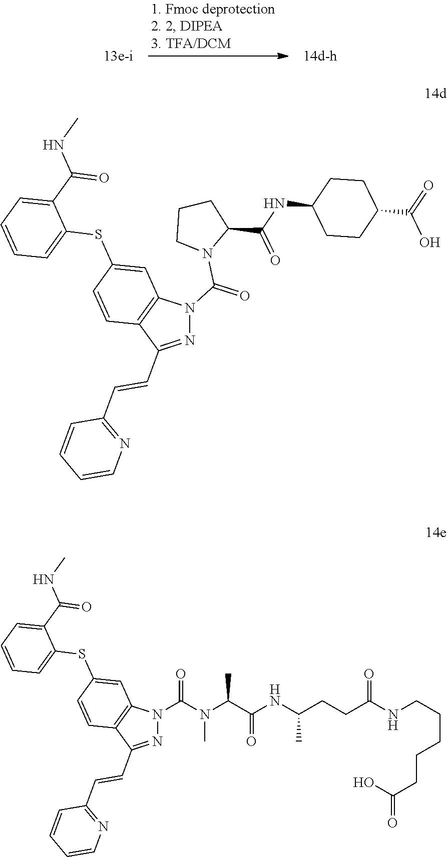 [SMILES: CNC(=O)c1ccccc1Sc1ccc2c(/C=C/c3ccccn3)nn(C(=O)N3CCC[C@H]3C(=O)N[C@H]3CC[C@H](C(=O)O)CC3)c2c1 ; CNC(=O)c1ccccc1Sc1ccc2c(/C=C/c3ccccn3)nn(C(=O)N(C)[C@@H](C)C(=O)N[C@@H](C)CCC(=O)NCCCCCC(=O)O)c2c1]

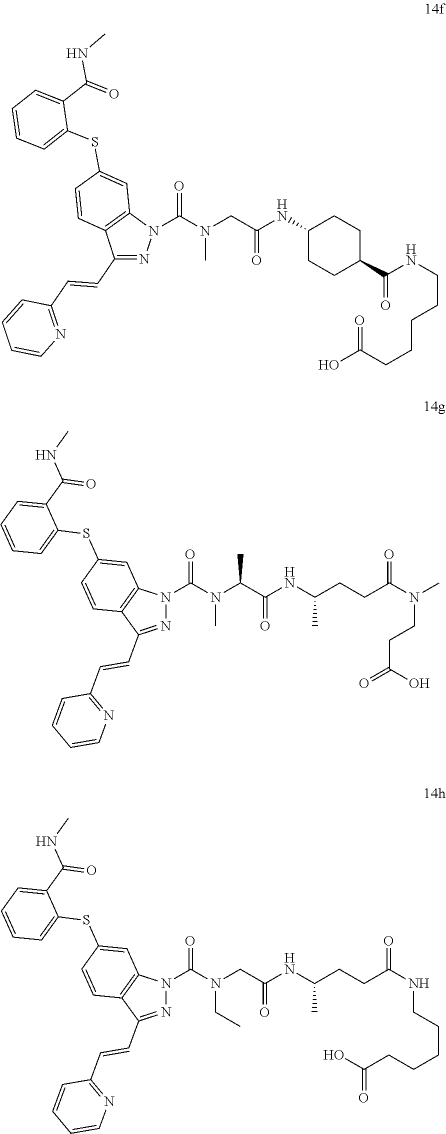 [SMILES: CNC(=O)c1ccccc1Sc1ccc2c(/C=C/c3ccccn3)nn(C(=O)N(C)CC(=O)N[C@H]3CC[C@H](C(=O)NCCCCCC(=O)O)CC3)c2c1 ; CNC(=O)c1ccccc1Sc1ccc2c(/C=C/c3ccccn3)nn(C(=O)N(C)[C@@H](C)C(=O)N[C@@H](C)CCC(=O)N(C)CCC(=O)O)c2c1 ; CCN(CC(=O)N[C@@H](C)CCC(=O)NCCCCCC(=O)O)C(=O)n1nc(/C=C/c2ccccn2)c2ccc(Sc3ccccc3C(=O)NC)cc21]

The conjugates 14d-h were prepared from their respective resin-loaded Fmoc-protected amines 13e-i, which were treated with 96:2:2 DMF/piperidine/DBU (5 ml) and shaken for 15 min at RT. The filtrate was drained and the procedure repeated twice before washing of the resin with DMF (5 times). The resin was then treated with a suspension of 2 in DMF and DIPEA. The mixture was shaken at RT for between 90 and 200 min before being washed with DMF (5 times) and DCM (5 times). The resin was treated with 1:9TFA/DCM and shaken at RT for 10 min. The filtrate was collected and this was repeated at least once. The volatiles were removed from the combined filtrates in vacuo to give the acid.

14d: 13e: 493 mg, 0.453 mmol, 2: 266 mg, 0.453 mmol, DMF: 3.5 mL, DIPEA: 485 μL, 2.72 mmol.

Yield: 331 mg (95%, TFA salt). MS: m/z=653.29 [M+H]⁺, (calculated=653.25).

14e: 13f: 195 mg, 0.16 mmol, 2: 119 mg, 0.20 mmol, DMF: 1.5 mL, DIPEA: 173 μL, 0.97 mmol.

Yield: 136 mg (quant., TFA salt). MS: m/z=728.36 [M+H], (calculated=728.32).

14f: 13g: 312 mg, 0.26 mmol, 2: 188 mg, 0.32 mmol, DMF: 2.3 mL, DIPEA: 275 μL, 1.54 mmol.

Yield: 274 mg (quant., TFA salt). MS: m/z=740.34 [M+H]⁺, (calculated=740.32).

14g: 13h: 170 mg, 0.15 mmol, 2: 108 mg, 0.18 mmol, DMF: 1.3 mL DIPEA: 158 μL, 0.88 mmol.

Yield: 124 mg (quant., TFA salt). MS: m/z=700.32 [M+H]⁺, (calculated=700.29).

14h: 13i: 201 mg, 0.17 mmol 2: 123 mg, 0.21 mmol, DMF: 1.5 mL DIPEA: 0.18 mL, 1.00 mmol.

Yield: 155 mg (quant., TFA salt). MS: m/z=728.34 [M+H]⁺, (calculated=728.32).

### Example 35: Synthesis of Compounds 15a-f

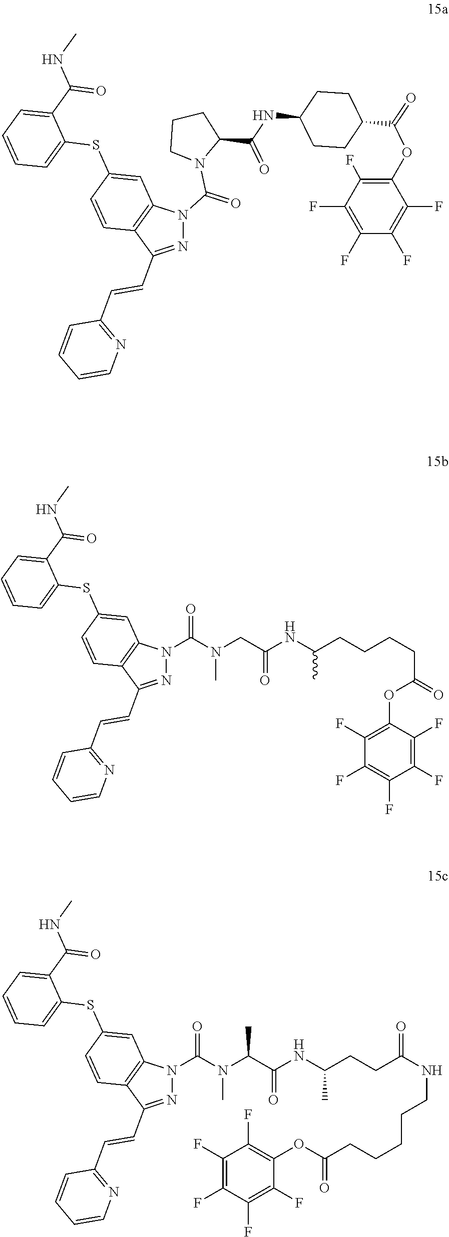 [SMILES: CNC(=O)c1ccccc1Sc1ccc2c(/C=C/c3ccccn3)nn(C(=O)N3CCC[C@H]3C(=O)N[C@H]3CC[C@H](C(=O)Oc4c(F)c(F)c(F)c(F)c4F)CC3)c2c1 ; CNC(=O)c1ccccc1Sc1ccc2c(/C=C/c3ccccn3)nn(C(=O)N(C)CC(=O)NC(C)CCCCC(=O)Oc3c(F)c(F)c(F)c(F)c3F)c2c1 ; CNC(=O)c1ccccc1Sc1ccc2c(/C=C/c3ccccn3)nn(C(=O)N(C)[C@@H](C)C(=O)N[C@@H](C)CCC(=O)NCCCCCC(=O)Oc3c(F)c(F)c(F)c(F)c3F)c2c1]

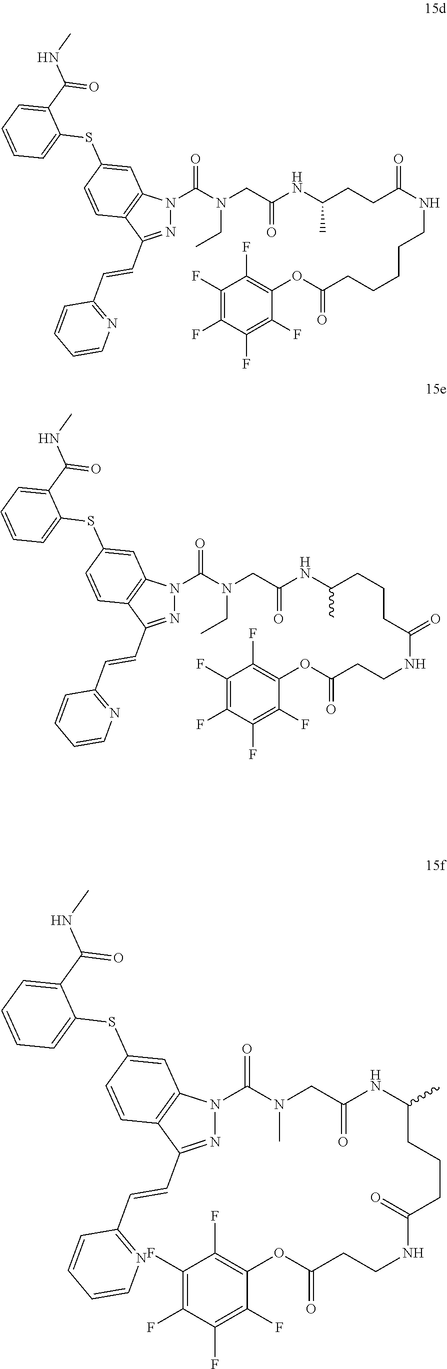

The respective acid selected from 14a-e, h was dissolved in DCM and bis(pentafluorophenyl) carbonate was added. DIPEA was added and the reaction stirred at RT. Once the reaction was complete it was quenched with TFA and the product purified by flash chromatography (THF/ethyl acetate).

15a: DCM: 4.0 mL, Bis(pentafluorophenyl) carbonate: 213 mg, 0.54 mmol, DIPEA: 377 μL, 2.16 mmol, 14d: 331 mg, 0.43 mmol, TFA: 165 μL, 2.16 mmol.

Yield: 273 mg (68%, TFA salt). MS: m/z 819.34=[M+H]⁺, (calculated=819.23).

15b: DCM: 2.0 mL, Bis(pentafluorophenyl) carbonate: 70 mg, 0.178 mmol, DIPEA: 130 μL, 0.746 mmol, 14a: 111 mg, 0.149 mmol, TFA: 57 μL, 0.746 mmol.

Yield: 122 mg (90%, TFA salt). MS: m/z 795.25=[M+H]⁺, (calculated=795.23).

15c: DCM: 1.50 mL, Bis(pentafluorophenyl) carbonate: 91 mg, 0.23 mmol, DIPEA: 162 μL, 0.93 mmol, 14e: 156 mg, 0.19 mmol, TFA: 71 μL, 0.93 mmol.

Yield: 94 mg (50%, TFA salt). MS: m/z 894.30=[M+H]⁺, (calculated=894.30).

15d: DCM: 1.50 mL, Bis(pentafluorophenyl) carbonate: 91 mg, 0.23 mmol, DIPEA: 161 μL, 0.92 mmol, 14h: 155 mg, 0.18 mmol, TFA: 71 μL, 0.92 mmol.

Yield: 105 mg (56%, TFA salt). MS: m/z 894.31=[M+H]⁺, (calculated=894.30).

15e: DCM: 2.00 mL, Bis(pentafluorophenyl) carbonate: 58 mg, 0.147 mmol, DIPEA: 107 μL, 0.61 mmol, 14b: 100 mg, 0.122 mmol, TFA: 47 μL, 0.61 mmol.

Yield: 77 mg (64%. TFA salt). MS: m/z=866.26 [M+H], (calculated=866.27).

15f: DCM: 2.00 mL, Bis(pentafluorophenyl) carbonate: 59 mg, 0.151 mmol, DIPEA: 110 μL, 0.63 mmol, 14c: 101 mg, 0.126 mmol, TFA: 48 μL, 0.63 mmol.

Yield: 96 mg (79%, TFA salt). MS: m/z=852.21 [M+H]⁺, (calculated=852.26).

### Example 36: Synthesis of Compound 15g

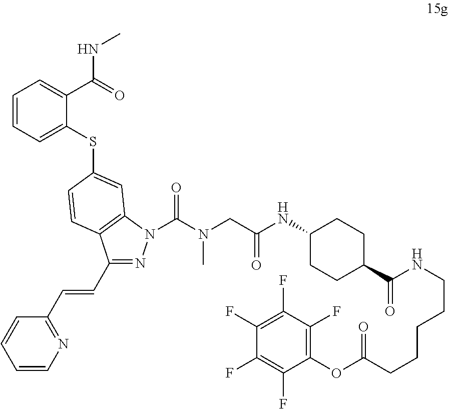 [SMILES: CNC(=O)c1ccccc1Sc1ccc2c(/C=C/c3ccccn3)nn(C(=O)N(C)CC(=O)N[C@H]3CC[C@H](C(=O)NCCCCCC(=O)Oc4c(F)c(F)c(F)c(F)c4F)CC3)c2c1]

To a solution of 14f (274 mg, 0.32 mmol) in DCM (2.5 mL) was added bis(pentafluorophenyl) carbonate (158 mg, 0.40 mmol) followed by DIPEA (280 μL, 1.60 mmol). Further DCM (2.5 mL) and DIPEA (280 μL, 1.60 mmol) were added to the suspension. acetonitrile (1 mL) and DMF (2 mL) were added. The suspension was stirred at RT for 1 d. The mixture was filtered, and the precipitate washed with DCM. The combined filtrates were washed with water, dried (Na₂SO₄), filtered and concentrated in vacuo. The concentrate was diluted with DCM before addition of TFA (245 μL, 3.18 mmol) and the product purified by flash chromatography (THF/ethyl acetate).

Yield: 66 mg (20% TFA salt)

MS: m/z 906.41=[M+H]⁺, (calculated=906.30).

### Example 37: Synthesis of Compound 15h

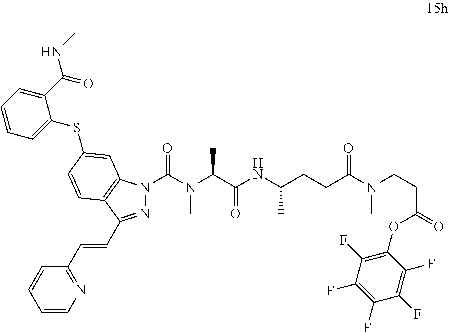 [SMILES: CNC(=O)c1ccccc1Sc1ccc2c(/C=C/c3ccccn3)nn(C(=O)N(C)[C@@H](C)C(=O)N[C@@H](C)CCC(=O)N(C)CCC(=O)Oc3c(F)c(F)c(F)c(F)c3F)c2c1]

To a solution of 14g (124 mg, 0.15 mmol) in DCM (1.5 mL) was added Bis(pentafluorophenyl) carbonate (75 mg, 0.19 mmol) followed by DIPEA (133 μL, 0.76 mmol). After stirring at RT for 3 h further Bis(pentafluorophenyl) carbonate (19 mg, 0.05 mmol) was added, and after a further 1 h DIPEA (65 μL, 0.37 mmol) was added. The mixture was left to stir at RT for another 18 h. The product was purified directly by flash chromatography (THF/ethyl acetate).

Yield: 24 mg (16%, TFA salt)

MS: m/z 866.30=[M+H]⁺, (calculated=866.27).

### Example 38: Synthesis of Compounds 16a-q

Various hydrogels (amine content of 0.564-0.934 mmol/g) were reacted with Axitinib-linker-conjugates according to the following scheme:

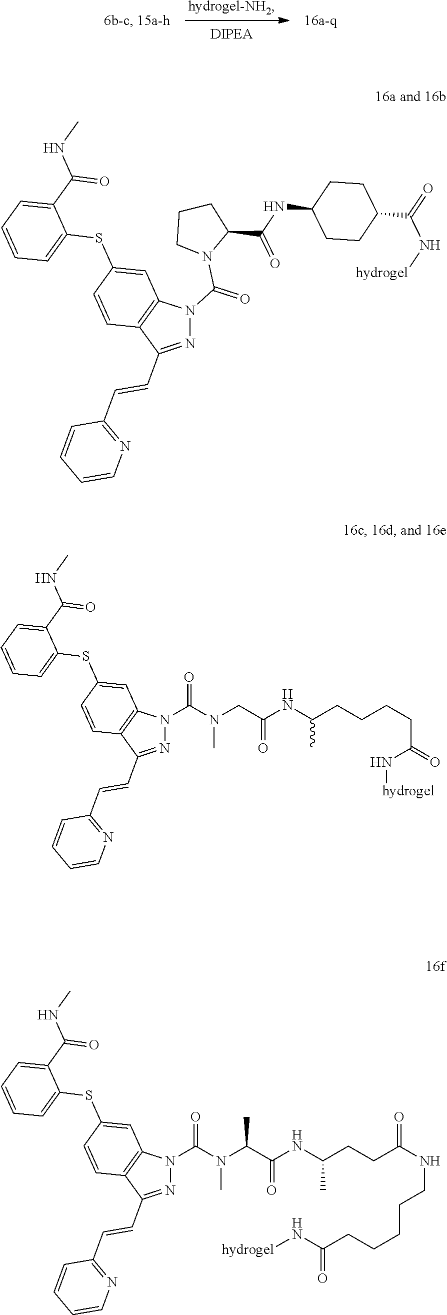

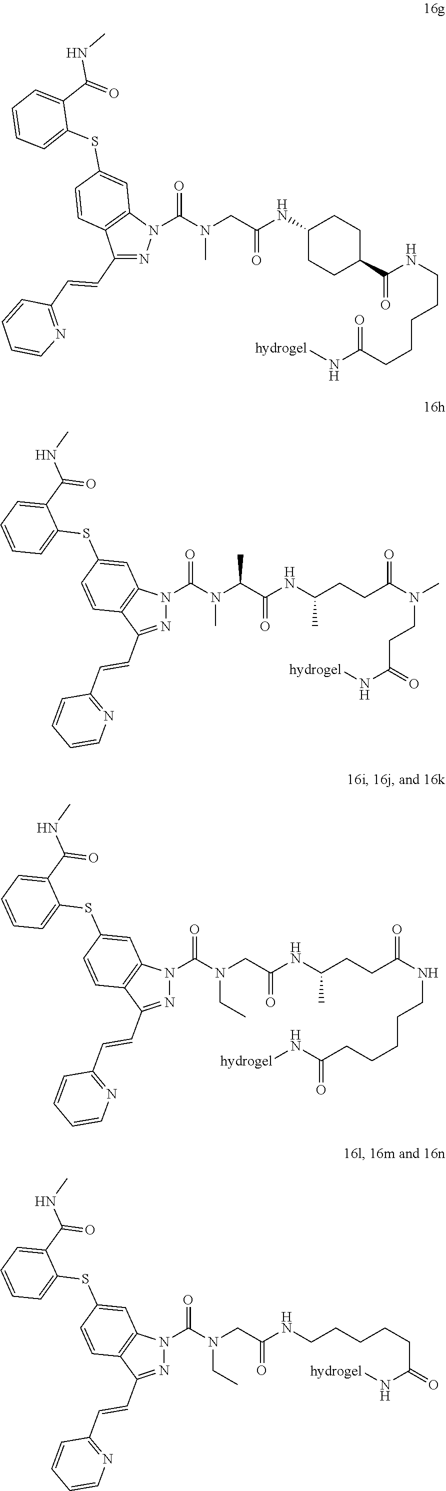

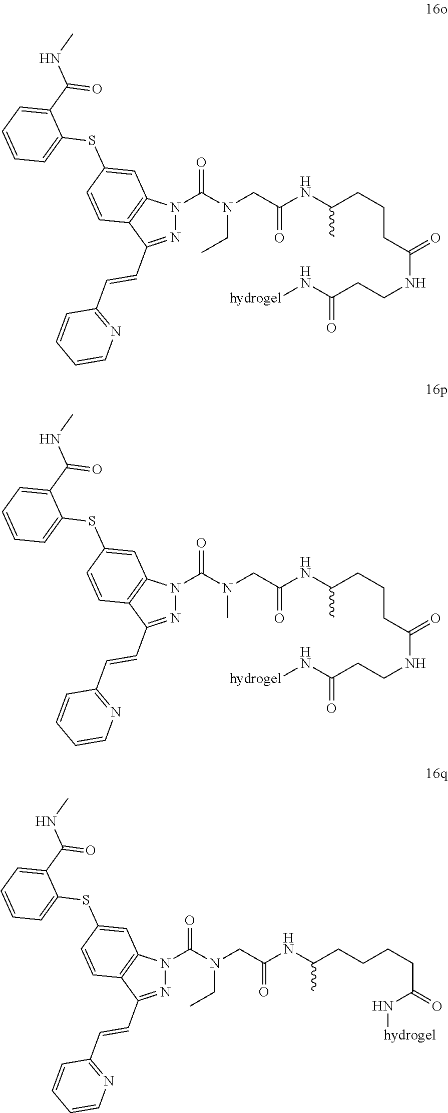

The hydrogel was swollen in 1% DIPEA in DMF in a syringe reactor containing a PE frit. The syringe reactor was 3 times filled, shaken for 1 min and drained. A PFP-ester selected from 6b-c or 15a-h was dissolved in DMF and DIPEA was added. The solution was drawn into the syringe containing the hydrogel. The syringe was shaken for longer than 16 h at RT. The syringe was drained, and the hydrogel was washed several times with DMF, then water, then pH 5.5 20 mM sodium succinate aqueous buffer. A hydrogel suspension in pH 5.5 aqueous buffer was obtained. The proportion of amines from the hydrogel that were conjugated was determined by comparing the determined drug content of the product with the amine content of the starting amine hydrogels.

16a: HG-6: 21 mg, DIPEA: 12.1 μL, 15a: 29 mg yield: suspension, 94% Axitinib loading, 17.93 mg/mL axitinib in hydrogel suspension.

16b: HG-12: 20 mg, DIPEA: 10.8 μL, 15a: 27 mg yield: suspension, 69% Axitinib loading, 11.69 mg/mL axitinib in hydrogel suspension.

16c: HG-11: 19 mg, DIPEA: 9.3 μL, 15b: 17 mg yield: suspension, 100% Axitinib loading, 16.24 mg/mL axitinib in hydrogel suspension.

16d: HG-8: 20 mg, DIPEA: 11.7 μL, 15b: 22 mg yield: suspension, 95% Axitinib loading, 16.10 mg/mL axitinib in hydrogel suspension.

16e: HG-13: 16 mg, DIPEA: 9.5 μL, 15b: 18 mg yield: suspension, 97% Axitinib loading, 18.63 mg/mL axitinib in hydrogel suspension.

16f: HG-11: 19 mg, DIPEA: 9.5 μL, 15c: 20 mg yield: suspension, 100% Axitinib loading, 16.43 mg/mL axitinib in hydrogel suspension.

16g: HG-11: 20 mg, DIPEA: 10.0 μL, 15g: 21 mg yield: suspension, 95% Axitnib loading, 14.03 mg/mL axitinib in hydrogel suspension.

16h: HG-11: 20 mg, DIPEA: 9.9 μL, 15h: 24 mg yield: suspension, 84% Axintib loading, 11.17 mg/mL axitinib in hydrogel suspension.

16i: HG-11: 20 mg, DIPEA: 9.9 μL, 15d: 20 mg yield: suspension, 94% Axitinib loading, 14.66 mg/mL axitinib in hydrogel suspension.

16j: HG-8: 20 mg, DIPEA: 11.8 μL, 15d: 25 mg yield: suspension, 96% Axitinib loading, 15.72 mg/mL axitinib in hydrogel suspension.

16k: HG-13: 15 mg, DIPEA: 9.1 μL, 15d: 19 mg yield: suspension, 100% Axitinib loading, 19.46 mg/mL axitinib in hydrogel suspension.

16l: HG-4: 15 mg, DIPEA: 8.5 μL, 6b: 15 mg yield: suspension, 98% Axitinib loading, 18.27 mg/mL axitinib in hydrogel suspension.

16m: HG-11: 21 mg, DIPEA: 10.2 μL, 6b: 19 mg yield: suspension, 100% Axitinib loading, 15.60 mg/mL axitinib in hydrogel suspension.

16n: HG-14: 20 mg, DIPEA: 16.2 μL, 6b: 32 mg yield: suspension, 81% Axitinib loading, 21.15 mg/mL axitinib in hydrogel suspension.

16o: HG-13: 16 mg, DIPEA: 9.4 μL, 15e: 19 mg yield: suspension, 99% Axitinib loading, 19.28 mg/mL axitinib in hydrogel suspension.

16p: HG-13: 16 mg, DIPEA: 9.7 μL, 15f: 19 mg yield: suspension, 100% Axitinib loading, 20.66 mg/mL axitinib in hydrogel suspension.

16q: HG-15: 20 mg, DIPEA: 10.6 μL, 6c: 21 mg yield: suspension, 93% Axitinib loading, 15.75 mg/mL axitinib in hydrogel suspension.

### Example 39: In Vitro Release Kinetics

The cleavage rate of the reversible bond from conjugates 12a-d and 16a-p was monitored at 37° C. in aqueous buffer (condition A: pH 7.4 60 mM sodium phosphate, 1% acetonitrile, B: pH 7.4 48 mM sodium phosphate, 20% acetonitrile, 0.1% Pluronic F68, C: pH 7.4 48 mM sodium phosphate with 16 mM L-Methionine 2.4 mM EDTA, 0.1% pluronic and 20% acetonitrile, D: pH 7.0 48 mM sodium phosphate with 16 mM L-Methionine 2.4 mM EDTA, 0.1% pluronic and 20% acetonitrile, E: pH 7.4 60 mM sodium phosphate, F: pH 7.4 48 mM sodium phosphate, 20% acetonitrile). For soluble examples disappearance of the conjugate was determined by LCMS (UV detection) and fitted with curve fitting software to obtain the half-life of the release. For insoluble examples (hydrogels) the increase in released axitinib in the supernatant was determined by LCMS (UV detection) and used as input for the curve fitting software to obtain the half-life of the release. Release rates at pH 7.4 for conjugates only incubated at pH 7.0 are estimated to increase of a factor of 2 to 3.

| Compound | t_{1/2} | | pH | Buffer | Released product |
|---|---|---|---|---|---|
| 6a* | 4.6 | d | 7.4 | F | axitinib |
| 11a* | 4.0 | d | 7.4 | F | axitinib |
| 11b* | 11.5 | d | 7.4 | F | axitinib |
| 11c* | 47 | d | 7.4 | F | axitinib |
| 12a | 14 | h | 7.4 | A | axitinib |
| 12b | 19 | h | 7.4 | E | axitinib |
| 12c | 8 | h | 7.4 | E | axitinib |
| 12d | 1.9 | d | 7.4 | E | axitinib |
| 16b | 107 | d | 7.0 | D | axitinib |
| 16b | 181 | d | 7.4 | C | axitinib |
| 16c | 28 | d | 7.0 | D | axitinib |
| 16c | 12 | d | 7.4 | C | axitinib |
| 16d | 26 | d | 7.0 | D | axitinib |
| 16e | 30 | d | 7.0 | D | axitinib |
| 16f | 49 | d | 7.0 | D | axitinib |
| 16f | 20 | d | 7.4 | C | axitinib |
| 16g | 17 | d | 7.0 | D | axitinib |
| 16g | 7 | d | 7.4 | C | axitinib |
| 16h | 51 | d | 7.0 | D | axitinib |
| 16h | 21 | d | 7.4 | C | axitinib |
| 16i | 27 | d | 7.0 | D | axitinib |
| 16i | 12 | d | 7.4 | C | axitinib |
| 16j | 24 | d | 7.0 | D | axitinib |
| 16k | 30 | d | 7.0 | D | axitinib |
| 16l | 17 | d | 7.0 | D | axitinib |
| 16m | 16 | d | 7.0 | D | axitinib |
| 16m | 7 | d | 7.4 | C | axitinib |
| 16n | 8 | d | 7.4 | B | axitinib |
| 16o | 43 | d | 7.0 | D | axitinib |
| 16p | 15 | d | 7.0 | D | axitinib |
| 16q | 38 | d | 7.4 | C | axitinib |

The compounds marked with “*” are not in accordance with the present invention as they were for efficiency reasons not linked to a moiety Z. Nevertheless, they show the release half-lives of such moieties -L¹-.

### Example 40: Synthesis of Compounds 16r-t

The hydrogel HG-17 was reacted with Axitinib-linker-conjugates according to the following scheme:

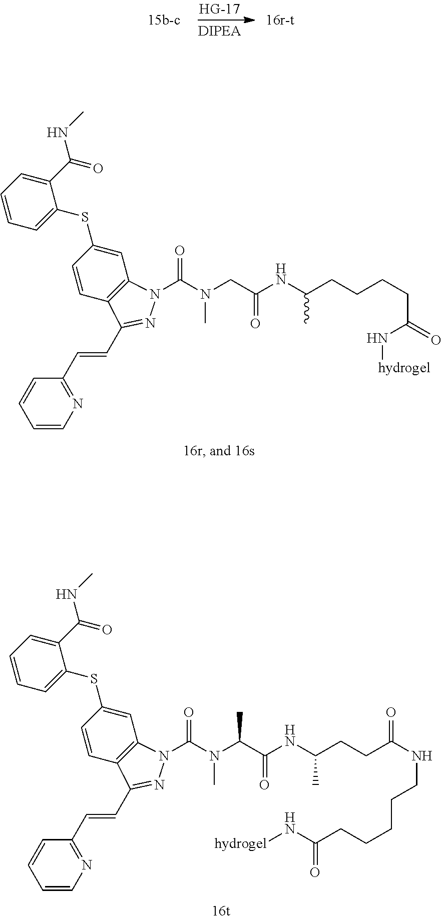

The hydrogel was swollen in 1% DIPEA in DMF in a syringe reactor containing a PE frit. The syringe reactor was 3 times filled, shaken for 1 min and drained. A PFP-ester selected from 15b or 15c was dissolved in DMF and DIPEA was added. The solution was drawn into the syringe containing the hydrogel. The syringe was shaken for longer than 16 h at RT. The syringe was drained, and the hydrogel was washed several times with DMF, then water, then pH 5.5 20 mM sodium succinate 77 g/l trehalose dihydrate, 0.2% Pluronic F-68 aqueous buffer. A hydrogel suspension in pH 5.5 aqueous buffer was obtained. The proportion of amines from the hydrogel that were conjugated was determined by comparing the determined drug content of the product with the amine content of the starting amine hydrogels.

16r: HG-17: 24 mg, DIPEA: 17.8 μL, 15b: 15 mg yield: suspension, 73% Axitinib loading, 17.63 mg/mL axitinib in hydrogel suspension.

16s: HG-17: 24 mg, DIPEA: 18.1 μL, 15b: 20 mg yield: suspension, 94% Axitinib loading, 22.40 mg/mL axitinib in hydrogel suspension.

16t: HG-17: 24 mg, DIPEA: 18.2 μL, 15c: 17 mg yield: suspension, 62% Axitinib loading, 14.87 mg/mL axitinib in hydrogel suspension.

### Example 41: In Vitro Release Kinetics

The cleavage rate of the reversible bond from conjugates 16r-t was monitored at 37° C. in pH 7.0 48 mM sodium phosphate buffer with 16 mM L-Methionine 2.4 mM EDTA, 0.1% pluronic and 20% acetonitrile. The increase in released axitinib in the supernatant was determined by LCMS (UV detection) and used as input for the curve fitting software to obtain the half-life of the release. The release rates at pH 7.4 for these conjugates are estimated to be faster by a factor of 2 to 3.

| Compound | t_{1/2} | Released product |
|---|---|---|
| 16r | 20 d | axitinib |
| 16s | 34 d | axitinib |
| 16t | 31 d | axitinib |

### Example 41

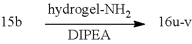

For 16u, a PEG based amino hydrogel is synthesized as described in example 3 of WO2011/012715A1 using a backbone synthesized using Boc-L-Lys(Boc)-OH as described in example 1 of WO2011/012715A1, and a 2 kDa PEG based crosslinker that is synthesized using adipic acid as described in example 2 of WO2011/012715A1. The hydrogel is then modified with lysine using Fmoc-L-Lys(Fmoc)-OH as described in example 5 of WO2011/042450A1 to give a hydrogel with an amine content of 0.700 mmol/g. The hydrogel is swollen in 10% DIPEA in DMF in a syringe reactor fitted with a frit and washed three times with a 1% DIPEA/DMF solution. 15b (1.8 eq. per hydrogel amine) is dissolved in DMF and DIPEA (5.0 eq.) is added. The solution is drawn into the hydrogel-containing reactor and shaken for 16 h at rt. The syringe is drained, the hydrogel washed several times with DMF, washed several times with water, then washed several times with pH 5.5 20 mM sodium succinate, 77 g/l trehalose dihydrate, 0.2% Pluronic F-68 aqueous buffer. A hydrogel suspension in pH 5.5 aqueous buffer where the Axitinib loading is greater than 95% is obtained.

The hydrogel 16v is prepared as described for 16u, but Boc-D-Lys(Boc)-OH is used for the backbone synthesis instead of Boc-L-Lys(Boc)-OH, and Fmoc-D-Lys(Fmoc)-OH instead of Fmoc-L-Lys(Fmoc)-OH is used for the lysine modification.

### Abbreviations

ACHC aminocyclohexane carboxylic acid
Ahx 6-aminohexanoic acid
aq. aqueous
Bn benzyl
Boc tert-butyloxycarbonyl
DBU 1,8-diazabicyclo (5.4.0)undec-7-ene
DCM dichloromethane
DIC N,N′-diisopropylcarbodiimide,
DIPEA diisopropylethylamine
DMAP dimethylaminopyridine
DMF dimethylformamide
eq equivalent
EDC 1-ethyl-3-(3-dimethylaminopropyl)carbodiimide
Fmoc fluorenylmethyloxycarbonyl
HATU O-(7-Azabenzotriazol-1-yl)-N,N,N′,N′-tetramethyluronium-hexafluorphosphat
HFIP 1,1,1,3,3,3-hexafluoroisopropanol
HOBt 1-hydroxybenzotriazole
HPLC high performance liquid chromatography
KOH Potassium hydroxide
LC liquid chromatography
LCMS liquid chromatography mass spectrometry
LCMS/MS liquid chromatography tandem mass spectrometry
LLOQ lower limit of quantification
LPLC low pressure liquid chromatography
MeOH methanol
MRM multi reaction monitoring
MS mass spectrometry
NMP N-Methyl-2-pyrrolidon
PEG polyethylene glycol
PFP pentafluorophenyl
PK pharmacokinetics
PNP para-nitrophenyl
PyBOP benzotriazol-1-yl-oxytripyrrolidinophosphonium hexafluorophosphate
QQQ triple quadrupole mass spectrometer
RP reversed phase
RT room temperature
Sar sarcosine
sat. saturated
tBu and t-Bu tert.-butyl
TES triethylsilane
TFA trifluoroacetic acid
THF tetrahydrofurane
UHPLC ultra high performance liquid chromatography
UPLC ultra performance liquid chromatography
UPLC-MS ultra performance liquid chromatography coupled to mass spectrometry

1. A tyrosine kinase inhibitor (“TKI”) conjugate or a pharmaceutically acceptable salt thereof, wherein said conjugate comprises at least one TKI moiety -D covalently conjugated via at least one moiety -L¹-L²- to a polymeric moiety Z, wherein -L¹- is covalently and reversibly conjugated to -D and -L²- is covalently conjugated to Z and wherein -L¹- is a linker moiety and -L²- is a chemical bond or a spacer moiety.

2. The TKI conjugate of claim 1, wherein Z comprises a polymer.

3. The TKI conjugate of claim 2, wherein Z is a hydrogel.

4. The TKI conjugate of claim 1, wherein Z is a PEG-based or hyaluronic acid-based hydrogel.

5. The TKI conjugate of claim 1, wherein Z is a PEG-based hydrogel.

6. The TKI conjugate of claim 1, wherein -D is selected from the group consisting of receptor tyrosine kinase inhibitors, intracellular kinase inhibitors, cyclin dependent kinase inhibitors, phosphoinositide-3-kinase (PI3K) inhibitors, mitogen-activated protein kinase inhibitors, inhibitors of nuclear factor kappa-β kinase (IKK), and Wee-1 inhibitors.

7. The TKI conjugate of claim 1, wherein -D is selected from the group consisting of lenvatinib, axitinib, cobimetinib, crizotinib, tivantinib, copanlisib and cabozantinib.

8. The TKI conjugate of claim 1, wherein the TKI conjugate further comprises non-TKI moieties -D.

9. The TKI conjugate of claim 8, wherein the non-TKI moieties -D are selected from the group consisting of cytotoxic/chemotherapeutic agents, immune checkpoint inhibitors or antagonists, immune agonists, multi-specific drugs, antibody-drug conjugates (ADC), radionuclides or targeted radionuclide therapeutics, DNA damage repair inhibitors, tumor metabolism inhibitors, pattern recognition receptor agonists, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, and hormones including hormone peptides and synthetic hormones.

10. The TKI conjugate of claim 1, wherein -L¹- is of formula (IX) 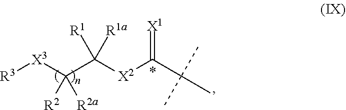
wherein
the dashed line indicates the attachment to a π-electron-pair-donating heteroaromatic N of -D;
n is an integer selected from the group consisting of 0, 1, 2, 3 and 4;
═X¹ is selected from the group consisting of ═O, ═S and ═N(R⁴);
—X²— is selected from the group consisting of —O—, —S—, —N(R⁵)— and —C(R⁶)(R^{6a})—;
—X³— is selected from the group consisting of 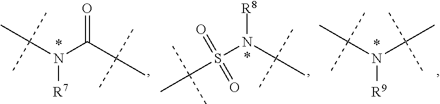
C(R¹⁰)(R^{10a})—, —C(R¹¹)(R^{11a})—C(R¹²)(R^{12a})—, —O— and —C(O)—;
—R¹, —R^{1a}, —R⁶, —R^{6a}, —R¹⁰, —R^{10a}, —R¹¹, —R^{11a}, —R¹², —R^{12a} and each of —R² and —R^{2a} are independently selected from the group consisting of —H, —C(O)OH, halogen, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
—R³, —R⁴, —R⁵, —R⁷, —R⁸ and —R⁹ are independently selected from the group consisting of —H, -T, —CN, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different;
and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R¹³, which are the same or different;
wherein —R¹³ is selected from the group consisting of —H, —NO₂, —OCH₃, —CN, —N(R¹⁴)(R^{14a}), —OH, —C(O)OH and C₁₋₆ alkyl; wherein C₁₋₆ alkyl is optionally substituted with one or more halogen, which are the same or different;
wherein —R¹⁴ and —R^{14a} are independently selected from the group consisting of —H and C₁₋₆ alkyl; wherein C₁₋₆ alkyl is optionally substituted with one or more halogen, which are the same or different;
optionally, one or more of the pairs —R¹/—R^{1a}, —R²/—R^{2a}, two adjacent R², —R⁶/—R^{6a}, —R¹⁰/—R^{10a}, —R¹¹/—R^{11a}, —R¹²/—R^{12a} and —R³/—R⁹ are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁵, —R¹/—R⁶, —R¹/—R⁹, —R¹/—R¹⁰, —R²/—R⁵, —R³/—R^{6a}, —R⁴/—R⁵, —R⁴/—R⁶, —R⁵/—R¹⁰, and —R⁶/—R¹⁰ are joined together with the atoms to which they are attached to form a ring -A-;
wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, 3 and 4;
optionally, two adjacent —R² form a carbon-carbon double bond provided that n is selected from the group consisting of 2, 3 and 4;
provided that if —X²— is —N(R⁵)—, —X³— is selected from the group consisting of 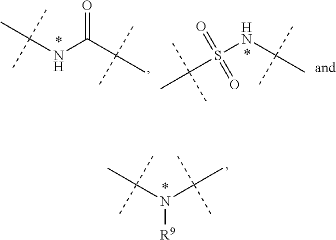
and the distance between the nitrogen atom marked with an asterisk and the carbon atom marked with an asterisk in formula (IX) is 5, 6 or 7 atoms and if present the carbon-carbon double bond formed between —R¹ and —R² or two adjacent —R² is in a cis configuration; and
wherein -L¹- is substituted with -L²- and wherein -L¹- is optionally further substituted.

11. The TKI conjugate of claim 1, wherein -L¹- is of formula (IX′): 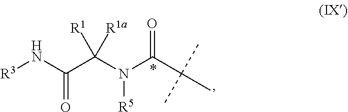
wherein the dashed line indicates the attachment to a π-electron-pair-donating heteroaromatic N of -D; and
—R¹, —R^{1a}, —R³ and —R⁵ are used as defined in claim 10;
optionally, the pair —R¹/—R^{1a} is joined together with the atom to which they are attached to form a C₃₋₁₀ cycloalkyl, 3- to 10-membered heterocyclyl or an 8- to 11-membered heterobicyclyl; and
optionally, the pair —R¹/—R⁵ is joined together with the atoms to which they are attached to form a 3- to 10-membered heterocyclyl or 8- to 11-membered heterobicyclyl.

12. The TKI conjugate of claim 1, wherein -L²- is a spacer moiety.

13. The TKI conjugate of claim 1, wherein moiety -L¹-L²- is selected from the group consisting of 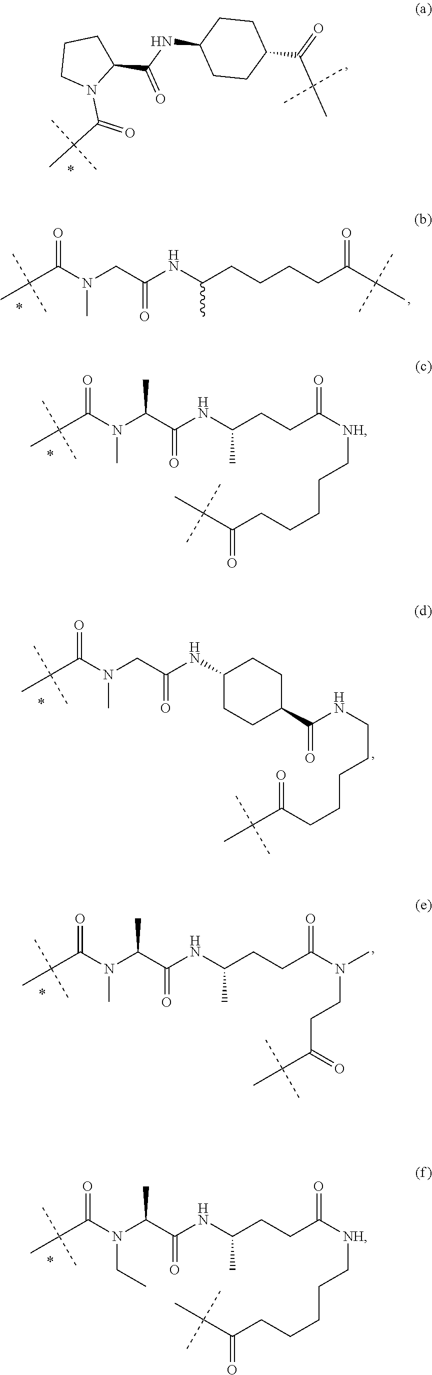 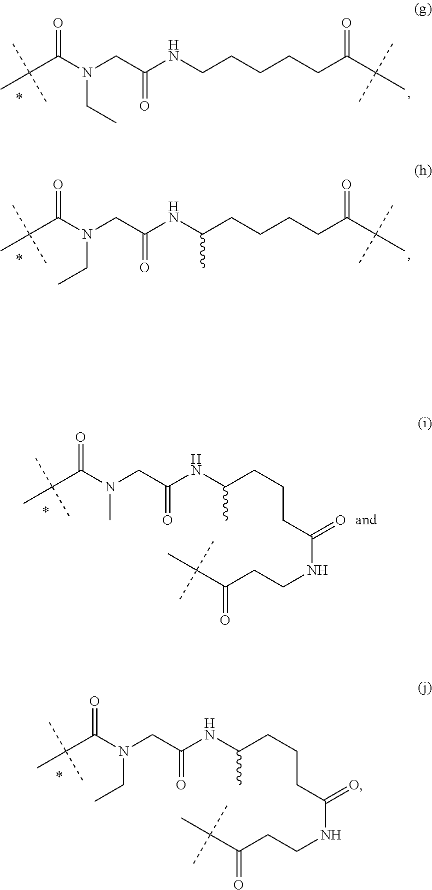
wherein
the dashed line marked with the asterisk indicates attachment to a π-electron-pair-donating heteroaromatic N of -D and the unmarked dashed line indicates attachment to Z.

14. A pharmaceutical composition comprising the TKI conjugate of claim 1 and at least one excipient.

15. A method of treating a mammalian patient in need of the treatment of one or more diseases which can be treated with a TKI drug, comprising the step of administering to said patient in need thereof a therapeutically effective amount of the TKI conjugate or a pharmaceutically acceptable salt thereof of claim 1.

16. The method of claim 15, wherein the disease is a cell-proliferation disorder.

17. The method of claim 16, wherein the cell-proliferation disorder is cancer.

18-19. (canceled)


SEQUENCE LISTING (2 sequences)

SEQ ID NO: 1 - protein, 132 aa, Artificial Sequence
  aldesleukin mutein
PTSSSTKKTQLQLEHLLLDLQMILNGINNYKNPKLTCMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - protein, 133 aa, Artificial Sequence
  aldesleukin mutein
APTSSSTKKTQLQLEHLLLDLQMILNGINNYKNPKLTCMLTFKFYMPKKATELKHLQCLEEELKPLEEVLNLAQSKNFHLRPRDLISNINVIVLELKGSETTFMCEYADETATIVEFLNRWITFSQSIISTLT